SEQUENCE LISTING (1003 sequences)

SEQ ID NO: 1 - DNA, 10 bp, synthetic construct
  misc_feature 1..10: note = Description of Artificial Sequence: Synthetic oligonucleotide
  source 1..10: mol_type = other DNA
gctacataat

SEQ ID NO: 2 - DNA, 10 bp, synthetic construct
  misc_feature 1..10: note = Description of Artificial Sequence: Synthetic oligonucleotide
  source 1..10: mol_type = other DNA
atgttacaca

SEQ ID NO: 3 - DNA, 10 bp, synthetic construct
  misc_feature 1..10: note = Description of Artificial Sequence: Synthetic oligonucleotide
  source 1..10: mol_type = other DNA
tggggcccaa

SEQ ID NO: 4 - DNA, 10 bp, synthetic construct
  misc_feature 1..10: note = Description of Artificial Sequence: Synthetic oligonucleotide
  source 1..10: mol_type = other DNA
tagtttatcc

SEQ ID NO: 5 - DNA, 10 bp, synthetic construct
  misc_feature 1..10: note = Description of Artificial Sequence: Synthetic oligonucleotide
  source 1..10: mol_type = other DNA
accccgtctt

SEQ ID NO: 6 - DNA, 10 bp, synthetic construct
  misc_feature 1..10: note = Description of Artificial Sequence: Synthetic oligonucleotide
  source 1..10: mol_type = other DNA
ccggccatca

SEQ ID NO: 7 - DNA, 10 bp, synthetic construct
  misc_feature 1..10: note = Description of Artificial Sequence: Synthetic oligonucleotide
  source 1..10: mol_type = other DNA
gagcttgctc

SEQ ID NO: 8 - DNA, 10 bp, synthetic construct
  misc_feature 1..10: note = Description of Artificial Sequence: Synthetic oligonucleotide
  source 1..10: mol_type = other DNA
acgttctata

SEQ ID NO: 9 - DNA, 10 bp, synthetic construct
  misc_feature 1..10: note = Description of Artificial Sequence: Synthetic oligonucleotide
  source 1..10: mol_type = other DNA
tacagcaaaa

SEQ ID NO: 10 - DNA, 10 bp, synthetic construct
  misc_feature 1..10: note = Description of Artificial Sequence: Synthetic oligonucleotide
  source 1..10: mol_type = other DNA
gttaggtggt

SEQ ID NO: 11 - DNA, 10 bp, synthetic construct
  misc_feature 1..10: note = Description of Artificial Sequence: Synthetic oligonucleotide
  source 1..10: mol_type = other DNA
ggagaccgac

SEQ ID NO: 12 - DNA, 10 bp, synthetic construct
  misc_feature 1..10: note = Description of Artificial Sequence: Synthetic oligonucleotide
  source 1..10: mol_type = other DNA
tggccccttg

SEQ ID NO: 13 - DNA, 10 bp, synthetic construct
  misc_feature 1..10: note = Description of Artificial Sequence: Synthetic oligonucleotide
  source 1..10: mol_type = other DNA
tggccgtaag

SEQ ID NO: 14 - DNA, 10 bp, synthetic construct
  misc_feature 1..10: note = Description of Artificial Sequence: Synthetic oligonucleotide
  source 1..10: mol_type = other DNA
cgttcgtcaa

SEQ ID NO: 15 - DNA, 10 bp, synthetic construct
  misc_feature 1..10: note = Description of Artificial Sequence: Synthetic oligonucleotide
  source 1..10: mol_type = other DNA
cggacgtgga

SEQ ID NO: 16 - DNA, 10 bp, synthetic construct
  misc_feature 1..10: note = Description of Artificial Sequence: Synthetic oligonucleotide
  source 1..10: mol_type = other DNA
agagggggca

SEQ ID NO: 17 - DNA, 10 bp, synthetic construct
  misc_feature 1..10: note = Description of Artificial Sequence: Synthetic oligonucleotide
  source 1..10: mol_type = other DNA
gttcaggtcg

SEQ ID NO: 18 - DNA, 10 bp, synthetic construct
  misc_feature 1..10: note = Description of Artificial Sequence: Synthetic oligonucleotide
  source 1..10: mol_type = other DNA
ctcgcaagag

SEQ ID NO: 19 - DNA, 10 bp, synthetic construct
  misc_feature 1..10: note = Description of Artificial Sequence: Synthetic oligonucleotide
  source 1..10: mol_type = other DNA
gcaacgactt

SEQ ID NO: 20 - DNA, 10 bp, synthetic construct
  misc_feature 1..10: note = Description of Artificial Sequence: Synthetic oligonucleotide
  source 1..10: mol_type = other DNA
gccatccatc

SEQ ID NO: 21 - DNA, 10 bp, synthetic construct
  misc_feature 1..10: note = Description of Artificial Sequence: Synthetic oligonucleotide
  source 1..10: mol_type = other DNA
ttccgagcag

SEQ ID NO: 22 - DNA, 10 bp, synthetic construct
  misc_feature 1..10: note = Description of Artificial Sequence: Synthetic oligonucleotide
  source 1..10: mol_type = other DNA
cttctggaca

SEQ ID NO: 23 - DNA, 10 bp, synthetic construct
  misc_feature 1..10: note = Description of Artificial Sequence: Synthetic oligonucleotide
  source 1..10: mol_type = other DNA
aacattagac

SEQ ID NO: 24 - DNA, 10 bp, synthetic construct
  misc_feature 1..10: note = Description of Artificial Sequence: Synthetic oligonucleotide
  source 1..10: mol_type = other DNA
aagcaatagt

SEQ ID NO: 25 - DNA, 10 bp, synthetic construct
  misc_feature 1..10: note = Description of Artificial Sequence: Synthetic oligonucleotide
  source 1..10: mol_type = other DNA
agggtaagac

SEQ ID NO: 26 - DNA, 10 bp, synthetic construct
  misc_feature 1..10: note = Description of Artificial Sequence: Synthetic oligonucleotide
  source 1..10: mol_type = other DNA
cgttgtcttg

SEQ ID NO: 27 - DNA, 10 bp, synthetic construct
  misc_feature 1..10: note = Description of Artificial Sequence: Synthetic oligonucleotide
  source 1..10: mol_type = other DNA
tttccccgcc

SEQ ID NO: 28 - DNA, 10 bp, synthetic construct
  misc_feature 1..10: note = Description of Artificial Sequence: Synthetic oligonucleotide
  source 1..10: mol_type = other DNA
cgaatggatc

SEQ ID NO: 29 - DNA, 10 bp, synthetic construct
  misc_feature 1..10: note = Description of Artificial Sequence: Synthetic oligonucleotide
  source 1..10: mol_type = other DNA
catcacttgc

SEQ ID NO: 30 - DNA, 10 bp, synthetic construct
  misc_feature 1..10: note = Description of Artificial Sequence: Synthetic oligonucleotide
  source 1..10: mol_type = other DNA
ctctcgcact

SEQ ID NO: 31 - DNA, 10 bp, synthetic construct
  misc_feature 1..10: note = Description of Artificial Sequence: Synthetic oligonucleotide
  source 1..10: mol_type = other DNA
gttcacgtgc

SEQ ID NO: 32 - DNA, 10 bp, synthetic construct
  misc_feature 1..10: note = Description of Artificial Sequence: Synthetic oligonucleotide
  source 1..10: mol_type = other DNA
aataagcctg

SEQ ID NO: 33 - DNA, 10 bp, synthetic construct
  misc_feature 1..10: note = Description of Artificial Sequence: Synthetic oligonucleotide
  source 1..10: mol_type = other DNA
gttaacaatt

SEQ ID NO: 34 - DNA, 10 bp, synthetic construct
  misc_feature 1..10: note = Description of Artificial Sequence: Synthetic oligonucleotide
  source 1..10: mol_type = other DNA
attcagatcc

SEQ ID NO: 35 - DNA, 10 bp, synthetic construct
  misc_feature 1..10: note = Description of Artificial Sequence: Synthetic oligonucleotide
  source 1..10: mol_type = other DNA
cctgctgatt

SEQ ID NO: 36 - DNA, 10 bp, synthetic construct
  misc_feature 1..10: note = Description of Artificial Sequence: Synthetic oligonucleotide
  source 1..10: mol_type = other DNA
cttggtcata

SEQ ID NO: 37 - DNA, 10 bp, synthetic construct
  misc_feature 1..10: note = Description of Artificial Sequence: Synthetic oligonucleotide
  source 1..10: mol_type = other DNA
tcttcctgtt

SEQ ID NO: 38 - DNA, 10 bp, synthetic construct
  misc_feature 1..10: note = Description of Artificial Sequence: Synthetic oligonucleotide
  source 1..10: mol_type = other DNA
actgccatgg

SEQ ID NO: 39 - DNA, 10 bp, synthetic construct
  misc_feature 1..10: note = Description of Artificial Sequence: Synthetic oligonucleotide
  source 1..10: mol_type = other DNA
catgtatagt

SEQ ID NO: 40 - DNA, 10 bp, synthetic construct
  misc_feature 1..10: note = Description of Artificial Sequence: Synthetic oligonucleotide
  source 1..10: mol_type = other DNA
ggtagcggca

SEQ ID NO: 41 - DNA, 10 bp, synthetic construct
  misc_feature 1..10: note = Description of Artificial Sequence: Synthetic oligonucleotide
  source 1..10: mol_type = other DNA
tcactctaac

SEQ ID NO: 42 - DNA, 10 bp, synthetic construct
  misc_feature 1..10: note = Description of Artificial Sequence: Synthetic oligonucleotide
  source 1..10: mol_type = other DNA
aaggtgcacc

SEQ ID NO: 43 - DNA, 10 bp, synthetic construct
  misc_feature 1..10: note = Description of Artificial Sequence: Synthetic oligonucleotide
  source 1..10: mol_type = other DNA
aatgctcgtt

SEQ ID NO: 44 - DNA, 10 bp, synthetic construct
  misc_feature 1..10: note = Description of Artificial Sequence: Synthetic oligonucleotide
  source 1..10: mol_type = other DNA
tgtctagaaa

SEQ ID NO: 45 - DNA, 10 bp, synthetic construct
  misc_feature 1..10: note = Description of Artificial Sequence: Synthetic oligonucleotide
  source 1..10: mol_type = other DNA
ctgcctgcct

SEQ ID NO: 46 - DNA, 10 bp, synthetic construct
  misc_feature 1..10: note = Description of Artificial Sequence: Synthetic oligonucleotide
  source 1..10: mol_type = other DNA
actataaaag

SEQ ID NO: 47 - DNA, 10 bp, synthetic construct
  misc_feature 1..10: note = Description of Artificial Sequence: Synthetic oligonucleotide
  source 1..10: mol_type = other DNA
tagtatcgag

SEQ ID NO: 48 - DNA, 10 bp, synthetic construct
  misc_feature 1..10: note = Description of Artificial Sequence: Synthetic oligonucleotide
  source 1..10: mol_type = other DNA
atcgcagtcc

SEQ ID NO: 49 - DNA, 10 bp, synthetic construct
  misc_feature 1..10: note = Description of Artificial Sequence: Synthetic oligonucleotide
  source 1..10: mol_type = other DNA
tcatcagaac

SEQ ID NO: 50 - DNA, 10 bp, synthetic construct
  misc_feature 1..10: note = Description of Artificial Sequence: Synthetic oligonucleotide
  source 1..10: mol_type = other DNA
tcctagacgc

SEQ ID NO: 51 - DNA, 10 bp, synthetic construct
  misc_feature 1..10: note = Description of Artificial Sequence: Synthetic oligonucleotide
  source 1..10: mol_type = other DNA
gccgggcggg

SEQ ID NO: 52 - DNA, 10 bp, synthetic construct
  misc_feature 1..10: note = Description of Artificial Sequence: Synthetic oligonucleotide
  source 1..10: mol_type = other DNA
gcccagaaga

SEQ ID NO: 53 - DNA, 10 bp, synthetic construct
  misc_feature 1..10: note = Description of Artificial Sequence: Synthetic oligonucleotide
  source 1..10: mol_type = other DNA
cttagagctg

SEQ ID NO: 54 - DNA, 10 bp, synthetic construct
  misc_feature 1..10: note = Description of Artificial Sequence: Synthetic oligonucleotide
  source 1..10: mol_type = other DNA
gtctgcgctt

SEQ ID NO: 55 - DNA, 10 bp, synthetic construct
  misc_feature 1..10: note = Description of Artificial Sequence: Synthetic oligonucleotide
  source 1..10: mol_type = other DNA
cgccgtcctt

SEQ ID NO: 56 - DNA, 10 bp, synthetic construct
  misc_feature 1..10: note = Description of Artificial Sequence: Synthetic oligonucleotide
  source 1..10: mol_type = other DNA
tttatctgct

SEQ ID NO: 57 - DNA, 10 bp, synthetic construct
  misc_feature 1..10: note = Description of Artificial Sequence: Synthetic oligonucleotide
  source 1..10: mol_type = other DNA
tgcttcggag

SEQ ID NO: 58 - DNA, 10 bp, synthetic construct
  misc_feature 1..10: note = Description of Artificial Sequence: Synthetic oligonucleotide
  source 1..10: mol_type = other DNA
ggggagaatg

SEQ ID NO: 59 - DNA, 10 bp, synthetic construct
  misc_feature 1..10: note = Description of Artificial Sequence: Synthetic oligonucleotide
  source 1..10: mol_type = other DNA
gtggtaagtg

SEQ ID NO: 60 - DNA, 10 bp, synthetic construct
  misc_feature 1..10: note = Description of Artificial Sequence: Synthetic oligonucleotide
  source 1..10: mol_type = other DNA
gaaattagta

SEQ ID NO: 61 - DNA, 10 bp, synthetic construct
  misc_feature 1..10: note = Description of Artificial Sequence: Synthetic oligonucleotide
  source 1..10: mol_type = other DNA
gctatcctaa

SEQ ID NO: 62 - DNA, 10 bp, synthetic construct
  misc_feature 1..10: note = Description of Artificial Sequence: Synthetic oligonucleotide
  source 1..10: mol_type = other DNA
atctgtacga

SEQ ID NO: 63 - DNA, 10 bp, synthetic construct
  misc_feature 1..10: note = Description of Artificial Sequence: Synthetic oligonucleotide
  source 1..10: mol_type = other DNA
agttcggggc

SEQ ID NO: 64 - DNA, 10 bp, synthetic construct
  misc_feature 1..10: note = Description of Artificial Sequence: Synthetic oligonucleotide
  source 1..10: mol_type = other DNA
cgagtctgtc

SEQ ID NO: 65 - DNA, 10 bp, synthetic construct
  misc_feature 1..10: note = Description of Artificial Sequence: Synthetic oligonucleotide
  source 1..10: mol_type = other DNA
atcctacgca

SEQ ID NO: 66 - DNA, 10 bp, synthetic construct
  misc_feature 1..10: note = Description of Artificial Sequence: Synthetic oligonucleotide
  source 1..10: mol_type = other DNA
atggtggata

SEQ ID NO: 67 - DNA, 10 bp, synthetic construct
  misc_feature 1..10: note = Description of Artificial Sequence: Synthetic oligonucleotide
  source 1..10: mol_type = other DNA
cctctaacta

SEQ ID NO: 68 - DNA, 10 bp, synthetic construct
  misc_feature 1..10: note = Description of Artificial Sequence: Synthetic oligonucleotide
  source 1..10: mol_type = other DNA
atagctgcac

SEQ ID NO: 69 - DNA, 10 bp, synthetic construct
  misc_feature 1..10: note = Description of Artificial Sequence: Synthetic oligonucleotide
  source 1..10: mol_type = other DNA
gacagaattt

SEQ ID NO: 70 - DNA, 10 bp, synthetic construct
  misc_feature 1..10: note = Description of Artificial Sequence: Synthetic oligonucleotide
  source 1..10: mol_type = other DNA
caattggcat

SEQ ID NO: 71 - DNA, 10 bp, synthetic construct
  misc_feature 1..10: note = Description of Artificial Sequence: Synthetic oligonucleotide
  source 1..10: mol_type = other DNA
tctagtagac

SEQ ID NO: 72 - DNA, 10 bp, synthetic construct
  misc_feature 1..10: note = Description of Artificial Sequence: Synthetic oligonucleotide
  source 1..10: mol_type = other DNA
ttattcatgg

SEQ ID NO: 73 - DNA, 10 bp, synthetic construct
  misc_feature 1..10: note = Description of Artificial Sequence: Synthetic oligonucleotide
  source 1..10: mol_type = other DNA
ttggcaaccg

SEQ ID NO: 74 - DNA, 10 bp, synthetic construct
  misc_feature 1..10: note = Description of Artificial Sequence: Synthetic oligonucleotide
  source 1..10: mol_type = other DNA
cataatacat

SEQ ID NO: 75 - DNA, 10 bp, synthetic construct
  misc_feature 1..10: note = Description of Artificial Sequence: Synthetic oligonucleotide
  source 1..10: mol_type = other DNA
acagactcac

SEQ ID NO: 76 - DNA, 10 bp, synthetic construct
  misc_feature 1..10: note = Description of Artificial Sequence: Synthetic oligonucleotide
  source 1..10: mol_type = other DNA
gcgatgctgc

SEQ ID NO: 77 - DNA, 10 bp, synthetic construct
  misc_feature 1..10: note = Description of Artificial Sequence: Synthetic oligonucleotide
  source 1..10: mol_type = other DNA
catctttgcc

SEQ ID NO: 78 - DNA, 10 bp, synthetic construct
  misc_feature 1..10: note = Description of Artificial Sequence: Synthetic oligonucleotide
  source 1..10: mol_type = other DNA
gtgactccag

SEQ ID NO: 79 - DNA, 10 bp, synthetic construct
  misc_feature 1..10: note = Description of Artificial Sequence: Synthetic oligonucleotide
  source 1..10: mol_type = other DNA
ggacgagtct

SEQ ID NO: 80 - DNA, 10 bp, synthetic construct
  misc_feature 1..10: note = Description of Artificial Sequence: Synthetic oligonucleotide
  source 1..10: mol_type = other DNA
tagtggcgtg

SEQ ID NO: 81 - DNA, 10 bp, synthetic construct
  misc_feature 1..10: note = Description of Artificial Sequence: Synthetic oligonucleotide
  source 1..10: mol_type = other DNA
aacgcagctt

SEQ ID NO: 82 - DNA, 10 bp, synthetic construct
  misc_feature 1..10: note = Description of Artificial Sequence: Synthetic oligonucleotide
  source 1..10: mol_type = other DNA
agaacaggtg

SEQ ID NO: 83 - DNA, 10 bp, synthetic construct
  misc_feature 1..10: note = Description of Artificial Sequence: Synthetic oligonucleotide
  source 1..10: mol_type = other DNA
aggctatgtt

SEQ ID NO: 84 - DNA, 10 bp, synthetic construct
  misc_feature 1..10: note = Description of Artificial Sequence: Synthetic oligonucleotide
  source 1..10: mol_type = other DNA
cctggatctt

SEQ ID NO: 85 - DNA, 10 bp, synthetic construct
  misc_feature 1..10: note = Description of Artificial Sequence: Synthetic oligonucleotide
  source 1..10: mol_type = other DNA
ctagccggcc

SEQ ID NO: 86 - DNA, 10 bp, synthetic construct
  misc_feature 1..10: note = Description of Artificial Sequence: Synthetic oligonucleotide
  source 1..10: mol_type = other DNA
accagttatc

SEQ ID NO: 87 - DNA, 10 bp, synthetic construct
  misc_feature 1..10: note = Description of Artificial Sequence: Synthetic oligonucleotide
  source 1..10: mol_type = other DNA
acgttatagc

SEQ ID NO: 88 - DNA, 10 bp, synthetic construct
  misc_feature 1..10: note = Description of Artificial Sequence: Synthetic oligonucleotide
  source 1..10: mol_type = other DNA
tcgagtttga

SEQ ID NO: 89 - DNA, 10 bp, synthetic construct
  misc_feature 1..10: note = Description of Artificial Sequence: Synthetic oligonucleotide
  source 1..10: mol_type = other DNA
tgaagcgagc

SEQ ID NO: 90 - DNA, 10 bp, synthetic construct
  misc_feature 1..10: note = Description of Artificial Sequence: Synthetic oligonucleotide
  source 1..10: mol_type = other DNA
gactggcgaa

SEQ ID NO: 91 - DNA, 10 bp, synthetic construct
  misc_feature 1..10: note = Description of Artificial Sequence: Synthetic oligonucleotide
  source 1..10: mol_type = other DNA
gatggaccta

SEQ ID NO: 92 - DNA, 10 bp, synthetic construct
  misc_feature 1..10: note = Description of Artificial Sequence: Synthetic oligonucleotide
  source 1..10: mol_type = other DNA
gtccacaacg

SEQ ID NO: 93 - DNA, 10 bp, synthetic construct
  misc_feature 1..10: note = Description of Artificial Sequence: Synthetic oligonucleotide
  source 1..10: mol_type = other DNA
cctccccaga

SEQ ID NO: 94 - DNA, 10 bp, synthetic construct
  misc_feature 1..10: note = Description of Artificial Sequence: Synthetic oligonucleotide
  source 1..10: mol_type = other DNA
ttatgacgcc

SEQ ID NO: 95 - DNA, 10 bp, synthetic construct
  misc_feature 1..10: note = Description of Artificial Sequence: Synthetic oligonucleotide
  source 1..10: mol_type = other DNA
cttgatccgt

SEQ ID NO: 96 - DNA, 10 bp, synthetic construct
  misc_feature 1..10: note = Description of Artificial Sequence: Synthetic oligonucleotide
  source 1..10: mol_type = other DNA
aatgcgcaat

SEQ ID NO: 97 - DNA, 10 bp, synthetic construct
  misc_feature 1..10: note = Description of Artificial Sequence: Synthetic oligonucleotide
  source 1..10: mol_type = other DNA
gtacccctca

SEQ ID NO: 98 - DNA, 10 bp, synthetic construct
  misc_feature 1..10: note = Description of Artificial Sequence: Synthetic oligonucleotide
  source 1..10: mol_type = other DNA
cgacagctcg

SEQ ID NO: 99 - DNA, 10 bp, synthetic construct
  misc_feature 1..10: note = Description of Artificial Sequence: Synthetic oligonucleotide
  source 1..10: mol_type = other DNA
tgacctggct

SEQ ID NO: 100 - DNA, 10 bp, synthetic construct
  misc_feature 1..10: note = Description of Artificial Sequence: Synthetic oligonucleotide
  source 1..10: mol_type = other DNA
ttcatagccc

SEQ ID NO: 101 - DNA, 10 bp, synthetic construct
  misc_feature 1..10: note = Description of Artificial Sequence: Synthetic oligonucleotide
  source 1..10: mol_type = other DNA
cccaagagaa

SEQ ID NO: 102 - DNA, 10 bp, synthetic construct
  misc_feature 1..10: note = Description of Artificial Sequence: Synthetic oligonucleotide
  source 1..10: mol_type = other DNA
aaacgaagta

SEQ ID NO: 103 - DNA, 10 bp, synthetic construct
  misc_feature 1..10: note = Description of Artificial Sequence: Synthetic oligonucleotide
  source 1..10: mol_type = other DNA
gacgtttaca

SEQ ID NO: 104 - DNA, 10 bp, synthetic construct
  misc_feature 1..10: note = Description of Artificial Sequence: Synthetic oligonucleotide
  source 1..10: mol_type = other DNA
gatcgatttg

SEQ ID NO: 105 - DNA, 10 bp, synthetic construct
  misc_feature 1..10: note = Description of Artificial Sequence: Synthetic oligonucleotide
  source 1..10: mol_type = other DNA
cactgtcacc

SEQ ID NO: 106 - DNA, 10 bp, synthetic construct
  misc_feature 1..10: note = Description of Artificial Sequence: Synthetic oligonucleotide
  source 1..10: mol_type = other DNA
tgtgagagtt

SEQ ID NO: 107 - DNA, 10 bp, synthetic construct
  misc_feature 1..10: note = Description of Artificial Sequence: Synthetic oligonucleotide
  source 1..10: mol_type = other DNA
gacgtaacct

SEQ ID NO: 108 - DNA, 10 bp, synthetic construct
  misc_feature 1..10: note = Description of Artificial Sequence: Synthetic oligonucleotide
  source 1..10: mol_type = other DNA
cagactctgc

SEQ ID NO: 109 - DNA, 10 bp, synthetic construct
  misc_feature 1..10: note = Description of Artificial Sequence: Synthetic oligonucleotide
  source 1..10: mol_type = other DNA
tatgccaata

SEQ ID NO: 110 - DNA, 10 bp, synthetic construct
  misc_feature 1..10: note = Description of Artificial Sequence: Synthetic oligonucleotide
  source 1..10: mol_type = other DNA
acaggtgatg

SEQ ID NO: 111 - DNA, 10 bp, synthetic construct
  misc_feature 1..10: note = Description of Artificial Sequence: Synthetic oligonucleotide
  source 1..10: mol_type = other DNA
gtcatcgcgt

SEQ ID NO: 112 - DNA, 10 bp, synthetic construct
  misc_feature 1..10: note = Description of Artificial Sequence: Synthetic oligonucleotide
  source 1..10: mol_type = other DNA
tcttataaac

SEQ ID NO: 113 - DNA, 10 bp, synthetic construct
  misc_feature 1..10: note = Description of Artificial Sequence: Synthetic oligonucleotide
  source 1..10: mol_type = other DNA
gtgtagactg

SEQ ID NO: 114 - DNA, 10 bp, synthetic construct
  misc_feature 1..10: note = Description of Artificial Sequence: Synthetic oligonucleotide
  source 1..10: mol_type = other DNA
aaacaaccgg

SEQ ID NO: 115 - DNA, 10 bp, synthetic construct
  misc_feature 1..10: note = Description of Artificial Sequence: Synthetic oligonucleotide
  source 1..10: mol_type = other DNA
atcctgtacc

SEQ ID NO: 116 - DNA, 10 bp, synthetic construct
  misc_feature 1..10: note = Description of Artificial Sequence: Synthetic oligonucleotide
  source 1..10: mol_type = other DNA
ttataagaat

SEQ ID NO: 117 - DNA, 10 bp, synthetic construct
  misc_feature 1..10: note = Description of Artificial Sequence: Synthetic oligonucleotide
  source 1..10: mol_type = other DNA
ataagtaggc

SEQ ID NO: 118 - DNA, 10 bp, synthetic construct
  misc_feature 1..10: note = Description of Artificial Sequence: Synthetic oligonucleotide
  source 1..10: mol_type = other DNA
tctcgtaagg

SEQ ID NO: 119 - DNA, 10 bp, synthetic construct
  misc_feature 1..10: note = Description of Artificial Sequence: Synthetic oligonucleotide
  source 1..10: mol_type = other DNA
gatccgccgc

SEQ ID NO: 120 - DNA, 10 bp, synthetic construct
  misc_feature 1..10: note = Description of Artificial Sequence: Synthetic oligonucleotide
  source 1..10: mol_type = other DNA
tgtcaggttt

SEQ ID NO: 121 - DNA, 10 bp, synthetic construct
  misc_feature 1..10: note = Description of Artificial Sequence: Synthetic oligonucleotide
  source 1..10: mol_type = other DNA
tccgaagccc

SEQ ID NO: 122 - DNA, 10 bp, synthetic construct
  misc_feature 1..10: note = Description of Artificial Sequence: Synthetic oligonucleotide
  source 1..10: mol_type = other DNA
tccatgtcca

SEQ ID NO: 123 - DNA, 10 bp, synthetic construct
  misc_feature 1..10: note = Description of Artificial Sequence: Synthetic oligonucleotide
  source 1..10: mol_type = other DNA
gtgatggtac

SEQ ID NO: 124 - DNA, 10 bp, synthetic construct
  misc_feature 1..10: note = Description of Artificial Sequence: Synthetic oligonucleotide
  source 1..10: mol_type = other DNA
ctccacatac

SEQ ID NO: 125 - DNA, 10 bp, synthetic construct
  misc_feature 1..10: note = Description of Artificial Sequence: Synthetic oligonucleotide
  source 1..10: mol_type = other DNA
ttcggatgag

SEQ ID NO: 126 - DNA, 10 bp, synthetic construct
  misc_feature 1..10: note = Description of Artificial Sequence: Synthetic oligonucleotide
  source 1..10: mol_type = other DNA
acgacatcgc

SEQ ID NO: 127 - DNA, 10 bp, synthetic construct
  misc_feature 1..10: note = Description of Artificial Sequence: Synthetic oligonucleotide
  source 1..10: mol_type = other DNA
gagatgcaca

SEQ ID NO: 128 - DNA, 10 bp, synthetic construct
  misc_feature 1..10: note = Description of Artificial Sequence: Synthetic oligonucleotide
  source 1..10: mol_type = other DNA
tttgtatggc

SEQ ID NO: 129 - DNA, 10 bp, synthetic construct
  misc_feature 1..10: note = Description of Artificial Sequence: Synthetic oligonucleotide
  source 1..10: mol_type = other DNA
cttttctaga

SEQ ID NO: 130 - DNA, 10 bp, synthetic construct
  misc_feature 1..10: note = Description of Artificial Sequence: Synthetic oligonucleotide
  source 1..10: mol_type = other DNA
agtctaatca

SEQ ID NO: 131 - DNA, 10 bp, synthetic construct
  misc_feature 1..10: note = Description of Artificial Sequence: Synthetic oligonucleotide
  source 1..10: mol_type = other DNA
gacttagcca

SEQ ID NO: 132 - DNA, 10 bp, synthetic construct
  misc_feature 1..10: note = Description of Artificial Sequence: Synthetic oligonucleotide
  source 1..10: mol_type = other DNA
tatcacagta

SEQ ID NO: 133 - DNA, 10 bp, synthetic construct
  misc_feature 1..10: note = Description of Artificial Sequence: Synthetic oligonucleotide
  source 1..10: mol_type = other DNA
aagctcgagt

SEQ ID NO: 134 - DNA, 10 bp, synthetic construct
  misc_feature 1..10: note = Description of Artificial Sequence: Synthetic oligonucleotide
  source 1..10: mol_type = other DNA
tgttacgaca

SEQ ID NO: 135 - DNA, 10 bp, synthetic construct
  misc_feature 1..10: note = Description of Artificial Sequence: Synthetic oligonucleotide
  source 1..10: mol_type = other DNA
aaggatagtc

SEQ ID NO: 136 - DNA, 10 bp, synthetic construct
  misc_feature 1..10: note = Description of Artificial Sequence: Synthetic oligonucleotide
  source 1..10: mol_type = other DNA
gcacttagcc

SEQ ID NO: 137 - DNA, 10 bp, synthetic construct
  misc_feature 1..10: note = Description of Artificial Sequence: Synthetic oligonucleotide
  source 1..10: mol_type = other DNA
gagggatccg

SEQ ID NO: 138 - DNA, 10 bp, synthetic construct
  misc_feature 1..10: note = Description of Artificial Sequence: Synthetic oligonucleotide
  source 1..10: mol_type = other DNA
attctagaag

SEQ ID NO: 139 - DNA, 10 bp, synthetic construct
  misc_feature 1..10: note = Description of Artificial Sequence: Synthetic oligonucleotide
  source 1..10: mol_type = other DNA
gataactgat

SEQ ID NO: 140 - DNA, 10 bp, synthetic construct
  misc_feature 1..10: note = Description of Artificial Sequence: Synthetic oligonucleotide
  source 1..10: mol_type = other DNA
atctgactgt

SEQ ID NO: 141 - DNA, 10 bp, synthetic construct
  misc_feature 1..10: note = Description of Artificial Sequence: Synthetic oligonucleotide
  source 1..10: mol_type = other DNA
caaagcgaac

SEQ ID NO: 142 - DNA, 10 bp, synthetic construct
  misc_feature 1..10: note = Description of Artificial Sequence: Synthetic oligonucleotide
  source 1..10: mol_type = other DNA
gaaattgcga

SEQ ID NO: 143 - DNA, 10 bp, synthetic construct
  misc_feature 1..10: note = Description of Artificial Sequence: Synthetic oligonucleotide
  source 1..10: mol_type = other DNA
gggtccagtc

SEQ ID NO: 144 - DNA, 10 bp, synthetic construct
  misc_feature 1..10: note = Description of Artificial Sequence: Synthetic oligonucleotide
  source 1..10: mol_type = other DNA
atcaggtagc

SEQ ID NO: 145 - DNA, 10 bp, synthetic construct
  misc_feature 1..10: note = Description of Artificial Sequence: Synthetic oligonucleotide
  source 1..10: mol_type = other DNA
gaaaggtcct

SEQ ID NO: 146 - DNA, 10 bp, synthetic construct
  misc_feature 1..10: note = Description of Artificial Sequence: Synthetic oligonucleotide
  source 1..10: mol_type = other DNA
ggctaccaca

SEQ ID NO: 147 - DNA, 10 bp, synthetic construct
  misc_feature 1..10: note = Description of Artificial Sequence: Synthetic oligonucleotide
  source 1..10: mol_type = other DNA
ttattgctga

SEQ ID NO: 148 - DNA, 10 bp, synthetic construct
  misc_feature 1..10: note = Description of Artificial Sequence: Synthetic oligonucleotide
  source 1..10: mol_type = other DNA
cgccgcgttt

SEQ ID NO: 149 - DNA, 10 bp, synthetic construct
  misc_feature 1..10: note = Description of Artificial Sequence: Synthetic oligonucleotide
  source 1..10: mol_type = other DNA
ttttcaaaag

SEQ ID NO: 150 - DNA, 10 bp, synthetic construct
  misc_feature 1..10: note = Description of Artificial Sequence: Synthetic oligonucleotide
  source 1..10: mol_type = other DNA
ctgggctaaa

SEQ ID NO: 151 - DNA, 10 bp, synthetic construct
  misc_feature 1..10: note = Description of Artificial Sequence: Synthetic oligonucleotide
  source 1..10: mol_type = other DNA
cccgatgaga

SEQ ID NO: 152 - DNA, 10 bp, synthetic construct
  misc_feature 1..10: note = Description of Artificial Sequence: Synthetic oligonucleotide
  source 1..10: mol_type = other DNA
tgggaaatat

SEQ ID NO: 153 - DNA, 10 bp, synthetic construct
  misc_feature 1..10: note = Description of Artificial Sequence: Synthetic oligonucleotide
  source 1..10: mol_type = other DNA
gtacgagcgg

SEQ ID NO: 154 - DNA, 10 bp, synthetic construct
  misc_feature 1..10: note = Description of Artificial Sequence: Synthetic oligonucleotide
  source 1..10: mol_type = other DNA
gcgtgcagct

SEQ ID NO: 155 - DNA, 10 bp, synthetic construct
  misc_feature 1..10: note = Description of Artificial Sequence: Synthetic oligonucleotide
  source 1..10: mol_type = other DNA
agtctgcgga

SEQ ID NO: 156 - DNA, 10 bp, synthetic construct
  misc_feature 1..10: note = Description of Artificial Sequence: Synthetic oligonucleotide
  source 1..10: mol_type = other DNA
taactattta

SEQ ID NO: 157 - DNA, 10 bp, synthetic construct
  misc_feature 1..10: note = Description of Artificial Sequence: Synthetic oligonucleotide
  source 1..10: mol_type = other DNA
gagttgccgg

SEQ ID NO: 158 - DNA, 10 bp, synthetic construct
  misc_feature 1..10: note = Description of Artificial Sequence: Synthetic oligonucleotide
  source 1..10: mol_type = other DNA
cagcccggcg

SEQ ID NO: 159 - DNA, 10 bp, synthetic construct
  misc_feature 1..10: note = Description of Artificial Sequence: Synthetic oligonucleotide
  source 1..10: mol_type = other DNA
tcacctacat

SEQ ID NO: 160 - DNA, 10 bp, synthetic construct
  misc_feature 1..10: note = Description of Artificial Sequence: Synthetic oligonucleotide
  source 1..10: mol_type = other DNA
agtggctaac

SEQ ID NO: 161 - DNA, 10 bp, synthetic construct
  misc_feature 1..10: note = Description of Artificial Sequence: Synthetic oligonucleotide
  source 1..10: mol_type = other DNA
agaatgtgag

SEQ ID NO: 162 - DNA, 10 bp, synthetic construct
  misc_feature 1..10: note = Description of Artificial Sequence: Synthetic oligonucleotide
  source 1..10: mol_type = other DNA
tagtttcgca

SEQ ID NO: 163 - DNA, 10 bp, synthetic construct
  misc_feature 1..10: note = Description of Artificial Sequence: Synthetic oligonucleotide
  source 1..10: mol_type = other DNA
cttcatttct

SEQ ID NO: 164 - DNA, 10 bp, synthetic construct
  misc_feature 1..10: note = Description of Artificial Sequence: Synthetic oligonucleotide
  source 1..10: mol_type = other DNA
gccatgatat

SEQ ID NO: 165 - DNA, 10 bp, synthetic construct
  misc_feature 1..10: note = Description of Artificial Sequence: Synthetic oligonucleotide
  source 1..10: mol_type = other DNA
acggcaaatc

SEQ ID NO: 166 - DNA, 10 bp, synthetic construct
  misc_feature 1..10: note = Description of Artificial Sequence: Synthetic oligonucleotide
  source 1..10: mol_type = other DNA
atcgatagta

SEQ ID NO: 167 - DNA, 10 bp, synthetic construct
  misc_feature 1..10: note = Description of Artificial Sequence: Synthetic oligonucleotide
  source 1..10: mol_type = other DNA
cctaaaggca

SEQ ID NO: 168 - DNA, 10 bp, synthetic construct
  misc_feature 1..10: note = Description of Artificial Sequence: Synthetic oligonucleotide
  source 1..10: mol_type = other DNA
tacgagcggt

SEQ ID NO: 169 - DNA, 10 bp, synthetic construct
  misc_feature 1..10: note = Description of Artificial Sequence: Synthetic oligonucleotide
  source 1..10: mol_type = other DNA
tttgtcgtcg

SEQ ID NO: 170 - DNA, 10 bp, synthetic construct
  misc_feature 1..10: note = Description of Artificial Sequence: Synthetic oligonucleotide
  source 1..10: mol_type = other DNA
tacaagcttg

SEQ ID NO: 171 - DNA, 10 bp, synthetic construct
  misc_feature 1..10: note = Description of Artificial Sequence: Synthetic oligonucleotide
  source 1..10: mol_type = other DNA
gaccaacacg

SEQ ID NO: 172 - DNA, 10 bp, synthetic construct
  misc_feature 1..10: note = Description of Artificial Sequence: Synthetic oligonucleotide
  source 1..10: mol_type = other DNA
gaacgacgaa

SEQ ID NO: 173 - DNA, 10 bp, synthetic construct
  misc_feature 1..10: note = Description of Artificial Sequence: Synthetic oligonucleotide
  source 1..10: mol_type = other DNA
tcggaacgca

SEQ ID NO: 174 - DNA, 10 bp, synthetic construct
  misc_feature 1..10: note = Description of Artificial Sequence: Synthetic oligonucleotide
  source 1..10: mol_type = other DNA
atccggtggt

SEQ ID NO: 175 - DNA, 10 bp, synthetic construct
  misc_feature 1..10: note = Description of Artificial Sequence: Synthetic oligonucleotide
  source 1..10: mol_type = other DNA
taaaacgtag

SEQ ID NO: 176 - DNA, 10 bp, synthetic construct
  misc_feature 1..10: note = Description of Artificial Sequence: Synthetic oligonucleotide
  source 1..10: mol_type = other DNA
tatgtgagcc

SEQ ID NO: 177 - DNA, 10 bp, synthetic construct
  misc_feature 1..10: note = Description of Artificial Sequence: Synthetic oligonucleotide
  source 1..10: mol_type = other DNA
gaggcatcga

SEQ ID NO: 178 - DNA, 10 bp, synthetic construct
  misc_feature 1..10: note = Description of Artificial Sequence: Synthetic oligonucleotide
  source 1..10: mol_type = other DNA
gaatgggtgg

SEQ ID NO: 179 - DNA, 10 bp, synthetic construct
  misc_feature 1..10: note = Description of Artificial Sequence: Synthetic oligonucleotide
  source 1..10: mol_type = other DNA
aacgacacaa

SEQ ID NO: 180 - DNA, 10 bp, synthetic construct
  misc_feature 1..10: note = Description of Artificial Sequence: Synthetic oligonucleotide
  source 1..10: mol_type = other DNA
gtacgatgca

SEQ ID NO: 181 - DNA, 10 bp, synthetic construct
  misc_feature 1..10: note = Description of Artificial Sequence: Synthetic oligonucleotide
  source 1..10: mol_type = other DNA
agaaggcgcc

SEQ ID NO: 182 - DNA, 10 bp, synthetic construct
  misc_feature 1..10: note = Description of Artificial Sequence: Synthetic oligonucleotide
  source 1..10: mol_type = other DNA
ccgcaatgga

SEQ ID NO: 183 - DNA, 10 bp, synthetic construct
  misc_feature 1..10: note = Description of Artificial Sequence: Synthetic oligonucleotide
  source 1..10: mol_type = other DNA
tacggatttt

SEQ ID NO: 184 - DNA, 10 bp, synthetic construct
  misc_feature 1..10: note = Description of Artificial Sequence: Synthetic oligonucleotide
  source 1..10: mol_type = other DNA
gtcgttagct

SEQ ID NO: 185 - DNA, 10 bp, synthetic construct
  misc_feature 1..10: note = Description of Artificial Sequence: Synthetic oligonucleotide
  source 1..10: mol_type = other DNA
ggactagggc

SEQ ID NO: 186 - DNA, 10 bp, synthetic construct
  misc_feature 1..10: note = Description of Artificial Sequence: Synthetic oligonucleotide
  source 1..10: mol_type = other DNA
attggtattc

SEQ ID NO: 187 - DNA, 10 bp, synthetic construct
  misc_feature 1..10: note = Description of Artificial Sequence: Synthetic oligonucleotide
  source 1..10: mol_type = other DNA
atcccagaga

SEQ ID NO: 188 - DNA, 10 bp, synthetic construct
  misc_feature 1..10: note = Description of Artificial Sequence: Synthetic oligonucleotide
  source 1..10: mol_type = other DNA
gtcccagctc

SEQ ID NO: 189 - DNA, 10 bp, synthetic construct
  misc_feature 1..10: note = Description of Artificial Sequence: Synthetic oligonucleotide
  source 1..10: mol_type = other DNA
cacgaggaat

SEQ ID NO: 190 - DNA, 10 bp, synthetic construct
  misc_feature 1..10: note = Description of Artificial Sequence: Synthetic oligonucleotide
  source 1..10: mol_type = other DNA
tacaattgca

SEQ ID NO: 191 - DNA, 10 bp, synthetic construct
  misc_feature 1..10: note = Description of Artificial Sequence: Synthetic oligonucleotide
  source 1..10: mol_type = other DNA
attcctgaat

SEQ ID NO: 192 - DNA, 10 bp, synthetic construct
  misc_feature 1..10: note = Description of Artificial Sequence: Synthetic oligonucleotide
  source 1..10: mol_type = other DNA
tagcgaggcg

SEQ ID NO: 193 - DNA, 10 bp, synthetic construct
  misc_feature 1..10: note = Description of Artificial Sequence: Synthetic oligonucleotide
  source 1..10: mol_type = other DNA
ctggatgggc

SEQ ID NO: 194 - DNA, 10 bp, synthetic construct
  misc_feature 1..10: note = Description of Artificial Sequence: Synthetic oligonucleotide
  source 1..10: mol_type = other DNA
gcgacggcca

SEQ ID NO: 195 - DNA, 10 bp, synthetic construct
  misc_feature 1..10: note = Description of Artificial Sequence: Synthetic oligonucleotide
  source 1..10: mol_type = other DNA
acctgcacaa

SEQ ID NO: 196 - DNA, 10 bp, synthetic construct
  misc_feature 1..10: note = Description of Artificial Sequence: Synthetic oligonucleotide
  source 1..10: mol_type = other DNA
catgacagac

SEQ ID NO: 197 - DNA, 10 bp, synthetic construct
  misc_feature 1..10: note = Description of Artificial Sequence: Synthetic oligonucleotide
  source 1..10: mol_type = other DNA
ttaccaacgt

SEQ ID NO: 198 - DNA, 10 bp, synthetic construct
  misc_feature 1..10: note = Description of Artificial Sequence: Synthetic oligonucleotide
  source 1..10: mol_type = other DNA
caggtgtgtg

SEQ ID NO: 199 - DNA, 10 bp, synthetic construct
  misc_feature 1..10: note = Description of Artificial Sequence: Synthetic oligonucleotide
  source 1..10: mol_type = other DNA
cgagggacgg

SEQ ID NO: 200 - DNA, 10 bp, synthetic construct
  misc_feature 1..10: note = Description of Artificial Sequence: Synthetic oligonucleotide
  source 1..10: mol_type = other DNA
cgtctcggta

SEQ ID NO: 201 - DNA, 10 bp, synthetic construct
  misc_feature 1..10: note = Description of Artificial Sequence: Synthetic oligonucleotide
  source 1..10: mol_type = other DNA
taagctatct

SEQ ID NO: 202 - DNA, 10 bp, synthetic construct
  misc_feature 1..10: note = Description of Artificial Sequence: Synthetic oligonucleotide
  source 1..10: mol_type = other DNA
tactccccta

SEQ ID NO: 203 - DNA, 10 bp, synthetic construct
  misc_feature 1..10: note = Description of Artificial Sequence: Synthetic oligonucleotide
  source 1..10: mol_type = other DNA
ttatattcat

SEQ ID NO: 204 - DNA, 10 bp, synthetic construct
  misc_feature 1..10: note = Description of Artificial Sequence: Synthetic oligonucleotide
  source 1..10: mol_type = other DNA
agcgatctgc

SEQ ID NO: 205 - DNA, 10 bp, synthetic construct
  misc_feature 1..10: note = Description of Artificial Sequence: Synthetic oligonucleotide
  source 1..10: mol_type = other DNA
tcttctgatc

SEQ ID NO: 206 - DNA, 10 bp, synthetic construct
  misc_feature 1..10: note = Description of Artificial Sequence: Synthetic oligonucleotide
  source 1..10: mol_type = other DNA
atagttccca

SEQ ID NO: 207 - DNA, 10 bp, synthetic construct
  misc_feature 1..10: note = Description of Artificial Sequence: Synthetic oligonucleotide
  source 1..10: mol_type = other DNA
tttacgggtg

SEQ ID NO: 208 - DNA, 10 bp, synthetic construct
  misc_feature 1..10: note = Description of Artificial Sequence: Synthetic oligonucleotide
  source 1..10: mol_type = other DNA
gtgtcccctg

SEQ ID NO: 209 - DNA, 10 bp, synthetic construct
  misc_feature 1..10: note = Description of Artificial Sequence: Synthetic oligonucleotide
  source 1..10: mol_type = other DNA
gcgggggtcg

SEQ ID NO: 210 - DNA, 10 bp, synthetic construct
  misc_feature 1..10: note = Description of Artificial Sequence: Synthetic oligonucleotide
  source 1..10: mol_type = other DNA
cattgatcta

SEQ ID NO: 211 - DNA, 10 bp, synthetic construct
  misc_feature 1..10: note = Description of Artificial Sequence: Synthetic oligonucleotide
  source 1..10: mol_type = other DNA
agggacggtg

SEQ ID NO: 212 - DNA, 10 bp, synthetic construct
  misc_feature 1..10: note = Description of Artificial Sequence: Synthetic oligonucleotide
  source 1..10: mol_type = other DNA
cagttacttt

SEQ ID NO: 213 - DNA, 10 bp, synthetic construct
  misc_feature 1..10: note = Description of Artificial Sequence: Synthetic oligonucleotide
  source 1..10: mol_type = other DNA
ccatacttcc

SEQ ID NO: 214 - DNA, 10 bp, synthetic construct
  misc_feature 1..10: note = Description of Artificial Sequence: Synthetic oligonucleotide
  source 1..10: mol_type = other DNA
atcagaatta

SEQ ID NO: 215 - DNA, 10 bp, synthetic construct
  misc_feature 1..10: note = Description of Artificial Sequence: Synthetic oligonucleotide
  source 1..10: mol_type = other DNA
aaactaggca

SEQ ID NO: 216 - DNA, 10 bp, synthetic construct
  misc_feature 1..10: note = Description of Artificial Sequence: Synthetic oligonucleotide
  source 1..10: mol_type = other DNA
aatgtcgttg

SEQ ID NO: 217 - DNA, 10 bp, synthetic construct
  misc_feature 1..10: note = Description of Artificial Sequence: Synthetic oligonucleotide
  source 1..10: mol_type = other DNA
cacatgggtc

SEQ ID NO: 218 - DNA, 10 bp, synthetic construct
  misc_feature 1..10: note = Description of Artificial Sequence: Synthetic oligonucleotide
  source 1..10: mol_type = other DNA
ggtcgctggt

SEQ ID NO: 219 - DNA, 10 bp, synthetic construct
  misc_feature 1..10: note = Description of Artificial Sequence: Synthetic oligonucleotide
  source 1..10: mol_type = other DNA
actgtattac

SEQ ID NO: 220 - DNA, 10 bp, synthetic construct
  misc_feature 1..10: note = Description of Artificial Sequence: Synthetic oligonucleotide
  source 1..10: mol_type = other DNA
ccgagacgcg

SEQ ID NO: 221 - DNA, 10 bp, synthetic construct
  misc_feature 1..10: note = Description of Artificial Sequence: Synthetic oligonucleotide
  source 1..10: mol_type = other DNA
actccaaccc

SEQ ID NO: 222 - DNA, 10 bp, synthetic construct
  misc_feature 1..10: note = Description of Artificial Sequence: Synthetic oligonucleotide
  source 1..10: mol_type = other DNA
atattacaag

SEQ ID NO: 223 - DNA, 10 bp, synthetic construct
  misc_feature 1..10: note = Description of Artificial Sequence: Synthetic oligonucleotide
  source 1..10: mol_type = other DNA
ccatggatag

SEQ ID NO: 224 - DNA, 10 bp, synthetic construct
  misc_feature 1..10: note = Description of Artificial Sequence: Synthetic oligonucleotide
  source 1..10: mol_type = other DNA
ccgtctcaat

SEQ ID NO: 225 - DNA, 10 bp, synthetic construct
  misc_feature 1..10: note = Description of Artificial Sequence: Synthetic oligonucleotide
  source 1..10: mol_type = other DNA
gatcgtcggg

SEQ ID NO: 226 - DNA, 10 bp, synthetic construct
  misc_feature 1..10: note = Description of Artificial Sequence: Synthetic oligonucleotide
  source 1..10: mol_type = other DNA
tcttgttttg

SEQ ID NO: 227 - DNA, 10 bp, synthetic construct
  misc_feature 1..10: note = Description of Artificial Sequence: Synthetic oligonucleotide
  source 1..10: mol_type = other DNA
aatattgctc

SEQ ID NO: 228 - DNA, 10 bp, synthetic construct
  misc_feature 1..10: note = Description of Artificial Sequence: Synthetic oligonucleotide
  source 1..10: mol_type = other DNA
aacgtcgtct

SEQ ID NO: 229 - DNA, 10 bp, synthetic construct
  misc_feature 1..10: note = Description of Artificial Sequence: Synthetic oligonucleotide
  source 1..10: mol_type = other DNA
aatatttttg

SEQ ID NO: 230 - DNA, 10 bp, synthetic construct
  misc_feature 1..10: note = Description of Artificial Sequence: Synthetic oligonucleotide
  source 1..10: mol_type = other DNA
cgtaacgtgc

SEQ ID NO: 231 - DNA, 10 bp, synthetic construct
  misc_feature 1..10: note = Description of Artificial Sequence: Synthetic oligonucleotide
  source 1..10: mol_type = other DNA
gcgtggttat

SEQ ID NO: 232 - DNA, 10 bp, synthetic construct
  misc_feature 1..10: note = Description of Artificial Sequence: Synthetic oligonucleotide
  source 1..10: mol_type = other DNA
caaaacatta

SEQ ID NO: 233 - DNA, 10 bp, synthetic construct
  misc_feature 1..10: note = Description of Artificial Sequence: Synthetic oligonucleotide
  source 1..10: mol_type = other DNA
cgtatcctga

SEQ ID NO: 234 - DNA, 10 bp, synthetic construct
  misc_feature 1..10: note = Description of Artificial Sequence: Synthetic oligonucleotide
  source 1..10: mol_type = other DNA
tcgcttacaa

SEQ ID NO: 235 - DNA, 10 bp, synthetic construct
  misc_feature 1..10: note = Description of Artificial Sequence: Synthetic oligonucleotide
  source 1..10: mol_type = other DNA
tccattgtgt

SEQ ID NO: 236 - DNA, 10 bp, synthetic construct
  misc_feature 1..10: note = Description of Artificial Sequence: Synthetic oligonucleotide
  source 1..10: mol_type = other DNA
gcccccattc

SEQ ID NO: 237 - DNA, 10 bp, synthetic construct
  misc_feature 1..10: note = Description of Artificial Sequence: Synthetic oligonucleotide
  source 1..10: mol_type = other DNA
tgacgtctat

SEQ ID NO: 238 - DNA, 10 bp, synthetic construct
  misc_feature 1..10: note = Description of Artificial Sequence: Synthetic oligonucleotide
  source 1..10: mol_type = other DNA
tgggccgagg

SEQ ID NO: 239 - DNA, 10 bp, synthetic construct
  misc_feature 1..10: note = Description of Artificial Sequence: Synthetic oligonucleotide
  source 1..10: mol_type = other DNA
aagtgtcaag

SEQ ID NO: 240 - DNA, 10 bp, synthetic construct
  misc_feature 1..10: note = Description of Artificial Sequence: Synthetic oligonucleotide
  source 1..10: mol_type = other DNA
gacagtagag

SEQ ID NO: 241 - DNA, 10 bp, synthetic construct
  misc_feature 1..10: note = Description of Artificial Sequence: Synthetic oligonucleotide
  source 1..10: mol_type = other DNA
cgcagccatc

SEQ ID NO: 242 - DNA, 10 bp, synthetic construct
  misc_feature 1..10: note = Description of Artificial Sequence: Synthetic oligonucleotide
  source 1..10: mol_type = other DNA
gaggcagaac

SEQ ID NO: 243 - DNA, 10 bp, synthetic construct
  misc_feature 1..10: note = Description of Artificial Sequence: Synthetic oligonucleotide
  source 1..10: mol_type = other DNA
gttgaaattg

SEQ ID NO: 244 - DNA, 10 bp, synthetic construct
  misc_feature 1..10: note = Description of Artificial Sequence: Synthetic oligonucleotide
  source 1..10: mol_type = other DNA
atctgataaa

SEQ ID NO: 245 - DNA, 10 bp, synthetic construct
  misc_feature 1..10: note = Description of Artificial Sequence: Synthetic oligonucleotide
  source 1..10: mol_type = other DNA
agctgtctct

SEQ ID NO: 246 - DNA, 10 bp, synthetic construct
  misc_feature 1..10: note = Description of Artificial Sequence: Synthetic oligonucleotide
  source 1..10: mol_type = other DNA
ttttaggtta

SEQ ID NO: 247 - DNA, 10 bp, synthetic construct
  misc_feature 1..10: note = Description of Artificial Sequence: Synthetic oligonucleotide
  source 1..10: mol_type = other DNA
tatctgtccg

SEQ ID NO: 248 - DNA, 10 bp, synthetic construct
  misc_feature 1..10: note = Description of Artificial Sequence: Synthetic oligonucleotide
  source 1..10: mol_type = other DNA
aaaacatatg

SEQ ID NO: 249 - DNA, 10 bp, synthetic construct
  misc_feature 1..10: note = Description of Artificial Sequence: Synthetic oligonucleotide
  source 1..10: mol_type = other DNA
gtaaagaaga

SEQ ID NO: 250 - DNA, 10 bp, synthetic construct
  misc_feature 1..10: note = Description of Artificial Sequence: Synthetic oligonucleotide
  source 1..10: mol_type = other DNA
tcgacgtgca

SEQ ID NO: 251 - DNA, 10 bp, synthetic construct
  misc_feature 1..10: note = Description of Artificial Sequence: Synthetic oligonucleotide
  source 1..10: mol_type = other DNA
tagatcttaa

SEQ ID NO: 252 - DNA, 10 bp, synthetic construct
  misc_feature 1..10: note = Description of Artificial Sequence: Synthetic oligonucleotide
  source 1..10: mol_type = other DNA
cactggtcac

SEQ ID NO: 253 - DNA, 10 bp, synthetic construct
  misc_feature 1..10: note = Description of Artificial Sequence: Synthetic oligonucleotide
  source 1..10: mol_type = other DNA
attctgatgt

SEQ ID NO: 254 - DNA, 10 bp, synthetic construct
  misc_feature 1..10: note = Description of Artificial Sequence: Synthetic oligonucleotide
  source 1..10: mol_type = other DNA
atggccctga

SEQ ID NO: 255 - DNA, 10 bp, synthetic construct
  misc_feature 1..10: note = Description of Artificial Sequence: Synthetic oligonucleotide
  source 1..10: mol_type = other DNA
ggtgatgaga

SEQ ID NO: 256 - DNA, 10 bp, synthetic construct
  misc_feature 1..10: note = Description of Artificial Sequence: Synthetic oligonucleotide
  source 1..10: mol_type = other DNA
caccgtgggg

SEQ ID NO: 257 - DNA, 10 bp, synthetic construct
  misc_feature 1..10: note = Description of Artificial Sequence: Synthetic oligonucleotide
  source 1..10: mol_type = other DNA
gcttgctcgg

SEQ ID NO: 258 - DNA, 10 bp, synthetic construct
  misc_feature 1..10: note = Description of Artificial Sequence: Synthetic oligonucleotide
  source 1..10: mol_type = other DNA
ccagttgaac

SEQ ID NO: 259 - DNA, 10 bp, synthetic construct
  misc_feature 1..10: note = Description of Artificial Sequence: Synthetic oligonucleotide
  source 1..10: mol_type = other DNA
cgtctgtacc

SEQ ID NO: 260 - DNA, 10 bp, synthetic construct
  misc_feature 1..10: note = Description of Artificial Sequence: Synthetic oligonucleotide
  source 1..10: mol_type = other DNA
ccaacgcggc

SEQ ID NO: 261 - DNA, 10 bp, synthetic construct
  misc_feature 1..10: note = Description of Artificial Sequence: Synthetic oligonucleotide
  source 1..10: mol_type = other DNA
acgtgatcga

SEQ ID NO: 262 - DNA, 10 bp, synthetic construct
  misc_feature 1..10: note = Description of Artificial Sequence: Synthetic oligonucleotide
  source 1..10: mol_type = other DNA
ccatcgaatc

SEQ ID NO: 263 - DNA, 10 bp, synthetic construct
  misc_feature 1..10: note = Description of Artificial Sequence: Synthetic oligonucleotide
  source 1..10: mol_type = other DNA
cggtgtctgc

SEQ ID NO: 264 - DNA, 10 bp, synthetic construct
  misc_feature 1..10: note = Description of Artificial Sequence: Synthetic oligonucleotide
  source 1..10: mol_type = other DNA
aaaccacctc

SEQ ID NO: 265 - DNA, 10 bp, synthetic construct
  misc_feature 1..10: note = Description of Artificial Sequence: Synthetic oligonucleotide
  source 1..10: mol_type = other DNA
tcaatgttcc

SEQ ID NO: 266 - DNA, 10 bp, synthetic construct
  misc_feature 1..10: note = Description of Artificial Sequence: Synthetic oligonucleotide
  source 1..10: mol_type = other DNA
ttcgacatgt

SEQ ID NO: 267 - DNA, 10 bp, synthetic construct
  misc_feature 1..10: note = Description of Artificial Sequence: Synthetic oligonucleotide
  source 1..10: mol_type = other DNA
aggcacgata

SEQ ID NO: 268 - DNA, 10 bp, synthetic construct
  misc_feature 1..10: note = Description of Artificial Sequence: Synthetic oligonucleotide
  source 1..10: mol_type = other DNA
cacgagatca

SEQ ID NO: 269 - DNA, 10 bp, synthetic construct
  misc_feature 1..10: note = Description of Artificial Sequence: Synthetic oligonucleotide
  source 1..10: mol_type = other DNA
catgctgggg

SEQ ID NO: 270 - DNA, 10 bp, synthetic construct
  misc_feature 1..10: note = Description of Artificial Sequence: Synthetic oligonucleotide
  source 1..10: mol_type = other DNA
taccatggtt

SEQ ID NO: 271 - DNA, 10 bp, synthetic construct
  misc_feature 1..10: note = Description of Artificial Sequence: Synthetic oligonucleotide
  source 1..10: mol_type = other DNA
ttgcccatat

SEQ ID NO: 272 - DNA, 10 bp, synthetic construct
  misc_feature 1..10: note = Description of Artificial Sequence: Synthetic oligonucleotide
  source 1..10: mol_type = other DNA
tgcacattcg

SEQ ID NO: 273 - DNA, 10 bp, synthetic construct
  misc_feature 1..10: note = Description of Artificial Sequence: Synthetic oligonucleotide
  source 1..10: mol_type = other DNA
gttatgttgg

SEQ ID NO: 274 - DNA, 10 bp, synthetic construct
  misc_feature 1..10: note = Description of Artificial Sequence: Synthetic oligonucleotide
  source 1..10: mol_type = other DNA
tgagttatga

SEQ ID NO: 275 - DNA, 10 bp, synthetic construct
  misc_feature 1..10: note = Description of Artificial Sequence: Synthetic oligonucleotide
  source 1..10: mol_type = other DNA
gatggccccc

SEQ ID NO: 276 - DNA, 10 bp, synthetic construct
  misc_feature 1..10: note = Description of Artificial Sequence: Synthetic oligonucleotide
  source 1..10: mol_type = other DNA
gatgggttac

SEQ ID NO: 277 - DNA, 10 bp, synthetic construct
  misc_feature 1..10: note = Description of Artificial Sequence: Synthetic oligonucleotide
  source 1..10: mol_type = other DNA
agctacgttg

SEQ ID NO: 278 - DNA, 10 bp, synthetic construct
  misc_feature 1..10: note = Description of Artificial Sequence: Synthetic oligonucleotide
  source 1..10: mol_type = other DNA
accccatgca

SEQ ID NO: 279 - DNA, 10 bp, synthetic construct
  misc_feature 1..10: note = Description of Artificial Sequence: Synthetic oligonucleotide
  source 1..10: mol_type = other DNA
tactaccgtt

SEQ ID NO: 280 - DNA, 10 bp, synthetic construct
  misc_feature 1..10: note = Description of Artificial Sequence: Synthetic oligonucleotide
  source 1..10: mol_type = other DNA
tcgcttctac

SEQ ID NO: 281 - DNA, 10 bp, synthetic construct
  misc_feature 1..10: note = Description of Artificial Sequence: Synthetic oligonucleotide
  source 1..10: mol_type = other DNA
ctggcagtgc

SEQ ID NO: 282 - DNA, 10 bp, synthetic construct
  misc_feature 1..10: note = Description of Artificial Sequence: Synthetic oligonucleotide
  source 1..10: mol_type = other DNA
tctatatata

SEQ ID NO: 283 - DNA, 10 bp, synthetic construct
  misc_feature 1..10: note = Description of Artificial Sequence: Synthetic oligonucleotide
  source 1..10: mol_type = other DNA
ggattagttc

SEQ ID NO: 284 - DNA, 10 bp, synthetic construct
  misc_feature 1..10: note = Description of Artificial Sequence: Synthetic oligonucleotide
  source 1..10: mol_type = other DNA
gtgttacgct

SEQ ID NO: 285 - DNA, 10 bp, synthetic construct
  misc_feature 1..10: note = Description of Artificial Sequence: Synthetic oligonucleotide
  source 1..10: mol_type = other DNA
tcgactccgt

SEQ ID NO: 286 - DNA, 10 bp, synthetic construct
  misc_feature 1..10: note = Description of Artificial Sequence: Synthetic oligonucleotide
  source 1..10: mol_type = other DNA
ggtagcaggc

SEQ ID NO: 287 - DNA, 10 bp, synthetic construct
  misc_feature 1..10: note = Description of Artificial Sequence: Synthetic oligonucleotide
  source 1..10: mol_type = other DNA
tattggattc

SEQ ID NO: 288 - DNA, 10 bp, synthetic construct
  misc_feature 1..10: note = Description of Artificial Sequence: Synthetic oligonucleotide
  source 1..10: mol_type = other DNA
gttcgatcga

SEQ ID NO: 289 - DNA, 10 bp, synthetic construct
  misc_feature 1..10: note = Description of Artificial Sequence: Synthetic oligonucleotide
  source 1..10: mol_type = other DNA
atattaatat

SEQ ID NO: 290 - DNA, 10 bp, synthetic construct
  misc_feature 1..10: note = Description of Artificial Sequence: Synthetic oligonucleotide
  source 1..10: mol_type = other DNA
agaacgattg

SEQ ID NO: 291 - DNA, 10 bp, synthetic construct
  misc_feature 1..10: note = Description of Artificial Sequence: Synthetic oligonucleotide
  source 1..10: mol_type = other DNA
gtaaagtgta

SEQ ID NO: 292 - DNA, 10 bp, synthetic construct
  misc_feature 1..10: note = Description of Artificial Sequence: Synthetic oligonucleotide
  source 1..10: mol_type = other DNA
cccatgtgcc

SEQ ID NO: 293 - DNA, 10 bp, synthetic construct
  misc_feature 1..10: note = Description of Artificial Sequence: Synthetic oligonucleotide
  source 1..10: mol_type = other DNA
gtggcctcgc

SEQ ID NO: 294 - DNA, 10 bp, synthetic construct
  misc_feature 1..10: note = Description of Artificial Sequence: Synthetic oligonucleotide
  source 1..10: mol_type = other DNA
gacactagga

SEQ ID NO: 295 - DNA, 10 bp, synthetic construct
  misc_feature 1..10: note = Description of Artificial Sequence: Synthetic oligonucleotide
  source 1..10: mol_type = other DNA
atattctgac

SEQ ID NO: 296 - DNA, 10 bp, synthetic construct
  misc_feature 1..10: note = Description of Artificial Sequence: Synthetic oligonucleotide
  source 1..10: mol_type = other DNA
taagtagacg

SEQ ID NO: 297 - DNA, 10 bp, synthetic construct
  misc_feature 1..10: note = Description of Artificial Sequence: Synthetic oligonucleotide
  source 1..10: mol_type = other DNA
taacggtcta

SEQ ID NO: 298 - DNA, 10 bp, synthetic construct
  misc_feature 1..10: note = Description of Artificial Sequence: Synthetic oligonucleotide
  source 1..10: mol_type = other DNA
tagtttcatt

SEQ ID NO: 299 - DNA, 10 bp, synthetic construct
  misc_feature 1..10: note = Description of Artificial Sequence: Synthetic oligonucleotide
  source 1..10: mol_type = other DNA
ttggatccga

SEQ ID NO: 300 - DNA, 10 bp, synthetic construct
  misc_feature 1..10: note = Description of Artificial Sequence: Synthetic oligonucleotide
  source 1..10: mol_type = other DNA
cgtgacaacc

SEQ ID NO: 301 - DNA, 10 bp, synthetic construct
  misc_feature 1..10: note = Description of Artificial Sequence: Synthetic oligonucleotide
  source 1..10: mol_type = other DNA
cgcgctcaga

SEQ ID NO: 302 - DNA, 10 bp, synthetic construct
  misc_feature 1..10: note = Description of Artificial Sequence: Synthetic oligonucleotide
  source 1..10: mol_type = other DNA
cgttcttaat

SEQ ID NO: 303 - DNA, 10 bp, synthetic construct
  misc_feature 1..10: note = Description of Artificial Sequence: Synthetic oligonucleotide
  source 1..10: mol_type = other DNA
acaagagttt

SEQ ID NO: 304 - DNA, 10 bp, synthetic construct
  misc_feature 1..10: note = Description of Artificial Sequence: Synthetic oligonucleotide
  source 1..10: mol_type = other DNA
agggttatag

SEQ ID NO: 305 - DNA, 10 bp, synthetic construct
  misc_feature 1..10: note = Description of Artificial Sequence: Synthetic oligonucleotide
  source 1..10: mol_type = other DNA
accacgactc

SEQ ID NO: 306 - DNA, 10 bp, synthetic construct
  misc_feature 1..10: note = Description of Artificial Sequence: Synthetic oligonucleotide
  source 1..10: mol_type = other DNA
gtactcgggg

SEQ ID NO: 307 - DNA, 10 bp, synthetic construct
  misc_feature 1..10: note = Description of Artificial Sequence: Synthetic oligonucleotide
  source 1..10: mol_type = other DNA
acaaatatct

SEQ ID NO: 308 - DNA, 10 bp, synthetic construct
  misc_feature 1..10: note = Description of Artificial Sequence: Synthetic oligonucleotide
  source 1..10: mol_type = other DNA
gatcggggtg

SEQ ID NO: 309 - DNA, 10 bp, synthetic construct
  misc_feature 1..10: note = Description of Artificial Sequence: Synthetic oligonucleotide
  source 1..10: mol_type = other DNA
atgtaactcc

SEQ ID NO: 310 - DNA, 10 bp, synthetic construct
  misc_feature 1..10: note = Description of Artificial Sequence: Synthetic oligonucleotide
  source 1..10: mol_type = other DNA
atgaagaagc

SEQ ID NO: 311 - DNA, 10 bp, synthetic construct
  misc_feature 1..10: note = Description of Artificial Sequence: Synthetic oligonucleotide
  source 1..10: mol_type = other DNA
atgtattgtc

SEQ ID NO: 312 - DNA, 10 bp, synthetic construct
  misc_feature 1..10: note = Description of Artificial Sequence: Synthetic oligonucleotide
  source 1..10: mol_type = other DNA
tgcattggaa

SEQ ID NO: 313 - DNA, 10 bp, synthetic construct
  misc_feature 1..10: note = Description of Artificial Sequence: Synthetic oligonucleotide
  source 1..10: mol_type = other DNA
gcggacgatc

SEQ ID NO: 314 - DNA, 10 bp, synthetic construct
  misc_feature 1..10: note = Description of Artificial Sequence: Synthetic oligonucleotide
  source 1..10: mol_type = other DNA
ccgtacttga

SEQ ID NO: 315 - DNA, 10 bp, synthetic construct
  misc_feature 1..10: note = Description of Artificial Sequence: Synthetic oligonucleotide
  source 1..10: mol_type = other DNA
tttgcccccg

SEQ ID NO: 316 - DNA, 10 bp, synthetic construct
  misc_feature 1..10: note = Description of Artificial Sequence: Synthetic oligonucleotide
  source 1..10: mol_type = other DNA
acctcacgcg

SEQ ID NO: 317 - DNA, 10 bp, synthetic construct
  misc_feature 1..10: note = Description of Artificial Sequence: Synthetic oligonucleotide
  source 1..10: mol_type = other DNA
attaaggggc

SEQ ID NO: 318 - DNA, 10 bp, synthetic construct
  misc_feature 1..10: note = Description of Artificial Sequence: Synthetic oligonucleotide
  source 1..10: mol_type = other DNA
cgtggacatg

SEQ ID NO: 319 - DNA, 10 bp, synthetic construct
  misc_feature 1..10: note = Description of Artificial Sequence: Synthetic oligonucleotide
  source 1..10: mol_type = other DNA
ttagcccttc

SEQ ID NO: 320 - DNA, 10 bp, synthetic construct
  misc_feature 1..10: note = Description of Artificial Sequence: Synthetic oligonucleotide
  source 1..10: mol_type = other DNA
cgagagtttg

SEQ ID NO: 321 - DNA, 10 bp, synthetic construct
  misc_feature 1..10: note = Description of Artificial Sequence: Synthetic oligonucleotide
  source 1..10: mol_type = other DNA
tgcatcctct

SEQ ID NO: 322 - DNA, 10 bp, synthetic construct
  misc_feature 1..10: note = Description of Artificial Sequence: Synthetic oligonucleotide
  source 1..10: mol_type = other DNA
tgcgattccg

SEQ ID NO: 323 - DNA, 10 bp, synthetic construct
  misc_feature 1..10: note = Description of Artificial Sequence: Synthetic oligonucleotide
  source 1..10: mol_type = other DNA
ttattacgtt

SEQ ID NO: 324 - DNA, 10 bp, synthetic construct
  misc_feature 1..10: note = Description of Artificial Sequence: Synthetic oligonucleotide
  source 1..10: mol_type = other DNA
tgatgtggtt

SEQ ID NO: 325 - DNA, 10 bp, synthetic construct
  misc_feature 1..10: note = Description of Artificial Sequence: Synthetic oligonucleotide
  source 1..10: mol_type = other DNA
gggcgtcaat

SEQ ID NO: 326 - DNA, 10 bp, synthetic construct
  misc_feature 1..10: note = Description of Artificial Sequence: Synthetic oligonucleotide
  source 1..10: mol_type = other DNA
cccttgaaat

SEQ ID NO: 327 - DNA, 10 bp, synthetic construct
  misc_feature 1..10: note = Description of Artificial Sequence: Synthetic oligonucleotide
  source 1..10: mol_type = other DNA
tctttggggc

SEQ ID NO: 328 - DNA, 10 bp, synthetic construct
  misc_feature 1..10: note = Description of Artificial Sequence: Synthetic oligonucleotide
  source 1..10: mol_type = other DNA
accggcaggc

SEQ ID NO: 329 - DNA, 10 bp, synthetic construct
  misc_feature 1..10: note = Description of Artificial Sequence: Synthetic oligonucleotide
  source 1..10: mol_type = other DNA
gctaaaatct

SEQ ID NO: 330 - DNA, 10 bp, synthetic construct
  misc_feature 1..10: note = Description of Artificial Sequence: Synthetic oligonucleotide
  source 1..10: mol_type = other DNA
gccgttgacg

SEQ ID NO: 331 - DNA, 10 bp, synthetic construct
  misc_feature 1..10: note = Description of Artificial Sequence: Synthetic oligonucleotide
  source 1..10: mol_type = other DNA
ggagttgttg

SEQ ID NO: 332 - DNA, 10 bp, synthetic construct
  misc_feature 1..10: note = Description of Artificial Sequence: Synthetic oligonucleotide
  source 1..10: mol_type = other DNA
tacttgagaa

SEQ ID NO: 333 - DNA, 10 bp, synthetic construct
  misc_feature 1..10: note = Description of Artificial Sequence: Synthetic oligonucleotide
  source 1..10: mol_type = other DNA
cgggtgcgct

SEQ ID NO: 334 - DNA, 10 bp, synthetic construct
  misc_feature 1..10: note = Description of Artificial Sequence: Synthetic oligonucleotide
  source 1..10: mol_type = other DNA
aaaagcgtct

SEQ ID NO: 335 - DNA, 10 bp, synthetic construct
  misc_feature 1..10: note = Description of Artificial Sequence: Synthetic oligonucleotide
  source 1..10: mol_type = other DNA
gtaaagatag

SEQ ID NO: 336 - DNA, 10 bp, synthetic construct
  misc_feature 1..10: note = Description of Artificial Sequence: Synthetic oligonucleotide
  source 1..10: mol_type = other DNA
gcctggtcag

SEQ ID NO: 337 - DNA, 10 bp, synthetic construct
  misc_feature 1..10: note = Description of Artificial Sequence: Synthetic oligonucleotide
  source 1..10: mol_type = other DNA
ggcaaaaagg

SEQ ID NO: 338 - DNA, 10 bp, synthetic construct
  misc_feature 1..10: note = Description of Artificial Sequence: Synthetic oligonucleotide
  source 1..10: mol_type = other DNA
acccttctct

SEQ ID NO: 339 - DNA, 10 bp, synthetic construct
  misc_feature 1..10: note = Description of Artificial Sequence: Synthetic oligonucleotide
  source 1..10: mol_type = other DNA
tcacatagtg

SEQ ID NO: 340 - DNA, 10 bp, synthetic construct
  misc_feature 1..10: note = Description of Artificial Sequence: Synthetic oligonucleotide
  source 1..10: mol_type = other DNA
tcgtctgtgc

SEQ ID NO: 341 - DNA, 10 bp, synthetic construct
  misc_feature 1..10: note = Description of Artificial Sequence: Synthetic oligonucleotide
  source 1..10: mol_type = other DNA
tgctcggatc

SEQ ID NO: 342 - DNA, 10 bp, synthetic construct
  misc_feature 1..10: note = Description of Artificial Sequence: Synthetic oligonucleotide
  source 1..10: mol_type = other DNA
agcagtcccg

SEQ ID NO: 343 - DNA, 10 bp, synthetic construct
  misc_feature 1..10: note = Description of Artificial Sequence: Synthetic oligonucleotide
  source 1..10: mol_type = other DNA
tttgggctgt

SEQ ID NO: 344 - DNA, 10 bp, synthetic construct
  misc_feature 1..10: note = Description of Artificial Sequence: Synthetic oligonucleotide
  source 1..10: mol_type = other DNA
ctcacgatct

SEQ ID NO: 345 - DNA, 10 bp, synthetic construct
  misc_feature 1..10: note = Description of Artificial Sequence: Synthetic oligonucleotide
  source 1..10: mol_type = other DNA
tggcgcatac

SEQ ID NO: 346 - DNA, 10 bp, synthetic construct
  misc_feature 1..10: note = Description of Artificial Sequence: Synthetic oligonucleotide
  source 1..10: mol_type = other DNA
gcaattgaaa

SEQ ID NO: 347 - DNA, 10 bp, synthetic construct
  misc_feature 1..10: note = Description of Artificial Sequence: Synthetic oligonucleotide
  source 1..10: mol_type = other DNA
tcgggagacg

SEQ ID NO: 348 - DNA, 10 bp, synthetic construct
  misc_feature 1..10: note = Description of Artificial Sequence: Synthetic oligonucleotide
  source 1..10: mol_type = other DNA
cccggcgaaa

SEQ ID NO: 349 - DNA, 10 bp, synthetic construct
  misc_feature 1..10: note = Description of Artificial Sequence: Synthetic oligonucleotide
  source 1..10: mol_type = other DNA
tgatgcggaa

SEQ ID NO: 350 - DNA, 10 bp, synthetic construct
  misc_feature 1..10: note = Description of Artificial Sequence: Synthetic oligonucleotide
  source 1..10: mol_type = other DNA
aactgaggcg

SEQ ID NO: 351 - DNA, 10 bp, synthetic construct
  misc_feature 1..10: note = Description of Artificial Sequence: Synthetic oligonucleotide
  source 1..10: mol_type = other DNA
catattattt

SEQ ID NO: 352 - DNA, 10 bp, synthetic construct
  misc_feature 1..10: note = Description of Artificial Sequence: Synthetic oligonucleotide
  source 1..10: mol_type = other DNA
aaaagtcatt

SEQ ID NO: 353 - DNA, 10 bp, synthetic construct
  misc_feature 1..10: note = Description of Artificial Sequence: Synthetic oligonucleotide
  source 1..10: mol_type = other DNA
aagcggtgag

SEQ ID NO: 354 - DNA, 10 bp, synthetic construct
  misc_feature 1..10: note = Description of Artificial Sequence: Synthetic oligonucleotide
  source 1..10: mol_type = other DNA
aaggtaatca

SEQ ID NO: 355 - DNA, 10 bp, synthetic construct
  misc_feature 1..10: note = Description of Artificial Sequence: Synthetic oligonucleotide
  source 1..10: mol_type = other DNA
ctgacactta

SEQ ID NO: 356 - DNA, 10 bp, synthetic construct
  misc_feature 1..10: note = Description of Artificial Sequence: Synthetic oligonucleotide
  source 1..10: mol_type = other DNA
ctgttttcta

SEQ ID NO: 357 - DNA, 10 bp, synthetic construct
  misc_feature 1..10: note = Description of Artificial Sequence: Synthetic oligonucleotide
  source 1..10: mol_type = other DNA
cacatggcag

SEQ ID NO: 358 - DNA, 10 bp, synthetic construct
  misc_feature 1..10: note = Description of Artificial Sequence: Synthetic oligonucleotide
  source 1..10: mol_type = other DNA
ttcaatccgg

SEQ ID NO: 359 - DNA, 10 bp, synthetic construct
  misc_feature 1..10: note = Description of Artificial Sequence: Synthetic oligonucleotide
  source 1..10: mol_type = other DNA
tgtccggcat

SEQ ID NO: 360 - DNA, 10 bp, synthetic construct
  misc_feature 1..10: note = Description of Artificial Sequence: Synthetic oligonucleotide
  source 1..10: mol_type = other DNA
tggtaccgtg

SEQ ID NO: 361 - DNA, 10 bp, synthetic construct
  misc_feature 1..10: note = Description of Artificial Sequence: Synthetic oligonucleotide
  source 1..10: mol_type = other DNA
aagagatatt

SEQ ID NO: 362 - DNA, 10 bp, synthetic construct
  misc_feature 1..10: note = Description of Artificial Sequence: Synthetic oligonucleotide
  source 1..10: mol_type = other DNA
gatgtactac

SEQ ID NO: 363 - DNA, 10 bp, synthetic construct
  misc_feature 1..10: note = Description of Artificial Sequence: Synthetic oligonucleotide
  source 1..10: mol_type = other DNA
gaaatggaat

SEQ ID NO: 364 - DNA, 10 bp, synthetic construct
  misc_feature 1..10: note = Description of Artificial Sequence: Synthetic oligonucleotide
  source 1..10: mol_type = other DNA
ttaaaatact

SEQ ID NO: 365 - DNA, 10 bp, synthetic construct
  misc_feature 1..10: note = Description of Artificial Sequence: Synthetic oligonucleotide
  source 1..10: mol_type = other DNA
tgaccggaac

SEQ ID NO: 366 - DNA, 10 bp, synthetic construct
  misc_feature 1..10: note = Description of Artificial Sequence: Synthetic oligonucleotide
  source 1..10: mol_type = other DNA
gtcgccgcaa

SEQ ID NO: 367 - DNA, 10 bp, synthetic construct
  misc_feature 1..10: note = Description of Artificial Sequence: Synthetic oligonucleotide
  source 1..10: mol_type = other DNA
taggataccg

SEQ ID NO: 368 - DNA, 10 bp, synthetic construct
  misc_feature 1..10: note = Description of Artificial Sequence: Synthetic oligonucleotide
  source 1..10: mol_type = other DNA
agtccaattg

SEQ ID NO: 369 - DNA, 10 bp, synthetic construct
  misc_feature 1..10: note = Description of Artificial Sequence: Synthetic oligonucleotide
  source 1..10: mol_type = other DNA
gggggctata

SEQ ID NO: 370 - DNA, 10 bp, synthetic construct
  misc_feature 1..10: note = Description of Artificial Sequence: Synthetic oligonucleotide
  source 1..10: mol_type = other DNA
accttcagtt

SEQ ID NO: 371 - DNA, 10 bp, synthetic construct
  misc_feature 1..10: note = Description of Artificial Sequence: Synthetic oligonucleotide
  source 1..10: mol_type = other DNA
atggcaagta

SEQ ID NO: 372 - DNA, 10 bp, synthetic construct
  misc_feature 1..10: note = Description of Artificial Sequence: Synthetic oligonucleotide
  source 1..10: mol_type = other DNA
agaatgtttt

SEQ ID NO: 373 - DNA, 10 bp, synthetic construct
  misc_feature 1..10: note = Description of Artificial Sequence: Synthetic oligonucleotide
  source 1..10: mol_type = other DNA
agttcgtttg

SEQ ID NO: 374 - DNA, 10 bp, synthetic construct
  misc_feature 1..10: note = Description of Artificial Sequence: Synthetic oligonucleotide
  source 1..10: mol_type = other DNA
cactactgac

SEQ ID NO: 375 - DNA, 10 bp, synthetic construct
  misc_feature 1..10: note = Description of Artificial Sequence: Synthetic oligonucleotide
  source 1..10: mol_type = other DNA
gatcaagagc

SEQ ID NO: 376 - DNA, 10 bp, synthetic construct
  misc_feature 1..10: note = Description of Artificial Sequence: Synthetic oligonucleotide
  source 1..10: mol_type = other DNA
atttatcgag

SEQ ID NO: 377 - DNA, 10 bp, synthetic construct
  misc_feature 1..10: note = Description of Artificial Sequence: Synthetic oligonucleotide
  source 1..10: mol_type = other DNA
cctttttcca

SEQ ID NO: 378 - DNA, 10 bp, synthetic construct
  misc_feature 1..10: note = Description of Artificial Sequence: Synthetic oligonucleotide
  source 1..10: mol_type = other DNA
gcacagaggt

SEQ ID NO: 379 - DNA, 10 bp, synthetic construct
  misc_feature 1..10: note = Description of Artificial Sequence: Synthetic oligonucleotide
  source 1..10: mol_type = other DNA
tgatctgaat

SEQ ID NO: 380 - DNA, 10 bp, synthetic construct
  misc_feature 1..10: note = Description of Artificial Sequence: Synthetic oligonucleotide
  source 1..10: mol_type = other DNA
gttggaggga

SEQ ID NO: 381 - DNA, 10 bp, synthetic construct
  misc_feature 1..10: note = Description of Artificial Sequence: Synthetic oligonucleotide
  source 1..10: mol_type = other DNA
ttttgaaggt

SEQ ID NO: 382 - DNA, 10 bp, synthetic construct
  misc_feature 1..10: note = Description of Artificial Sequence: Synthetic oligonucleotide
  source 1..10: mol_type = other DNA
taagtcctaa

SEQ ID NO: 383 - DNA, 10 bp, synthetic construct
  misc_feature 1..10: note = Description of Artificial Sequence: Synthetic oligonucleotide
  source 1..10: mol_type = other DNA
ggtgttaggg

SEQ ID NO: 384 - DNA, 10 bp, synthetic construct
  misc_feature 1..10: note = Description of Artificial Sequence: Synthetic oligonucleotide
  source 1..10: mol_type = other DNA
tgtatgcacc

SEQ ID NO: 385 - DNA, 10 bp, synthetic construct
  misc_feature 1..10: note = Description of Artificial Sequence: Synthetic oligonucleotide
  source 1..10: mol_type = other DNA
ccgtgccatt

SEQ ID NO: 386 - DNA, 10 bp, synthetic construct
  misc_feature 1..10: note = Description of Artificial Sequence: Synthetic oligonucleotide
  source 1..10: mol_type = other DNA
gaaatcaccc

SEQ ID NO: 387 - DNA, 10 bp, synthetic construct
  misc_feature 1..10: note = Description of Artificial Sequence: Synthetic oligonucleotide
  source 1..10: mol_type = other DNA
tttgcacgtg

SEQ ID NO: 388 - DNA, 10 bp, synthetic construct
  misc_feature 1..10: note = Description of Artificial Sequence: Synthetic oligonucleotide
  source 1..10: mol_type = other DNA
cgtctgtttt

SEQ ID NO: 389 - DNA, 10 bp, synthetic construct
  misc_feature 1..10: note = Description of Artificial Sequence: Synthetic oligonucleotide
  source 1..10: mol_type = other DNA
ctacaccaca

SEQ ID NO: 390 - DNA, 10 bp, synthetic construct
  misc_feature 1..10: note = Description of Artificial Sequence: Synthetic oligonucleotide
  source 1..10: mol_type = other DNA
tgctacaggg

SEQ ID NO: 391 - DNA, 10 bp, synthetic construct
  misc_feature 1..10: note = Description of Artificial Sequence: Synthetic oligonucleotide
  source 1..10: mol_type = other DNA
gggaatatat

SEQ ID NO: 392 - DNA, 10 bp, synthetic construct
  misc_feature 1..10: note = Description of Artificial Sequence: Synthetic oligonucleotide
  source 1..10: mol_type = other DNA
tcatgtattt

SEQ ID NO: 393 - DNA, 10 bp, synthetic construct
  misc_feature 1..10: note = Description of Artificial Sequence: Synthetic oligonucleotide
  source 1..10: mol_type = other DNA
tctccgttta

SEQ ID NO: 394 - DNA, 10 bp, synthetic construct
  misc_feature 1..10: note = Description of Artificial Sequence: Synthetic oligonucleotide
  source 1..10: mol_type = other DNA
tacctctcgc

SEQ ID NO: 395 - DNA, 10 bp, synthetic construct
  misc_feature 1..10: note = Description of Artificial Sequence: Synthetic oligonucleotide
  source 1..10: mol_type = other DNA
gcttcaaccg

SEQ ID NO: 396 - DNA, 10 bp, synthetic construct
  misc_feature 1..10: note = Description of Artificial Sequence: Synthetic oligonucleotide
  source 1..10: mol_type = other DNA
atgaagctac

SEQ ID NO: 397 - DNA, 10 bp, synthetic construct
  misc_feature 1..10: note = Description of Artificial Sequence: Synthetic oligonucleotide
  source 1..10: mol_type = other DNA
cggtacaact

SEQ ID NO: 398 - DNA, 10 bp, synthetic construct
  misc_feature 1..10: note = Description of Artificial Sequence: Synthetic oligonucleotide
  source 1..10: mol_type = other DNA
gtgtggtcgt

SEQ ID NO: 399 - DNA, 10 bp, synthetic construct
  misc_feature 1..10: note = Description of Artificial Sequence: Synthetic oligonucleotide
  source 1..10: mol_type = other DNA
ggggtcatgt

SEQ ID NO: 400 - DNA, 10 bp, synthetic construct
  misc_feature 1..10: note = Description of Artificial Sequence: Synthetic oligonucleotide
  source 1..10: mol_type = other DNA
aggcagccca

SEQ ID NO: 401 - DNA, 10 bp, synthetic construct
  misc_feature 1..10: note = Description of Artificial Sequence: Synthetic oligonucleotide
  source 1..10: mol_type = other DNA
caagcacgat

SEQ ID NO: 402 - DNA, 10 bp, synthetic construct
  misc_feature 1..10: note = Description of Artificial Sequence: Synthetic oligonucleotide
  source 1..10: mol_type = other DNA
tcaaatggat

SEQ ID NO: 403 - DNA, 10 bp, synthetic construct
  misc_feature 1..10: note = Description of Artificial Sequence: Synthetic oligonucleotide
  source 1..10: mol_type = other DNA
ggactgaata

SEQ ID NO: 404 - DNA, 10 bp, synthetic construct
  misc_feature 1..10: note = Description of Artificial Sequence: Synthetic oligonucleotide
  source 1..10: mol_type = other DNA
ccgtagacgt

SEQ ID NO: 405 - DNA, 10 bp, synthetic construct
  misc_feature 1..10: note = Description of Artificial Sequence: Synthetic oligonucleotide
  source 1..10: mol_type = other DNA
cggcgtaccg

SEQ ID NO: 406 - DNA, 10 bp, synthetic construct
  misc_feature 1..10: note = Description of Artificial Sequence: Synthetic oligonucleotide
  source 1..10: mol_type = other DNA
ggcggcgccc

SEQ ID NO: 407 - DNA, 10 bp, synthetic construct
  misc_feature 1..10: note = Description of Artificial Sequence: Synthetic oligonucleotide
  source 1..10: mol_type = other DNA
agacttgatc

SEQ ID NO: 408 - DNA, 10 bp, synthetic construct
  misc_feature 1..10: note = Description of Artificial Sequence: Synthetic oligonucleotide
  source 1..10: mol_type = other DNA
accttgcaca

SEQ ID NO: 409 - DNA, 10 bp, synthetic construct
  misc_feature 1..10: note = Description of Artificial Sequence: Synthetic oligonucleotide
  source 1..10: mol_type = other DNA
taaggtgagt

SEQ ID NO: 410 - DNA, 10 bp, synthetic construct
  misc_feature 1..10: note = Description of Artificial Sequence: Synthetic oligonucleotide
  source 1..10: mol_type = other DNA
ttgttgtttc

SEQ ID NO: 411 - DNA, 10 bp, synthetic construct
  misc_feature 1..10: note = Description of Artificial Sequence: Synthetic oligonucleotide
  source 1..10: mol_type = other DNA
gagggaatac

SEQ ID NO: 412 - DNA, 10 bp, synthetic construct
  misc_feature 1..10: note = Description of Artificial Sequence: Synthetic oligonucleotide
  source 1..10: mol_type = other DNA
ctcgtacgcg

SEQ ID NO: 413 - DNA, 10 bp, synthetic construct
  misc_feature 1..10: note = Description of Artificial Sequence: Synthetic oligonucleotide
  source 1..10: mol_type = other DNA
ccgcggttta

SEQ ID NO: 414 - DNA, 10 bp, synthetic construct
  misc_feature 1..10: note = Description of Artificial Sequence: Synthetic oligonucleotide
  source 1..10: mol_type = other DNA
ttaaagttaa

SEQ ID NO: 415 - DNA, 10 bp, synthetic construct
  misc_feature 1..10: note = Description of Artificial Sequence: Synthetic oligonucleotide
  source 1..10: mol_type = other DNA
gcatatgggt

SEQ ID NO: 416 - DNA, 10 bp, synthetic construct
  misc_feature 1..10: note = Description of Artificial Sequence: Synthetic oligonucleotide
  source 1..10: mol_type = other DNA
agtctgagcc

SEQ ID NO: 417 - DNA, 10 bp, synthetic construct
  misc_feature 1..10: note = Description of Artificial Sequence: Synthetic oligonucleotide
  source 1..10: mol_type = other DNA
tgtcggttcg

SEQ ID NO: 418 - DNA, 10 bp, synthetic construct
  misc_feature 1..10: note = Description of Artificial Sequence: Synthetic oligonucleotide
  source 1..10: mol_type = other DNA
ggtctcaacc

SEQ ID NO: 419 - DNA, 10 bp, synthetic construct
  misc_feature 1..10: note = Description of Artificial Sequence: Synthetic oligonucleotide
  source 1..10: mol_type = other DNA
gtaacggcat

SEQ ID NO: 420 - DNA, 10 bp, synthetic construct
  misc_feature 1..10: note = Description of Artificial Sequence: Synthetic oligonucleotide
  source 1..10: mol_type = other DNA
acactgagaa

SEQ ID NO: 421 - DNA, 10 bp, synthetic construct
  misc_feature 1..10: note = Description of Artificial Sequence: Synthetic oligonucleotide
  source 1..10: mol_type = other DNA
cccaacgtcg

SEQ ID NO: 422 - DNA, 10 bp, synthetic construct
  misc_feature 1..10: note = Description of Artificial Sequence: Synthetic oligonucleotide
  source 1..10: mol_type = other DNA
aagaaactgc

SEQ ID NO: 423 - DNA, 10 bp, synthetic construct
  misc_feature 1..10: note = Description of Artificial Sequence: Synthetic oligonucleotide
  source 1..10: mol_type = other DNA
accagcccac

SEQ ID NO: 424 - DNA, 10 bp, synthetic construct
  misc_feature 1..10: note = Description of Artificial Sequence: Synthetic oligonucleotide
  source 1..10: mol_type = other DNA
tgtagttact

SEQ ID NO: 425 - DNA, 10 bp, synthetic construct
  misc_feature 1..10: note = Description of Artificial Sequence: Synthetic oligonucleotide
  source 1..10: mol_type = other DNA
ggctagaggc

SEQ ID NO: 426 - DNA, 10 bp, synthetic construct
  misc_feature 1..10: note = Description of Artificial Sequence: Synthetic oligonucleotide
  source 1..10: mol_type = other DNA
gttcggcaga

SEQ ID NO: 427 - DNA, 10 bp, synthetic construct
  misc_feature 1..10: note = Description of Artificial Sequence: Synthetic oligonucleotide
  source 1..10: mol_type = other DNA
ccaaaataga

SEQ ID NO: 428 - DNA, 10 bp, synthetic construct
  misc_feature 1..10: note = Description of Artificial Sequence: Synthetic oligonucleotide
  source 1..10: mol_type = other DNA
cccatataac

SEQ ID NO: 429 - DNA, 10 bp, synthetic construct
  misc_feature 1..10: note = Description of Artificial Sequence: Synthetic oligonucleotide
  source 1..10: mol_type = other DNA
gtcactaccg

SEQ ID NO: 430 - DNA, 10 bp, synthetic construct
  misc_feature 1..10: note = Description of Artificial Sequence: Synthetic oligonucleotide
  source 1..10: mol_type = other DNA
gtagtgtggc

SEQ ID NO: 431 - DNA, 10 bp, synthetic construct
  misc_feature 1..10: note = Description of Artificial Sequence: Synthetic oligonucleotide
  source 1..10: mol_type = other DNA
caatctcata

SEQ ID NO: 432 - DNA, 10 bp, synthetic construct
  misc_feature 1..10: note = Description of Artificial Sequence: Synthetic oligonucleotide
  source 1..10: mol_type = other DNA
ccatgttata

SEQ ID NO: 433 - DNA, 10 bp, synthetic construct
  misc_feature 1..10: note = Description of Artificial Sequence: Synthetic oligonucleotide
  source 1..10: mol_type = other DNA
taagcagtgg

SEQ ID NO: 434 - DNA, 10 bp, synthetic construct
  misc_feature 1..10: note = Description of Artificial Sequence: Synthetic oligonucleotide
  source 1..10: mol_type = other DNA
tcggcggcta

SEQ ID NO: 435 - DNA, 10 bp, synthetic construct
  misc_feature 1..10: note = Description of Artificial Sequence: Synthetic oligonucleotide
  source 1..10: mol_type = other DNA
tattaaatgc

SEQ ID NO: 436 - DNA, 10 bp, synthetic construct
  misc_feature 1..10: note = Description of Artificial Sequence: Synthetic oligonucleotide
  source 1..10: mol_type = other DNA
gtcgccatta

SEQ ID NO: 437 - DNA, 10 bp, synthetic construct
  misc_feature 1..10: note = Description of Artificial Sequence: Synthetic oligonucleotide
  source 1..10: mol_type = other DNA
ggcgtcgttc

SEQ ID NO: 438 - DNA, 10 bp, synthetic construct
  misc_feature 1..10: note = Description of Artificial Sequence: Synthetic oligonucleotide
  source 1..10: mol_type = other DNA
ctagtagata

SEQ ID NO: 439 - DNA, 10 bp, synthetic construct
  misc_feature 1..10: note = Description of Artificial Sequence: Synthetic oligonucleotide
  source 1..10: mol_type = other DNA
tcgtcagtat

SEQ ID NO: 440 - DNA, 10 bp, synthetic construct
  misc_feature 1..10: note = Description of Artificial Sequence: Synthetic oligonucleotide
  source 1..10: mol_type = other DNA
ggggtatcgg

SEQ ID NO: 441 - DNA, 10 bp, synthetic construct
  misc_feature 1..10: note = Description of Artificial Sequence: Synthetic oligonucleotide
  source 1..10: mol_type = other DNA
tgctctgcca

SEQ ID NO: 442 - DNA, 10 bp, synthetic construct
  misc_feature 1..10: note = Description of Artificial Sequence: Synthetic oligonucleotide
  source 1..10: mol_type = other DNA
tgccgtaact

SEQ ID NO: 443 - DNA, 10 bp, synthetic construct
  misc_feature 1..10: note = Description of Artificial Sequence: Synthetic oligonucleotide
  source 1..10: mol_type = other DNA
cggtacaggc

SEQ ID NO: 444 - DNA, 10 bp, synthetic construct
  misc_feature 1..10: note = Description of Artificial Sequence: Synthetic oligonucleotide
  source 1..10: mol_type = other DNA
tcctaatttg

SEQ ID NO: 445 - DNA, 10 bp, synthetic construct
  misc_feature 1..10: note = Description of Artificial Sequence: Synthetic oligonucleotide
  source 1..10: mol_type = other DNA
tctttctgga

SEQ ID NO: 446 - DNA, 10 bp, synthetic construct
  misc_feature 1..10: note = Description of Artificial Sequence: Synthetic oligonucleotide
  source 1..10: mol_type = other DNA
ccgcgacttg

SEQ ID NO: 447 - DNA, 10 bp, synthetic construct
  misc_feature 1..10: note = Description of Artificial Sequence: Synthetic oligonucleotide
  source 1..10: mol_type = other DNA
acctatagcg

SEQ ID NO: 448 - DNA, 10 bp, synthetic construct
  misc_feature 1..10: note = Description of Artificial Sequence: Synthetic oligonucleotide
  source 1..10: mol_type = other DNA
gccggcacct

SEQ ID NO: 449 - DNA, 10 bp, synthetic construct
  misc_feature 1..10: note = Description of Artificial Sequence: Synthetic oligonucleotide
  source 1..10: mol_type = other DNA
tttgataggc

SEQ ID NO: 450 - DNA, 10 bp, synthetic construct
  misc_feature 1..10: note = Description of Artificial Sequence: Synthetic oligonucleotide
  source 1..10: mol_type = other DNA
actgtgagct

SEQ ID NO: 451 - DNA, 10 bp, synthetic construct
  misc_feature 1..10: note = Description of Artificial Sequence: Synthetic oligonucleotide
  source 1..10: mol_type = other DNA
ttatcgttca

SEQ ID NO: 452 - DNA, 10 bp, synthetic construct
  misc_feature 1..10: note = Description of Artificial Sequence: Synthetic oligonucleotide
  source 1..10: mol_type = other DNA
actagtggcc

SEQ ID NO: 453 - DNA, 10 bp, synthetic construct
  misc_feature 1..10: note = Description of Artificial Sequence: Synthetic oligonucleotide
  source 1..10: mol_type = other DNA
cctccgtggt

SEQ ID NO: 454 - DNA, 10 bp, synthetic construct
  misc_feature 1..10: note = Description of Artificial Sequence: Synthetic oligonucleotide
  source 1..10: mol_type = other DNA
ttagggtatg

SEQ ID NO: 455 - DNA, 10 bp, synthetic construct
  misc_feature 1..10: note = Description of Artificial Sequence: Synthetic oligonucleotide
  source 1..10: mol_type = other DNA
gaatcaggcg

SEQ ID NO: 456 - DNA, 10 bp, synthetic construct
  misc_feature 1..10: note = Description of Artificial Sequence: Synthetic oligonucleotide
  source 1..10: mol_type = other DNA
ggctgaccaa

SEQ ID NO: 457 - DNA, 10 bp, synthetic construct
  misc_feature 1..10: note = Description of Artificial Sequence: Synthetic oligonucleotide
  source 1..10: mol_type = other DNA
tgccagaccg

SEQ ID NO: 458 - DNA, 10 bp, synthetic construct
  misc_feature 1..10: note = Description of Artificial Sequence: Synthetic oligonucleotide
  source 1..10: mol_type = other DNA
tccctacgcg

SEQ ID NO: 459 - DNA, 10 bp, synthetic construct
  misc_feature 1..10: note = Description of Artificial Sequence: Synthetic oligonucleotide
  source 1..10: mol_type = other DNA
tccgctggag

SEQ ID NO: 460 - DNA, 10 bp, synthetic construct
  misc_feature 1..10: note = Description of Artificial Sequence: Synthetic oligonucleotide
  source 1..10: mol_type = other DNA
ggatcaaaac

SEQ ID NO: 461 - DNA, 10 bp, synthetic construct
  misc_feature 1..10: note = Description of Artificial Sequence: Synthetic oligonucleotide
  source 1..10: mol_type = other DNA
ttcacctcac

SEQ ID NO: 462 - DNA, 10 bp, synthetic construct
  misc_feature 1..10: note = Description of Artificial Sequence: Synthetic oligonucleotide
  source 1..10: mol_type = other DNA
gacacacggc

SEQ ID NO: 463 - DNA, 10 bp, synthetic construct
  misc_feature 1..10: note = Description of Artificial Sequence: Synthetic oligonucleotide
  source 1..10: mol_type = other DNA
tgggcgatta

SEQ ID NO: 464 - DNA, 10 bp, synthetic construct
  misc_feature 1..10: note = Description of Artificial Sequence: Synthetic oligonucleotide
  source 1..10: mol_type = other DNA
taagatcttc

SEQ ID NO: 465 - DNA, 10 bp, synthetic construct
  misc_feature 1..10: note = Description of Artificial Sequence: Synthetic oligonucleotide
  source 1..10: mol_type = other DNA
ctccgactac

SEQ ID NO: 466 - DNA, 10 bp, synthetic construct
  misc_feature 1..10: note = Description of Artificial Sequence: Synthetic oligonucleotide
  source 1..10: mol_type = other DNA
gggccatcat

SEQ ID NO: 467 - DNA, 10 bp, synthetic construct
  misc_feature 1..10: note = Description of Artificial Sequence: Synthetic oligonucleotide
  source 1..10: mol_type = other DNA
tcaggccaga

SEQ ID NO: 468 - DNA, 10 bp, synthetic construct
  misc_feature 1..10: note = Description of Artificial Sequence: Synthetic oligonucleotide
  source 1..10: mol_type = other DNA
cttgtggggc

SEQ ID NO: 469 - DNA, 10 bp, synthetic construct
  misc_feature 1..10: note = Description of Artificial Sequence: Synthetic oligonucleotide
  source 1..10: mol_type = other DNA
agatagtctg

SEQ ID NO: 470 - DNA, 10 bp, synthetic construct
  misc_feature 1..10: note = Description of Artificial Sequence: Synthetic oligonucleotide
  source 1..10: mol_type = other DNA
gcgtcaaagt

SEQ ID NO: 471 - DNA, 10 bp, synthetic construct
  misc_feature 1..10: note = Description of Artificial Sequence: Synthetic oligonucleotide
  source 1..10: mol_type = other DNA
acgaaaattt

SEQ ID NO: 472 - DNA, 10 bp, synthetic construct
  misc_feature 1..10: note = Description of Artificial Sequence: Synthetic oligonucleotide
  source 1..10: mol_type = other DNA
gagtctggtg

SEQ ID NO: 473 - DNA, 10 bp, synthetic construct
  misc_feature 1..10: note = Description of Artificial Sequence: Synthetic oligonucleotide
  source 1..10: mol_type = other DNA
atcgagcgac

SEQ ID NO: 474 - DNA, 10 bp, synthetic construct
  misc_feature 1..10: note = Description of Artificial Sequence: Synthetic oligonucleotide
  source 1..10: mol_type = other DNA
ggtcctcaga

SEQ ID NO: 475 - DNA, 10 bp, synthetic construct
  misc_feature 1..10: note = Description of Artificial Sequence: Synthetic oligonucleotide
  source 1..10: mol_type = other DNA
tgattttgtc

SEQ ID NO: 476 - DNA, 10 bp, synthetic construct
  misc_feature 1..10: note = Description of Artificial Sequence: Synthetic oligonucleotide
  source 1..10: mol_type = other DNA
gcatttctca

SEQ ID NO: 477 - DNA, 10 bp, synthetic construct
  misc_feature 1..10: note = Description of Artificial Sequence: Synthetic oligonucleotide
  source 1..10: mol_type = other DNA
gcatgccagt

SEQ ID NO: 478 - DNA, 10 bp, synthetic construct
  misc_feature 1..10: note = Description of Artificial Sequence: Synthetic oligonucleotide
  source 1..10: mol_type = other DNA
attagacgac

SEQ ID NO: 479 - DNA, 10 bp, synthetic construct
  misc_feature 1..10: note = Description of Artificial Sequence: Synthetic oligonucleotide
  source 1..10: mol_type = other DNA
aaagcccata

SEQ ID NO: 480 - DNA, 10 bp, synthetic construct
  misc_feature 1..10: note = Description of Artificial Sequence: Synthetic oligonucleotide
  source 1..10: mol_type = other DNA
cactacattc

SEQ ID NO: 481 - DNA, 10 bp, synthetic construct
  misc_feature 1..10: note = Description of Artificial Sequence: Synthetic oligonucleotide
  source 1..10: mol_type = other DNA
cacggtttct

SEQ ID NO: 482 - DNA, 10 bp, synthetic construct
  misc_feature 1..10: note = Description of Artificial Sequence: Synthetic oligonucleotide
  source 1..10: mol_type = other DNA
cccaccagtg

SEQ ID NO: 483 - DNA, 10 bp, synthetic construct
  misc_feature 1..10: note = Description of Artificial Sequence: Synthetic oligonucleotide
  source 1..10: mol_type = other DNA
ctcacttgtc

SEQ ID NO: 484 - DNA, 10 bp, synthetic construct
  misc_feature 1..10: note = Description of Artificial Sequence: Synthetic oligonucleotide
  source 1..10: mol_type = other DNA
gatagactct

SEQ ID NO: 485 - DNA, 10 bp, synthetic construct
  misc_feature 1..10: note = Description of Artificial Sequence: Synthetic oligonucleotide
  source 1..10: mol_type = other DNA
atttccattt

SEQ ID NO: 486 - DNA, 10 bp, synthetic construct
  misc_feature 1..10: note = Description of Artificial Sequence: Synthetic oligonucleotide
  source 1..10: mol_type = other DNA
atatgtggcc

SEQ ID NO: 487 - DNA, 10 bp, synthetic construct
  misc_feature 1..10: note = Description of Artificial Sequence: Synthetic oligonucleotide
  source 1..10: mol_type = other DNA
cgggacgaac

SEQ ID NO: 488 - DNA, 10 bp, synthetic construct
  misc_feature 1..10: note = Description of Artificial Sequence: Synthetic oligonucleotide
  source 1..10: mol_type = other DNA
agaaccgtga

SEQ ID NO: 489 - DNA, 10 bp, synthetic construct
  misc_feature 1..10: note = Description of Artificial Sequence: Synthetic oligonucleotide
  source 1..10: mol_type = other DNA
tagtgtactg

SEQ ID NO: 490 - DNA, 10 bp, synthetic construct
  misc_feature 1..10: note = Description of Artificial Sequence: Synthetic oligonucleotide
  source 1..10: mol_type = other DNA
aactaatcga

SEQ ID NO: 491 - DNA, 10 bp, synthetic construct
  misc_feature 1..10: note = Description of Artificial Sequence: Synthetic oligonucleotide
  source 1..10: mol_type = other DNA
cgaagtgacg

SEQ ID NO: 492 - DNA, 10 bp, synthetic construct
  misc_feature 1..10: note = Description of Artificial Sequence: Synthetic oligonucleotide
  source 1..10: mol_type = other DNA
cggagcctcg

SEQ ID NO: 493 - DNA, 10 bp, synthetic construct
  misc_feature 1..10: note = Description of Artificial Sequence: Synthetic oligonucleotide
  source 1..10: mol_type = other DNA
atcacacgag

SEQ ID NO: 494 - DNA, 10 bp, synthetic construct
  misc_feature 1..10: note = Description of Artificial Sequence: Synthetic oligonucleotide
  source 1..10: mol_type = other DNA
cgacgagttc

SEQ ID NO: 495 - DNA, 10 bp, synthetic construct
  misc_feature 1..10: note = Description of Artificial Sequence: Synthetic oligonucleotide
  source 1..10: mol_type = other DNA
gcttcccgtg

SEQ ID NO: 496 - DNA, 10 bp, synthetic construct
  misc_feature 1..10: note = Description of Artificial Sequence: Synthetic oligonucleotide
  source 1..10: mol_type = other DNA
gattcatacc

SEQ ID NO: 497 - DNA, 10 bp, synthetic construct
  misc_feature 1..10: note = Description of Artificial Sequence: Synthetic oligonucleotide
  source 1..10: mol_type = other DNA
gagagaagcg

SEQ ID NO: 498 - DNA, 10 bp, synthetic construct
  misc_feature 1..10: note = Description of Artificial Sequence: Synthetic oligonucleotide
  source 1..10: mol_type = other DNA
gaagtggcct

SEQ ID NO: 499 - DNA, 10 bp, synthetic construct
  misc_feature 1..10: note = Description of Artificial Sequence: Synthetic oligonucleotide
  source 1..10: mol_type = other DNA
ggacgacgcc

SEQ ID NO: 500 - DNA, 10 bp, synthetic construct
  misc_feature 1..10: note = Description of Artificial Sequence: Synthetic oligonucleotide
  source 1..10: mol_type = other DNA
tagggtctca

SEQ ID NO: 501 - DNA, 10 bp, synthetic construct
  misc_feature 1..10: note = Description of Artificial Sequence: Synthetic oligonucleotide
  source 1..10: mol_type = other DNA
aactacaggt

SEQ ID NO: 502 - DNA, 10 bp, synthetic construct
  misc_feature 1..10: note = Description of Artificial Sequence: Synthetic oligonucleotide
  source 1..10: mol_type = other DNA
gtggcctgtg

SEQ ID NO: 503 - DNA, 10 bp, synthetic construct
  misc_feature 1..10: note = Description of Artificial Sequence: Synthetic oligonucleotide
  source 1..10: mol_type = other DNA
ctttaccagc

SEQ ID NO: 504 - DNA, 10 bp, synthetic construct
  misc_feature 1..10: note = Description of Artificial Sequence: Synthetic oligonucleotide
  source 1..10: mol_type = other DNA
cgcgttactg

SEQ ID NO: 505 - DNA, 10 bp, synthetic construct
  misc_feature 1..10: note = Description of Artificial Sequence: Synthetic oligonucleotide
  source 1..10: mol_type = other DNA
ttgctcccgt

SEQ ID NO: 506 - DNA, 10 bp, synthetic construct
  misc_feature 1..10: note = Description of Artificial Sequence: Synthetic oligonucleotide
  source 1..10: mol_type = other DNA
catcaaacaa

SEQ ID NO: 507 - DNA, 10 bp, synthetic construct
  misc_feature 1..10: note = Description of Artificial Sequence: Synthetic oligonucleotide
  source 1..10: mol_type = other DNA
gctttatgat

SEQ ID NO: 508 - DNA, 10 bp, synthetic construct
  misc_feature 1..10: note = Description of Artificial Sequence: Synthetic oligonucleotide
  source 1..10: mol_type = other DNA
ctgcatactg

SEQ ID NO: 509 - DNA, 10 bp, synthetic construct
  misc_feature 1..10: note = Description of Artificial Sequence: Synthetic oligonucleotide
  source 1..10: mol_type = other DNA
ggtggctcag

SEQ ID NO: 510 - DNA, 10 bp, synthetic construct
  misc_feature 1..10: note = Description of Artificial Sequence: Synthetic oligonucleotide
  source 1..10: mol_type = other DNA
ggacgatcaa

SEQ ID NO: 511 - DNA, 10 bp, synthetic construct
  misc_feature 1..10: note = Description of Artificial Sequence: Synthetic oligonucleotide
  source 1..10: mol_type = other DNA
ccgactggtg

SEQ ID NO: 512 - DNA, 10 bp, synthetic construct
  misc_feature 1..10: note = Description of Artificial Sequence: Synthetic oligonucleotide
  source 1..10: mol_type = other DNA
ggaacaaccg

SEQ ID NO: 513 - DNA, 10 bp, synthetic construct
  misc_feature 1..10: note = Description of Artificial Sequence: Synthetic oligonucleotide
  source 1..10: mol_type = other DNA
gaacgagacc

SEQ ID NO: 514 - DNA, 10 bp, synthetic construct
  misc_feature 1..10: note = Description of Artificial Sequence: Synthetic oligonucleotide
  source 1..10: mol_type = other DNA
caccaagaaa

SEQ ID NO: 515 - DNA, 10 bp, synthetic construct
  misc_feature 1..10: note = Description of Artificial Sequence: Synthetic oligonucleotide
  source 1..10: mol_type = other DNA
atgcattacc

SEQ ID NO: 516 - DNA, 10 bp, synthetic construct
  misc_feature 1..10: note = Description of Artificial Sequence: Synthetic oligonucleotide
  source 1..10: mol_type = other DNA
gtatcatgcc

SEQ ID NO: 517 - DNA, 10 bp, synthetic construct
  misc_feature 1..10: note = Description of Artificial Sequence: Synthetic oligonucleotide
  source 1..10: mol_type = other DNA
agtagatgtt

SEQ ID NO: 518 - DNA, 10 bp, synthetic construct
  misc_feature 1..10: note = Description of Artificial Sequence: Synthetic oligonucleotide
  source 1..10: mol_type = other DNA
ctctagatgt

SEQ ID NO: 519 - DNA, 10 bp, synthetic construct
  misc_feature 1..10: note = Description of Artificial Sequence: Synthetic oligonucleotide
  source 1..10: mol_type = other DNA
gctacttgtg

SEQ ID NO: 520 - DNA, 10 bp, synthetic construct
  misc_feature 1..10: note = Description of Artificial Sequence: Synthetic oligonucleotide
  source 1..10: mol_type = other DNA
tatgaaacgt

SEQ ID NO: 521 - DNA, 10 bp, synthetic construct
  misc_feature 1..10: note = Description of Artificial Sequence: Synthetic oligonucleotide
  source 1..10: mol_type = other DNA
cctcgttgat

SEQ ID NO: 522 - DNA, 10 bp, synthetic construct
  misc_feature 1..10: note = Description of Artificial Sequence: Synthetic oligonucleotide
  source 1..10: mol_type = other DNA
ctagagccat

SEQ ID NO: 523 - DNA, 10 bp, synthetic construct
  misc_feature 1..10: note = Description of Artificial Sequence: Synthetic oligonucleotide
  source 1..10: mol_type = other DNA
tagagttata

SEQ ID NO: 524 - DNA, 10 bp, synthetic construct
  misc_feature 1..10: note = Description of Artificial Sequence: Synthetic oligonucleotide
  source 1..10: mol_type = other DNA
aacgagaggc

SEQ ID NO: 525 - DNA, 10 bp, synthetic construct
  misc_feature 1..10: note = Description of Artificial Sequence: Synthetic oligonucleotide
  source 1..10: mol_type = other DNA
ggtctaccgt

SEQ ID NO: 526 - DNA, 10 bp, synthetic construct
  misc_feature 1..10: note = Description of Artificial Sequence: Synthetic oligonucleotide
  source 1..10: mol_type = other DNA
gccccctcac

SEQ ID NO: 527 - DNA, 10 bp, synthetic construct
  misc_feature 1..10: note = Description of Artificial Sequence: Synthetic oligonucleotide
  source 1..10: mol_type = other DNA
cataggaatt

SEQ ID NO: 528 - DNA, 10 bp, synthetic construct
  misc_feature 1..10: note = Description of Artificial Sequence: Synthetic oligonucleotide
  source 1..10: mol_type = other DNA
tccggctcgt

SEQ ID NO: 529 - DNA, 10 bp, synthetic construct
  misc_feature 1..10: note = Description of Artificial Sequence: Synthetic oligonucleotide
  source 1..10: mol_type = other DNA
tgagagtcgg

SEQ ID NO: 530 - DNA, 10 bp, synthetic construct
  misc_feature 1..10: note = Description of Artificial Sequence: Synthetic oligonucleotide
  source 1..10: mol_type = other DNA
cgtagaaata

SEQ ID NO: 531 - DNA, 10 bp, synthetic construct
  misc_feature 1..10: note = Description of Artificial Sequence: Synthetic oligonucleotide
  source 1..10: mol_type = other DNA
ctttacatga

SEQ ID NO: 532 - DNA, 10 bp, synthetic construct
  misc_feature 1..10: note = Description of Artificial Sequence: Synthetic oligonucleotide
  source 1..10: mol_type = other DNA
gagcgccgtc

SEQ ID NO: 533 - DNA, 10 bp, synthetic construct
  misc_feature 1..10: note = Description of Artificial Sequence: Synthetic oligonucleotide
  source 1..10: mol_type = other DNA
ggctctcggc

SEQ ID NO: 534 - DNA, 10 bp, synthetic construct
  misc_feature 1..10: note = Description of Artificial Sequence: Synthetic oligonucleotide
  source 1..10: mol_type = other DNA
agagcttgtt

SEQ ID NO: 535 - DNA, 10 bp, synthetic construct
  misc_feature 1..10: note = Description of Artificial Sequence: Synthetic oligonucleotide
  source 1..10: mol_type = other DNA
aatcagccac

SEQ ID NO: 536 - DNA, 10 bp, synthetic construct
  misc_feature 1..10: note = Description of Artificial Sequence: Synthetic oligonucleotide
  source 1..10: mol_type = other DNA
agaagagcca

SEQ ID NO: 537 - DNA, 10 bp, synthetic construct
  misc_feature 1..10: note = Description of Artificial Sequence: Synthetic oligonucleotide
  source 1..10: mol_type = other DNA
tcgtatgagt

SEQ ID NO: 538 - DNA, 10 bp, synthetic construct
  misc_feature 1..10: note = Description of Artificial Sequence: Synthetic oligonucleotide
  source 1..10: mol_type = other DNA
ttcttcctcg

SEQ ID NO: 539 - DNA, 10 bp, synthetic construct
  misc_feature 1..10: note = Description of Artificial Sequence: Synthetic oligonucleotide
  source 1..10: mol_type = other DNA
acacaaaagc

SEQ ID NO: 540 - DNA, 10 bp, synthetic construct
  misc_feature 1..10: note = Description of Artificial Sequence: Synthetic oligonucleotide
  source 1..10: mol_type = other DNA
cgcgggaccc

SEQ ID NO: 541 - DNA, 10 bp, synthetic construct
  misc_feature 1..10: note = Description of Artificial Sequence: Synthetic oligonucleotide
  source 1..10: mol_type = other DNA
gtcgcgacac

SEQ ID NO: 542 - DNA, 10 bp, synthetic construct
  misc_feature 1..10: note = Description of Artificial Sequence: Synthetic oligonucleotide
  source 1..10: mol_type = other DNA
ccggaggaaa

SEQ ID NO: 543 - DNA, 10 bp, synthetic construct
  misc_feature 1..10: note = Description of Artificial Sequence: Synthetic oligonucleotide
  source 1..10: mol_type = other DNA
cggcgtatga

SEQ ID NO: 544 - DNA, 10 bp, synthetic construct
  misc_feature 1..10: note = Description of Artificial Sequence: Synthetic oligonucleotide
  source 1..10: mol_type = other DNA
taggcattct

SEQ ID NO: 545 - DNA, 10 bp, synthetic construct
  misc_feature 1..10: note = Description of Artificial Sequence: Synthetic oligonucleotide
  source 1..10: mol_type = other DNA
aaaggaggga

SEQ ID NO: 546 - DNA, 10 bp, synthetic construct
  misc_feature 1..10: note = Description of Artificial Sequence: Synthetic oligonucleotide
  source 1..10: mol_type = other DNA
acctttacgg

SEQ ID NO: 547 - DNA, 10 bp, synthetic construct
  misc_feature 1..10: note = Description of Artificial Sequence: Synthetic oligonucleotide
  source 1..10: mol_type = other DNA
ctaccgttaa

SEQ ID NO: 548 - DNA, 10 bp, synthetic construct
  misc_feature 1..10: note = Description of Artificial Sequence: Synthetic oligonucleotide
  source 1..10: mol_type = other DNA
gagcttcgcc

SEQ ID NO: 549 - DNA, 10 bp, synthetic construct
  misc_feature 1..10: note = Description of Artificial Sequence: Synthetic oligonucleotide
  source 1..10: mol_type = other DNA
gccatagaag

SEQ ID NO: 550 - DNA, 10 bp, synthetic construct
  misc_feature 1..10: note = Description of Artificial Sequence: Synthetic oligonucleotide
  source 1..10: mol_type = other DNA
tttagcgtat

SEQ ID NO: 551 - DNA, 10 bp, synthetic construct
  misc_feature 1..10: note = Description of Artificial Sequence: Synthetic oligonucleotide
  source 1..10: mol_type = other DNA
gcaaacagat

SEQ ID NO: 552 - DNA, 10 bp, synthetic construct
  misc_feature 1..10: note = Description of Artificial Sequence: Synthetic oligonucleotide
  source 1..10: mol_type = other DNA
taggtcatgg

SEQ ID NO: 553 - DNA, 10 bp, synthetic construct
  misc_feature 1..10: note = Description of Artificial Sequence: Synthetic oligonucleotide
  source 1..10: mol_type = other DNA
ctctaacaga

SEQ ID NO: 554 - DNA, 10 bp, synthetic construct
  misc_feature 1..10: note = Description of Artificial Sequence: Synthetic oligonucleotide
  source 1..10: mol_type = other DNA
ggctcatgaa

SEQ ID NO: 555 - DNA, 10 bp, synthetic construct
  misc_feature 1..10: note = Description of Artificial Sequence: Synthetic oligonucleotide
  source 1..10: mol_type = other DNA
caatgtctca

SEQ ID NO: 556 - DNA, 10 bp, synthetic construct
  misc_feature 1..10: note = Description of Artificial Sequence: Synthetic oligonucleotide
  source 1..10: mol_type = other DNA
tgatcgtatt

SEQ ID NO: 557 - DNA, 10 bp, synthetic construct
  misc_feature 1..10: note = Description of Artificial Sequence: Synthetic oligonucleotide
  source 1..10: mol_type = other DNA
gcgcttttca

SEQ ID NO: 558 - DNA, 10 bp, synthetic construct
  misc_feature 1..10: note = Description of Artificial Sequence: Synthetic oligonucleotide
  source 1..10: mol_type = other DNA
aagattatat

SEQ ID NO: 559 - DNA, 10 bp, synthetic construct
  misc_feature 1..10: note = Description of Artificial Sequence: Synthetic oligonucleotide
  source 1..10: mol_type = other DNA
actagctgac

SEQ ID NO: 560 - DNA, 10 bp, synthetic construct
  misc_feature 1..10: note = Description of Artificial Sequence: Synthetic oligonucleotide
  source 1..10: mol_type = other DNA
ggtgagctca

SEQ ID NO: 561 - DNA, 10 bp, synthetic construct
  misc_feature 1..10: note = Description of Artificial Sequence: Synthetic oligonucleotide
  source 1..10: mol_type = other DNA
cgctttcgct

SEQ ID NO: 562 - DNA, 10 bp, synthetic construct
  misc_feature 1..10: note = Description of Artificial Sequence: Synthetic oligonucleotide
  source 1..10: mol_type = other DNA
tgattcaaaa

SEQ ID NO: 563 - DNA, 10 bp, synthetic construct
  misc_feature 1..10: note = Description of Artificial Sequence: Synthetic oligonucleotide
  source 1..10: mol_type = other DNA
actgaacagg

SEQ ID NO: 564 - DNA, 10 bp, synthetic construct
  misc_feature 1..10: note = Description of Artificial Sequence: Synthetic oligonucleotide
  source 1..10: mol_type = other DNA
attcgagcta

SEQ ID NO: 565 - DNA, 10 bp, synthetic construct
  misc_feature 1..10: note = Description of Artificial Sequence: Synthetic oligonucleotide
  source 1..10: mol_type = other DNA
tgtaggctaa

SEQ ID NO: 566 - DNA, 10 bp, synthetic construct
  misc_feature 1..10: note = Description of Artificial Sequence: Synthetic oligonucleotide
  source 1..10: mol_type = other DNA
acaaagcttt

SEQ ID NO: 567 - DNA, 10 bp, synthetic construct
  misc_feature 1..10: note = Description of Artificial Sequence: Synthetic oligonucleotide
  source 1..10: mol_type = other DNA
gcccgaggga

SEQ ID NO: 568 - DNA, 10 bp, synthetic construct
  misc_feature 1..10: note = Description of Artificial Sequence: Synthetic oligonucleotide
  source 1..10: mol_type = other DNA
gcccgctggg

SEQ ID NO: 569 - DNA, 10 bp, synthetic construct
  misc_feature 1..10: note = Description of Artificial Sequence: Synthetic oligonucleotide
  source 1..10: mol_type = other DNA
accccgctga

SEQ ID NO: 570 - DNA, 10 bp, synthetic construct
  misc_feature 1..10: note = Description of Artificial Sequence: Synthetic oligonucleotide
  source 1..10: mol_type = other DNA
cttatgccct

SEQ ID NO: 571 - DNA, 10 bp, synthetic construct
  misc_feature 1..10: note = Description of Artificial Sequence: Synthetic oligonucleotide
  source 1..10: mol_type = other DNA
ccgccatagc

SEQ ID NO: 572 - DNA, 10 bp, synthetic construct
  misc_feature 1..10: note = Description of Artificial Sequence: Synthetic oligonucleotide
  source 1..10: mol_type = other DNA
cttaatgatt

SEQ ID NO: 573 - DNA, 10 bp, synthetic construct
  misc_feature 1..10: note = Description of Artificial Sequence: Synthetic oligonucleotide
  source 1..10: mol_type = other DNA
cagtccacaa

SEQ ID NO: 574 - DNA, 10 bp, synthetic construct
  misc_feature 1..10: note = Description of Artificial Sequence: Synthetic oligonucleotide
  source 1..10: mol_type = other DNA
atggacggac

SEQ ID NO: 575 - DNA, 10 bp, synthetic construct
  misc_feature 1..10: note = Description of Artificial Sequence: Synthetic oligonucleotide
  source 1..10: mol_type = other DNA
cggcctctcg

SEQ ID NO: 576 - DNA, 10 bp, synthetic construct
  misc_feature 1..10: note = Description of Artificial Sequence: Synthetic oligonucleotide
  source 1..10: mol_type = other DNA
tagtcgccat

SEQ ID NO: 577 - DNA, 10 bp, synthetic construct
  misc_feature 1..10: note = Description of Artificial Sequence: Synthetic oligonucleotide
  source 1..10: mol_type = other DNA
gttgatcttc

SEQ ID NO: 578 - DNA, 10 bp, synthetic construct
  misc_feature 1..10: note = Description of Artificial Sequence: Synthetic oligonucleotide
  source 1..10: mol_type = other DNA
acttgccaag

SEQ ID NO: 579 - DNA, 10 bp, synthetic construct
  misc_feature 1..10: note = Description of Artificial Sequence: Synthetic oligonucleotide
  source 1..10: mol_type = other DNA
atgactggtt

SEQ ID NO: 580 - DNA, 10 bp, synthetic construct
  misc_feature 1..10: note = Description of Artificial Sequence: Synthetic oligonucleotide
  source 1..10: mol_type = other DNA
tgtcgtagga

SEQ ID NO: 581 - DNA, 10 bp, synthetic construct
  misc_feature 1..10: note = Description of Artificial Sequence: Synthetic oligonucleotide
  source 1..10: mol_type = other DNA
agcaaacacg

SEQ ID NO: 582 - DNA, 10 bp, synthetic construct
  misc_feature 1..10: note = Description of Artificial Sequence: Synthetic oligonucleotide
  source 1..10: mol_type = other DNA
tactgatgaa

SEQ ID NO: 583 - DNA, 10 bp, synthetic construct
  misc_feature 1..10: note = Description of Artificial Sequence: Synthetic oligonucleotide
  source 1..10: mol_type = other DNA
gtatcccata

SEQ ID NO: 584 - DNA, 10 bp, synthetic construct
  misc_feature 1..10: note = Description of Artificial Sequence: Synthetic oligonucleotide
  source 1..10: mol_type = other DNA
tagccaggtt

SEQ ID NO: 585 - DNA, 10 bp, synthetic construct
  misc_feature 1..10: note = Description of Artificial Sequence: Synthetic oligonucleotide
  source 1..10: mol_type = other DNA
cgtgtggcga

SEQ ID NO: 586 - DNA, 10 bp, synthetic construct
  misc_feature 1..10: note = Description of Artificial Sequence: Synthetic oligonucleotide
  source 1..10: mol_type = other DNA
atcgaattgc

SEQ ID NO: 587 - DNA, 10 bp, synthetic construct
  misc_feature 1..10: note = Description of Artificial Sequence: Synthetic oligonucleotide
  source 1..10: mol_type = other DNA
ccccaatatt

SEQ ID NO: 588 - DNA, 10 bp, synthetic construct
  misc_feature 1..10: note = Description of Artificial Sequence: Synthetic oligonucleotide
  source 1..10: mol_type = other DNA
cccgtttctc

SEQ ID NO: 589 - DNA, 10 bp, synthetic construct
  misc_feature 1..10: note = Description of Artificial Sequence: Synthetic oligonucleotide
  source 1..10: mol_type = other DNA
tccgcatcta

SEQ ID NO: 590 - DNA, 10 bp, synthetic construct
  misc_feature 1..10: note = Description of Artificial Sequence: Synthetic oligonucleotide
  source 1..10: mol_type = other DNA
caagcctcat

SEQ ID NO: 591 - DNA, 10 bp, synthetic construct
  misc_feature 1..10: note = Description of Artificial Sequence: Synthetic oligonucleotide
  source 1..10: mol_type = other DNA
tttcaatccc

SEQ ID NO: 592 - DNA, 10 bp, synthetic construct
  misc_feature 1..10: note = Description of Artificial Sequence: Synthetic oligonucleotide
  source 1..10: mol_type = other DNA
ccttcccatc

SEQ ID NO: 593 - DNA, 10 bp, synthetic construct
  misc_feature 1..10: note = Description of Artificial Sequence: Synthetic oligonucleotide
  source 1..10: mol_type = other DNA
aggtacaaga

SEQ ID NO: 594 - DNA, 10 bp, synthetic construct
  misc_feature 1..10: note = Description of Artificial Sequence: Synthetic oligonucleotide
  source 1..10: mol_type = other DNA
gtgtaatgga

SEQ ID NO: 595 - DNA, 10 bp, synthetic construct
  misc_feature 1..10: note = Description of Artificial Sequence: Synthetic oligonucleotide
  source 1..10: mol_type = other DNA
aaactgagct

SEQ ID NO: 596 - DNA, 10 bp, synthetic construct
  misc_feature 1..10: note = Description of Artificial Sequence: Synthetic oligonucleotide
  source 1..10: mol_type = other DNA
atctctgccc

SEQ ID NO: 597 - DNA, 10 bp, synthetic construct
  misc_feature 1..10: note = Description of Artificial Sequence: Synthetic oligonucleotide
  source 1..10: mol_type = other DNA
cgacatttgc

SEQ ID NO: 598 - DNA, 10 bp, synthetic construct
  misc_feature 1..10: note = Description of Artificial Sequence: Synthetic oligonucleotide
  source 1..10: mol_type = other DNA
tgtgaacccg

SEQ ID NO: 599 - DNA, 10 bp, synthetic construct
  misc_feature 1..10: note = Description of Artificial Sequence: Synthetic oligonucleotide
  source 1..10: mol_type = other DNA
tgacacccca

SEQ ID NO: 600 - DNA, 10 bp, synthetic construct
  misc_feature 1..10: note = Description of Artificial Sequence: Synthetic oligonucleotide
  source 1..10: mol_type = other DNA
taggccaaag

SEQ ID NO: 601 - DNA, 10 bp, synthetic construct
  misc_feature 1..10: note = Description of Artificial Sequence: Synthetic oligonucleotide
  source 1..10: mol_type = other DNA
gaaattgtag

SEQ ID NO: 602 - DNA, 10 bp, synthetic construct
  misc_feature 1..10: note = Description of Artificial Sequence: Synthetic oligonucleotide
  source 1..10: mol_type = other DNA
gcgtctgatt

SEQ ID NO: 603 - DNA, 10 bp, synthetic construct
  misc_feature 1..10: note = Description of Artificial Sequence: Synthetic oligonucleotide
  source 1..10: mol_type = other DNA
tctcattgtt

SEQ ID NO: 604 - DNA, 10 bp, synthetic construct
  misc_feature 1..10: note = Description of Artificial Sequence: Synthetic oligonucleotide
  source 1..10: mol_type = other DNA
ctgacatctc

SEQ ID NO: 605 - DNA, 10 bp, synthetic construct
  misc_feature 1..10: note = Description of Artificial Sequence: Synthetic oligonucleotide
  source 1..10: mol_type = other DNA
gtatccagtg

SEQ ID NO: 606 - DNA, 10 bp, synthetic construct
  misc_feature 1..10: note = Description of Artificial Sequence: Synthetic oligonucleotide
  source 1..10: mol_type = other DNA
gatggccgtt

SEQ ID NO: 607 - DNA, 10 bp, synthetic construct
  misc_feature 1..10: note = Description of Artificial Sequence: Synthetic oligonucleotide
  source 1..10: mol_type = other DNA
tcaccctctc

SEQ ID NO: 608 - DNA, 10 bp, synthetic construct
  misc_feature 1..10: note = Description of Artificial Sequence: Synthetic oligonucleotide
  source 1..10: mol_type = other DNA
ggcactattc

SEQ ID NO: 609 - DNA, 10 bp, synthetic construct
  misc_feature 1..10: note = Description of Artificial Sequence: Synthetic oligonucleotide
  source 1..10: mol_type = other DNA
aaataactgt

SEQ ID NO: 610 - DNA, 10 bp, synthetic construct
  misc_feature 1..10: note = Description of Artificial Sequence: Synthetic oligonucleotide
  source 1..10: mol_type = other DNA
cagctccatt

SEQ ID NO: 611 - DNA, 10 bp, synthetic construct
  misc_feature 1..10: note = Description of Artificial Sequence: Synthetic oligonucleotide
  source 1..10: mol_type = other DNA
ctcttgactc

SEQ ID NO: 612 - DNA, 10 bp, synthetic construct
  misc_feature 1..10: note = Description of Artificial Sequence: Synthetic oligonucleotide
  source 1..10: mol_type = other DNA
tttcctatac

SEQ ID NO: 613 - DNA, 10 bp, synthetic construct
  misc_feature 1..10: note = Description of Artificial Sequence: Synthetic oligonucleotide
  source 1..10: mol_type = other DNA
ccatacccga

SEQ ID NO: 614 - DNA, 10 bp, synthetic construct
  misc_feature 1..10: note = Description of Artificial Sequence: Synthetic oligonucleotide
  source 1..10: mol_type = other DNA
tcgccgagcg

SEQ ID NO: 615 - DNA, 10 bp, synthetic construct
  misc_feature 1..10: note = Description of Artificial Sequence: Synthetic oligonucleotide
  source 1..10: mol_type = other DNA
cgctgaagcc

SEQ ID NO: 616 - DNA, 10 bp, synthetic construct
  misc_feature 1..10: note = Description of Artificial Sequence: Synthetic oligonucleotide
  source 1..10: mol_type = other DNA
tctggcccca

SEQ ID NO: 617 - DNA, 10 bp, synthetic construct
  misc_feature 1..10: note = Description of Artificial Sequence: Synthetic oligonucleotide
  source 1..10: mol_type = other DNA
gctacattga

SEQ ID NO: 618 - DNA, 10 bp, synthetic construct
  misc_feature 1..10: note = Description of Artificial Sequence: Synthetic oligonucleotide
  source 1..10: mol_type = other DNA
cgcatcataa

SEQ ID NO: 619 - DNA, 10 bp, synthetic construct
  misc_feature 1..10: note = Description of Artificial Sequence: Synthetic oligonucleotide
  source 1..10: mol_type = other DNA
gcaaagggcc

SEQ ID NO: 620 - DNA, 10 bp, synthetic construct
  misc_feature 1..10: note = Description of Artificial Sequence: Synthetic oligonucleotide
  source 1..10: mol_type = other DNA
aacggcgcag

SEQ ID NO: 621 - DNA, 10 bp, synthetic construct
  misc_feature 1..10: note = Description of Artificial Sequence: Synthetic oligonucleotide
  source 1..10: mol_type = other DNA
cgactgacat

SEQ ID NO: 622 - DNA, 10 bp, synthetic construct
  misc_feature 1..10: note = Description of Artificial Sequence: Synthetic oligonucleotide
  source 1..10: mol_type = other DNA
atgacagggc

SEQ ID NO: 623 - DNA, 10 bp, synthetic construct
  misc_feature 1..10: note = Description of Artificial Sequence: Synthetic oligonucleotide
  source 1..10: mol_type = other DNA
caagttctcc

SEQ ID NO: 624 - DNA, 10 bp, synthetic construct
  misc_feature 1..10: note = Description of Artificial Sequence: Synthetic oligonucleotide
  source 1..10: mol_type = other DNA
tcgccgcttt

SEQ ID NO: 625 - DNA, 10 bp, synthetic construct
  misc_feature 1..10: note = Description of Artificial Sequence: Synthetic oligonucleotide
  source 1..10: mol_type = other DNA
atgccggaaa

SEQ ID NO: 626 - DNA, 10 bp, synthetic construct
  misc_feature 1..10: note = Description of Artificial Sequence: Synthetic oligonucleotide
  source 1..10: mol_type = other DNA
gcggttacta

SEQ ID NO: 627 - DNA, 10 bp, synthetic construct
  misc_feature 1..10: note = Description of Artificial Sequence: Synthetic oligonucleotide
  source 1..10: mol_type = other DNA
gacattacaa

SEQ ID NO: 628 - DNA, 10 bp, synthetic construct
  misc_feature 1..10: note = Description of Artificial Sequence: Synthetic oligonucleotide
  source 1..10: mol_type = other DNA
cagagagggc

SEQ ID NO: 629 - DNA, 10 bp, synthetic construct
  misc_feature 1..10: note = Description of Artificial Sequence: Synthetic oligonucleotide
  source 1..10: mol_type = other DNA
gcaccgcctc

SEQ ID NO: 630 - DNA, 10 bp, synthetic construct
  misc_feature 1..10: note = Description of Artificial Sequence: Synthetic oligonucleotide
  source 1..10: mol_type = other DNA
cggtccgagc

SEQ ID NO: 631 - DNA, 10 bp, synthetic construct
  misc_feature 1..10: note = Description of Artificial Sequence: Synthetic oligonucleotide
  source 1..10: mol_type = other DNA
tgtccggtgc

SEQ ID NO: 632 - DNA, 10 bp, synthetic construct
  misc_feature 1..10: note = Description of Artificial Sequence: Synthetic oligonucleotide
  source 1..10: mol_type = other DNA
ggtcggttgc

SEQ ID NO: 633 - DNA, 10 bp, synthetic construct
  misc_feature 1..10: note = Description of Artificial Sequence: Synthetic oligonucleotide
  source 1..10: mol_type = other DNA
gctcagctaa

SEQ ID NO: 634 - DNA, 10 bp, synthetic construct
  misc_feature 1..10: note = Description of Artificial Sequence: Synthetic oligonucleotide
  source 1..10: mol_type = other DNA
agcagttcgt

SEQ ID NO: 635 - DNA, 10 bp, synthetic construct
  misc_feature 1..10: note = Description of Artificial Sequence: Synthetic oligonucleotide
  source 1..10: mol_type = other DNA
aaatcgatga

SEQ ID NO: 636 - DNA, 10 bp, synthetic construct
  misc_feature 1..10: note = Description of Artificial Sequence: Synthetic oligonucleotide
  source 1..10: mol_type = other DNA
gctcggtatg

SEQ ID NO: 637 - DNA, 10 bp, synthetic construct
  misc_feature 1..10: note = Description of Artificial Sequence: Synthetic oligonucleotide
  source 1..10: mol_type = other DNA
cccgccgcgg

SEQ ID NO: 638 - DNA, 10 bp, synthetic construct
  misc_feature 1..10: note = Description of Artificial Sequence: Synthetic oligonucleotide
  source 1..10: mol_type = other DNA
gtgtgatagg

SEQ ID NO: 639 - DNA, 10 bp, synthetic construct
  misc_feature 1..10: note = Description of Artificial Sequence: Synthetic oligonucleotide
  source 1..10: mol_type = other DNA
ttggactcca

SEQ ID NO: 640 - DNA, 10 bp, synthetic construct
  misc_feature 1..10: note = Description of Artificial Sequence: Synthetic oligonucleotide
  source 1..10: mol_type = other DNA
tgcttatcta

SEQ ID NO: 641 - DNA, 10 bp, synthetic construct
  misc_feature 1..10: note = Description of Artificial Sequence: Synthetic oligonucleotide
  source 1..10: mol_type = other DNA
caaaaggcgt

SEQ ID NO: 642 - DNA, 10 bp, synthetic construct
  misc_feature 1..10: note = Description of Artificial Sequence: Synthetic oligonucleotide
  source 1..10: mol_type = other DNA
tagggggcct

SEQ ID NO: 643 - DNA, 10 bp, synthetic construct
  misc_feature 1..10: note = Description of Artificial Sequence: Synthetic oligonucleotide
  source 1..10: mol_type = other DNA
aagtattaat

SEQ ID NO: 644 - DNA, 10 bp, synthetic construct
  misc_feature 1..10: note = Description of Artificial Sequence: Synthetic oligonucleotide
  source 1..10: mol_type = other DNA
gtttagcccg

SEQ ID NO: 645 - DNA, 10 bp, synthetic construct
  misc_feature 1..10: note = Description of Artificial Sequence: Synthetic oligonucleotide
  source 1..10: mol_type = other DNA
cgctaatatg

SEQ ID NO: 646 - DNA, 10 bp, synthetic construct
  misc_feature 1..10: note = Description of Artificial Sequence: Synthetic oligonucleotide
  source 1..10: mol_type = other DNA
acaacacgtt

SEQ ID NO: 647 - DNA, 10 bp, synthetic construct
  misc_feature 1..10: note = Description of Artificial Sequence: Synthetic oligonucleotide
  source 1..10: mol_type = other DNA
agagatgctc

SEQ ID NO: 648 - DNA, 10 bp, synthetic construct
  misc_feature 1..10: note = Description of Artificial Sequence: Synthetic oligonucleotide
  source 1..10: mol_type = other DNA
tgcctgatat

SEQ ID NO: 649 - DNA, 10 bp, synthetic construct
  misc_feature 1..10: note = Description of Artificial Sequence: Synthetic oligonucleotide
  source 1..10: mol_type = other DNA
cttgtaagta

SEQ ID NO: 650 - DNA, 10 bp, synthetic construct
  misc_feature 1..10: note = Description of Artificial Sequence: Synthetic oligonucleotide
  source 1..10: mol_type = other DNA
catattgccg

SEQ ID NO: 651 - DNA, 10 bp, synthetic construct
  misc_feature 1..10: note = Description of Artificial Sequence: Synthetic oligonucleotide
  source 1..10: mol_type = other DNA
cttagaaagt

SEQ ID NO: 652 - DNA, 10 bp, synthetic construct
  misc_feature 1..10: note = Description of Artificial Sequence: Synthetic oligonucleotide
  source 1..10: mol_type = other DNA
atgttgtatt

SEQ ID NO: 653 - DNA, 10 bp, synthetic construct
  misc_feature 1..10: note = Description of Artificial Sequence: Synthetic oligonucleotide
  source 1..10: mol_type = other DNA
cgcattgaag

SEQ ID NO: 654 - DNA, 10 bp, synthetic construct
  misc_feature 1..10: note = Description of Artificial Sequence: Synthetic oligonucleotide
  source 1..10: mol_type = other DNA
ttatgttggt

SEQ ID NO: 655 - DNA, 10 bp, synthetic construct
  misc_feature 1..10: note = Description of Artificial Sequence: Synthetic oligonucleotide
  source 1..10: mol_type = other DNA
tcgcctcaga

SEQ ID NO: 656 - DNA, 10 bp, synthetic construct
  misc_feature 1..10: note = Description of Artificial Sequence: Synthetic oligonucleotide
  source 1..10: mol_type = other DNA
ttcgttgagg

SEQ ID NO: 657 - DNA, 10 bp, synthetic construct
  misc_feature 1..10: note = Description of Artificial Sequence: Synthetic oligonucleotide
  source 1..10: mol_type = other DNA
ggtgccgggc

SEQ ID NO: 658 - DNA, 10 bp, synthetic construct
  misc_feature 1..10: note = Description of Artificial Sequence: Synthetic oligonucleotide
  source 1..10: mol_type = other DNA
accattgtaa

SEQ ID NO: 659 - DNA, 10 bp, synthetic construct
  misc_feature 1..10: note = Description of Artificial Sequence: Synthetic oligonucleotide
  source 1..10: mol_type = other DNA
ttgattgtca

SEQ ID NO: 660 - DNA, 10 bp, synthetic construct
  misc_feature 1..10: note = Description of Artificial Sequence: Synthetic oligonucleotide
  source 1..10: mol_type = other DNA
cggctcacct

SEQ ID NO: 661 - DNA, 10 bp, synthetic construct
  misc_feature 1..10: note = Description of Artificial Sequence: Synthetic oligonucleotide
  source 1..10: mol_type = other DNA
ctatcacatg

SEQ ID NO: 662 - DNA, 10 bp, synthetic construct
  misc_feature 1..10: note = Description of Artificial Sequence: Synthetic oligonucleotide
  source 1..10: mol_type = other DNA
gtagacagaa

SEQ ID NO: 663 - DNA, 10 bp, synthetic construct
  misc_feature 1..10: note = Description of Artificial Sequence: Synthetic oligonucleotide
  source 1..10: mol_type = other DNA
cctttaccaa

SEQ ID NO: 664 - DNA, 10 bp, synthetic construct
  misc_feature 1..10: note = Description of Artificial Sequence: Synthetic oligonucleotide
  source 1..10: mol_type = other DNA
gcacatcgac

SEQ ID NO: 665 - DNA, 10 bp, synthetic construct
  misc_feature 1..10: note = Description of Artificial Sequence: Synthetic oligonucleotide
  source 1..10: mol_type = other DNA
tctcactttc

SEQ ID NO: 666 - DNA, 10 bp, synthetic construct
  misc_feature 1..10: note = Description of Artificial Sequence: Synthetic oligonucleotide
  source 1..10: mol_type = other DNA
ttcgagtact

SEQ ID NO: 667 - DNA, 10 bp, synthetic construct
  misc_feature 1..10: note = Description of Artificial Sequence: Synthetic oligonucleotide
  source 1..10: mol_type = other DNA
tagaagagca

SEQ ID NO: 668 - DNA, 10 bp, synthetic construct
  misc_feature 1..10: note = Description of Artificial Sequence: Synthetic oligonucleotide
  source 1..10: mol_type = other DNA
aaccccacca

SEQ ID NO: 669 - DNA, 10 bp, synthetic construct
  misc_feature 1..10: note = Description of Artificial Sequence: Synthetic oligonucleotide
  source 1..10: mol_type = other DNA
ctgtatcagt

SEQ ID NO: 670 - DNA, 10 bp, synthetic construct
  misc_feature 1..10: note = Description of Artificial Sequence: Synthetic oligonucleotide
  source 1..10: mol_type = other DNA
acataatgag

SEQ ID NO: 671 - DNA, 10 bp, synthetic construct
  misc_feature 1..10: note = Description of Artificial Sequence: Synthetic oligonucleotide
  source 1..10: mol_type = other DNA
agccttccgc

SEQ ID NO: 672 - DNA, 10 bp, synthetic construct
  misc_feature 1..10: note = Description of Artificial Sequence: Synthetic oligonucleotide
  source 1..10: mol_type = other DNA
cagtgctttt

SEQ ID NO: 673 - DNA, 10 bp, synthetic construct
  misc_feature 1..10: note = Description of Artificial Sequence: Synthetic oligonucleotide
  source 1..10: mol_type = other DNA
tagtccgtgt

SEQ ID NO: 674 - DNA, 10 bp, synthetic construct
  misc_feature 1..10: note = Description of Artificial Sequence: Synthetic oligonucleotide
  source 1..10: mol_type = other DNA
cggaatcggt

SEQ ID NO: 675 - DNA, 10 bp, synthetic construct
  misc_feature 1..10: note = Description of Artificial Sequence: Synthetic oligonucleotide
  source 1..10: mol_type = other DNA
cttgcggaga

SEQ ID NO: 676 - DNA, 10 bp, synthetic construct
  misc_feature 1..10: note = Description of Artificial Sequence: Synthetic oligonucleotide
  source 1..10: mol_type = other DNA
aaaaatttgg

SEQ ID NO: 677 - DNA, 10 bp, synthetic construct
  misc_feature 1..10: note = Description of Artificial Sequence: Synthetic oligonucleotide
  source 1..10: mol_type = other DNA
tgttttccgc

SEQ ID NO: 678 - DNA, 10 bp, synthetic construct
  misc_feature 1..10: note = Description of Artificial Sequence: Synthetic oligonucleotide
  source 1..10: mol_type = other DNA
atgctaggcg

SEQ ID NO: 679 - DNA, 10 bp, synthetic construct
  misc_feature 1..10: note = Description of Artificial Sequence: Synthetic oligonucleotide
  source 1..10: mol_type = other DNA
gactaatttc

SEQ ID NO: 680 - DNA, 10 bp, synthetic construct
  misc_feature 1..10: note = Description of Artificial Sequence: Synthetic oligonucleotide
  source 1..10: mol_type = other DNA
ctgtagtaac

SEQ ID NO: 681 - DNA, 10 bp, synthetic construct
  misc_feature 1..10: note = Description of Artificial Sequence: Synthetic oligonucleotide
  source 1..10: mol_type = other DNA
cggatgactt

SEQ ID NO: 682 - DNA, 10 bp, synthetic construct
  misc_feature 1..10: note = Description of Artificial Sequence: Synthetic oligonucleotide
  source 1..10: mol_type = other DNA
tcagagtgga

SEQ ID NO: 683 - DNA, 10 bp, synthetic construct
  misc_feature 1..10: note = Description of Artificial Sequence: Synthetic oligonucleotide
  source 1..10: mol_type = other DNA
caaaatagcg

SEQ ID NO: 684 - DNA, 10 bp, synthetic construct
  misc_feature 1..10: note = Description of Artificial Sequence: Synthetic oligonucleotide
  source 1..10: mol_type = other DNA
gaagaagaag

SEQ ID NO: 685 - DNA, 10 bp, synthetic construct
  misc_feature 1..10: note = Description of Artificial Sequence: Synthetic oligonucleotide
  source 1..10: mol_type = other DNA
cacccgcacg

SEQ ID NO: 686 - DNA, 10 bp, synthetic construct
  misc_feature 1..10: note = Description of Artificial Sequence: Synthetic oligonucleotide
  source 1..10: mol_type = other DNA
acgatgcccg

SEQ ID NO: 687 - DNA, 10 bp, synthetic construct
  misc_feature 1..10: note = Description of Artificial Sequence: Synthetic oligonucleotide
  source 1..10: mol_type = other DNA
cctactacac

SEQ ID NO: 688 - DNA, 10 bp, synthetic construct
  misc_feature 1..10: note = Description of Artificial Sequence: Synthetic oligonucleotide
  source 1..10: mol_type = other DNA
attgaaacaa

SEQ ID NO: 689 - DNA, 10 bp, synthetic construct
  misc_feature 1..10: note = Description of Artificial Sequence: Synthetic oligonucleotide
  source 1..10: mol_type = other DNA
gaccgaagat

SEQ ID NO: 690 - DNA, 10 bp, synthetic construct
  misc_feature 1..10: note = Description of Artificial Sequence: Synthetic oligonucleotide
  source 1..10: mol_type = other DNA
acggcctgaa

SEQ ID NO: 691 - DNA, 10 bp, synthetic construct
  misc_feature 1..10: note = Description of Artificial Sequence: Synthetic oligonucleotide
  source 1..10: mol_type = other DNA
aggggaggtc

SEQ ID NO: 692 - DNA, 10 bp, synthetic construct
  misc_feature 1..10: note = Description of Artificial Sequence: Synthetic oligonucleotide
  source 1..10: mol_type = other DNA
caatcaactt

SEQ ID NO: 693 - DNA, 10 bp, synthetic construct
  misc_feature 1..10: note = Description of Artificial Sequence: Synthetic oligonucleotide
  source 1..10: mol_type = other DNA
ggacaaccga

SEQ ID NO: 694 - DNA, 10 bp, synthetic construct
  misc_feature 1..10: note = Description of Artificial Sequence: Synthetic oligonucleotide
  source 1..10: mol_type = other DNA
tccctaaggc

SEQ ID NO: 695 - DNA, 10 bp, synthetic construct
  misc_feature 1..10: note = Description of Artificial Sequence: Synthetic oligonucleotide
  source 1..10: mol_type = other DNA
gttctacacg

SEQ ID NO: 696 - DNA, 10 bp, synthetic construct
  misc_feature 1..10: note = Description of Artificial Sequence: Synthetic oligonucleotide
  source 1..10: mol_type = other DNA
actaaccagt

SEQ ID NO: 697 - DNA, 10 bp, synthetic construct
  misc_feature 1..10: note = Description of Artificial Sequence: Synthetic oligonucleotide
  source 1..10: mol_type = other DNA
gaagctggat

SEQ ID NO: 698 - DNA, 10 bp, synthetic construct
  misc_feature 1..10: note = Description of Artificial Sequence: Synthetic oligonucleotide
  source 1..10: mol_type = other DNA
ggaaccatgg

SEQ ID NO: 699 - DNA, 10 bp, synthetic construct
  misc_feature 1..10: note = Description of Artificial Sequence: Synthetic oligonucleotide
  source 1..10: mol_type = other DNA
ctctacctgg

SEQ ID NO: 700 - DNA, 10 bp, synthetic construct
  misc_feature 1..10: note = Description of Artificial Sequence: Synthetic oligonucleotide
  source 1..10: mol_type = other DNA
taatgcctgc

SEQ ID NO: 701 - DNA, 10 bp, synthetic construct
  misc_feature 1..10: note = Description of Artificial Sequence: Synthetic oligonucleotide
  source 1..10: mol_type = other DNA
taaaggcaat

SEQ ID NO: 702 - DNA, 10 bp, synthetic construct
  misc_feature 1..10: note = Description of Artificial Sequence: Synthetic oligonucleotide
  source 1..10: mol_type = other DNA
cgcctgggaa

SEQ ID NO: 703 - DNA, 10 bp, synthetic construct
  misc_feature 1..10: note = Description of Artificial Sequence: Synthetic oligonucleotide
  source 1..10: mol_type = other DNA
tcttggggaa

SEQ ID NO: 704 - DNA, 10 bp, synthetic construct
  misc_feature 1..10: note = Description of Artificial Sequence: Synthetic oligonucleotide
  source 1..10: mol_type = other DNA
agagagagag

SEQ ID NO: 705 - DNA, 10 bp, synthetic construct
  misc_feature 1..10: note = Description of Artificial Sequence: Synthetic oligonucleotide
  source 1..10: mol_type = other DNA
gcgttggcgc

SEQ ID NO: 706 - DNA, 10 bp, synthetic construct
  misc_feature 1..10: note = Description of Artificial Sequence: Synthetic oligonucleotide
  source 1..10: mol_type = other DNA
ttacgacaga

SEQ ID NO: 707 - DNA, 10 bp, synthetic construct
  misc_feature 1..10: note = Description of Artificial Sequence: Synthetic oligonucleotide
  source 1..10: mol_type = other DNA
ggaactctta

SEQ ID NO: 708 - DNA, 10 bp, synthetic construct
  misc_feature 1..10: note = Description of Artificial Sequence: Synthetic oligonucleotide
  source 1..10: mol_type = other DNA
gattgtggag

SEQ ID NO: 709 - DNA, 10 bp, synthetic construct
  misc_feature 1..10: note = Description of Artificial Sequence: Synthetic oligonucleotide
  source 1..10: mol_type = other DNA
gggcactgat

SEQ ID NO: 710 - DNA, 10 bp, synthetic construct
  misc_feature 1..10: note = Description of Artificial Sequence: Synthetic oligonucleotide
  source 1..10: mol_type = other DNA
agacgcacca

SEQ ID NO: 711 - DNA, 10 bp, synthetic construct
  misc_feature 1..10: note = Description of Artificial Sequence: Synthetic oligonucleotide
  source 1..10: mol_type = other DNA
ccaattataa

SEQ ID NO: 712 - DNA, 10 bp, synthetic construct
  misc_feature 1..10: note = Description of Artificial Sequence: Synthetic oligonucleotide
  source 1..10: mol_type = other DNA
tagagacgca

SEQ ID NO: 713 - DNA, 10 bp, synthetic construct
  misc_feature 1..10: note = Description of Artificial Sequence: Synthetic oligonucleotide
  source 1..10: mol_type = other DNA
cctcttgtcg

SEQ ID NO: 714 - DNA, 10 bp, synthetic construct
  misc_feature 1..10: note = Description of Artificial Sequence: Synthetic oligonucleotide
  source 1..10: mol_type = other DNA
gaggaagctc

SEQ ID NO: 715 - DNA, 10 bp, synthetic construct
  misc_feature 1..10: note = Description of Artificial Sequence: Synthetic oligonucleotide
  source 1..10: mol_type = other DNA
agtcccgagt

SEQ ID NO: 716 - DNA, 10 bp, synthetic construct
  misc_feature 1..10: note = Description of Artificial Sequence: Synthetic oligonucleotide
  source 1..10: mol_type = other DNA
tgcttgcagt

SEQ ID NO: 717 - DNA, 10 bp, synthetic construct
  misc_feature 1..10: note = Description of Artificial Sequence: Synthetic oligonucleotide
  source 1..10: mol_type = other DNA
cccacttccc

SEQ ID NO: 718 - DNA, 10 bp, synthetic construct
  misc_feature 1..10: note = Description of Artificial Sequence: Synthetic oligonucleotide
  source 1..10: mol_type = other DNA
cgttgccgcg

SEQ ID NO: 719 - DNA, 10 bp, synthetic construct
  misc_feature 1..10: note = Description of Artificial Sequence: Synthetic oligonucleotide
  source 1..10: mol_type = other DNA
cccctggttc

SEQ ID NO: 720 - DNA, 10 bp, synthetic construct
  misc_feature 1..10: note = Description of Artificial Sequence: Synthetic oligonucleotide
  source 1..10: mol_type = other DNA
acgaccaata

SEQ ID NO: 721 - DNA, 10 bp, synthetic construct
  misc_feature 1..10: note = Description of Artificial Sequence: Synthetic oligonucleotide
  source 1..10: mol_type = other DNA
cttagggttc

SEQ ID NO: 722 - DNA, 10 bp, synthetic construct
  misc_feature 1..10: note = Description of Artificial Sequence: Synthetic oligonucleotide
  source 1..10: mol_type = other DNA
aaacatatca

SEQ ID NO: 723 - DNA, 10 bp, synthetic construct
  misc_feature 1..10: note = Description of Artificial Sequence: Synthetic oligonucleotide
  source 1..10: mol_type = other DNA
gggtcgtaga

SEQ ID NO: 724 - DNA, 10 bp, synthetic construct
  misc_feature 1..10: note = Description of Artificial Sequence: Synthetic oligonucleotide
  source 1..10: mol_type = other DNA
ctccgtagcg

SEQ ID NO: 725 - DNA, 10 bp, synthetic construct
  misc_feature 1..10: note = Description of Artificial Sequence: Synthetic oligonucleotide
  source 1..10: mol_type = other DNA
ctggtcataa

SEQ ID NO: 726 - DNA, 10 bp, synthetic construct
  misc_feature 1..10: note = Description of Artificial Sequence: Synthetic oligonucleotide
  source 1..10: mol_type = other DNA
ttgacagatc

SEQ ID NO: 727 - DNA, 10 bp, synthetic construct
  misc_feature 1..10: note = Description of Artificial Sequence: Synthetic oligonucleotide
  source 1..10: mol_type = other DNA
gagtaaagtc

SEQ ID NO: 728 - DNA, 10 bp, synthetic construct
  misc_feature 1..10: note = Description of Artificial Sequence: Synthetic oligonucleotide
  source 1..10: mol_type = other DNA
atatgggctt

SEQ ID NO: 729 - DNA, 10 bp, synthetic construct
  misc_feature 1..10: note = Description of Artificial Sequence: Synthetic oligonucleotide
  source 1..10: mol_type = other DNA
tacaactact

SEQ ID NO: 730 - DNA, 10 bp, synthetic construct
  misc_feature 1..10: note = Description of Artificial Sequence: Synthetic oligonucleotide
  source 1..10: mol_type = other DNA
aattcagccg

SEQ ID NO: 731 - DNA, 10 bp, synthetic construct
  misc_feature 1..10: note = Description of Artificial Sequence: Synthetic oligonucleotide
  source 1..10: mol_type = other DNA
gattgtacta

SEQ ID NO: 732 - DNA, 10 bp, synthetic construct
  misc_feature 1..10: note = Description of Artificial Sequence: Synthetic oligonucleotide
  source 1..10: mol_type = other DNA
tcgtaatgcg

SEQ ID NO: 733 - DNA, 10 bp, synthetic construct
  misc_feature 1..10: note = Description of Artificial Sequence: Synthetic oligonucleotide
  source 1..10: mol_type = other DNA
cgataactgc

SEQ ID NO: 734 - DNA, 10 bp, synthetic construct
  misc_feature 1..10: note = Description of Artificial Sequence: Synthetic oligonucleotide
  source 1..10: mol_type = other DNA
aacttggcgg

SEQ ID NO: 735 - DNA, 10 bp, synthetic construct
  misc_feature 1..10: note = Description of Artificial Sequence: Synthetic oligonucleotide
  source 1..10: mol_type = other DNA
cgtggatgta

SEQ ID NO: 736 - DNA, 10 bp, synthetic construct
  misc_feature 1..10: note = Description of Artificial Sequence: Synthetic oligonucleotide
  source 1..10: mol_type = other DNA
ccttcccgaa

SEQ ID NO: 737 - DNA, 10 bp, synthetic construct
  misc_feature 1..10: note = Description of Artificial Sequence: Synthetic oligonucleotide
  source 1..10: mol_type = other DNA
ctaaacccgt

SEQ ID NO: 738 - DNA, 10 bp, synthetic construct
  misc_feature 1..10: note = Description of Artificial Sequence: Synthetic oligonucleotide
  source 1..10: mol_type = other DNA
caacattccc

SEQ ID NO: 739 - DNA, 10 bp, synthetic construct
  misc_feature 1..10: note = Description of Artificial Sequence: Synthetic oligonucleotide
  source 1..10: mol_type = other DNA
cttaccctct

SEQ ID NO: 740 - DNA, 10 bp, synthetic construct
  misc_feature 1..10: note = Description of Artificial Sequence: Synthetic oligonucleotide
  source 1..10: mol_type = other DNA
ggaaagttct

SEQ ID NO: 741 - DNA, 10 bp, synthetic construct
  misc_feature 1..10: note = Description of Artificial Sequence: Synthetic oligonucleotide
  source 1..10: mol_type = other DNA
cggattggct

SEQ ID NO: 742 - DNA, 10 bp, synthetic construct
  misc_feature 1..10: note = Description of Artificial Sequence: Synthetic oligonucleotide
  source 1..10: mol_type = other DNA
aatgtagggc

SEQ ID NO: 743 - DNA, 10 bp, synthetic construct
  misc_feature 1..10: note = Description of Artificial Sequence: Synthetic oligonucleotide
  source 1..10: mol_type = other DNA
aatgaatcgc

SEQ ID NO: 744 - DNA, 10 bp, synthetic construct
  misc_feature 1..10: note = Description of Artificial Sequence: Synthetic oligonucleotide
  source 1..10: mol_type = other DNA
atcatacacc

SEQ ID NO: 745 - DNA, 10 bp, synthetic construct
  misc_feature 1..10: note = Description of Artificial Sequence: Synthetic oligonucleotide
  source 1..10: mol_type = other DNA
agttgggcag

SEQ ID NO: 746 - DNA, 10 bp, synthetic construct
  misc_feature 1..10: note = Description of Artificial Sequence: Synthetic oligonucleotide
  source 1..10: mol_type = other DNA
agaagaaggg

SEQ ID NO: 747 - DNA, 10 bp, synthetic construct
  misc_feature 1..10: note = Description of Artificial Sequence: Synthetic oligonucleotide
  source 1..10: mol_type = other DNA
gcgtgcgcta

SEQ ID NO: 748 - DNA, 10 bp, synthetic construct
  misc_feature 1..10: note = Description of Artificial Sequence: Synthetic oligonucleotide
  source 1..10: mol_type = other DNA
ccccgataaa

SEQ ID NO: 749 - DNA, 10 bp, synthetic construct
  misc_feature 1..10: note = Description of Artificial Sequence: Synthetic oligonucleotide
  source 1..10: mol_type = other DNA
taccaagtgc

SEQ ID NO: 750 - DNA, 10 bp, synthetic construct
  misc_feature 1..10: note = Description of Artificial Sequence: Synthetic oligonucleotide
  source 1..10: mol_type = other DNA
tgtgttttcg

SEQ ID NO: 751 - DNA, 10 bp, synthetic construct
  misc_feature 1..10: note = Description of Artificial Sequence: Synthetic oligonucleotide
  source 1..10: mol_type = other DNA
cccagatgtc

SEQ ID NO: 752 - DNA, 10 bp, synthetic construct
  misc_feature 1..10: note = Description of Artificial Sequence: Synthetic oligonucleotide
  source 1..10: mol_type = other DNA
gcgagcttcc

SEQ ID NO: 753 - DNA, 10 bp, synthetic construct
  misc_feature 1..10: note = Description of Artificial Sequence: Synthetic oligonucleotide
  source 1..10: mol_type = other DNA
gtgtcacgta

SEQ ID NO: 754 - DNA, 10 bp, synthetic construct
  misc_feature 1..10: note = Description of Artificial Sequence: Synthetic oligonucleotide
  source 1..10: mol_type = other DNA
ataggccgag

SEQ ID NO: 755 - DNA, 10 bp, synthetic construct
  misc_feature 1..10: note = Description of Artificial Sequence: Synthetic oligonucleotide
  source 1..10: mol_type = other DNA
gagctaccag

SEQ ID NO: 756 - DNA, 10 bp, synthetic construct
  misc_feature 1..10: note = Description of Artificial Sequence: Synthetic oligonucleotide
  source 1..10: mol_type = other DNA
cgcggcggag

SEQ ID NO: 757 - DNA, 10 bp, synthetic construct
  misc_feature 1..10: note = Description of Artificial Sequence: Synthetic oligonucleotide
  source 1..10: mol_type = other DNA
tcttgcacga

SEQ ID NO: 758 - DNA, 10 bp, synthetic construct
  misc_feature 1..10: note = Description of Artificial Sequence: Synthetic oligonucleotide
  source 1..10: mol_type = other DNA
tgccctaaag

SEQ ID NO: 759 - DNA, 10 bp, synthetic construct
  misc_feature 1..10: note = Description of Artificial Sequence: Synthetic oligonucleotide
  source 1..10: mol_type = other DNA
ttgcgctttg

SEQ ID NO: 760 - DNA, 10 bp, synthetic construct
  misc_feature 1..10: note = Description of Artificial Sequence: Synthetic oligonucleotide
  source 1..10: mol_type = other DNA
catataaagg

SEQ ID NO: 761 - DNA, 10 bp, synthetic construct
  misc_feature 1..10: note = Description of Artificial Sequence: Synthetic oligonucleotide
  source 1..10: mol_type = other DNA
aatagcgaat

SEQ ID NO: 762 - DNA, 10 bp, synthetic construct
  misc_feature 1..10: note = Description of Artificial Sequence: Synthetic oligonucleotide
  source 1..10: mol_type = other DNA
tacgctaagg

SEQ ID NO: 763 - DNA, 10 bp, synthetic construct
  misc_feature 1..10: note = Description of Artificial Sequence: Synthetic oligonucleotide
  source 1..10: mol_type = other DNA
acttagttcg

SEQ ID NO: 764 - DNA, 10 bp, synthetic construct
  misc_feature 1..10: note = Description of Artificial Sequence: Synthetic oligonucleotide
  source 1..10: mol_type = other DNA
cgtgcggaac

SEQ ID NO: 765 - DNA, 10 bp, synthetic construct
  misc_feature 1..10: note = Description of Artificial Sequence: Synthetic oligonucleotide
  source 1..10: mol_type = other DNA
acccgattcg

SEQ ID NO: 766 - DNA, 10 bp, synthetic construct
  misc_feature 1..10: note = Description of Artificial Sequence: Synthetic oligonucleotide
  source 1..10: mol_type = other DNA
tgcagagttt

SEQ ID NO: 767 - DNA, 10 bp, synthetic construct
  misc_feature 1..10: note = Description of Artificial Sequence: Synthetic oligonucleotide
  source 1..10: mol_type = other DNA
gaatcattag

SEQ ID NO: 768 - DNA, 10 bp, synthetic construct
  misc_feature 1..10: note = Description of Artificial Sequence: Synthetic oligonucleotide
  source 1..10: mol_type = other DNA
agtacactgg

SEQ ID NO: 769 - DNA, 10 bp, synthetic construct
  misc_feature 1..10: note = Description of Artificial Sequence: Synthetic oligonucleotide
  source 1..10: mol_type = other DNA
ttgtgcggtt

SEQ ID NO: 770 - DNA, 10 bp, synthetic construct
  misc_feature 1..10: note = Description of Artificial Sequence: Synthetic oligonucleotide
  source 1..10: mol_type = other DNA
atgacatgca

SEQ ID NO: 771 - DNA, 10 bp, synthetic construct
  misc_feature 1..10: note = Description of Artificial Sequence: Synthetic oligonucleotide
  source 1..10: mol_type = other DNA
ttctcggacg

SEQ ID NO: 772 - DNA, 10 bp, synthetic construct
  misc_feature 1..10: note = Description of Artificial Sequence: Synthetic oligonucleotide
  source 1..10: mol_type = other DNA
agattgaaga

SEQ ID NO: 773 - DNA, 10 bp, synthetic construct
  misc_feature 1..10: note = Description of Artificial Sequence: Synthetic oligonucleotide
  source 1..10: mol_type = other DNA
ggcggactgt

SEQ ID NO: 774 - DNA, 10 bp, synthetic construct
  misc_feature 1..10: note = Description of Artificial Sequence: Synthetic oligonucleotide
  source 1..10: mol_type = other DNA
tttatggtaa

SEQ ID NO: 775 - DNA, 10 bp, synthetic construct
  misc_feature 1..10: note = Description of Artificial Sequence: Synthetic oligonucleotide
  source 1..10: mol_type = other DNA
cagtagggtg

SEQ ID NO: 776 - DNA, 10 bp, synthetic construct
  misc_feature 1..10: note = Description of Artificial Sequence: Synthetic oligonucleotide
  source 1..10: mol_type = other DNA
gacaggcaag

SEQ ID NO: 777 - DNA, 10 bp, synthetic construct
  misc_feature 1..10: note = Description of Artificial Sequence: Synthetic oligonucleotide
  source 1..10: mol_type = other DNA
gatgtgtcgt

SEQ ID NO: 778 - DNA, 10 bp, synthetic construct
  misc_feature 1..10: note = Description of Artificial Sequence: Synthetic oligonucleotide
  source 1..10: mol_type = other DNA
acttgacgga

SEQ ID NO: 779 - DNA, 10 bp, synthetic construct
  misc_feature 1..10: note = Description of Artificial Sequence: Synthetic oligonucleotide
  source 1..10: mol_type = other DNA
aagtccgaaa

SEQ ID NO: 780 - DNA, 10 bp, synthetic construct
  misc_feature 1..10: note = Description of Artificial Sequence: Synthetic oligonucleotide
  source 1..10: mol_type = other DNA
tgggtgtagg

SEQ ID NO: 781 - DNA, 10 bp, synthetic construct
  misc_feature 1..10: note = Description of Artificial Sequence: Synthetic oligonucleotide
  source 1..10: mol_type = other DNA
acttaccgcg

SEQ ID NO: 782 - DNA, 10 bp, synthetic construct
  misc_feature 1..10: note = Description of Artificial Sequence: Synthetic oligonucleotide
  source 1..10: mol_type = other DNA
ctgtgcaccc

SEQ ID NO: 783 - DNA, 10 bp, synthetic construct
  misc_feature 1..10: note = Description of Artificial Sequence: Synthetic oligonucleotide
  source 1..10: mol_type = other DNA
attgctctct

SEQ ID NO: 784 - DNA, 10 bp, synthetic construct
  misc_feature 1..10: note = Description of Artificial Sequence: Synthetic oligonucleotide
  source 1..10: mol_type = other DNA
cagaagacaa

SEQ ID NO: 785 - DNA, 10 bp, synthetic construct
  misc_feature 1..10: note = Description of Artificial Sequence: Synthetic oligonucleotide
  source 1..10: mol_type = other DNA
ttacgctata

SEQ ID NO: 786 - DNA, 10 bp, synthetic construct
  misc_feature 1..10: note = Description of Artificial Sequence: Synthetic oligonucleotide
  source 1..10: mol_type = other DNA
acgtggaaat

SEQ ID NO: 787 - DNA, 10 bp, synthetic construct
  misc_feature 1..10: note = Description of Artificial Sequence: Synthetic oligonucleotide
  source 1..10: mol_type = other DNA
tgaggctggt

SEQ ID NO: 788 - DNA, 10 bp, synthetic construct
  misc_feature 1..10: note = Description of Artificial Sequence: Synthetic oligonucleotide
  source 1..10: mol_type = other DNA
attatgagat

SEQ ID NO: 789 - DNA, 10 bp, synthetic construct
  misc_feature 1..10: note = Description of Artificial Sequence: Synthetic oligonucleotide
  source 1..10: mol_type = other DNA
gacttgtagt

SEQ ID NO: 790 - DNA, 10 bp, synthetic construct
  misc_feature 1..10: note = Description of Artificial Sequence: Synthetic oligonucleotide
  source 1..10: mol_type = other DNA
tcgctgagga

SEQ ID NO: 791 - DNA, 10 bp, synthetic construct
  misc_feature 1..10: note = Description of Artificial Sequence: Synthetic oligonucleotide
  source 1..10: mol_type = other DNA
cccaactcta

SEQ ID NO: 792 - DNA, 10 bp, synthetic construct
  misc_feature 1..10: note = Description of Artificial Sequence: Synthetic oligonucleotide
  source 1..10: mol_type = other DNA
gatagggagg

SEQ ID NO: 793 - DNA, 10 bp, synthetic construct
  misc_feature 1..10: note = Description of Artificial Sequence: Synthetic oligonucleotide
  source 1..10: mol_type = other DNA
tagaaatcag

SEQ ID NO: 794 - DNA, 10 bp, synthetic construct
  misc_feature 1..10: note = Description of Artificial Sequence: Synthetic oligonucleotide
  source 1..10: mol_type = other DNA
gtcgctagaa

SEQ ID NO: 795 - DNA, 10 bp, synthetic construct
  misc_feature 1..10: note = Description of Artificial Sequence: Synthetic oligonucleotide
  source 1..10: mol_type = other DNA
aaaatagaaa

SEQ ID NO: 796 - DNA, 10 bp, synthetic construct
  misc_feature 1..10: note = Description of Artificial Sequence: Synthetic oligonucleotide
  source 1..10: mol_type = other DNA
gctcctgggt

SEQ ID NO: 797 - DNA, 10 bp, synthetic construct
  misc_feature 1..10: note = Description of Artificial Sequence: Synthetic oligonucleotide
  source 1..10: mol_type = other DNA
cgcgctcgcg

SEQ ID NO: 798 - DNA, 10 bp, synthetic construct
  misc_feature 1..10: note = Description of Artificial Sequence: Synthetic oligonucleotide
  source 1..10: mol_type = other DNA
ggcaaacgca

SEQ ID NO: 799 - DNA, 10 bp, synthetic construct
  misc_feature 1..10: note = Description of Artificial Sequence: Synthetic oligonucleotide
  source 1..10: mol_type = other DNA
tttactacct

SEQ ID NO: 800 - DNA, 10 bp, synthetic construct
  misc_feature 1..10: note = Description of Artificial Sequence: Synthetic oligonucleotide
  source 1..10: mol_type = other DNA
atcctaaact

SEQ ID NO: 801 - DNA, 10 bp, synthetic construct
  misc_feature 1..10: note = Description of Artificial Sequence: Synthetic oligonucleotide
  source 1..10: mol_type = other DNA
ctccgtatgt

SEQ ID NO: 802 - DNA, 10 bp, synthetic construct
  misc_feature 1..10: note = Description of Artificial Sequence: Synthetic oligonucleotide
  source 1..10: mol_type = other DNA
tatcgtccag

SEQ ID NO: 803 - DNA, 10 bp, synthetic construct
  misc_feature 1..10: note = Description of Artificial Sequence: Synthetic oligonucleotide
  source 1..10: mol_type = other DNA
gccggcggta

SEQ ID NO: 804 - DNA, 10 bp, synthetic construct
  misc_feature 1..10: note = Description of Artificial Sequence: Synthetic oligonucleotide
  source 1..10: mol_type = other DNA
tgctccattt

SEQ ID NO: 805 - DNA, 10 bp, synthetic construct
  misc_feature 1..10: note = Description of Artificial Sequence: Synthetic oligonucleotide
  source 1..10: mol_type = other DNA
tggctgttgt

SEQ ID NO: 806 - DNA, 10 bp, synthetic construct
  misc_feature 1..10: note = Description of Artificial Sequence: Synthetic oligonucleotide
  source 1..10: mol_type = other DNA
tactgcgcaa

SEQ ID NO: 807 - DNA, 10 bp, synthetic construct
  misc_feature 1..10: note = Description of Artificial Sequence: Synthetic oligonucleotide
  source 1..10: mol_type = other DNA
tatacggctt

SEQ ID NO: 808 - DNA, 10 bp, synthetic construct
  misc_feature 1..10: note = Description of Artificial Sequence: Synthetic oligonucleotide
  source 1..10: mol_type = other DNA
ggttattacc

SEQ ID NO: 809 - DNA, 10 bp, synthetic construct
  misc_feature 1..10: note = Description of Artificial Sequence: Synthetic oligonucleotide
  source 1..10: mol_type = other DNA
atcaggagga

SEQ ID NO: 810 - DNA, 10 bp, synthetic construct
  misc_feature 1..10: note = Description of Artificial Sequence: Synthetic oligonucleotide
  source 1..10: mol_type = other DNA
ctattgccag

SEQ ID NO: 811 - DNA, 10 bp, synthetic construct
  misc_feature 1..10: note = Description of Artificial Sequence: Synthetic oligonucleotide
  source 1..10: mol_type = other DNA
acgtacacac

SEQ ID NO: 812 - DNA, 10 bp, synthetic construct
  misc_feature 1..10: note = Description of Artificial Sequence: Synthetic oligonucleotide
  source 1..10: mol_type = other DNA
cagcctagct

SEQ ID NO: 813 - DNA, 10 bp, synthetic construct
  misc_feature 1..10: note = Description of Artificial Sequence: Synthetic oligonucleotide
  source 1..10: mol_type = other DNA
gaaaaacaac

SEQ ID NO: 814 - DNA, 10 bp, synthetic construct
  misc_feature 1..10: note = Description of Artificial Sequence: Synthetic oligonucleotide
  source 1..10: mol_type = other DNA
cgttcagtta

SEQ ID NO: 815 - DNA, 10 bp, synthetic construct
  misc_feature 1..10: note = Description of Artificial Sequence: Synthetic oligonucleotide
  source 1..10: mol_type = other DNA
caatcagaat

SEQ ID NO: 816 - DNA, 10 bp, synthetic construct
  misc_feature 1..10: note = Description of Artificial Sequence: Synthetic oligonucleotide
  source 1..10: mol_type = other DNA
gggctactct

SEQ ID NO: 817 - DNA, 10 bp, synthetic construct
  misc_feature 1..10: note = Description of Artificial Sequence: Synthetic oligonucleotide
  source 1..10: mol_type = other DNA
ccccattggg

SEQ ID NO: 818 - DNA, 10 bp, synthetic construct
  misc_feature 1..10: note = Description of Artificial Sequence: Synthetic oligonucleotide
  source 1..10: mol_type = other DNA
tagggaacgg

SEQ ID NO: 819 - DNA, 10 bp, synthetic construct
  misc_feature 1..10: note = Description of Artificial Sequence: Synthetic oligonucleotide
  source 1..10: mol_type = other DNA
cagctgatac

SEQ ID NO: 820 - DNA, 10 bp, synthetic construct
  misc_feature 1..10: note = Description of Artificial Sequence: Synthetic oligonucleotide
  source 1..10: mol_type = other DNA
attcctgtga

SEQ ID NO: 821 - DNA, 10 bp, synthetic construct
  misc_feature 1..10: note = Description of Artificial Sequence: Synthetic oligonucleotide
  source 1..10: mol_type = other DNA
tcagagccgt

SEQ ID NO: 822 - DNA, 10 bp, synthetic construct
  misc_feature 1..10: note = Description of Artificial Sequence: Synthetic oligonucleotide
  source 1..10: mol_type = other DNA
catgaaaagc

SEQ ID NO: 823 - DNA, 10 bp, synthetic construct
  misc_feature 1..10: note = Description of Artificial Sequence: Synthetic oligonucleotide
  source 1..10: mol_type = other DNA
tgacctgtga

SEQ ID NO: 824 - DNA, 10 bp, synthetic construct
  misc_feature 1..10: note = Description of Artificial Sequence: Synthetic oligonucleotide
  source 1..10: mol_type = other DNA
gcattagcag

SEQ ID NO: 825 - DNA, 10 bp, synthetic construct
  misc_feature 1..10: note = Description of Artificial Sequence: Synthetic oligonucleotide
  source 1..10: mol_type = other DNA
gacagaacca

SEQ ID NO: 826 - DNA, 10 bp, synthetic construct
  misc_feature 1..10: note = Description of Artificial Sequence: Synthetic oligonucleotide
  source 1..10: mol_type = other DNA
tccagtatat

SEQ ID NO: 827 - DNA, 10 bp, synthetic construct
  misc_feature 1..10: note = Description of Artificial Sequence: Synthetic oligonucleotide
  source 1..10: mol_type = other DNA
tgttccgcta

SEQ ID NO: 828 - DNA, 10 bp, synthetic construct
  misc_feature 1..10: note = Description of Artificial Sequence: Synthetic oligonucleotide
  source 1..10: mol_type = other DNA
gatatccatt

SEQ ID NO: 829 - DNA, 10 bp, synthetic construct
  misc_feature 1..10: note = Description of Artificial Sequence: Synthetic oligonucleotide
  source 1..10: mol_type = other DNA
catatggacc

SEQ ID NO: 830 - DNA, 10 bp, synthetic construct
  misc_feature 1..10: note = Description of Artificial Sequence: Synthetic oligonucleotide
  source 1..10: mol_type = other DNA
gatatagtaa

SEQ ID NO: 831 - DNA, 10 bp, synthetic construct
  misc_feature 1..10: note = Description of Artificial Sequence: Synthetic oligonucleotide
  source 1..10: mol_type = other DNA
cacctttttt

SEQ ID NO: 832 - DNA, 10 bp, synthetic construct
  misc_feature 1..10: note = Description of Artificial Sequence: Synthetic oligonucleotide
  source 1..10: mol_type = other DNA
agcttgcggg

SEQ ID NO: 833 - DNA, 10 bp, synthetic construct
  misc_feature 1..10: note = Description of Artificial Sequence: Synthetic oligonucleotide
  source 1..10: mol_type = other DNA
cgcacaggga

SEQ ID NO: 834 - DNA, 10 bp, synthetic construct
  misc_feature 1..10: note = Description of Artificial Sequence: Synthetic oligonucleotide
  source 1..10: mol_type = other DNA
tctgggtgct

SEQ ID NO: 835 - DNA, 10 bp, synthetic construct
  misc_feature 1..10: note = Description of Artificial Sequence: Synthetic oligonucleotide
  source 1..10: mol_type = other DNA
tgagtcgttt

SEQ ID NO: 836 - DNA, 10 bp, synthetic construct
  misc_feature 1..10: note = Description of Artificial Sequence: Synthetic oligonucleotide
  source 1..10: mol_type = other DNA
ttacaatgtg

SEQ ID NO: 837 - DNA, 10 bp, synthetic construct
  misc_feature 1..10: note = Description of Artificial Sequence: Synthetic oligonucleotide
  source 1..10: mol_type = other DNA
cttgcaaaca

SEQ ID NO: 838 - DNA, 10 bp, synthetic construct
  misc_feature 1..10: note = Description of Artificial Sequence: Synthetic oligonucleotide
  source 1..10: mol_type = other DNA
tgtcgagctg

SEQ ID NO: 839 - DNA, 10 bp, synthetic construct
  misc_feature 1..10: note = Description of Artificial Sequence: Synthetic oligonucleotide
  source 1..10: mol_type = other DNA
actttaacct

SEQ ID NO: 840 - DNA, 10 bp, synthetic construct
  misc_feature 1..10: note = Description of Artificial Sequence: Synthetic oligonucleotide
  source 1..10: mol_type = other DNA
atataagtgc

SEQ ID NO: 841 - DNA, 10 bp, synthetic construct
  misc_feature 1..10: note = Description of Artificial Sequence: Synthetic oligonucleotide
  source 1..10: mol_type = other DNA
ggaagggcgt

SEQ ID NO: 842 - DNA, 10 bp, synthetic construct
  misc_feature 1..10: note = Description of Artificial Sequence: Synthetic oligonucleotide
  source 1..10: mol_type = other DNA
tttgacttga

SEQ ID NO: 843 - DNA, 10 bp, synthetic construct
  misc_feature 1..10: note = Description of Artificial Sequence: Synthetic oligonucleotide
  source 1..10: mol_type = other DNA
gtataaacgg

SEQ ID NO: 844 - DNA, 10 bp, synthetic construct
  misc_feature 1..10: note = Description of Artificial Sequence: Synthetic oligonucleotide
  source 1..10: mol_type = other DNA
taaccggatg

SEQ ID NO: 845 - DNA, 10 bp, synthetic construct
  misc_feature 1..10: note = Description of Artificial Sequence: Synthetic oligonucleotide
  source 1..10: mol_type = other DNA
ttctcatcag

SEQ ID NO: 846 - DNA, 10 bp, synthetic construct
  misc_feature 1..10: note = Description of Artificial Sequence: Synthetic oligonucleotide
  source 1..10: mol_type = other DNA
ctcggttacg

SEQ ID NO: 847 - DNA, 10 bp, synthetic construct
  misc_feature 1..10: note = Description of Artificial Sequence: Synthetic oligonucleotide
  source 1..10: mol_type = other DNA
atatggttct

SEQ ID NO: 848 - DNA, 10 bp, synthetic construct
  misc_feature 1..10: note = Description of Artificial Sequence: Synthetic oligonucleotide
  source 1..10: mol_type = other DNA
cgcccccgaa

SEQ ID NO: 849 - DNA, 10 bp, synthetic construct
  misc_feature 1..10: note = Description of Artificial Sequence: Synthetic oligonucleotide
  source 1..10: mol_type = other DNA
acctcgatcg

SEQ ID NO: 850 - DNA, 10 bp, synthetic construct
  misc_feature 1..10: note = Description of Artificial Sequence: Synthetic oligonucleotide
  source 1..10: mol_type = other DNA
ctcgaataat

SEQ ID NO: 851 - DNA, 10 bp, synthetic construct
  misc_feature 1..10: note = Description of Artificial Sequence: Synthetic oligonucleotide
  source 1..10: mol_type = other DNA
gcccgagctt

SEQ ID NO: 852 - DNA, 10 bp, synthetic construct
  misc_feature 1..10: note = Description of Artificial Sequence: Synthetic oligonucleotide
  source 1..10: mol_type = other DNA
aacagtcaac

SEQ ID NO: 853 - DNA, 10 bp, synthetic construct
  misc_feature 1..10: note = Description of Artificial Sequence: Synthetic oligonucleotide
  source 1..10: mol_type = other DNA
ctggaacctc

SEQ ID NO: 854 - DNA, 10 bp, synthetic construct
  misc_feature 1..10: note = Description of Artificial Sequence: Synthetic oligonucleotide
  source 1..10: mol_type = other DNA
aataacgggg

SEQ ID NO: 855 - DNA, 10 bp, synthetic construct
  misc_feature 1..10: note = Description of Artificial Sequence: Synthetic oligonucleotide
  source 1..10: mol_type = other DNA
acgccccact

SEQ ID NO: 856 - DNA, 10 bp, synthetic construct
  misc_feature 1..10: note = Description of Artificial Sequence: Synthetic oligonucleotide
  source 1..10: mol_type = other DNA
ggcaacatga

SEQ ID NO: 857 - DNA, 10 bp, synthetic construct
  misc_feature 1..10: note = Description of Artificial Sequence: Synthetic oligonucleotide
  source 1..10: mol_type = other DNA
gctatttcgc

SEQ ID NO: 858 - DNA, 10 bp, synthetic construct
  misc_feature 1..10: note = Description of Artificial Sequence: Synthetic oligonucleotide
  source 1..10: mol_type = other DNA
ttccacttta

SEQ ID NO: 859 - DNA, 10 bp, synthetic construct
  misc_feature 1..10: note = Description of Artificial Sequence: Synthetic oligonucleotide
  source 1..10: mol_type = other DNA
gccgatggat

SEQ ID NO: 860 - DNA, 10 bp, synthetic construct
  misc_feature 1..10: note = Description of Artificial Sequence: Synthetic oligonucleotide
  source 1..10: mol_type = other DNA
aagttggtaa

SEQ ID NO: 861 - DNA, 10 bp, synthetic construct
  misc_feature 1..10: note = Description of Artificial Sequence: Synthetic oligonucleotide
  source 1..10: mol_type = other DNA
cactagctag

SEQ ID NO: 862 - DNA, 10 bp, synthetic construct
  misc_feature 1..10: note = Description of Artificial Sequence: Synthetic oligonucleotide
  source 1..10: mol_type = other DNA
acatgcccct

SEQ ID NO: 863 - DNA, 10 bp, synthetic construct
  misc_feature 1..10: note = Description of Artificial Sequence: Synthetic oligonucleotide
  source 1..10: mol_type = other DNA
ttcattactc

SEQ ID NO: 864 - DNA, 10 bp, synthetic construct
  misc_feature 1..10: note = Description of Artificial Sequence: Synthetic oligonucleotide
  source 1..10: mol_type = other DNA
ggtttaatat

SEQ ID NO: 865 - DNA, 10 bp, synthetic construct
  misc_feature 1..10: note = Description of Artificial Sequence: Synthetic oligonucleotide
  source 1..10: mol_type = other DNA
cctgcagtga

SEQ ID NO: 866 - DNA, 10 bp, synthetic construct
  misc_feature 1..10: note = Description of Artificial Sequence: Synthetic oligonucleotide
  source 1..10: mol_type = other DNA
tctttaagtt

SEQ ID NO: 867 - DNA, 10 bp, synthetic construct
  misc_feature 1..10: note = Description of Artificial Sequence: Synthetic oligonucleotide
  source 1..10: mol_type = other DNA
tggcgatcga

SEQ ID NO: 868 - DNA, 10 bp, synthetic construct
  misc_feature 1..10: note = Description of Artificial Sequence: Synthetic oligonucleotide
  source 1..10: mol_type = other DNA
ctttttagct

SEQ ID NO: 869 - DNA, 10 bp, synthetic construct
  misc_feature 1..10: note = Description of Artificial Sequence: Synthetic oligonucleotide
  source 1..10: mol_type = other DNA
cccagtctct

SEQ ID NO: 870 - DNA, 10 bp, synthetic construct
  misc_feature 1..10: note = Description of Artificial Sequence: Synthetic oligonucleotide
  source 1..10: mol_type = other DNA
aaatgtttcg

SEQ ID NO: 871 - DNA, 10 bp, synthetic construct
  misc_feature 1..10: note = Description of Artificial Sequence: Synthetic oligonucleotide
  source 1..10: mol_type = other DNA
atataagacg

SEQ ID NO: 872 - DNA, 10 bp, synthetic construct
  misc_feature 1..10: note = Description of Artificial Sequence: Synthetic oligonucleotide
  source 1..10: mol_type = other DNA
tcactttaca

SEQ ID NO: 873 - DNA, 10 bp, synthetic construct
  misc_feature 1..10: note = Description of Artificial Sequence: Synthetic oligonucleotide
  source 1..10: mol_type = other DNA
cctggcgccc

SEQ ID NO: 874 - DNA, 10 bp, synthetic construct
  misc_feature 1..10: note = Description of Artificial Sequence: Synthetic oligonucleotide
  source 1..10: mol_type = other DNA
ggattactgg

SEQ ID NO: 875 - DNA, 10 bp, synthetic construct
  misc_feature 1..10: note = Description of Artificial Sequence: Synthetic oligonucleotide
  source 1..10: mol_type = other DNA
gaatgatctt

SEQ ID NO: 876 - DNA, 10 bp, synthetic construct
  misc_feature 1..10: note = Description of Artificial Sequence: Synthetic oligonucleotide
  source 1..10: mol_type = other DNA
gctcggatcg

SEQ ID NO: 877 - DNA, 10 bp, synthetic construct
  misc_feature 1..10: note = Description of Artificial Sequence: Synthetic oligonucleotide
  source 1..10: mol_type = other DNA
cagctgcgag

SEQ ID NO: 878 - DNA, 10 bp, synthetic construct
  misc_feature 1..10: note = Description of Artificial Sequence: Synthetic oligonucleotide
  source 1..10: mol_type = other DNA
acccttacta

SEQ ID NO: 879 - DNA, 10 bp, synthetic construct
  misc_feature 1..10: note = Description of Artificial Sequence: Synthetic oligonucleotide
  source 1..10: mol_type = other DNA
aggtgaaact

SEQ ID NO: 880 - DNA, 10 bp, synthetic construct
  misc_feature 1..10: note = Description of Artificial Sequence: Synthetic oligonucleotide
  source 1..10: mol_type = other DNA
cgaatttgat

SEQ ID NO: 881 - DNA, 10 bp, synthetic construct
  misc_feature 1..10: note = Description of Artificial Sequence: Synthetic oligonucleotide
  source 1..10: mol_type = other DNA
cgctgtgcgg

SEQ ID NO: 882 - DNA, 10 bp, synthetic construct
  misc_feature 1..10: note = Description of Artificial Sequence: Synthetic oligonucleotide
  source 1..10: mol_type = other DNA
ttaccgcacc

SEQ ID NO: 883 - DNA, 10 bp, synthetic construct
  misc_feature 1..10: note = Description of Artificial Sequence: Synthetic oligonucleotide
  source 1..10: mol_type = other DNA
ggaatcttaa

SEQ ID NO: 884 - DNA, 10 bp, synthetic construct
  misc_feature 1..10: note = Description of Artificial Sequence: Synthetic oligonucleotide
  source 1..10: mol_type = other DNA
ctcaacaccc

SEQ ID NO: 885 - DNA, 10 bp, synthetic construct
  misc_feature 1..10: note = Description of Artificial Sequence: Synthetic oligonucleotide
  source 1..10: mol_type = other DNA
cgtgcccttg

SEQ ID NO: 886 - DNA, 10 bp, synthetic construct
  misc_feature 1..10: note = Description of Artificial Sequence: Synthetic oligonucleotide
  source 1..10: mol_type = other DNA
gcaggctcga

SEQ ID NO: 887 - DNA, 10 bp, synthetic construct
  misc_feature 1..10: note = Description of Artificial Sequence: Synthetic oligonucleotide
  source 1..10: mol_type = other DNA
accaacgaag

SEQ ID NO: 888 - DNA, 10 bp, synthetic construct
  misc_feature 1..10: note = Description of Artificial Sequence: Synthetic oligonucleotide
  source 1..10: mol_type = other DNA
cctgtaattt

SEQ ID NO: 889 - DNA, 10 bp, synthetic construct
  misc_feature 1..10: note = Description of Artificial Sequence: Synthetic oligonucleotide
  source 1..10: mol_type = other DNA
gggtgggatg

SEQ ID NO: 890 - DNA, 10 bp, synthetic construct
  misc_feature 1..10: note = Description of Artificial Sequence: Synthetic oligonucleotide
  source 1..10: mol_type = other DNA
ttgctcaccg

SEQ ID NO: 891 - DNA, 10 bp, synthetic construct
  misc_feature 1..10: note = Description of Artificial Sequence: Synthetic oligonucleotide
  source 1..10: mol_type = other DNA
ttacgaccac

SEQ ID NO: 892 - DNA, 10 bp, synthetic construct
  misc_feature 1..10: note = Description of Artificial Sequence: Synthetic oligonucleotide
  source 1..10: mol_type = other DNA
ttttctaacc

SEQ ID NO: 893 - DNA, 10 bp, synthetic construct
  misc_feature 1..10: note = Description of Artificial Sequence: Synthetic oligonucleotide
  source 1..10: mol_type = other DNA
gctttagata

SEQ ID NO: 894 - DNA, 10 bp, synthetic construct
  misc_feature 1..10: note = Description of Artificial Sequence: Synthetic oligonucleotide
  source 1..10: mol_type = other DNA
cacgtattgg

SEQ ID NO: 895 - DNA, 10 bp, synthetic construct
  misc_feature 1..10: note = Description of Artificial Sequence: Synthetic oligonucleotide
  source 1..10: mol_type = other DNA
aaatatctcc

SEQ ID NO: 896 - DNA, 10 bp, synthetic construct
  misc_feature 1..10: note = Description of Artificial Sequence: Synthetic oligonucleotide
  source 1..10: mol_type = other DNA
gctggaaaac

SEQ ID NO: 897 - DNA, 10 bp, synthetic construct
  misc_feature 1..10: note = Description of Artificial Sequence: Synthetic oligonucleotide
  source 1..10: mol_type = other DNA
gagcgcatta

SEQ ID NO: 898 - DNA, 10 bp, synthetic construct
  misc_feature 1..10: note = Description of Artificial Sequence: Synthetic oligonucleotide
  source 1..10: mol_type = other DNA
gtggaggggt

SEQ ID NO: 899 - DNA, 10 bp, synthetic construct
  misc_feature 1..10: note = Description of Artificial Sequence: Synthetic oligonucleotide
  source 1..10: mol_type = other DNA
tccactggga

SEQ ID NO: 900 - DNA, 10 bp, synthetic construct
  misc_feature 1..10: note = Description of Artificial Sequence: Synthetic oligonucleotide
  source 1..10: mol_type = other DNA
caatagcgga

SEQ ID NO: 901 - DNA, 10 bp, synthetic construct
  misc_feature 1..10: note = Description of Artificial Sequence: Synthetic oligonucleotide
  source 1..10: mol_type = other DNA
catctagttt

SEQ ID NO: 902 - DNA, 10 bp, synthetic construct
  misc_feature 1..10: note = Description of Artificial Sequence: Synthetic oligonucleotide
  source 1..10: mol_type = other DNA
gaagttccgg

SEQ ID NO: 903 - DNA, 10 bp, synthetic construct
  misc_feature 1..10: note = Description of Artificial Sequence: Synthetic oligonucleotide
  source 1..10: mol_type = other DNA
agcgagattc

SEQ ID NO: 904 - DNA, 10 bp, synthetic construct
  misc_feature 1..10: note = Description of Artificial Sequence: Synthetic oligonucleotide
  source 1..10: mol_type = other DNA
ttaaggtcgg

SEQ ID NO: 905 - DNA, 10 bp, synthetic construct
  misc_feature 1..10: note = Description of Artificial Sequence: Synthetic oligonucleotide
  source 1..10: mol_type = other DNA
aatggttagg

SEQ ID NO: 906 - DNA, 10 bp, synthetic construct
  misc_feature 1..10: note = Description of Artificial Sequence: Synthetic oligonucleotide
  source 1..10: mol_type = other DNA
cgttattata

SEQ ID NO: 907 - DNA, 10 bp, synthetic construct
  misc_feature 1..10: note = Description of Artificial Sequence: Synthetic oligonucleotide
  source 1..10: mol_type = other DNA
acggaaagga

SEQ ID NO: 908 - DNA, 10 bp, synthetic construct
  misc_feature 1..10: note = Description of Artificial Sequence: Synthetic oligonucleotide
  source 1..10: mol_type = other DNA
ccttgtcccg

SEQ ID NO: 909 - DNA, 10 bp, synthetic construct
  misc_feature 1..10: note = Description of Artificial Sequence: Synthetic oligonucleotide
  source 1..10: mol_type = other DNA
atactttttt

SEQ ID NO: 910 - DNA, 10 bp, synthetic construct
  misc_feature 1..10: note = Description of Artificial Sequence: Synthetic oligonucleotide
  source 1..10: mol_type = other DNA
ctgggtctgg

SEQ ID NO: 911 - DNA, 10 bp, synthetic construct
  misc_feature 1..10: note = Description of Artificial Sequence: Synthetic oligonucleotide
  source 1..10: mol_type = other DNA
aaccattgcg

SEQ ID NO: 912 - DNA, 10 bp, synthetic construct
  misc_feature 1..10: note = Description of Artificial Sequence: Synthetic oligonucleotide
  source 1..10: mol_type = other DNA
agaccgggcc

SEQ ID NO: 913 - DNA, 10 bp, synthetic construct
  misc_feature 1..10: note = Description of Artificial Sequence: Synthetic oligonucleotide
  source 1..10: mol_type = other DNA
tgggacacac

SEQ ID NO: 914 - DNA, 10 bp, synthetic construct
  misc_feature 1..10: note = Description of Artificial Sequence: Synthetic oligonucleotide
  source 1..10: mol_type = other DNA
tgcgcagttg

SEQ ID NO: 915 - DNA, 10 bp, synthetic construct
  misc_feature 1..10: note = Description of Artificial Sequence: Synthetic oligonucleotide
  source 1..10: mol_type = other DNA
cgttcgcctt

SEQ ID NO: 916 - DNA, 10 bp, synthetic construct
  misc_feature 1..10: note = Description of Artificial Sequence: Synthetic oligonucleotide
  source 1..10: mol_type = other DNA
tctcactcgt

SEQ ID NO: 917 - DNA, 10 bp, synthetic construct
  misc_feature 1..10: note = Description of Artificial Sequence: Synthetic oligonucleotide
  source 1..10: mol_type = other DNA
acaccgacgt

SEQ ID NO: 918 - DNA, 10 bp, synthetic construct
  misc_feature 1..10: note = Description of Artificial Sequence: Synthetic oligonucleotide
  source 1..10: mol_type = other DNA
ttcagcccct

SEQ ID NO: 919 - DNA, 10 bp, synthetic construct
  misc_feature 1..10: note = Description of Artificial Sequence: Synthetic oligonucleotide
  source 1..10: mol_type = other DNA
aggcgactaa

SEQ ID NO: 920 - DNA, 10 bp, synthetic construct
  misc_feature 1..10: note = Description of Artificial Sequence: Synthetic oligonucleotide
  source 1..10: mol_type = other DNA
tgctatcaag

SEQ ID NO: 921 - DNA, 10 bp, synthetic construct
  misc_feature 1..10: note = Description of Artificial Sequence: Synthetic oligonucleotide
  source 1..10: mol_type = other DNA
gtccagtagc

SEQ ID NO: 922 - DNA, 10 bp, synthetic construct
  misc_feature 1..10: note = Description of Artificial Sequence: Synthetic oligonucleotide
  source 1..10: mol_type = other DNA
cgtgtgggcg

SEQ ID NO: 923 - DNA, 10 bp, synthetic construct
  misc_feature 1..10: note = Description of Artificial Sequence: Synthetic oligonucleotide
  source 1..10: mol_type = other DNA
gtggttctcc

SEQ ID NO: 924 - DNA, 10 bp, synthetic construct
  misc_feature 1..10: note = Description of Artificial Sequence: Synthetic oligonucleotide
  source 1..10: mol_type = other DNA
gcagccgacg

SEQ ID NO: 925 - DNA, 10 bp, synthetic construct
  misc_feature 1..10: note = Description of Artificial Sequence: Synthetic oligonucleotide
  source 1..10: mol_type = other DNA
gctgtccacg

SEQ ID NO: 926 - DNA, 10 bp, synthetic construct
  misc_feature 1..10: note = Description of Artificial Sequence: Synthetic oligonucleotide
  source 1..10: mol_type = other DNA
cgacactcat

SEQ ID NO: 927 - DNA, 10 bp, synthetic construct
  misc_feature 1..10: note = Description of Artificial Sequence: Synthetic oligonucleotide
  source 1..10: mol_type = other DNA
catggcacct

SEQ ID NO: 928 - DNA, 10 bp, synthetic construct
  misc_feature 1..10: note = Description of Artificial Sequence: Synthetic oligonucleotide
  source 1..10: mol_type = other DNA
tgtgacgtgt

SEQ ID NO: 929 - DNA, 10 bp, synthetic construct
  misc_feature 1..10: note = Description of Artificial Sequence: Synthetic oligonucleotide
  source 1..10: mol_type = other DNA
tttggactaa

SEQ ID NO: 930 - DNA, 10 bp, synthetic construct
  misc_feature 1..10: note = Description of Artificial Sequence: Synthetic oligonucleotide
  source 1..10: mol_type = other DNA
ttcatgcccg

SEQ ID NO: 931 - DNA, 10 bp, synthetic construct
  misc_feature 1..10: note = Description of Artificial Sequence: Synthetic oligonucleotide
  source 1..10: mol_type = other DNA
ttgatcgtgg

SEQ ID NO: 932 - DNA, 10 bp, synthetic construct
  misc_feature 1..10: note = Description of Artificial Sequence: Synthetic oligonucleotide
  source 1..10: mol_type = other DNA
tagcatagga

SEQ ID NO: 933 - DNA, 10 bp, synthetic construct
  misc_feature 1..10: note = Description of Artificial Sequence: Synthetic oligonucleotide
  source 1..10: mol_type = other DNA
gtagttgcaa

SEQ ID NO: 934 - DNA, 10 bp, synthetic construct
  misc_feature 1..10: note = Description of Artificial Sequence: Synthetic oligonucleotide
  source 1..10: mol_type = other DNA
gggacagcta

SEQ ID NO: 935 - DNA, 10 bp, synthetic construct
  misc_feature 1..10: note = Description of Artificial Sequence: Synthetic oligonucleotide
  source 1..10: mol_type = other DNA
aaacccccaa

SEQ ID NO: 936 - DNA, 10 bp, synthetic construct
  misc_feature 1..10: note = Description of Artificial Sequence: Synthetic oligonucleotide
  source 1..10: mol_type = other DNA
actctcacaa

SEQ ID NO: 937 - DNA, 10 bp, synthetic construct
  misc_feature 1..10: note = Description of Artificial Sequence: Synthetic oligonucleotide
  source 1..10: mol_type = other DNA
atcattgcca

SEQ ID NO: 938 - DNA, 10 bp, synthetic construct
  misc_feature 1..10: note = Description of Artificial Sequence: Synthetic oligonucleotide
  source 1..10: mol_type = other DNA
ccagtttgcg

SEQ ID NO: 939 - DNA, 10 bp, synthetic construct
  misc_feature 1..10: note = Description of Artificial Sequence: Synthetic oligonucleotide
  source 1..10: mol_type = other DNA
acattagtca

SEQ ID NO: 940 - DNA, 10 bp, synthetic construct
  misc_feature 1..10: note = Description of Artificial Sequence: Synthetic oligonucleotide
  source 1..10: mol_type = other DNA
ctccagggta

SEQ ID NO: 941 - DNA, 10 bp, synthetic construct
  misc_feature 1..10: note = Description of Artificial Sequence: Synthetic oligonucleotide
  source 1..10: mol_type = other DNA
gaagggccaa

SEQ ID NO: 942 - DNA, 10 bp, synthetic construct
  misc_feature 1..10: note = Description of Artificial Sequence: Synthetic oligonucleotide
  source 1..10: mol_type = other DNA
cagtctcccc

SEQ ID NO: 943 - DNA, 10 bp, synthetic construct
  misc_feature 1..10: note = Description of Artificial Sequence: Synthetic oligonucleotide
  source 1..10: mol_type = other DNA
gagacattcc

SEQ ID NO: 944 - DNA, 10 bp, synthetic construct
  misc_feature 1..10: note = Description of Artificial Sequence: Synthetic oligonucleotide
  source 1..10: mol_type = other DNA
aacggtgttg

SEQ ID NO: 945 - DNA, 10 bp, synthetic construct
  misc_feature 1..10: note = Description of Artificial Sequence: Synthetic oligonucleotide
  source 1..10: mol_type = other DNA
agcattatca

SEQ ID NO: 946 - DNA, 10 bp, synthetic construct
  misc_feature 1..10: note = Description of Artificial Sequence: Synthetic oligonucleotide
  source 1..10: mol_type = other DNA
ctataccgag

SEQ ID NO: 947 - DNA, 10 bp, synthetic construct
  misc_feature 1..10: note = Description of Artificial Sequence: Synthetic oligonucleotide
  source 1..10: mol_type = other DNA
aactggatca

SEQ ID NO: 948 - DNA, 10 bp, synthetic construct
  misc_feature 1..10: note = Description of Artificial Sequence: Synthetic oligonucleotide
  source 1..10: mol_type = other DNA
gtcttgtcgg

SEQ ID NO: 949 - DNA, 10 bp, synthetic construct
  misc_feature 1..10: note = Description of Artificial Sequence: Synthetic oligonucleotide
  source 1..10: mol_type = other DNA
gacgagccgc

SEQ ID NO: 950 - DNA, 10 bp, synthetic construct
  misc_feature 1..10: note = Description of Artificial Sequence: Synthetic oligonucleotide
  source 1..10: mol_type = other DNA
ggaacactgt

SEQ ID NO: 951 - DNA, 10 bp, synthetic construct
  misc_feature 1..10: note = Description of Artificial Sequence: Synthetic oligonucleotide
  source 1..10: mol_type = other DNA
taaatgcgtt

SEQ ID NO: 952 - DNA, 10 bp, synthetic construct
  misc_feature 1..10: note = Description of Artificial Sequence: Synthetic oligonucleotide
  source 1..10: mol_type = other DNA
gcgaacacag

SEQ ID NO: 953 - DNA, 10 bp, synthetic construct
  misc_feature 1..10: note = Description of Artificial Sequence: Synthetic oligonucleotide
  source 1..10: mol_type = other DNA
ttctctcaac

SEQ ID NO: 954 - DNA, 10 bp, synthetic construct
  misc_feature 1..10: note = Description of Artificial Sequence: Synthetic oligonucleotide
  source 1..10: mol_type = other DNA
gtcgtactga

SEQ ID NO: 955 - DNA, 10 bp, synthetic construct
  misc_feature 1..10: note = Description of Artificial Sequence: Synthetic oligonucleotide
  source 1..10: mol_type = other DNA
tgtggcgtaa

SEQ ID NO: 956 - DNA, 10 bp, synthetic construct
  misc_feature 1..10: note = Description of Artificial Sequence: Synthetic oligonucleotide
  source 1..10: mol_type = other DNA
tgagcggcgt

SEQ ID NO: 957 - DNA, 10 bp, synthetic construct
  misc_feature 1..10: note = Description of Artificial Sequence: Synthetic oligonucleotide
  source 1..10: mol_type = other DNA
cctcgtgaac

SEQ ID NO: 958 - DNA, 10 bp, synthetic construct
  misc_feature 1..10: note = Description of Artificial Sequence: Synthetic oligonucleotide
  source 1..10: mol_type = other DNA
gagcaatgaa

SEQ ID NO: 959 - DNA, 10 bp, synthetic construct
  misc_feature 1..10: note = Description of Artificial Sequence: Synthetic oligonucleotide
  source 1..10: mol_type = other DNA
cgagacctaa

SEQ ID NO: 960 - DNA, 10 bp, synthetic construct
  misc_feature 1..10: note = Description of Artificial Sequence: Synthetic oligonucleotide
  source 1..10: mol_type = other DNA
aactgagcgc

SEQ ID NO: 961 - DNA, 10 bp, synthetic construct
  misc_feature 1..10: note = Description of Artificial Sequence: Synthetic oligonucleotide
  source 1..10: mol_type = other DNA
taaagctcgt

SEQ ID NO: 962 - DNA, 10 bp, synthetic construct
  misc_feature 1..10: note = Description of Artificial Sequence: Synthetic oligonucleotide
  source 1..10: mol_type = other DNA
ctctttacgt

SEQ ID NO: 963 - DNA, 10 bp, synthetic construct
  misc_feature 1..10: note = Description of Artificial Sequence: Synthetic oligonucleotide
  source 1..10: mol_type = other DNA
ccccgtggaa

SEQ ID NO: 964 - DNA, 10 bp, synthetic construct
  misc_feature 1..10: note = Description of Artificial Sequence: Synthetic oligonucleotide
  source 1..10: mol_type = other DNA
tcggttcgtc

SEQ ID NO: 965 - DNA, 10 bp, synthetic construct
  misc_feature 1..10: note = Description of Artificial Sequence: Synthetic oligonucleotide
  source 1..10: mol_type = other DNA
ctgcttacac

SEQ ID NO: 966 - DNA, 10 bp, synthetic construct
  misc_feature 1..10: note = Description of Artificial Sequence: Synthetic oligonucleotide
  source 1..10: mol_type = other DNA
acaccgtaat

SEQ ID NO: 967 - DNA, 10 bp, synthetic construct
  misc_feature 1..10: note = Description of Artificial Sequence: Synthetic oligonucleotide
  source 1..10: mol_type = other DNA
cctggtcggc

SEQ ID NO: 968 - DNA, 10 bp, synthetic construct
  misc_feature 1..10: note = Description of Artificial Sequence: Synthetic oligonucleotide
  source 1..10: mol_type = other DNA
ggttatttgg

SEQ ID NO: 969 - DNA, 10 bp, synthetic construct
  misc_feature 1..10: note = Description of Artificial Sequence: Synthetic oligonucleotide
  source 1..10: mol_type = other DNA
gcaactgagt

SEQ ID NO: 970 - DNA, 10 bp, synthetic construct
  misc_feature 1..10: note = Description of Artificial Sequence: Synthetic oligonucleotide
  source 1..10: mol_type = other DNA
ataaggcctc

SEQ ID NO: 971 - DNA, 10 bp, synthetic construct
  misc_feature 1..10: note = Description of Artificial Sequence: Synthetic oligonucleotide
  source 1..10: mol_type = other DNA
cgtgcgaagg

SEQ ID NO: 972 - DNA, 10 bp, synthetic construct
  misc_feature 1..10: note = Description of Artificial Sequence: Synthetic oligonucleotide
  source 1..10: mol_type = other DNA
gtcacacact

SEQ ID NO: 973 - DNA, 10 bp, synthetic construct
  misc_feature 1..10: note = Description of Artificial Sequence: Synthetic oligonucleotide
  source 1..10: mol_type = other DNA
catacggcaa

SEQ ID NO: 974 - DNA, 10 bp, synthetic construct
  misc_feature 1..10: note = Description of Artificial Sequence: Synthetic oligonucleotide
  source 1..10: mol_type = other DNA
gaactgccca

SEQ ID NO: 975 - DNA, 10 bp, synthetic construct
  misc_feature 1..10: note = Description of Artificial Sequence: Synthetic oligonucleotide
  source 1..10: mol_type = other DNA
aatatgtgaa

SEQ ID NO: 976 - DNA, 10 bp, synthetic construct
  misc_feature 1..10: note = Description of Artificial Sequence: Synthetic oligonucleotide
  source 1..10: mol_type = other DNA
ccgatcctgt

SEQ ID NO: 977 - DNA, 10 bp, synthetic construct
  misc_feature 1..10: note = Description of Artificial Sequence: Synthetic oligonucleotide
  source 1..10: mol_type = other DNA
caaagagcct

SEQ ID NO: 978 - DNA, 10 bp, synthetic construct
  misc_feature 1..10: note = Description of Artificial Sequence: Synthetic oligonucleotide
  source 1..10: mol_type = other DNA
taacttagag

SEQ ID NO: 979 - DNA, 10 bp, synthetic construct
  misc_feature 1..10: note = Description of Artificial Sequence: Synthetic oligonucleotide
  source 1..10: mol_type = other DNA
cagcatgtag

SEQ ID NO: 980 - DNA, 10 bp, synthetic construct
  misc_feature 1..10: note = Description of Artificial Sequence: Synthetic oligonucleotide
  source 1..10: mol_type = other DNA
ccccatgcag

SEQ ID NO: 981 - DNA, 10 bp, synthetic construct
  misc_feature 1..10: note = Description of Artificial Sequence: Synthetic oligonucleotide
  source 1..10: mol_type = other DNA
tctgaaccac

SEQ ID NO: 982 - DNA, 10 bp, synthetic construct
  misc_feature 1..10: note = Description of Artificial Sequence: Synthetic oligonucleotide
  source 1..10: mol_type = other DNA
gcgtgcaaaa

SEQ ID NO: 983 - DNA, 10 bp, synthetic construct
  misc_feature 1..10: note = Description of Artificial Sequence: Synthetic oligonucleotide
  source 1..10: mol_type = other DNA
gctagtaccg

SEQ ID NO: 984 - DNA, 10 bp, synthetic construct
  misc_feature 1..10: note = Description of Artificial Sequence: Synthetic oligonucleotide
  source 1..10: mol_type = other DNA
tttcccgcgc

SEQ ID NO: 985 - DNA, 10 bp, synthetic construct
  misc_feature 1..10: note = Description of Artificial Sequence: Synthetic oligonucleotide
  source 1..10: mol_type = other DNA
ccttagtagg

SEQ ID NO: 986 - DNA, 10 bp, synthetic construct
  misc_feature 1..10: note = Description of Artificial Sequence: Synthetic oligonucleotide
  source 1..10: mol_type = other DNA
ttgtgtcttg

SEQ ID NO: 987 - DNA, 10 bp, synthetic construct
  misc_feature 1..10: note = Description of Artificial Sequence: Synthetic oligonucleotide
  source 1..10: mol_type = other DNA
gcaacgaagc

SEQ ID NO: 988 - DNA, 10 bp, synthetic construct
  misc_feature 1..10: note = Description of Artificial Sequence: Synthetic oligonucleotide
  source 1..10: mol_type = other DNA
tgaaaccctt

SEQ ID NO: 989 - DNA, 10 bp, synthetic construct
  misc_feature 1..10: note = Description of Artificial Sequence: Synthetic oligonucleotide
  source 1..10: mol_type = other DNA
ttctacgatc

SEQ ID NO: 990 - DNA, 10 bp, synthetic construct
  misc_feature 1..10: note = Description of Artificial Sequence: Synthetic oligonucleotide
  source 1..10: mol_type = other DNA
attaaaggtg

SEQ ID NO: 991 - DNA, 10 bp, synthetic construct
  misc_feature 1..10: note = Description of Artificial Sequence: Synthetic oligonucleotide
  source 1..10: mol_type = other DNA
tatctaacgg

SEQ ID NO: 992 - DNA, 10 bp, synthetic construct
  misc_feature 1..10: note = Description of Artificial Sequence: Synthetic oligonucleotide
  source 1..10: mol_type = other DNA
agtgctcctg

SEQ ID NO: 993 - DNA, 10 bp, synthetic construct
  misc_feature 1..10: note = Description of Artificial Sequence: Synthetic oligonucleotide
  source 1..10: mol_type = other DNA
ccgtccctct

SEQ ID NO: 994 - DNA, 10 bp, synthetic construct
  misc_feature 1..10: note = Description of Artificial Sequence: Synthetic oligonucleotide
  source 1..10: mol_type = other DNA
ctaacgagcg

SEQ ID NO: 995 - DNA, 10 bp, synthetic construct
  misc_feature 1..10: note = Description of Artificial Sequence: Synthetic oligonucleotide
  source 1..10: mol_type = other DNA
aagtccggct

SEQ ID NO: 996 - DNA, 10 bp, synthetic construct
  misc_feature 1..10: note = Description of Artificial Sequence: Synthetic oligonucleotide
  source 1..10: mol_type = other DNA
ggcgtataag

SEQ ID NO: 997 - DNA, 10 bp, synthetic construct
  misc_feature 1..10: note = Description of Artificial Sequence: Synthetic oligonucleotide
  source 1..10: mol_type = other DNA
agatattagg

SEQ ID NO: 998 - DNA, 10 bp, synthetic construct
  misc_feature 1..10: note = Description of Artificial Sequence: Synthetic oligonucleotide
  source 1..10: mol_type = other DNA
tcctaacagc

SEQ ID NO: 999 - DNA, 10 bp, synthetic construct
  misc_feature 1..10: note = Description of Artificial Sequence: Synthetic oligonucleotide
  source 1..10: mol_type = other DNA
gaggatacgc

SEQ ID NO: 1000 - DNA, 10 bp, synthetic construct
  misc_feature 1..10: note = Description of Artificial Sequence: Synthetic oligonucleotide
  source 1..10: mol_type = other DNA
cgctctttaa

SEQ ID NO: 1001 - DNA, 21 bp, synthetic construct
  misc_feature 1..21: note = Description of Artificial Sequence: Synthetic oligonucleotide
  source 1..21: mol_type = other DNA
agacgtgtgctcttccgatct

SEQ ID NO: 1002 - DNA, 20 bp, synthetic construct
  misc_feature 1..20: note = Description of Artificial Sequence: Synthetic oligonucleotide
  source 1..20: mol_type = other DNA
agatcggaagagcgtcgtgt

SEQ ID NO: 1003 - DNA, 61 bp, synthetic construct
  misc_feature 1..61: note = Description of Artificial Sequence: Synthetic oligonucleotide
  misc_difference 32..41: note = a, c, t, g, unknown or other
  misc_feature 32..41: note = This region represents the random sequence fragment
  source 1..61: mol_type = other DNA
agacgtgtgctcttccgatctgctacataatnnnnnnnnnnagatcggaagagcgtcgtgt

(19) United States
(12) Patent Application Publication
(10) Pub. No.: US 2024/0117339 A1
(43) Pub. Date: Apr. 11, 2024

(54) RAPID DESIGN, BUILD, TEST, AND LEARN TECHNOLOGIES FOR IDENTIFYING AND USING NON-VIRAL CARRIERS

(71) Applicant: BATTELLE MEMORIAL INSTITUTE, Columbus, OH (US)

(72) Inventors: Anthony D. DUONG, Columbus, OH (US); Danielle J. HUK, Hilliard, OH (US); Cherry GUPTA, Columbus, OH (US); Kenneth R. SIMS, Jr., Delaware, OH (US); Michael S. KOERIS, Irvine, CA (US); Zachary R. SHANK, London, OH (US); Ashlee J. COLBERT, Blacklick, OH (US); Andrea D. MCCUE, Columbus, OH (US); Emma K. SCHMITZ, Columbus, OH (US); Caleb T. HILLRICH, Columbus, OH (US); Shannon D. MILLER, Hilliard, OH (US); Joanna L. HOY, Columbus, OH (US)

(21) Appl. No.: 18/475,879

(22) Filed: Sep. 27, 2023

Related U.S. Application Data
(63) Continuation of application No. 17/715,784, filed on Apr. 7, 2022.
(60) Provisional application No. 63/172,069, filed on Apr. 7, 2021.

(51) Int. Cl. C12N 15/10; A61K 48/00; C12N 15/88

(52) U.S. Cl. CPC C12N 15/1065; A61K 48/0041; A61K 48/0091; C12N 15/88; C12Q 1/6804

(57) ABSTRACT

The disclosure relates to barcoded polymer nanoparticles for in vivo screening and for in vivo therapeutic delivery, and methods therefor. More particularly, the invention relates to polymer nanoparticles, such as reversible addition-fragmentation chain transfer (RAFT) polymer compositions, associated with polynucleotide barcodes, for therapeutic delivery, and for high throughput in vivo screening of drug delivery nanoparticles.

### CROSS-REFERENCE TO RELATED APPLICATIONS

This application is a continuation of U.S. patent application Ser. No. 17/715,784, filed Apr. 7, 2022, which claims priority under 35 U.S.C. § 119(e) to U.S. Provisional Application Ser. No. 63/172,069 filed on Apr. 7, 2021, the entire disclosure of each of which is incorporated herein by reference.

### SEQUENCE LISTING

The present application is being filed along with a Sequence Listing in XML format. The Sequence Listing is provided as a file entitled 920006-394895_SL.xml, created Sep. 28, 2023, and is 1,304,483 bytes in size. The information in the electronic format of the sequence listing is incorporated herein by reference in its entirety.

### FIELD OF THE DISCLOSURE

The disclosure relates to barcoded polymer nanoparticles for in vivo screening and for in vivo therapeutic delivery, and methods therefor. More particularly, the invention relates to polymer nanoparticles, such as reversible addition-fragmentation chain transfer (RAFT) polymer compositions, associated with polynucleotide barcodes, for therapeutic delivery, and for high throughput in vivo screening of drug delivery nanoparticles.

### BACKGROUND

Genetic medicines (including gene therapy, gene silencing, splicing regulators, and nuclease based gene editors) are poised to produce revolutionary treatments, including vaccines, infectious disease treatments, antimicrobial treatments, antiviral treatments, and most notably, genetic disease treatments. However, the in vivo delivery of these genetic medicine payloads to the specific tissues and cells that need to be treated, while avoiding tissues and cells that can reduce the efficacy or safety of the genetic medicine, poses a significant challenge. Additional challenges include the ability to deliver large genetic payloads or multiple payloads. Adeno-associated viruses (AAVs) are the most widely used tool for genetic medicine delivery, but AAVs are not able to deliver large genetic payloads or multiple payloads (such as the clustered regularly interspaced short palindromic repeats (CRISPR)/Cas9 system), and they sometimes trigger unwanted immune responses, including the generation of anti-AAV antibodies, a cell mediated response. Some of the immune responses caused by AAV in patients are potentially fatal immune responses.

Therapeutics based on the CRISPR/Cas9 system have an exceptional potential to treat a number of genetic diseases due to the capability of this system for precise and programmable gene editing. Gene editing and repair using the CRISPR/Cas9 system has two main mechanisms, including non-homologous end joining (NHEJ) which repairs the site of cut by inducing random indel mutation, and homology-directed repair (HDR), which repairs the cut site based on a pre-existing template. Because a pre-designed template can be used for HDR-directed repair, therapies based on this mechanism can be tailored to cure a large number of different genetic diseases. However, the main challenge is that HDR repair requires the delivery of CRISPR/Cas9, small guide RNA (sgRNA) and a donor DNA strand at the same time to a particular location. This requirement becomes particularly limiting for in vivo applications because ensuring co-delivery of multiple large molecules to the same targeted location is currently not feasible. For example, the Cas9 enzyme sequence and guide RNA complex is too large to fit into AAVs.

Thus, there is a need for effective non-viral delivery systems, including gene delivery systems. The current state-of-the-art non-viral gene delivery systems, such as liposomes, have many drawbacks such as poor biocompatibility and the inability to easily engineer or functionalize them. Additional concerns are that such non-viral gene delivery systems are easily degraded by various enzymes as they pass through intracellular or intercellular compartments, and these systems have not been able to package multiple large payloads.

The inventors have designed barcoded polymer nanoparticle (e.g., a polymer derived from a controlled living/radical polymerization such as a RAFT polymer) delivery compositions. These compositions have the advantage of being biocompatible, non-toxic, and can be programmed in many ways. For example, the barcoded polymer nanoparticle delivery compositions can be programmed to have functional groups that enable them to evade early degradation, that enable them to evade immune responses, and that enable intracellular imaging and controlled delivery of therapeutic genes and other therapeutic molecules. Thus, these non-viral delivery compositions can enhance the stability, safety, and/or efficacy of genetic medicine payloads and other payloads by providing immune evasion, tissue-directed intracellular delivery, and the ability to deliver large genetic payloads or multiple payloads.

The present disclosure combines these non-viral delivery compositions with rapid design, build, test, and learn (DBTL) technologies that will vastly accelerate gene delivery and address the disadvantages that exist in limited gene delivery vehicles. In addition to hastening editing therapies of today to transition through clinicals, it is anticipated that these technologies will enable the general delivery of larger more molecularly diverse genetic payloads, and other payloads, which will in turn, continue to improve treatments for genetic diseases and other diseases.

### SUMMARY

In some aspects, the disclosure provides for a composition comprising a non-viral delivery vehicle comprising one or more nanoparticle forming polymers, and a nucleic acid construct.

In some aspects, the disclosure provides for a method of in vivo screening for a desired polymer nanoparticle for use as a delivery vehicle, the method comprising (a) preparing a library comprising two or more types of polymer nanoparticles, wherein each polymer nanoparticle is associated with a nucleic acid construct comprising a different polynucleotide barcode, (b) administering the library to an animal, (c) removing cells or tissues from the animal, (d) isolating the nucleic acid constructs from the cells or the tissues of the animal, (e) detecting the nucleic acid constructs in the cells or the tissues of the animal, and (f) identifying the desired polymer nanoparticle for use as a delivery vehicle.

In some aspects, the disclosure provides for a method of treating a patient with a disease, the method comprising administering to the patient the polymer nanoparticle identified in the in vivo screening methods described herein, wherein the polymer nanoparticle further comprises a drug payload, such as a polynucleotide or a protein payload or a small molecule therapeutic payload, and treating the disease in the patient.

The following clauses, and combinations thereof, provide various additional illustrative aspects of the invention described herein. The various embodiments described in any other section of this patent application, including the section titled “DETAILED DESCRIPTION OF ILLUSTRATIVE EMBODIMENTS” and the “EXAMPLES” are applicable to any of the following embodiments of the invention described in the numbered clauses below.
1. A composition comprising:
   a. a non-viral delivery vehicle comprising one or more nanoparticle forming polymers, and
   b. a nucleic acid construct.
2. The composition of clause 1, wherein the non-viral delivery vehicle comprises a polymer nanoparticle.
3. The composition of clause 1 or 2, wherein the nucleic acid constructis associated with the non-viral delivery vehicle via an electrostatic interaction.
4. The composition of any one of clauses 1 to 3, wherein the nucleic acid construct is associated with the non-viral delivery vehicle by an electrostatic interaction of a positively charged polymer segment of the one or more nanoparticle forming polymers and a negatively charged polynucleotide segment of the nucleic acid construct.
5. The composition of any one of clauses 1 to 3, wherein the nucleic acid construct is associated with the non-viral delivery vehicle by a high affinity, non-covalent bond interaction between a biotin molecule on the 5′ and/or the 3′ end of the nucleic acid construct and a molecule that binds to biotin on the one or more nanoparticle forming polymers.
6. The composition of any one of clauses 1 to 3, wherein the nucleic acid construct is associated with the non-viral delivery vehicle by a covalent bond between a carboxy terminated polymer segment and the nucleic acid construct, wherein the nucleic acid construct comprises a primary amine on the 5′ and/or the 3′ end.
7. The composition of any one of the preceding clauses, wherein the nucleic acid construct comprises:
   a. two primer binding segments; and
   b. one or more unique polynucleotide barcodes between the two primer binding segments.
8. The composition of clause 7, wherein the primer binding segments range in length from about 15 base pairs to about 30 base pairs.
9. The composition of clause 7 or 8, wherein the primer binding segments are a universal primer binding set.
10. The composition of any one of clauses 7 to 9, wherein the one or more polynucleotide barcodes comprise unique sequences of 6-20 nucleotides in length.
11. The composition of clause 10, wherein the polynucleotide barcodes further comprise a hamming distance of at least 2-6 bases between any two unique polynucleotide barcode sequences.
12. The composition of any one of the preceding clauses, wherein the nucleic acid construct further comprises from about 6 to about 12 random bases at the 3′ end of the polynucleotide barcode.
13. The composition of clause 12, wherein the about 6 to about 12 random bases at the 3′ end of the polynucleotide barcode are for bioinformatic removal of PCR duplicates.
14. The composition of any one of the preceding clauses, wherein the nucleic acid construct ranges in length from about 42 nucleotides to about 210 nucleotides.
15. The composition of any one of the preceding clauses, wherein the one or more nanoparticle forming polymers comprise monomer units compatible with a controlled living/radical polymerization.
16. The composition of any one of the preceding clauses, wherein the one or more nanoparticle forming polymers are prepared by reversible-deactivation radical polymerization, atom transfer radical polymerization (ATRP), reversible addition fragmentation chain transfer polymerization (RAFT), iodine-transfer polymerization (ITP), selenium-centered radical-mediated polymerization, telluride-mediated polymerization (TERP), stibine-mediated polymerization, or ring-opening polymerization.
17. The composition of any one of the preceding clauses, wherein the one or more nanoparticle forming polymers are prepared from monomers containing a vinyl group.
18. The composition of any one of the preceding clauses, wherein the one or more nanoparticle forming polymers are prepared using a chain transfer agent such as those used in reversible addition-fragmentation chain transfer (RAFT).
19. The composition of any one of the preceding clauses, wherein the one or more nanoparticle forming polymers are RAFT block copolymers comprising
   a. a first terminus comprising a first capping unit derived from a first chain transfer agent in a RAFT copolymerization process;
   b. a first block prepared from one or more monomer units covalently attached to the first reactive functional unit, and having a molecular weight (Mₙ) in the range of about 1 kDa to about 500 kDa and a degree of polymerization in the range of about 10 to about 2500;
   c. optionally a second block prepared from one or more monomer units covalently attached to the first block, and having a molecular weight (Mₙ)in the range of about 1 kDa to about 500 kDa and a degree of polymerization in the range of about 20 to about 2000; and
   d. a second terminus comprising a second capping unit derived from a first or a second chain transfer agent.
20. The composition of clause 19, wherein the non-viral delivery vehicle has one or more of an overall molecular weight (Mₙ) in the range of about 25 kDa to about 60 kDa, an overall degree of polymerization in the range of about 700 to about 900, a target size in the range of about of about 10 to about 60 nm, and a maximum corona-to-core ratio (CCR) of about 1.5 to about 3.5.
21. The composition of clause 19 or 20, wherein the first block is prepared from one or more monomer units selected from the group consisting of 2-dimethylaminoethyl acrylate, 2-(diethylamino) ethyl methacrylate, 2-(diisopropylamino) ethyl methacrylate, butyl methacrylate, ethyl acrylic acid, propyl acrylic acid, (hydroxyethyl)methacrylate, and methyl methacrylate.
22. The composition of clause 19 or 20, wherein the first block is prepared from one of 2-ditnethylanainoethyl acrylate, 2-(diethylamino) ethyl methacrylate, 2-(diisopropylamino) ethyl methacrylate, butyl methacrylate, ethyl acrylic acid, propyl acrylic acid, (hydroxye.thyl)rnethacrylate., or methyl methacrylate.
23. The composition of any one of clauses 19 to 22, wherein the second block is prepared from one or more monomer units selected from the group consisting of 2-dimethylaminoethyl acrylate, 2-(diethylamino) ethyl methacrylate, 2-(diisopropylamino) ethyl methacrylate, butyl methacrylate, ethyl acrylic acid, propyl acrylic acid, (hydroxyethyl)methacrylate, and methyl methacrylate.
24. The composition of any one of clauses 19 to 23, wherein the second block is a random copolymer prepared from two different monomer units independently selected from the group consisting of 2-ditnethylatninoethyl acrylate, 2-(diethylamino) ethyl methacrylate, 2-(diisopropylamino) ethyl methacrylate, butyl methacrylate, ethyl acrylic acid, propyl acrylic acid, (hydroxyethyl)methacrylate, and methyl methacrylate.
25. The composition of any one of clauses 19 to 23, wherein the second block is a random copolymer prepared from three different monomer units independently selected from the group consisting of 2-dimethylaminoeth 1 acrylate, 2-(diethylamino) ethyl methacrylate, 2-(diisopropylamino) ethyl methacrylate, butyl methacrylate, ethyl acrylic acid, propyl acrylic acid, (hydroxyethyl)methacrylate, and methyl methacrylate.
26. The composition of any one of clauses 19 to 25, wherein the second block is a random copolymer prepared from 2-dimethylamirkoethyi acrylate, butyl methacrylate, and propyl acrylic acid; or 2-dimethylatninoethyl acrylate and butyl methacrylate; or 2-dimethylaminoethyl acrylate, butyl methacrylate, and ethyl acrylic acid.
27. The composition of any one of clauses 19 to 26, wherein each chain transfer agent is independently selected from the group consisting of bis(carboxymethyl)trithiocarbonate, bis(2-amino-2-oxoethyl) trithiocarbonate, bis[4-(2-hydroxyethoxycarbonyl)benzyl] trithiocarbonate, 4-cyano-4-(ethylsulfanylthiocarbonyl) sulfanylvpentanoic acid, 4-cyano-4-((phenylcarbonothioyl)thio)pentanoic acid, and 4-cyano-4-[(dodecylsulfanylthiocarbonyl)sulfanyl] pentanoic acid.
28. The composition of any one of clauses 19 to 27, wherein the first capping unit is of the formula

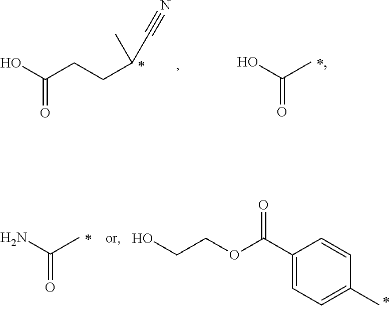

wherein * represents a point of covalent attachment to the first block.
29. The composition of any one of clauses 19 to 28, wherein the second capping unit is of the formula

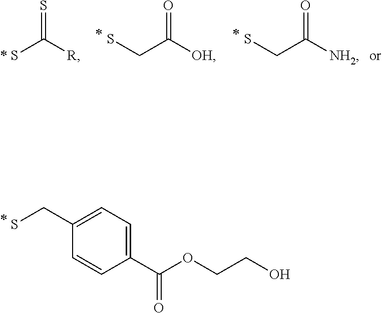

wherein * represents a point of covalent attachment to the second block, and R is —SC₂—C₁₂ alkyl or C₆H₅,
30. A method of in vivo screening for a desired polymer nanoparticle for use as a delivery vehicle, the method comprising (a) preparing a library comprising two or more types of polymer nanoparticles, wherein each polymer nanoparticle is associated with a nucleic acid construct comprising a different polynucleotide barcode, (b) administering the library to an animal, (c) removing cells or tissues from the animal, (d) isolating the nucleic acid constructs from the cells or the tissues of the animal, (e) detecting the nucleic acid constructs in the cells or the tissues of the animal, and (f) identifying the desired polymer nanoparticle for use as a delivery vehicle.
31. The method of clause 30 wherein the polymer nanoparticle associated with the nucleic acid construct is according to any one of clauses 1 to 29.
32. The method of clause 30 or 31 wherein the nucleic acid construct is detected by a method selected from the group consisting of the polymerase chain reaction (PCR), isothermal amplification, sequencing, or a combination thereof, to obtain nucleotide sequence data.
33. The method of any one of clauses 30 to 32, wherein the polymer nanoparticle is loaded with a payload.
34. The method of clause 33, wherein the payload is a luminescent molecule.
35. The method of clause 34, wherein the luminescence is used to track the biodistribution or cell uptake via imaging.
36. The method of any one of clauses 30 to 35, wherein the administration to the animal is via an intramuscular, an intravenous, an intraperitoneal, an oral, or a pulmonary route.
37. The method of any one of clauses 30 to 36, wherein the nucleic acid construct is isolated from the cells and the tissues by mixing with a first organic compound and incubating the organic phase with an aqueous phase of the cell or tissue sample, separating the organic phase from the aqueous phase, mixing the organic phase with a second organic compound, incubating the mixture, precipitating the nucleic acid construct from the mixture, removing the organic phase by evaporation, and resuspending the nucleic acid construct in an aqueous composition.
38. The method of clause 37, wherein the organic phase comprises phenol chloroform.
39. The method of clause 37, wherein the nucleic acid construct is separated from cationic material in the cells or tissues by titrating the aqueous composition of the nucleic acid contstruct to a pH of greater than 7.4.
40. The method of clause 30, wherein the nucleic acid construct is separated from material in the cells or tissues by binding the nucleic acid construct with a molecule with a binding affinity to the nucleic acid construct greater than the binding affinity to the cell or tissue material.
41. The method of clause 30, wherein the nucleic acid construct is separated from material in the cells or tissues via size exclusion chromatography.
42. The method of clause 30, wherein the nucleic acid construct is separated from material in the cells or tissues via dialysis or diafiltration.
43. The method of clause 30, wherein the nucleic acid construct is separated from material in the cells or tissues via filtration.
44. The method of clause 30, wherein the nucleic acid construct is separated from material in the cells or tissues by digesting proteins using an enzyme.
45. The method of clause 44 wherein the enzyme is Proteinase K.
46. The method of clause 30, wherein the nucleic acid constructs associated with the polymer nanoparticles are detected by first diluting the isolated nucleic acid constructs by a factor of at least 1000 times, and then amplifying the nucleic acid constructs by PCR using primers.
47. The method of clause 46, wherein the primers from the PCR step are enzymatically digested prior to detection of amplicons.
48. The method of clause 32, wherein the nucleotide sequence data is converted to fast Q files; and the fast Q files are mapped to known polynucleotide barcodes and the polynucleotide barcodes are enumerated.
49. A method of treating a patient with a disease, the method comprising administering to the patient the polymer nanoparticle identified in the in vivo screening method of any one of clauses 30 to 48, wherein the polymer nanoparticle further comprises a payload, and treating the disease in the patient.

### BRIEF DESCRIPTION OF THE DRAWINGS

FIG. 1 is a schematic diagram showing a simplified flow diagram illustrating a DBTL cycle for non-viral gene delivery development based on automated synthesis, high throughput testing, and machine learning design.

FIG. 2 is a schematic diagram showing an automated multiplexed synthesis of a large, diverse library of PNPs, with various size, charge and hydrophobicity to generate data for gene editing, cytotoxicity, and inflammation.

FIG. 3(a)-3(b) are schematic diagrams showing graph neural network architecture (3(a)) and Zeta potential prediction from SMILES input (3(b)).

FIG. 4 is an illustration of one representative example of a nucleic acid construct of the present disclosure, showing the length (in base pairs (bp)) of the primer binding segments (20 bp and 21 bp in the construct shown), the polynucleotide barcode (8 bp in the construct shown), and the random sequence fragment (7 bp in the construct shown) of the present disclosure.

FIGS. 5(a)-5(c) are a schematic drawing of nucleic acid construct labeling reaction methods using electrostatic loading reaction (FIG. 5(a)), avidin-streptavidin conjugation (FIG. 5(b)), and direct amidification (FIG. 5(c)).

FIG. 6 is an e-gel showing amplification of nucleic acid constructs electrostatically bound to polymer nanoparticles. The presence of the double band in the samples with nucleic acid construct confirms that the barcodes were attached to the PNP. The absence of the double band in the not test control (NTC) validates the positive result.

FIG. 7 is a series of e-gels showing DNA barcode amplification from a pooled sample of 96 unique barcodes, each attached to a prototype PNP. Each frame in the figure is one row of a 48 channel gel electrophoresis. The first column of each gel is a DNA latter, the bottom band of which is ˜100 bases. The bright band in each column near the 100 bp mark indicates amplicons coming from the barcodes.

FIG. 8. is an e-gel showing DNA barcode amplification from a pooled sample of 96 unique barcodes, attached to a prototype PNP, extracted after being spiked into a culture of HEK-293 cells. Each frame in the figure is one row of a 48 channel gel electrophoresis. The first column of each gel is a D=NA latter, the bottom band of which is ˜100 bases. The bright band in each column near the 100 bp mark indicates amplicons coming from the barcodes.

FIG. 9 is an e-gel showing DNA barcode amplification from 10 unique of PNPs with unique barcodes, after being spiked into HEK-293 cells. The presence of the double band is evidence of barcode amplification, present in the positive control sample known to have the barcodes, and not observed in the no test control (NTC) sample, which was phosphate buffered saline only.

FIG. 10 is a series of images showing each of the 10 unique barcoded PNPs from FIG. 7 were loaded with a plasmid expressing a fluorescent TdTomato protein. The loaded PNPs were each dosed into HEK-293 cells. After 48 hours, the cells were imaged via fluorescent microscopy with a Texas Red filter, and the images are shown above.

FIG. 11(a)-FIG. 11(e) show flow cytometry scatter plots depicting cell event distribution of HEK293T cells treated with a representative PNP carrying a td-tomato encoding fluorescent cargo plasmid (FIGS. 11(a)-11(c)), and heat maps depicting Transfection efficiency and viability of a library of 88 diverse PNPs (PNP Library Transfection Efficiency (FIG. 11(d)) and PNP Library Viability (FIG. 11(e))). In FIG. 11(a), the area under the curve denoted by the bar in the graph accounts for 84.17% of the cells.

FIGS. 12(a)-12(d) show gel images of PCR amplified barcodes extracted from indicated mouse tissues (FIGS. 12(a) and 12(b)); sequencing data demonstrating the ability to detect all 96 individual barcodes from a single mouse organ, where (a) denotes low dose and (b) denotes high dose (FIG. 12(c)); and a graph depicting relative abundance of each barcode in a single organ (FIG. 12(d)).

FIG. 13 is an electrophoresis gel showing a band corresponding to barcode (BC) amplicons produced from PCR performed on samples of PNPs with barcodes attached at various molar ratios of PNP to BC (i.e. moles of polymer divided by moles of barcode). The presence of the band for all barcoded PNP samples confirms that the barcode can be detected via PCR on PNPs labeled with barcodes at ratios of anywhere from 20:1 to 10,000:1 (moles PNP:moles BC).

### DETAILED DESCRIPTION OF ILLUSTRATIVE EMBODIMENTS

The invention relates to barcoded polymer nanoparticles for in vivo screening and for in vivo therapeutic delivery, and methods therefor. More particularly, the invention relates to polymer nanoparticles, such as a controlled living/radical polymerization products, such as reversible addition-fragmentation chain transfer (RAFT) polymer compositions, associated with polynucleotide barcodes, for therapeutic delivery, and for high throughput in vivo screening of drug delivery nanoparticles. In one embodiment, the payload can be a nucleic acid of 3 kB or more, or any other suitable payload, such as another polynucleotide or a protein or a small molecule therapeutic or a luminescent molecule.

The invention relates to the use of barcoded polymer nanoparticle compositions (e.g., a polymer nanoparticle derived from a controlled living/radical polymerization process, such as RAFT copolymers) as a platform with a high degree of tunability in structure and function, opportunities to protect payloads from adverse reactions or degradation by the immune system, and passive cell targeting via surface charge, or particle size. These delivery systems also lend themselves to computer-aided design, and they have suitable pathways to robust, commercial scale manufacturing processes with higher yields and fewer purification steps than viral delivery composition manufacturing processes.

In one embodiment a composition comprising a polymer nanoparticle (e.g., a polymer nanoparticle derived from a controlled living/radical polymerization process, such as RAFT polymer) associated with a nucleic acid construct is provided. In another embodiment, a method of in vivo screening to identity a desired polymer nanoparticle (e.g., a polymer nanoparticle derived from a controlled living/radical polymerization process, such as RAFT polymer) associated with a nucleic acid construct for use as a delivery vehicle is provided. In another embodiment, a method of treating a patient with a disease is provided comprising administering to the patient the polymer nanoparticle identified in the screening method.

In one embodiment, the method of in vivo screening for a desired polymer nanoparticle for use as a delivery vehicle comprises, (a) preparing a library comprising two or more types of polymer nanoparticles, wherein each polymer nanoparticle is associated with a nucleic acid construct comprising a different polynucleotide barcode, (b) administering the library to an animal, (c) removing cells or tissues from the animal, (d) isolating the nucleic acid constructs from the cells or tissues of the animal, (e) detecting the nucleic acid constructs in the cells or tissue of the animal, and (f) identifying the desired polymer nanoparticle for use as a delivery vehicle. In various embodiments, the nucleic acid construct can be detected by, for example, the polymerase chain reaction (PCR), isothermal amplification, or sequencing the nucleic acids in the cells or tissues of the animal.

In another embodiment, a method of treating a patient with a disease is provided, comprising administering to the patient the polymer nanoparticle identified in the in vivo screening method, wherein the polymer nanoparticle further comprises a drug payload, such as a polynucleotide or a protein payload, or a small molecule therapeutic or luminescent molecule payload, and treating the disease in the patient.

In various embodiments, any suitable route for administration of the library of polymer nanoparticles associated with nucleic acid constructs for the method of in vivo screening for the polymer nanoparticle associated with a nucleic acid construct, or for the method of treatment can be used including parenteral administration. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. In other embodiments, oral or pulmonary routes of administration can be used.

In one aspect, libraries of barcoded polymer nanoparticles can be pooled and concentrated before administration to the animal of the nucleic acid constructs associated with the polymer nanoparticles. Methods for library preparation and for sequencing are described in Green and Sambrook, “Molecular Cloning: A Laboratory Manual”, 4th Edition, Cold Spring Harbor Laboratory Press, (2012), incorporated herein by reference.

In various embodiments, cell or tissue samples may be analyzed for the presence of the polymer nanoparticle associated with the nucleic acid constructs described herein. The samples can be any tissue, cell, or fluid sample from an animal, for example, selected from the group consisting of urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, lymph fluid, whole blood, serum, plasma, or any tissue or cell sample from an animal Exemplary tissue or cell samples include brain tissue or cells, muscle tissue or cells, skin tissue or cells, heart tissue or cells, kidney tissue or cells, stomach tissue or cells, liver tissue or cells, urinary tract tissue or cells, gastrointestinal tract tissue or cells, head or neck tissue or cells, lung tissue or cells, reproductive tract tissue or cells, pancreatic tissue or cells, or any other tissue or cell type from an animal.

In one illustrative aspect for removing cells or tissues from the animal and isolating the nucleic acid constructs from the cells or tissues of the animal, the nucleic acid constructs are removed from cells or tissues of the animal. In various embodiments, nucleic acid constructs (e.g., DNA or RNA) obtained from the tissues or cells of the animal can be removed by rupturing the cells and isolating the nucleic acid constructs from the lysate. Techniques for rupturing cells and for isolation of nucleic acids are well-known in the art, and removal techniques include homogenization, such as by using a bead-beating technique. In other embodiments, the nucleic acid constructs may be isolated by rupturing cells using a detergent or a solvent, such as phenol-chloroform. In another aspect, the nucleic acid constructs may be separated from the lysate by physical methods including, but not limited to, centrifugation, dialysis, diafiltration, filtration, size exclusion, pressure techniques, digestion of proteins with Proteinase K, or by using a substance with an affinity for nucleic acids such as, for example, beads that bind nucleic acids.

In one illustrative embodiment, the nucleic acid constructs are removed from cells or tissues by treating with a mixture of an organic phase (e.g., phenol chloroform) and an aqueous phase (e.g., water). The organic phase (e.g., phenol chloroform) is isolated and the nucleic acid construct can be precipitated by raising the pH, for example, to pH 7.4. The organic phase (e.g., phenol chloroform) can be evaporated and the nucleic acid constructs can be suspended in water and diluted to appropriate concentrations for PCR and/or sequencing. In one embodiment, the isolated nucleic acid constructs are suspended in either water or a buffer after sufficient washing.

In other embodiments, commercial kits are available for isolation of the nucleic acid constructs, such as Qiagen™, Nuclisensm™, Wizard™ (Promega), QiaAmp 96 DNA Extraction Kit™ and a Qiacube HT™ instrument, and Promegam™. Methods for preparing nucleic acids for PCR and/or sequencing are also described in Green and Sambrook, “Molecular Cloning: A Laboratory Manual”, 4th Edition, Cold Spring Harbor Laboratory Press, (2012), incorporated herein by reference.

The polynucleotide barcodes can be detected by using, for example, the polymerase chain reaction (PCR), isothermic amplification, sequencing, and/or imaging. The polymerase chain reaction (PCR) has been developed to analyze nucleic acids in a laboratory. PCR evolved over the last decade into a new generation of devices and methods known as Next Generation Sequencing (NGS). NGS provides faster detection and amplification of nucleic acids at a cheaper price. The NGS devices and methods allow for rapid sequencing as the nucleic acids are amplified in massively parallel, high-throughput platforms.

In one illustrative aspect, the nucleic acid constructs can be sequenced, to detect the polynucleotide barcodes using any suitable sequencing method including Next Generation Sequencing (e.g., using Illumina, ThermoFisher, or PacBio or Oxford Nanopore Technologies sequencing platforms), sequencing by synthesis, pyrosequencing, nanopore sequencing, or modifications or combinations thereof can be used. In one embodiment, the sequencing can be amplicon sequencing. In another embodiment, the sequencing can be whole genome sequencing. In another embodiment, the sequencing can be exome/targeted hybridization sequencing. Methods for sequencing nucleic acids are also well-known in the art and are described in Sambrook et al., “Molecular Cloning: A Laboratory Manual”, Cold Spring Harbor Laboratory Press, incorporated herein by reference.

In one aspect, the nucleic acid construct can comprise a polynucleotide barcode and the barcode comprises a unique sequence not present in any known genome for identification of the polynucleotide barcode. In another embodiment, a set of different nucleic acid constructs with different polynucleotide barcodes (e.g., 88 or 96 different polynucleotide barcodes) can be used to allow for multiplexing of samples on one sequencing run.

In various embodiments, the polynucleotide barcodes can be from about 5 to about 35 base pairs in length, about 5 to about 34 base pairs in length, about 5 to about 33 base pairs in length, about 5 to about 32 base pairs in length, about 5 to about 31 base pairs in length, about 5 to about 30 base pairs in length, about 5 to about 29 base pairs in length, about 5 to about 28 base pairs in length, about 5 to about 27 base pairs in length, about 5 to about 26 base pairs in length, about 5 to about 25 base pairs in length, about 5 to about 24 base pairs in length, about 5 to about 23 base pairs in length, about 5 to about 22 base pairs in length, about 5 to about 21 base pairs in length, about 5 to about 20 base pairs in length, about 5 to about 19 base pairs in length, about 5 to about 18 base pairs in length, about 5 to about 17 base pairs in length, about 5 to about 16 base pairs in length, about 5 to about 15 base pairs in length, about 5 to 14 base pairs in length, about 5 to 13 base pairs in length, about 5 to 12 base pairs in length, about 5 to 11 base pairs in length, about 5 to 10 base pairs in length, about 5 to 9 base pairs in length, about 5 to 8 base pairs in length, about 6 to 10 base pairs in length, about 7 to 10 base pairs in length, about 8 to 10 base pairs in length, or about 6 to about 20 base pairs in length.

Various embodiments of polynucleotide barcodes are shown below in Table 1 (labeled “Polynucleotide Barcodes”). These polynucleotide barcodes can be used in the nucleic acid constructs alone or in combinations of, for example, two or more polynucleotide barcodes, three or more polynucleotide barcodes, four or more polynucleotide barcodes, etc. In the embodiment where more than one polynucleotide barcode is used, the hamming distance between the polynucleotide barcodes can be about 2 to about 6 nucleotides, or any suitable number of nucleotides can form a hamming distance, or no nucleotides are present between the polynucleotide barcodes.

TABLE 1

| Polynucleotide | |
|---|---|
| Barcodes | SEQ ID NO: |
| GCTACATAAT | 1 |
| ATGTTACACA | 2 |
| TGGGGCCCAA | 3 |
| TAGTTTATCC | 4 |
| ACCCCGTCTT | 5 |
| CCGGCCATCA | 6 |
| GAGCTTGCTC | 7 |
| ACGTTCTATA | 8 |
| TACAGCAAAA | 9 |
| GTTAGGTGGT | 10 |
| GGAGACCGAC | 11 |
| TGGCCCCTTG | 12 |
| TGGCCGTAAG | 13 |
| CGTTCGTCAA | 14 |
| CGGACGTGGA | 15 |
| AGAGGGGGCA | 16 |
| GTTCAGGTCG | 17 |
| CTCGCAAGAG | 18 |
| GCAACGACTT | 19 |
| GCCATCCATC | 20 |
| TTCCGAGCAG | 21 |
| CTTCTGGACA | 22 |
| AACATTAGAC | 23 |
| AAGCAATAGT | 24 |
| AGGGTAAGAC | 25 |
| CGTTGTCTTG | 26 |
| TTTCCCCGCC | 27 |
| CGAATGGATC | 28 |
| CATCACTTGC | 29 |
| CTCTCGCACT | 30 |
| GTTCACGTGC | 31 |
| AATAAGCCTG | 32 |
| GTTAACAATT | 33 |
| ATTCAGATCC | 34 |
| CCTGCTGATT | 35 |
| CTTGGTCATA | 36 |
| TCTTCCTGTT | 37 |
| ACTGCCATGG | 38 |
| CATGTATAGT | 39 |
| GGTAGCGGCA | 40 |
| TCACTCTAAC | 41 |
| AAGGTGCACC | 42 |
| AATGCTCGTT | 43 |
| TGTCTAGAAA | 44 |
| CTGCCTGCCT | 45 |
| ACTATAAAAG | 46 |
| TAGTATCGAG | 47 |
| ATCGCAGTCC | 48 |
| TCATCAGAAC | 49 |
| TCCTAGACGC | 50 |
| GCCGGGCGGG | 51 |
| GCCCAGAAGA | 52 |
| CTTAGAGCTG | 53 |
| GTCTGCGCTT | 54 |
| CGCCGTCCTT | 55 |
| TTTATCTGCT | 56 |
| TGCTTCGGAG | 57 |
| GGGGAGAATG | 58 |
| GTGGTAAGTG | 59 |
| GAAATTAGTA | 60 |
| GCTATCCTAA | 61 |
| ATCTGTACGA | 62 |
| AGTTCGGGGC | 63 |
| CGAGTCTGTC | 64 |
| ATCCTACGCA | 65 |
| ATGGTGGATA | 66 |
| CCTCTAACTA | 67 |
| ATAGCTGCAC | 68 |
| GACAGAATTT | 69 |
| CAATTGGCAT | 70 |
| TCTAGTAGAC | 71 |
| TTATTCATGG | 72 |
| TTGGCAACCG | 73 |
| CATAATACAT | 74 |
| ACAGACTCAC | 75 |
| GCGATGCTGC | 76 |
| CATCTTTGCC | 77 |
| GTGACTCCAG | 78 |
| GGACGAGTCT | 79 |
| TAGTGGCGTG | 80 |
| AACGCAGCTT | 81 |
| AGAACAGGTG | 82 |
| AGGCTATGTT | 83 |
| CCTGGATCTT | 84 |
| CTAGCCGGCC | 85 |
| ACCAGTTATC | 86 |
| ACGTTATAGC | 87 |
| TCGAGTTTGA | 88 |
| TGAAGCGAGC | 89 |
| GACTGGCGAA | 90 |
| GATGGACCTA | 91 |
| GTCCACAACG | 92 |
| CCTCCCCAGA | 93 |
| TTATGACGCC | 94 |
| CTTGATCCGT | 95 |
| AATGCGCAAT | 96 |
| GTACCCCTCA | 97 |
| CGACAGCTCG | 98 |
| TGACCTGGCT | 99 |
| TTCATAGCCC | 100 |
| CCCAAGAGAA | 101 |
| AAACGAAGTA | 102 |
| GACGTTTACA | 103 |
| GATCGATTTG | 104 |
| CACTGTCACC | 105 |
| TGTGAGAGTT | 106 |
| GACGTAACCT | 107 |
| CAGACTCTGC | 108 |
| TATGCCAATA | 109 |
| ACAGGTGATG | 110 |
| GTCATCGCGT | 111 |
| TCTTATAAAC | 112 |
| GTGTAGACTG | 113 |
| AAACAACCGG | 114 |
| ATCCTGTACC | 115 |
| TTATAAGAAT | 116 |
| ATAAGTAGGC | 117 |
| TCTCGTAAGG | 118 |
| GATCCGCCGC | 119 |
| TGTCAGGTTT | 120 |
| TCCGAAGCCC | 121 |
| TCCATGTCCA | 122 |
| GTGATGGTAC | 123 |
| CTCCACATAC | 124 |
| TTCGGATGAG | 125 |
| ACGACATCGC | 126 |
| GAGATGCACA | 127 |
| TTTGTATGGC | 128 |
| CTTTTCTAGA | 129 |
| AGTCTAATCA | 130 |
| GACTTAGCCA | 131 |
| TATCACAGTA | 132 |
| AAGCTCGAGT | 133 |
| TGTTACGACA | 134 |
| AAGGATAGTC | 135 |
| GCACTTAGCC | 136 |
| GAGGGATCCG | 137 |
| ATTCTAGAAG | 138 |
| GATAACTGAT | 139 |
| ATCTGACTGT | 140 |
| CAAAGCGAAC | 141 |
| GAAATTGCGA | 142 |
| GGGTCCAGTC | 143 |
| ATCAGGTAGC | 144 |
| GAAAGGTCCT | 145 |
| GGCTACCACA | 146 |
| TTATTGCTGA | 147 |
| CGCCGCGTTT | 148 |
| TTTTCAAAAG | 149 |
| CTGGGCTAAA | 150 |
| CCCGATGAGA | 151 |
| TGGGAAATAT | 152 |
| GTACGAGCGG | 153 |
| GCGTGCAGCT | 154 |
| AGTCTGCGGA | 155 |
| TAACTATTTA | 156 |
| GAGTTGCCGG | 157 |
| CAGCCCGGCG | 158 |
| TCACCTACAT | 159 |
| AGTGGCTAAC | 160 |
| AGAATGTGAG | 161 |
| TAGTTTCGCA | 162 |
| CTTCATTTCT | 163 |
| GCCATGATAT | 164 |
| ACGGCAAATC | 165 |
| ATCGATAGTA | 166 |
| CCTAAAGGCA | 167 |
| TACGAGCGGT | 168 |
| TTTGTCGTCG | 169 |
| TACAAGCTTG | 170 |
| GACCAACACG | 171 |
| GAACGACGAA | 172 |
| TCGGAACGCA | 173 |
| ATCCGGTGGT | 174 |
| TAAAACGTAG | 175 |
| TATGTGAGCC | 176 |
| GAGGCATCGA | 177 |
| GAATGGGTGG | 178 |
| AACGACACAA | 179 |
| GTACGATGCA | 180 |
| AGAAGGCGCC | 181 |
| CCGCAATGGA | 182 |
| TACGGATTTT | 183 |
| GTCGTTAGCT | 184 |
| GGACTAGGGC | 185 |
| ATTGGTATTC | 186 |
| ATCCCAGAGA | 187 |
| GTCCCAGCTC | 188 |
| CACGAGGAAT | 189 |
| TACAATTGCA | 190 |
| ATTCCTGAAT | 191 |
| TAGCGAGGCG | 192 |
| CTGGATGGGC | 193 |
| GCGACGGCCA | 194 |
| ACCTGCACAA | 195 |
| CATGACAGAC | 196 |
| TTACCAACGT | 197 |
| CAGGTGTGTG | 198 |
| CGAGGGACGG | 199 |
| CGTCTCGGTA | 200 |
| TAAGCTATCT | 201 |
| TACTCCCCTA | 202 |
| TTATATTCAT | 203 |
| AGCGATCTGC | 204 |
| TCTTCTGATC | 205 |
| ATAGTTCCCA | 206 |
| TTTACGGGTG | 207 |
| GTGTCCCCTG | 208 |
| GCGGGGGTCG | 209 |
| CATTGATCTA | 210 |
| AGGGACGGTG | 211 |
| CAGTTACTTT | 212 |
| CCATACTTCC | 213 |
| ATCAGAATTA | 214 |
| AAACTAGGCA | 215 |
| AATGTCGTTG | 216 |
| CACATGGGTC | 217 |
| GGTCGCTGGT | 218 |
| ACTGTATTAC | 219 |
| CCGAGACGCG | 220 |
| ACTCCAACCC | 221 |
| ATATTACAAG | 222 |
| CCATGGATAG | 223 |
| CCGTCTCAAT | 224 |
| GATCGTCGGG | 225 |
| TCTTGTTTTG | 226 |
| AATATTGCTC | 227 |
| AACGTCGTCT | 228 |
| AATATTTTTG | 229 |
| CGTAACGTGC | 230 |
| GCGTGGTTAT | 231 |
| CAAAACATTA | 232 |
| CGTATCCTGA | 233 |
| TCGCTTACAA | 234 |
| TCCATTGTGT | 235 |
| GCCCCCATTC | 236 |
| TGACGTCTAT | 237 |
| TGGGCCGAGG | 238 |
| AAGTGTCAAG | 239 |
| GACAGTAGAG | 240 |
| CGCAGCCATC | 241 |
| GAGGCAGAAC | 242 |
| GTTGAAATTG | 243 |
| ATCTGATAAA | 244 |
| AGCTGTCTCT | 245 |
| TTTTAGGTTA | 246 |
| TATCTGTCCG | 247 |
| AAAACATATG | 248 |
| GTAAAGAAGA | 249 |
| TCGACGTGCA | 250 |
| TAGATCTTAA | 251 |
| CACTGGTCAC | 252 |
| ATTCTGATGT | 253 |
| ATGGCCCTGA | 254 |
| GGTGATGAGA | 255 |
| CACCGTGGGG | 256 |
| GCTTGCTCGG | 257 |
| CCAGTTGAAC | 258 |
| CGTCTGTACC | 259 |
| CCAACGCGGC | 260 |
| ACGTGATCGA | 261 |
| CCATCGAATC | 262 |
| CGGTGTCTGC | 263 |
| AAACCACCTC | 264 |
| TCAATGTTCC | 265 |
| TTCGACATGT | 266 |
| AGGCACGATA | 267 |
| CACGAGATCA | 268 |
| CATGCTGGGG | 269 |
| TACCATGGTT | 270 |
| TTGCCCATAT | 271 |
| TGCACATTCG | 272 |
| GTTATGTTGG | 273 |
| TGAGTTATGA | 274 |
| GATGGCCCCC | 275 |
| GATGGGTTAC | 276 |
| AGCTACGTTG | 277 |
| ACCCCATGCA | 278 |
| TACTACCGTT | 279 |
| TCGCTTCTAC | 280 |
| CTGGCAGTGC | 281 |
| TCTATATATA | 282 |
| GGATTAGTTC | 283 |
| GTGTTACGCT | 284 |
| TCGACTCCGT | 285 |
| GGTAGCAGGC | 286 |
| TATTGGATTC | 287 |
| GTTCGATCGA | 288 |
| ATATTAATAT | 289 |
| AGAACGATTG | 290 |
| GTAAAGTGTA | 291 |
| CCCATGTGCC | 292 |
| GTGGCCTCGC | 293 |
| GACACTAGGA | 294 |
| ATATTCTGAC | 295 |
| TAAGTAGACG | 296 |
| TAACGGTCTA | 297 |
| TAGTTTCATT | 298 |
| TTGGATCCGA | 299 |
| CGTGACAACC | 300 |
| CGCGCTCAGA | 301 |
| CGTTCTTAAT | 302 |
| ACAAGAGTTT | 303 |
| AGGGTTATAG | 304 |
| ACCACGACTC | 305 |
| GTACTCGGGG | 306 |
| ACAAATATCT | 307 |
| GATCGGGGTG | 308 |
| ATGTAACTCC | 309 |
| ATGAAGAAGC | 310 |
| ATGTATTGTC | 311 |
| TGCATTGGAA | 312 |
| GCGGACGATC | 313 |
| CCGTACTTGA | 314 |
| TTTGCCCCCG | 315 |
| ACCTCACGCG | 316 |
| ATTAAGGGGC | 317 |
| CGTGGACATG | 318 |
| TTAGCCCTTC | 319 |
| CGAGAGTTTG | 320 |
| TGCATCCTCT | 321 |
| TGCGATTCCG | 322 |
| TTATTACGTT | 323 |
| TGATGTGGTT | 324 |
| GGGCGTCAAT | 325 |
| CCCTTGAAAT | 326 |
| TCTTTGGGGC | 327 |
| ACCGGCAGGC | 328 |
| GCTAAAATCT | 329 |
| GCCGTTGACG | 330 |
| GGAGTTGTTG | 331 |
| TACTTGAGAA | 332 |
| CGGGTGCGCT | 333 |
| AAAAGCGTCT | 334 |
| GTAAAGATAG | 335 |
| GCCTGGTCAG | 336 |
| GGCAAAAAGG | 337 |
| ACCCTTCTCT | 338 |
| TCACATAGTG | 339 |
| TCGTCTGTGC | 340 |
| TGCTCGGATC | 341 |
| AGCAGTCCCG | 342 |
| TTTGGGCTGT | 343 |
| CTCACGATCT | 344 |
| TGGCGCATAC | 345 |
| GCAATTGAAA | 346 |
| TCGGGAGACG | 347 |
| CCCGGCGAAA | 348 |
| TGATGCGGAA | 349 |
| AACTGAGGCG | 350 |
| CATATTATTT | 351 |
| AAAAGTCATT | 352 |
| AAGCGGTGAG | 353 |
| AAGGTAATCA | 354 |
| CTGACACTTA | 355 |
| CTGTTTTCTA | 356 |
| CACATGGCAG | 357 |
| TTCAATCCGG | 358 |
| TGTCCGGCAT | 359 |
| TGGTACCGTG | 360 |
| AAGAGATATT | 361 |
| GATGTACTAC | 362 |
| GAAATGGAAT | 363 |
| TTAAAATACT | 364 |
| TGACCGGAAC | 365 |
| GTCGCCGCAA | 366 |
| TAGGATACCG | 367 |
| AGTCCAATTG | 368 |
| GGGGGCTATA | 369 |
| ACCTTCAGTT | 370 |
| ATGGCAAGTA | 371 |
| AGAATGTTTT | 372 |
| AGTTCGTTTG | 373 |
| CACTACTGAC | 374 |
| GATCAAGAGC | 375 |
| ATTTATCGAG | 376 |
| CCTTTTTCCA | 377 |
| GCACAGAGGT | 378 |
| TGATCTGAAT | 379 |
| GTTGGAGGGA | 380 |
| TTTTGAAGGT | 381 |
| TAAGTCCTAA | 382 |
| GGTGTTAGGG | 383 |
| TGTATGCACC | 384 |
| CCGTGCCATT | 385 |
| GAAATCACCC | 386 |
| TTTGCACGTG | 387 |
| CGTCTGTTTT | 388 |
| CTACACCACA | 389 |
| TGCTACAGGG | 390 |
| GGGAATATAT | 391 |
| TCATGTATTT | 392 |
| TCTCCGTTTA | 393 |
| TACCTCTCGC | 394 |
| GCTTCAACCG | 395 |
| ATGAAGCTAC | 396 |
| CGGTACAACT | 397 |
| GTGTGGTCGT | 398 |
| GGGGTCATGT | 399 |
| AGGCAGCCCA | 400 |
| CAAGCACGAT | 401 |
| TCAAATGGAT | 402 |
| GGACTGAATA | 403 |
| CCGTAGACGT | 404 |
| CGGCGTACCG | 405 |
| GGCGGCGCCC | 406 |
| AGACTTGATC | 407 |
| ACCTTGCACA | 408 |
| TAAGGTGAGT | 409 |
| TTGTTGTTTC | 410 |
| GAGGGAATAC | 411 |
| CTCGTACGCG | 412 |
| CCGCGGTTTA | 413 |
| TTAAAGTTAA | 414 |
| GCATATGGGT | 415 |
| AGTCTGAGCC | 416 |
| TGTCGGTTCG | 417 |
| GGTCTCAACC | 418 |
| GTAACGGCAT | 419 |
| ACACTGAGAA | 420 |
| CCCAACGTCG | 421 |
| AAGAAACTGC | 422 |
| ACCAGCCCAC | 423 |
| TGTAGTTACT | 424 |
| GGCTAGAGGC | 425 |
| GTTCGGCAGA | 426 |
| CCAAAATAGA | 427 |
| CCCATATAAC | 428 |
| GTCACTACCG | 429 |
| GTAGTGTGGC | 430 |
| CAATCTCATA | 431 |
| CCATGTTATA | 432 |
| TAAGCAGTGG | 433 |
| TCGGCGGCTA | 434 |
| TATTAAATGC | 435 |
| GTCGCCATTA | 436 |
| GGCGTCGTTC | 437 |
| CTAGTAGATA | 438 |
| TCGTCAGTAT | 439 |
| GGGGTATCGG | 440 |
| TGCTCTGCCA | 441 |
| TGCCGTAACT | 442 |
| CGGTACAGGC | 443 |
| TCCTAATTTG | 444 |
| TCTTTCTGGA | 445 |
| CCGCGACTTG | 446 |
| ACCTATAGCG | 447 |
| GCCGGCACCT | 448 |
| TTTGATAGGC | 449 |
| ACTGTGAGCT | 450 |
| TTATCGTTCA | 451 |
| ACTAGTGGCC | 452 |
| CCTCCGTGGT | 453 |
| TTAGGGTATG | 454 |
| GAATCAGGCG | 455 |
| GGCTGACCAA | 456 |
| TGCCAGACCG | 457 |
| TCCCTACGCG | 458 |
| TCCGCTGGAG | 459 |
| GGATCAAAAC | 460 |
| TTCACCTCAC | 461 |
| GACACACGGC | 462 |
| TGGGCGATTA | 463 |
| TAAGATCTTC | 464 |
| CTCCGACTAC | 465 |
| GGGCCATCAT | 466 |
| TCAGGCCAGA | 467 |
| CTTGTGGGGC | 468 |
| AGATAGTCTG | 469 |
| GCGTCAAAGT | 470 |
| ACGAAAATTT | 471 |
| GAGTCTGGTG | 472 |
| ATCGAGCGAC | 473 |
| GGTCCTCAGA | 474 |
| TGATTTTGTC | 475 |
| GCATTTCTCA | 476 |
| GCATGCCAGT | 477 |
| ATTAGACGAC | 478 |
| AAAGCCCATA | 479 |
| CACTACATTC | 480 |
| CACGGTTTCT | 481 |
| CCCACCAGTG | 482 |
| CTCACTTGTC | 483 |
| GATAGACTCT | 484 |
| ATTTCCATTT | 485 |
| ATATGTGGCC | 486 |
| CGGGACGAAC | 487 |
| AGAACCGTGA | 488 |
| TAGTGTACTG | 489 |
| AACTAATCGA | 490 |
| CGAAGTGACG | 491 |
| CGGAGCCTCG | 492 |
| ATCACACGAG | 493 |
| CGACGAGTTC | 494 |
| GCTTCCCGTG | 495 |
| GATTCATACC | 496 |
| GAGAGAAGCG | 497 |
| GAAGTGGCCT | 498 |
| GGACGACGCC | 499 |
| TAGGGTCTCA | 500 |
| AACTACAGGT | 501 |
| GTGGCCTGTG | 502 |
| CTTTACCAGC | 503 |
| CGCGTTACTG | 504 |
| TTGCTCCCGT | 505 |
| CATCAAACAA | 506 |
| GCTTTATGAT | 507 |
| CTGCATACTG | 508 |
| GGTGGCTCAG | 509 |
| GGACGATCAA | 510 |
| CCGACTGGTG | 511 |
| GGAACAACCG | 512 |
| GAACGAGACC | 513 |
| CACCAAGAAA | 514 |
| ATGCATTACC | 515 |
| GTATCATGCC | 516 |
| AGTAGATGTT | 517 |
| CTCTAGATGT | 518 |
| GCTACTTGTG | 519 |
| TATGAAACGT | 520 |
| CCTCGTTGAT | 521 |
| CTAGAGCCAT | 522 |
| TAGAGTTATA | 523 |
| AACGAGAGGC | 524 |
| GGTCTACCGT | 525 |
| GCCCCCTCAC | 526 |
| CATAGGAATT | 527 |
| TCCGGCTCGT | 528 |
| TGAGAGTCGG | 529 |
| CGTAGAAATA | 530 |
| CTTTACATGA | 531 |
| GAGCGCCGTC | 532 |
| GGCTCTCGGC | 533 |
| AGAGCTTGTT | 534 |
| AATCAGCCAC | 535 |
| AGAAGAGCCA | 536 |
| TCGTATGAGT | 537 |
| TTCTTCCTCG | 538 |
| ACACAAAAGC | 539 |
| CGCGGGACCC | 540 |
| GTCGCGACAC | 541 |
| CCGGAGGAAA | 542 |
| CGGCGTATGA | 543 |
| TAGGCATTCT | 544 |
| AAAGGAGGGA | 545 |
| ACCTTTACGG | 546 |
| CTACCGTTAA | 547 |
| GAGCTTCGCC | 548 |
| GCCATAGAAG | 549 |
| TTTAGCGTAT | 550 |
| GCAAACAGAT | 551 |
| TAGGTCATGG | 552 |
| CTCTAACAGA | 553 |
| GGCTCATGAA | 554 |
| CAATGTCTCA | 555 |
| TGATCGTATT | 556 |
| GCGCTTTTCA | 557 |
| AAGATTATAT | 558 |
| ACTAGCTGAC | 559 |
| GGTGAGCTCA | 560 |
| CGCTTTCGCT | 561 |
| TGATTCAAAA | 562 |
| ACTGAACAGG | 563 |
| ATTCGAGCTA | 564 |
| TGTAGGCTAA | 565 |
| ACAAAGCTTT | 566 |
| GCCCGAGGGA | 567 |
| GCCCGCTGGG | 568 |
| ACCCCGCTGA | 569 |
| CTTATGCCCT | 570 |
| CCGCCATAGC | 571 |
| CTTAATGATT | 572 |
| CAGTCCACAA | 573 |
| ATGGACGGAC | 574 |
| CGGCCTCTCG | 575 |
| TAGTCGCCAT | 576 |
| GTTGATCTTC | 577 |
| ACTTGCCAAG | 578 |
| ATGACTGGTT | 579 |
| TGTCGTAGGA | 580 |
| AGCAAACACG | 581 |
| TACTGATGAA | 582 |
| GTATCCCATA | 583 |
| TAGCCAGGTT | 584 |
| CGTGTGGCGA | 585 |
| ATCGAATTGC | 586 |
| CCCCAATATT | 587 |
| CCCGTTTCTC | 588 |
| TCCGCATCTA | 589 |
| CAAGCCTCAT | 590 |
| TTTCAATCCC | 591 |
| CCTTCCCATC | 592 |
| AGGTACAAGA | 593 |
| GTGTAATGGA | 594 |
| AAACTGAGCT | 595 |
| ATCTCTGCCC | 596 |
| CGACATTTGC | 597 |
| TGTGAACCCG | 598 |
| TGACACCCCA | 599 |
| TAGGCCAAAG | 600 |
| GAAATTGTAG | 601 |
| GCGTCTGATT | 602 |
| TCTCATTGTT | 603 |
| CTGACATCTC | 604 |
| GTATCCAGTG | 605 |
| GATGGCCGTT | 606 |
| TCACCCTCTC | 607 |
| GGCACTATTC | 608 |
| AAATAACTGT | 609 |
| CAGCTCCATT | 610 |
| CTCTTGACTC | 611 |
| TTTCCTATAC | 612 |
| CCATACCCGA | 613 |
| TCGCCGAGCG | 614 |
| CGCTGAAGCC | 615 |
| TCTGGCCCCA | 616 |
| GCTACATTGA | 617 |
| CGCATCATAA | 618 |
| GCAAAGGGCC | 619 |
| AACGGCGCAG | 620 |
| CGACTGACAT | 621 |
| ATGACAGGGC | 622 |
| CAAGTTCTCC | 623 |
| TCGCCGCTTT | 624 |
| ATGCCGGAAA | 625 |
| GCGGTTACTA | 626 |
| GACATTACAA | 627 |
| CAGAGAGGGC | 628 |
| GCACCGCCTC | 629 |
| CGGTCCGAGC | 630 |
| TGTCCGGTGC | 631 |
| GGTCGGTTGC | 632 |
| GCTCAGCTAA | 633 |
| AGCAGTTCGT | 634 |
| AAATCGATGA | 635 |
| GCTCGGTATG | 636 |
| CCCGCCGCGG | 637 |
| GTGTGATAGG | 638 |
| TTGGACTCCA | 639 |
| TGCTTATCTA | 640 |
| CAAAAGGCGT | 641 |
| TAGGGGGCCT | 642 |
| AAGTATTAAT | 643 |
| GTTTAGCCCG | 644 |
| CGCTAATATG | 645 |
| ACAACACGTT | 646 |
| AGAGATGCTC | 647 |
| TGCCTGATAT | 648 |
| CTTGTAAGTA | 649 |
| CATATTGCCG | 650 |
| CTTAGAAAGT | 651 |
| ATGTTGTATT | 652 |
| CGCATTGAAG | 653 |
| TTATGTTGGT | 654 |
| TCGCCTCAGA | 655 |
| TTCGTTGAGG | 656 |
| GGTGCCGGGC | 657 |
| ACCATTGTAA | 658 |
| TTGATTGTCA | 659 |
| CGGCTCACCT | 660 |
| CTATCACATG | 661 |
| GTAGACAGAA | 662 |
| CCTTTACCAA | 663 |
| GCACATCGAC | 664 |
| TCTCACTTTC | 665 |
| TTCGAGTACT | 666 |
| TAGAAGAGCA | 667 |
| AACCCCACCA | 668 |
| CTGTATCAGT | 669 |
| ACATAATGAG | 670 |
| AGCCTTCCGC | 671 |
| CAGTGCTTTT | 672 |
| TAGTCCGTGT | 673 |
| CGGAATCGGT | 674 |
| CTTGCGGAGA | 675 |
| AAAAATTTGG | 676 |
| TGTTTTCCGC | 677 |
| ATGCTAGGCG | 678 |
| GACTAATTTC | 679 |
| CTGTAGTAAC | 680 |
| CGGATGACTT | 681 |
| TCAGAGTGGA | 682 |
| CAAAATAGCG | 683 |
| GAAGAAGAAG | 684 |
| CACCCGCACG | 685 |
| ACGATGCCCG | 686 |
| CCTACTACAC | 687 |
| ATTGAAACAA | 688 |
| GACCGAAGAT | 689 |
| ACGGCCTGAA | 690 |
| AGGGGAGGTC | 691 |
| CAATCAACTT | 692 |
| GGACAACCGA | 693 |
| TCCCTAAGGC | 694 |
| GTTCTACACG | 695 |
| ACTAACCAGT | 696 |
| GAAGCTGGAT | 697 |
| GGAACCATGG | 698 |
| CTCTACCTGG | 699 |
| TAATGCCTGC | 700 |
| TAAAGGCAAT | 701 |
| CGCCTGGGAA | 702 |
| TCTTGGGGAA | 703 |
| AGAGAGAGAG | 704 |
| GCGTTGGCGC | 705 |
| TTACGACAGA | 706 |
| GGAACTCTTA | 707 |
| GATTGTGGAG | 708 |
| GGGCACTGAT | 709 |
| AGACGCACCA | 710 |
| CCAATTATAA | 711 |
| TAGAGACGCA | 712 |
| CCTCTTGTCG | 713 |
| GAGGAAGCTC | 714 |
| AGTCCCGAGT | 715 |
| TGCTTGCAGT | 716 |
| CCCACTTCCC | 717 |
| CGTTGCCGCG | 718 |
| CCCCTGGTTC | 719 |
| ACGACCAATA | 720 |
| CTTAGGGTTC | 721 |
| AAACATATCA | 722 |
| GGGTCGTAGA | 723 |
| CTCCGTAGCG | 724 |
| CTGGTCATAA | 725 |
| TTGACAGATC | 726 |
| GAGTAAAGTC | 727 |
| ATATGGGCTT | 728 |
| TACAACTACT | 729 |
| AATTCAGCCG | 730 |
| GATTGTACTA | 731 |
| TCGTAATGCG | 732 |
| CGATAACTGC | 733 |
| AACTTGGCGG | 734 |
| CGTGGATGTA | 735 |
| CCTTCCCGAA | 736 |
| CTAAACCCGT | 737 |
| CAACATTCCC | 738 |
| CTTACCCTCT | 739 |
| GGAAAGTTCT | 740 |
| CGGATTGGCT | 741 |
| AATGTAGGGC | 742 |
| AATGAATCGC | 743 |
| ATCATACACC | 744 |
| AGTTGGGCAG | 745 |
| AGAAGAAGGG | 746 |
| GCGTGCGCTA | 747 |
| CCCCGATAAA | 748 |
| TACCAAGTGC | 749 |
| TGTGTTTTCG | 750 |
| CCCAGATGTC | 751 |
| GCGAGCTTCC | 752 |
| GTGTCACGTA | 753 |
| ATAGGCCGAG | 754 |
| GAGCTACCAG | 755 |
| CGCGGCGGAG | 756 |
| TCTTGCACGA | 757 |
| TGCCCTAAAG | 758 |
| TTGCGCTTTG | 759 |
| CATATAAAGG | 760 |
| AATAGCGAAT | 761 |
| TACGCTAAGG | 762 |
| ACTTAGTTCG | 763 |
| CGTGCGGAAC | 764 |
| ACCCGATTCG | 765 |
| TGCAGAGTTT | 766 |
| GAATCATTAG | 767 |
| AGTACACTGG | 768 |
| TTGTGCGGTT | 769 |
| ATGACATGCA | 770 |
| TTCTCGGACG | 771 |
| AGATTGAAGA | 772 |
| GGCGGACTGT | 773 |
| TTTATGGTAA | 774 |
| CAGTAGGGTG | 775 |
| GACAGGCAAG | 776 |
| GATGTGTCGT | 777 |
| ACTTGACGGA | 778 |
| AAGTCCGAAA | 779 |
| TGGGTGTAGG | 780 |
| ACTTACCGCG | 781 |
| CTGTGCACCC | 782 |
| ATTGCTCTCT | 783 |
| CAGAAGACAA | 784 |
| TTACGCTATA | 785 |
| ACGTGGAAAT | 786 |
| TGAGGCTGGT | 787 |
| ATTATGAGAT | 788 |
| GACTTGTAGT | 789 |
| TCGCTGAGGA | 790 |
| CCCAACTCTA | 791 |
| GATAGGGAGG | 792 |
| TAGAAATCAG | 793 |
| GTCGCTAGAA | 794 |
| AAAATAGAAA | 795 |
| GCTCCTGGGT | 796 |
| CGCGCTCGCG | 797 |
| GGCAAACGCA | 798 |
| TTTACTACCT | 799 |
| ATCCTAAACT | 800 |
| CTCCGTATGT | 801 |
| TATCGTCCAG | 802 |
| GCCGGCGGTA | 803 |
| TGCTCCATTT | 804 |
| TGGCTGTTGT | 805 |
| TACTGCGCAA | 806 |
| TATACGGCTT | 807 |
| GGTTATTACC | 808 |
| ATCAGGAGGA | 809 |
| CTATTGCCAG | 810 |
| ACGTACACAC | 811 |
| CAGCCTAGCT | 812 |
| GAAAAACAAC | 813 |
| CGTTCAGTTA | 814 |
| CAATCAGAAT | 815 |
| GGGCTACTCT | 816 |
| CCCCATTGGG | 817 |
| TAGGGAACGG | 818 |
| CAGCTGATAC | 819 |
| ATTCCTGTGA | 820 |
| TCAGAGCCGT | 821 |
| CATGAAAAGC | 822 |
| TGACCTGTGA | 823 |
| GCATTAGCAG | 824 |
| GACAGAACCA | 825 |
| TCCAGTATAT | 826 |
| TGTTCCGCTA | 827 |
| GATATCCATT | 828 |
| CATATGGACC | 829 |
| GATATAGTAA | 830 |
| CACCTTTTTT | 831 |
| AGCTTGCGGG | 832 |
| CGCACAGGGA | 833 |
| TCTGGGTGCT | 834 |
| TGAGTCGTTT | 835 |
| TTACAATGTG | 836 |
| CTTGCAAACA | 837 |
| TGTCGAGCTG | 838 |
| ACTTTAACCT | 839 |
| ATATAAGTGC | 840 |
| GGAAGGGCGT | 841 |
| TTTGACTTGA | 842 |
| GTATAAACGG | 843 |
| TAACCGGATG | 844 |
| TTCTCATCAG | 845 |
| CTCGGTTACG | 846 |
| ATATGGTTCT | 847 |
| CGCCCCCGAA | 848 |
| ACCTCGATCG | 849 |
| CTCGAATAAT | 850 |
| GCCCGAGCTT | 851 |
| AACAGTCAAC | 852 |
| CTGGAACCTC | 853 |
| AATAACGGGG | 854 |
| ACGCCCCACT | 855 |
| GGCAACATGA | 856 |
| GCTATTTCGC | 857 |
| TTCCACTTTA | 858 |
| GCCGATGGAT | 859 |
| AAGTTGGTAA | 860 |
| CACTAGCTAG | 861 |
| ACATGCCCCT | 862 |
| TTCATTACTC | 863 |
| GGTTTAATAT | 864 |
| CCTGCAGTGA | 865 |
| TCTTTAAGTT | 866 |
| TGGCGATCGA | 867 |
| CTTTTTAGCT | 868 |
| CCCAGTCTCT | 869 |
| AAATGTTTCG | 870 |
| ATATAAGACG | 871 |
| TCACTTTACA | 872 |
| CCTGGCGCCC | 873 |
| GGATTACTGG | 874 |
| GAATGATCTT | 875 |
| GCTCGGATCG | 876 |
| CAGCTGCGAG | 877 |
| ACCCTTACTA | 878 |
| AGGTGAAACT | 879 |
| CGAATTTGAT | 880 |
| CGCTGTGCGG | 881 |
| TTACCGCACC | 882 |
| GGAATCTTAA | 883 |
| CTCAACACCC | 884 |
| CGTGCCCTTG | 885 |
| GCAGGCTCGA | 886 |
| ACCAACGAAG | 887 |
| CCTGTAATTT | 888 |
| GGGTGGGATG | 889 |
| TTGCTCACCG | 890 |
| TTACGACCAC | 891 |
| TTTTCTAACC | 892 |
| GCTTTAGATA | 893 |
| CACGTATTGG | 894 |
| AAATATCTCC | 895 |
| GCTGGAAAAC | 896 |
| GAGCGCATTA | 897 |
| GTGGAGGGGT | 898 |
| TCCACTGGGA | 899 |
| CAATAGCGGA | 900 |
| CATCTAGTTT | 901 |
| GAAGTTCCGG | 902 |
| AGCGAGATTC | 903 |
| TTAAGGTCGG | 904 |
| AATGGTTAGG | 905 |
| CGTTATTATA | 906 |
| ACGGAAAGGA | 907 |
| CCTTGTCCCG | 908 |
| ATACTTTTTT | 909 |
| CTGGGTCTGG | 910 |
| AACCATTGCG | 911 |
| AGACCGGGCC | 912 |
| TGGGACACAC | 913 |
| TGCGCAGTTG | 914 |
| CGTTCGCCTT | 915 |
| TCTCACTCGT | 916 |
| ACACCGACGT | 917 |
| TTCAGCCCCT | 918 |
| AGGCGACTAA | 919 |
| TGCTATCAAG | 920 |
| GTCCAGTAGC | 921 |
| CGTGTGGGCG | 922 |
| GTGGTTCTCC | 923 |
| GCAGCCGACG | 924 |
| GCTGTCCACG | 925 |
| CGACACTCAT | 926 |
| CATGGCACCT | 927 |
| TGTGACGTGT | 928 |
| TTTGGACTAA | 929 |
| TTCATGCCCG | 930 |
| TTGATCGTGG | 931 |
| TAGCATAGGA | 932 |
| GTAGTTGCAA | 933 |
| GGGACAGCTA | 934 |
| AAACCCCCAA | 935 |
| ACTCTCACAA | 936 |
| ATCATTGCCA | 937 |
| CCAGTTTGCG | 938 |
| ACATTAGTCA | 939 |
| CTCCAGGGTA | 940 |
| GAAGGGCCAA | 941 |
| CAGTCTCCCC | 942 |
| GAGACATTCC | 943 |
| AACGGTGTTG | 944 |
| AGCATTATCA | 945 |
| CTATACCGAG | 946 |
| AACTGGATCA | 947 |
| GTCTTGTCGG | 948 |
| GACGAGCCGC | 949 |
| GGAACACTGT | 950 |
| TAAATGCGTT | 951 |
| GCGAACACAG | 952 |
| TTCTCTCAAC | 953 |
| GTCGTACTGA | 954 |
| TGTGGCGTAA | 955 |
| TGAGCGGCGT | 956 |
| CCTCGTGAAC | 957 |
| GAGCAATGAA | 958 |
| CGAGACCTAA | 959 |
| AACTGAGCGC | 960 |
| TAAAGCTCGT | 961 |
| CTCTTTACGT | 962 |
| CCCCGTGGAA | 963 |
| TCGGTTCGTC | 964 |
| CTGCTTACAC | 965 |
| ACACCGTAAT | 966 |
| CCTGGTCGGC | 967 |
| GGTTATTTGG | 968 |
| GCAACTGAGT | 969 |
| ATAAGGCCTC | 970 |
| CGTGCGAAGG | 971 |
| GTCACACACT | 972 |
| CATACGGCAA | 973 |
| GAACTGCCCA | 974 |
| AATATGTGAA | 975 |
| CCGATCCTGT | 976 |
| CAAAGAGCCT | 977 |
| TAACTTAGAG | 978 |
| CAGCATGTAG | 979 |
| CCCCATGCAG | 980 |
| TCTGAACCAC | 981 |
| GCGTGCAAAA | 982 |
| GCTAGTACCG | 983 |
| TTTCCCGCGC | 984 |
| CCTTAGTAGG | 985 |
| TTGTGTCTTG | 986 |
| GCAACGAAGC | 987 |
| TGAAACCCTT | 988 |
| TTCTACGATC | 989 |
| ATTAAAGGTG | 990 |
| TATCTAACGG | 991 |
| AGTGCTCCTG | 992 |
| CCGTCCCTCT | 993 |
| CTAACGAGCG | 994 |
| AAGTCCGGCT | 995 |
| GGCGTATAAG | 996 |
| AGATATTAGG | 997 |
| TCCTAACAGC | 998 |
| GAGGATACGC | 999 |
| CGCTCTTTAA | 1000 |
| ACCGGCAGGC | 328 |
| GCTAAAATCT | 329 |
| GCCGTTGACG | 330 |
| GGAGTTGTTG | 331 |
| TACTTGAGAA | 332 |
| CGGGTGCGCT | 333 |
| AAAAGCGTCT | 334 |
| GTAAAGATAG | 335 |
| GCCTGGTCAG | 336 |
| GGCAAAAAGG | 337 |
| ACCCTTCTCT | 338 |
| TCACATAGTG | 339 |
| TCGTCTGTGC | 340 |
| TGCTCGGATC | 341 |
| GGCGTATAAG | 996 |
| AGATATTAGG | 997 |
| TCCTAACAGC | 998 |
| GAGGATACGC | 999 |
| CGCTCTTTAA | 1000 |
| GGCGTATAAG | 996 |
| AGATATTAGG | 997 |
| TCCTAACAGC | 998 |
| GAGGATACGC | 999 |

In another embodiment, a random sequence fragment can be linked to the 5′ and/or the 3′ end of the polynucleotide barcode and the random sequence fragment can, for example, be used for bioinformatic removal of PCR duplicates. The random sequence fragment can also be used to add length to the nucleic acid construct and can serve as a marker for bioinformatic analysis to identify the beginning or the end of the polynucleotide barcode after sequencing. In another embodiment, the nucleic acid construct comprises at least a first and a second random sequence fragment, and the first random sequence fragment can be linked to the 5′ end of the polynucleotide barcode and the second random sequence fragment can be linked to the 3′ end of the polynucleotide barcode. In another embodiment, one or at least one random sequence fragment is linked to the 5′ and/or the 3′ end of the polynucleotide barcode. In one aspect, the random sequence fragments can be extended as needed to make the nucleic acid construct longer for different applications such as whole genome sequencing where short inserts may be lost.

In various embodiments, the random sequence fragments can be from about 5 to about 20 base pairs in length, about 5 to about 19 base pairs in length, about 5 to about 18 base pairs in length, about 5 to about 17 base pairs in length, about 5 to about 16 base pairs in length, about 5 to about 15 base pairs in length, about 5 to about 14 base pairs in length, about 5 to about 13 base pairs in length, about 5 to about 12 base pairs in length, about 5 to about 11 base pairs in length, about 5 to about 10 base pairs in length, about 5 to about 9 base pairs in length, about 5 to about 8 base pairs in length, about 6 to about 10 base pairs in length, about 7 to about 10 base pairs in length, or about 8 to about 10 base pairs in length.

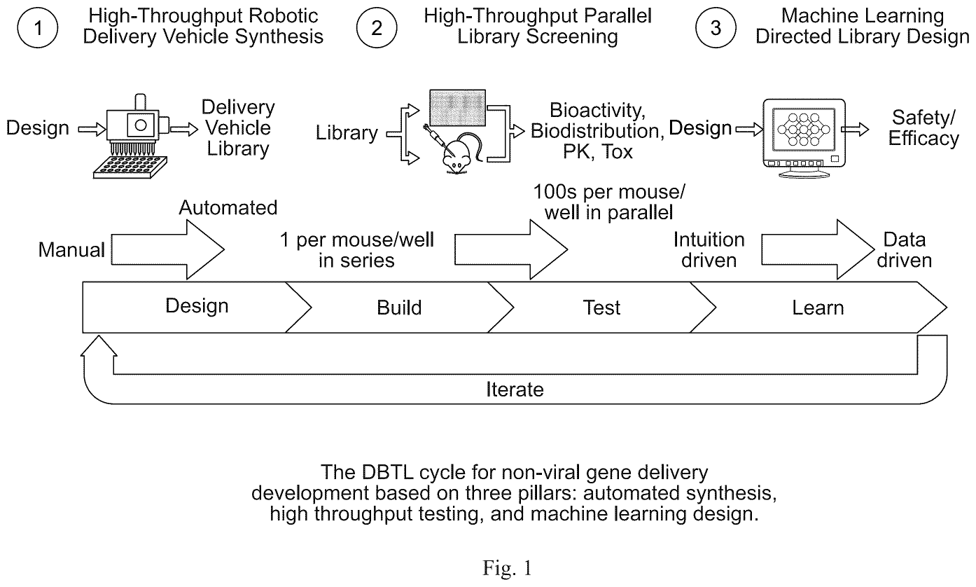

In another illustrative aspect, the polynucleotide barcode may be flanked by primer binding segments (i.e., directly or indirectly linked to the polynucleotide barcode) so that the nucleic acid construct comprising the polynucleotide barcode can be amplified during a polymerase chain reaction (PCR) and/or sequencing protocol. In one aspect, the primer binding segments can be useful for binding to one or more universal primers or a universal primer set. In one illustrative embodiment, the universal primers can contain overhang sequences that enable attachment of index adapters for sequencing. In this aspect, the primers can be any primers of interest. In this embodiment, the first primer binding segment can be linked at its 3′ end to the 5′ end of a first random sequence fragment and the second primer binding segment can be linked at its 5′ end to the 3′ end of a second random sequence fragment with the polynucleotide barcode between the random sequence fragments. In another embodiment, the first primer binding segment can be linked at its 3′ end to the 5′ end of the polynucleotide barcode and the second primer binding segment can be linked at its 5′ end to the 3′ end of a random sequence fragment (see FIG. 1 for an example) linked to the 3′ end of the polynucleotide barcode. In another embodiment, the first primer binding segment can be linked at its 3′ end to the 5′ end of a random sequence fragment and the second primer binding segment can be linked at its 5′ end to the 3′ end of the polynucleotide barcode where the polynucleotide barcode is linked at its 5′ end to the 3′ end of the random sequence fragment. In yet another embodiment, the first primer binding segment can be linked at its 3′ end to the 5′ end of the polynucleotide barcode and the second primer binding segment can be linked at its 5′ end to the 3′ end of the polynucleotide barcode.

In embodiments where primer binding segments are included in the nucleic acid construct, the primer binding segments can range in length from about 15 base pairs to about 30, from about 15 base pairs to about 29 base pairs, from about 15 base pairs to about 28 base pairs, from about 15 base pairs to about 26 base pairs, from about 15 base pairs to about 24 base pairs, from about 15 base pairs to about 22 base pairs, from about 15 base pairs to about 20 base pairs, 16 base pairs to about 28 base pairs, from about 16 base pairs to about 26 base pairs, from about 16 base pairs to about 24 base pairs, from about 16 base pairs to about 22 base pairs, from about 16 base pairs to about 20 base pairs, 17 base pairs to about 28 base pairs, from about 17 base pairs to about 26 base pairs, from about 17 base pairs to about 24 base pairs, from about 17 base pairs to about 22 base pairs, from about 17 base pairs to about 20 base pairs, 18 base pairs to about 28 base pairs, from about 18 base pairs to about 26 base pairs, from about 18 base pairs to about 24 base pairs, from about 18 base pairs to about 22 base pairs, or from about 18 base pairs to about 20 base pairs.

An exemplary sequence of a nucleic acid construct is shown below. The /5AmMC6/ is a 5′ amine modification for attachment to the polymer nanoparticle. The *'s are phosphorothioate bond modifications for stability. The A*G*A*CGTGTGCTCTTCCGATCT (SEQ ID NO: 1001) sequence is the 5′ primer binding segment sequence. The GCTACATAAT (SEQ ID NO: 1) is an exemplary barcode polynucleotide sequence. The N's represent the random sequence fragment. The AGATCGGAAGAGCGTCG*T*G*T (SEQ ID NO: 1002) is the 3′ primer binding segment sequence.

(SEQ ID NO: 1003)
/5AmMC6/A*G*A*CGTGTGCTCTTCCGATCTGCTACA
TAATNNNNNNNNNNAGATCGGAAGAGCGTCG*T*G*T

In all of the various embodiments described above, the entire nucleic acid construct can range in length from about 30 base pairs to about 240 base pairs, about 30 base pairs to about 230 base pairs, about 30 base pairs to about 220 base pairs, about 30 base pairs to about 210 base pairs, about 30 base pairs to about 200 base pairs, about 30 base pairs to about 190 base pairs, about 30 base pairs to about 180 base pairs, about 30 base pairs to about 170 base pairs, about 30 base pairs to about 160 base pairs, about 30 base pairs to about 150 base pairs, about 30 base pairs to about 140 base pairs, about 30 base pairs to about 130 base pairs, about 30 base pairs to about 120 base pairs, from about 30 base pairs to about 110 base pairs, from about 30 base pairs to about 100 base pairs, from about 30 base pairs to about 90 base pairs, from about 30 base pairs to about 80 base pairs, from about 30 base pairs to about 70 base pairs, from about 30 base pairs to about 60 base pairs, from about 30 base pairs to about 50 base pairs, from about 30 base pairs to about 40 base pairs, 40 base pairs to about 120 base pairs, from about 40 base pairs to about 110 base pairs, from about 40 base pairs to about 100 base pairs, from about 40 base pairs to about 90 base pairs, from about 40 base pairs to about 80 base pairs, from about 40 base pairs to about 70 base pairs, from about 40 base pairs to about 60 base pairs, from about 40 base pairs to about 50 base pairs, 50 base pairs to about 120 base pairs, from about 50 base pairs to about 110 base pairs, from about 50 base pairs to about 100 base pairs, from about 50 base pairs to about 90 base pairs, from about 50 base pairs to about 80 base pairs, from about 50 base pairs to about 70 base pairs, from about 50 base pairs to about 60 base pairs, or about 42 base pairs to about 210 base pairs.

The nucleic acid constructs are associated with the polymer nanoparticles, and exemplary polymer nanoparticle to nucleic acid construct ratio ranges are about 20:1 to about 10000:1, about 20:1 to about 9000:1, about 20:1 to about 8000:1, about 20:1 to about 7000:1, about 20:1 to about 6000:1, about 20:1 to about 5000:1, about 20:1 to about 4000:1, about 20:1 to about 3000:1, about 20:1 to about 2000:1, about 20:1 to about 1000:1, about 20:1 to about 900:1, about 20:1 to about 800:1, about 20:1 to about 700:1, about 20:1 to about 600:1, about 20:1 to about 500:1, about 20:1 to about 400:1, about 20:1 to about 300:1, about 20:1 to about 200:1, or about 20:1 to about 100:1.

In one illustrative aspect, the barcoded polymer nanoparticles may be used as delivery vehicles according to the present disclosure. In some embodiments, the non-viral delivery vehicle comprises one or more nanoparticle forming polymers. In some embodiments, the non-viral delivery vehicle comprises polymer nanoparticles. In some embodiments, the non-viral delivery vehicle is not a lipid based system. In some embodiments, the non-viral delivery vehicle comprises polymer nanoparticles made from controlled living/radical polymerization processes. It will be appreciated that the identity of the monomer units is not particularly limited so long as the monomer units being used are compatible with a controlled living/radical polymerization, such as reversible-deactivation radical polymerization, atom transfer radical polymerization (ATRP), reversible addition fragmentation chain transfer polymerization (RAFT), iodine-transfer polymerization (ITP), selenium-centered radical-mediated polymerization, telluride-mediated polymerization (TERP), stibine-mediated polymerization, ring-opening polymerization, or like polymerization processes. In some embodiments, the polymer nanoparticles may be made by RAFT copolymerization to synthesize a diverse set of block copolymers, and to screen their ability to form complexes with a payload. In one aspect, polymer nanoparticles (e.g., RAFT copolymers) may be produced by chemically bonding a payload to a constituent polymer, such as by the grafting of the payload onto RAFT copolymers using chain transfer agents, and subsequently assembling the polymers into a delivery vehicle.

In various embodiments, payloads may be combined with the polymer nanoparticles compositions using any or all of covalent bonds, electrostatic interactions, and ligand affinity interactions. In one aspect, covalent bonding methods include the use of EDC/NHS to form stable amide bonds between the payload and the polymer nanoparticles for improved stability (both “on the shelf” and in vivo), ease of separation and extraction, and sensitive detection. In another illustrative aspect, electrostatic bonding methods include the use of cationic polymer nanoparticles that electrostatically complex with the payload. In another embodiment, ligand affinity bonding includes the use of ligands such as avidin and biotin, both covalently bonded to the polymer nanoparticles and the payload via EDC/NHS chemistry to yield the stable combination of the payload and the polymer nanoparticles.

It will be appreciated that RAFT polymerization is generally known in the art. Suitable reagents, monomers, and conditions for RAFT polymerization previously investigated can be used in the copolymers, methods, and compositions described herein, such as those described in U.S. Pat. Nos. 9,006,193, 9,464,300, and 9,476,063, the disclosures of each of which are incorporated by reference in their entirety.

Chain transfer agents (CTAs) useful in connection with the present disclosure are known in the art. The identity of the CTA is not particularly limited. It will be appreciated that chain transfers steps that form the basis of RAFT polymerization involve a reversible transfer of a functional chain end-group (typically a thiocarbonylthio group, Z—C(═S)S—R) between chains and the propagating radicals. The overall process is comprised of the insertion of monomers between the R- and Z—C(═S)S-groups of a RAFT agent (CTA), which form the α and ω end-group of the majority of the resulting polymeric chains. Suitable CTAs for use in connection with the present disclosure include but are not limited to trithiocarbonates (Z═S-alkyl), dithiobenzoates (Z═Ph), dithiocarbamate (Z═N-alkyl), xanthates (Z═O-alkyl), and the like. (See, Sebastien Perrier, Macromolecules 2017 50 (19), 7433-7447) In some embodiments, RAFT copolymerization may be achieved using chain transfer agents (CTAs) containing one or more terminal carboxyl groups in order to obtain carboxy terminated polymers with ends available for bonding to the payload via the methods described above. In this embodiment, when the resulting mono or di-carboxy terminated polymer is dispersed in a low pH (e.g., a pH of less than 6) buffer, both ends of the polymer are exposed and available for labeling via EDC/NHS chemistry. In this embodiment, when the polymer is transferred to a physiological pH (˜pH 7), the core blocks self-assemble, encapsulating the payload in the hydrophobic core, to be released and exposed upon acidification in the endosomal compartment of a cell. In some embodiments, the first or second chain transfer agent can be selected from the group consisting of bis(carboxymethyl)trithiocarbonate, bis(2-amino-2-oxoethyl) trithiocarbonate, bis[4-(2-hydroxyethoxycarbonyl)benzyl] trithiocarbonate, 4--cyano--4- (ethylsulfanyithiocarbonyl) sulfanyhmentanoic acid, 4-cyano-4-((phenylcarbonothioyl)thio)pentanoic acid, and 4-cyano-4-[(dodecylsulfanylthiocarbonyl)sulfanyl] pentanoic acid, 4-cyano-4-(thiobenzoylthio)pentanoic acid, 2-cyano-2-propyl benzodithioate, cyanomethyl methyl(phenyl)carbamodithioate, 2-cyano-2-propyl dodecyl trithiocarbonate, 2-(dodecylthiocarbonothioylthio)-2-methylpropionic acid, cyanomethyl dodecyl trithiocarbonate, 2-cyano-2-propyl 4-cyanobenzodithioate, and the like.

It will be apprectiated that RAFT useful in connection with the present disclosure can be of a variety of polymer compositions. For example, RAFT polymers useful in connection with the present disclosure can be a randon block polymer comprising a single polymer block, or a diblock RAFT copolymer comprising two polymer blocks, or a triblock RAFT copolymer comprising three polymer blocks, or further numbers of blocks can be used. The skilled person will readily appreciate that the the preparation of block polymers by RAFT polymerization is known in the art and that such polymerization processes can be applied to the present disclosure. (See, Goby, et. al., Nat. Commun. 4:2505 doi: 10.1038/ncomms3505 (2013))

In some embodiments, RAFT copolymers as prepared herein can be described by the following structure:

CTACap-[Block 1]ₘ-[Block 2]ₙ-CTACap

where each CTACap is a capping unit derived from the chain transfer agent(s) used in the process for preparing the RAFT copolymer. The CTA used for preparing each of Block 1 and Block 2 can be the same or different. In some embodiments, the CTA used to prepare each of Block 1 and Block 2 is the same (e.g. macroCTA). In some embodiments, the CTA used to prepare each of Block 1 and Block 2 is different. In some embodiments, the CTA used to prepare one or both of Block 1 and Block 2 comprises a functional group for the covalent attachment of a biomolecule, drug, or label to the RAFT copolymer. In some embodiments, the covalent attachment can be via an ester or an amide bond. In some embodiments, the covalent attachment can be via EDC-NHS chemistry. In some embodiments, the first capping unit is of the formula

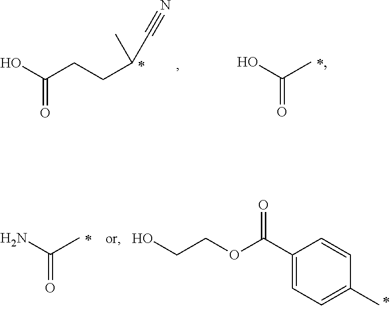

wherein * represents a point of covalent attachment to the first block. In some embodiments, the second capping unit is of the formula

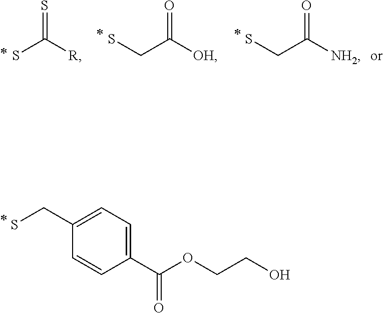

wherein * represents a point of covalent attachment to the second block, and R is —SC₂—C₁₂ alkyl or C₆H₅,

In some embodiments, the RAFT copolymer can be associated with a DNA molecule, in particular a nucleic acid construct of the present disclosure, via several methods including, electrostatic interaction, high affinity, non-covalent bond, avidin-streptavidin conjugation, or by direct covalent attachment through, for example, an amide bond. In some embodiments, the RAFT copolymer can be associated with a DNA molecule, in particular a nucleic acid construct of the present disclosure, via electrostatic interaction complexed with a biological molecule. In some embodiments, the RAFT copolymer can be associated with a DNA molecule, in particular a nucleic acid construct of the present disclosure, via electrostatic interaction complexed with a biological molecule. In some embodiments, the RAFT copolymer can be associated with a DNA molecule, in particular a nucleic acid construct of the present disclosure, via a high affinity, non-covalent bond, avidin-streptavidin conjugation. In some embodiments, the RAFT copolymer can be associated with a DNA molecule, in particular a nucleic acid construct of the present disclosure, by direct covalent attachment through, for example, an amide bond.

As shown in FIGS. 5(a)-5(e), the PNPs described herein can be associated with a nucleic acid construct of the present disclosure via electrostatic interaction, avidin-streptavidin conjugation, or by direct covalent attachment. Briefly, as shown in FIGS. 5(a)-5(e), the labels provided in the figure are as follows: 001. Polymer nanoparticle (PNP) with positively charged corona in the case of electrostatic loading. 002. Nucleic acid constructs with negative charges due to the phosphate groups. 003. Electrostatically loaded PNP-nucleic acid construct complex. 004. Carboxylate group on the terminal end of the polymer chains in the corona of the PNP. 005. Primary amine group on the 5′ end of the amine terminated nucleic acid construct. 006. Phosphate group on the 3′ end of the nucleic acid construct. 007. Amide bond formed in the direct amidification reaction between the amine terminal nucleic acid construct and the carboxylate terminated PNP. 008. Primary amine on the biotin bonding protein such as avidin. 010. Amide bond formed between the carboxylate group on the terminal end of the polymer chains in the corona of the PNP and the primary amine on the biotin bonding protein such as avidin. 011. Nucleic acid construct with a biotin functional group on the 5′ terminus. 012. Electrostatic coupling reaction that occurs when positively charged PNPs are mixed with negatively charged nucleic acid constructs. 013. Direct amidification reaction that is carried out via an EDA-NHC reaction between the carboxylate group on the terminal end of the polymer chains in the corona of the PNP and the primary amine on the amine terminated nucleic acid constructs. 014 Direct amidification reaction that is carried out via an EDA-NHC reaction between the carboxylate group on the terminal end of the polymer chains in the corona of the PNP and the primary amine on the biotin bonding protein such as avidin. 015. Coupling of the biotin on the 5′ end of the nucleic acid construct and the avidin conjugated to the carboxylate terminus on the corona of the PNPs.

In some embodiments, each of Block 1 and Block 2 can comprise one or more monomer units polymerized using a RAFT polymerization process. It will be appreciated that the identity of the monomer units is not particularly limited so long as the monomer units being used are compatible with a controlled living/radical polymerization, such as reversible-deactivation radical polymerization, atom transfer radical polymerization (ATRP), reversible addition fragmentation chain transfer polymerization (RAFT), iodine-transfer polymerization (ITP), selenium-centered radical-mediated polymerization, telluride-mediated polymerization (TERP), stibine-mediated polymerization, ring-opening polymerization, or like polymerization processes. Suitable monomer units include but are not limited to 2-dimethylaminoethyl acrylate (DMAEMA), 2-(diethylamino) ethyl methacrylate (DEAEMA), 2-(diisopropylamino) ethyl methacrylate (DPAEMA), butyl methacrylate (BMA), ethyl acrylic acid (EAA), propyl acrylic acid (PAA), (hydroxyethyl)methacrylate (HEMA), methyl methacrylate (MMA), Acrylic acid (AA), Acetoacetanilide (AAA), 4-Aminobenzonitrile (ABN), 9-Anthracenylmethyl acrylate (ACMA), 9-Anthracenylmethyl methacrylate (ACMMA), Aminoethyl methacrylate (AEM), 2-(2-aminoethylamino) ethyl methacrylate (AEAEMA), 4-(2-Acryloxyethoxy)-2-hydroxybenzophenone (AEHBP), 2-Aminoethyl methacrylate (AEMA), N-(2-Aminoethyl) methacrylamide (AEMAA), 3-amino-2-hydroxypropyl methacrylate (AEAHPMA), 3-aminopropyl methacrylamide (AHPMA), Allyl methacrylate (ALMA), Acrylamide (AM), Amidoamine (AMA), 3-Methacryl amido-3-methylbutanoic acid (AMBA), 2-Allyloxybenzaldehyde (AOBA), [2-(Acryloyloxy)ethyl]trimethylammonium chloride (AOETMA), 3-(Acryloyloxy)-2-hydroxypropyl methacrylate (AOHOPMA), 4-Aminophenethyl alcohol (APA), N-(3-Aminopropyl)methacrylamide (APMA), 5-(3-(Amino)-propoxy)-2-nitrobenzyl methacrylate (APNBMA), N-[N′-(2- aminoethyl)-2-aminoethyl]aspartamide (Asp(DET)), 2-Azidoethyl Methacrylate (AzEMA), 2,2′-Bithiophene (2-2-BTP), tert-Butyl acrylate (BA), Bromoacetaldehyde diethyl acetal (BAADA), N-(t-BOC-aminopropyl)methacrylamide (BAPMAA), tert-Butyl 2-bromoacrylate (BBA), 4-Butylbenzoyl chloride (BBC), 1,3-Butadiene (BD), 2-Butyl-2-ethyl-1,3-propanediol (BEPD), Di-tert-butyl iminodiacetate (BIDA), 3-(Bromomethyl)-5-((trimethylsilyl)ethynyl)benzenesulfonyl fluoride (BMTMSEBS), 2-(Benzyloxy)ethanol (BOE), 4-tert-Butoxystyrene (BOS), Branched polyethyleneimine (BPEI), 3-Bromo-5-((trimethylsilyl)ethynyl)benzenesulfonyl fluoride (BTMSEBS), ε-Caprolactone (CAP), Carboxybetaine methacrylate (CBMA), 2-Cyanoethyl N,N-diisopropylchlorophosphoramidite (CEDPCPP), N-Cyclohexylmaleimide (CHMI), 3-Chloro-2-hydroxypropyl methacrylate (CHPMA), Dodecyl acrylate (DA), N,N-Diallylacrylamide (DAA), Diallylmethylamine (DAMA), Diallyldimethylammonium chloride (DADMAC), 2,5-Diaminopyridine (DAP), 5,5′-Dibromo-4,4′-didodecyl-2,2′-bithiophene (DBDDBT), 5,5′-Dibromo-4,4′-ditetradecyl-2,2′-bithiophene (DBDTBT), 2,5-Dibromo-3-hexylthiophene (DBHTP), Dichloromethylvinylsilane (DCMVS), 2-(Diethylamino)ethanethiol hydrochloride (DEAET), 2-(Diethylamino)ethyl methacrylate (DEAEMA), Diethyl oxalpropionate (DEOP), DL-Lactide (DLL), N,N-dimethylamino-2-ethylmethacrylate) (DMA), N-[3-(N,N-dimethylamino)propyl]-methacrylamide (DMAPMA), [2-(Methacryloyloxy)ethyl]dimethyl-(3-sulfopropyl)ammonium hydroxide (DMAPS), N,N′-dimethylbutylamine (DMBA), N,N′-dimethylethanolamine (DMEA), N,N-dimethylamino-2-ethylacrylate or 2-(dimethylamino)ethyl acrylate (DMAEA), 1-(Dimethylamino)pyrrole (DMAP), 4,5-Dimethoxy-2-nitrobenzyl alcohol (DMONBA),3,4-Dimethoxythiophene (DMOT), 1,2-Dimyristoyl-sn-glycero-3-phosphocholine (DMPC), 2-deoxy-2-methacrylamido glucopyranose (DOMAAG), 1,2-Dioleoyl-sn-glycero-3-phosphocholine (DOPC), 1,2-Dioleoyl-sn-glycero-3-phosphoethanolamine (DOPE), 1,4,7,10-Tetraazacyclododecane-1,4,7,10-tetraacetic acid (DOTA), 1,2-Dioleoyl-3-trimethylammonium Propane (DOTAP), 1,2-Diphytanoyl-sn-glycero-3-phosphoethanolamine (DPyPE), 3-Dodecylthiophene (3-DT), N42-(2-pyridyldithio)lethyl methacrylamide (DTEMA), Ethyl acrylic acid (EAA), Ethyl 2-(bromomethyl)acrylate (EBMA), Ethyl 1-cyano-1-cyclopropanecarboxylate (ECCPC), 3,4-Ethylenedioxythiophene (EDOT), Ethylene glycol dimethacrylate (EGDMA), Ethylene glycol phenyl ether acrylate (EGPEA), Ethyl methacrylate (EMA), 3-(Fluorosulfonyl)-5-((trimethylsilyl)ethynyl)benzoic acid (3FTMSEBA), 3-Formyl-5-((trimethylsilyl)ethynyl)benzenesulfonyl fluoride (3FTMSEBS), 4-Formyl-2-((trimethylsilyl)ethynyl)benzenesulfonyl fluoride (4FTMSEBS), 5-Fluoro-2,3-thiophenedicarboxaldehyde (SFTPDCA), N-acetyl-D-galactose (GalNAc), N-acetyl-D-glucose (GlcNAc), Glycidyl methacrylate (GMA), Glycosylphosphatidylinositol (GPI), 5,7-Hexadecadiynoic acid (HDDA), 2-Hydroxyethyl methacrylate (HEMA), 1,1,1,3,3,3-Hexafluoroisopropyl acrylate (HFIPA), 1,1,4,7,10,10-Hexamethyltriethylenetetramine (HMTETA), 4-Hydroxybutyl acrylate (HOBA), 2-(4-Hydroxyphenylazo)benzoic acid (HPABA), 2,2,3,3,4,4,4-Heptafluorobutyl acrylate (HPFBA), N-(2-Hydroxypropyl)-Methacrylamide (HPMA), 2,2,3,4,4,4-Hexafluorobutyl acrylate (HXFBA), Isobornyl acrylate (IBA), 4-Iodobenzoyl chloride (IBC), Isobornyl methacrylate (IBMA), N-(Isobutoxymethyl)acrylamide (IBMAA), Isodecyl acrylate (IDA), Maleic anhydride (MA), [2-(Methacryloyloxy)ethyl]trimethylammonium chloride (MAETMA), Methacryloyl-L-Lysine (MAL), Methacryloxysuccinimide (MAS), Methacrylamidotrehalose (MAT), Methyl heptadecanoate (MHD), 1-Methyl-1H-indole-3-carbaldehyde (MICA), 2- N-Methylmaleimide (MMI), Methyl-5-norbornene-2,3-dicarboxylic anhydride (MNDCA), Methoxyethyl methacrylate (MOEMA), 2-(2-Methoxyethoxy)ethyl methacrylate (MOEOEMA), 3-Methyl-3-oxetanemethanol (MOM), 2-Methacryloyloxyethyl phosphorylcholine (MPC), Methyl 4-vinylbenzoate (MVB), 2-Naphthyl acrylate (NA), N-(Acryloxy)succinimide (NAS), o-Nitrobenzyl methacrylate (NBMA), N-Hydroxysuccinimide (NHS), N-(Methacryloxy) succinimide methacrylate (NHSMA), N-Isopropylacrylamide (NIPAM), 2-Naphthyl methacrylate (NMA), N-(Methacryloyloxy)succinimide (NMS), N-(n-Octadecyl)acrylamide (NODAA), 4-Nitro-N-propylbenzylamine hydrochloride (NPBAHC), Oligoethylene glycol methacrylate (OEGMA), Oligoethylenimine (OEI), 3-Phenylthiophene (3-PTP), Poly(N-methyl 4-vinylpyridine iodide) (P4VPQ), Poly(2-aminoethylmethacrylamide) (PAEMA), N-(N′-{N″-[N″′-(2-aminoethyl)-2-aminoethyl]-2-aminoethyl}-2- aminoethyl)-aspartamide (Asp(TEP)), 4-Pentenoic anhydride (PAN), Pentabromobenzyl acrylate (PBBA), Pentabromobenzyl methacrylate (PBBMA), Pentabromophenyl acrylate (PBPA), Pentabromophenyl methacrylate (PBPMA), Poly(ε-caprolactone) (PCL), trans-2-Phenylcyclopropyl isocyanate (PCPI), 1,5-Pentanediol (PD), 2-Phenyl-1,3-dioxan-5-ol (PDO), Pyridyl disulfide ethyl methacrylate (PDSEMA), Poly(ethylene glycol) (PEG), Poly(ethylene glycol) acrylate (PEGA), Poly(ethylene glycol) methyl ether methacrylate (PEGMEMA, Poly(ethylene glycol) ethyl ether methacrylate (PEGEEMA), Poly(ethylene glycol) methacrylate (PEGMA), Pentaethylenehexamine (PEHA), Poly(ethylenimine) (PEI), Pentaerythritol tetraacrylate (PETA), Pentafluorophenyl (PFP), Pentafluorophenyl acrylate (PFPA), Pentafluorophenyl methacrylate (PFPMA), Poly(glutamic acid) (PGA), Poly-(glycoamidoamine) (PGAA), Poly(glycidylbutylamine) (PGBA), Poly(glycidyl methacrylate) functionalized with ethanolamine (PGEA), Poly(glycidyl methacrylate) (PGMA), Poly(N-(2-Hydroxypropyl)methacrylamide) (PHPMA), Poly(lactic acid) (PLA), Poly(L-glutamate), (PLG), Poly(lactic-co-glycolic acid) (PLGA,), Poly(L-lysine) (PLL), Poly(L-lactic acid) (PLLA), Poly(lauryl methacrylate) (PLMA), Poly(methacrylic acid) (PMAA), Poly-(2-deoxy-2-methacrylamido glucopyranose) (PMAG), Poly-(methyl methacrylate) (PMMA), Poly(2-methacryloyloxyethyl phosphorylcholine) (PMPC), Poly[N-(3-(methacryloylamino) propyl)-N,N-dimethyl-N-(3-sulfopropyl) ammonium hydroxide] (PMPD), Poly(n-butyl acrylate) (PnBA), Poly(n-butyl methacrylate) (PnBMA), Poly(N-isopropyl acrylamide) (PNIPAM), Poly(oligoethylene glycol methacrylate) (POEGMA), Poly(propylene glycol) (PPG), Poly(propylenimine) (PPI), 3,4-Propylenedioxythiophene (ProDOT), Poly(styrene) (PS), Poly(sodium 4-styrenesulfonate) (PSS), Poly(tributyl-(4-vinylbenzyl)phosphonium chloride) (PTBP), Poly(triethyl-(4-vinylbenzyl)phosphonium chloride) (PTEP), Poly((2-trimethylamino)ethyl metacrylate chloride) (PTMAEMA), Poly((vinylbenzyl) trimethylammonium) (PVBTMA), Poly(2-vinyl-4,4-dimethylazlactone) (PVDMA), Poly(N-ethyl-4-vinylpyridinium bromide) (PVP), Quaternized Poly-DMAEMA (QPDMAEMA), Sulfobetaine methacrylate (SBMA), 3-Sulfopropyl methacrylate potassium salt (SPMAP), Thiocholesterol (TC), Thiophene-2,5-diboronic acid bis(pinacol) ester (TDABP), Triethylene glycol dimethacrylate (TEGDMA), Trifluoroethylene (TFE), 2,2,2-Trifluoroethyl acrylate (TFEA), 2,2,2-Trifluoroethyl methacrylate (TFEMA), Tetrahydrofurfuryl acrylate (THFA), Triallylisocyanurate (TIC), Trimethylene Carbonate (TMC), 4,4′-Trimethylenedipiperidine (TMDP), Trimethylolpropane tris(3-mercaptopropionate) (TMPTMP), (Trimethylsilyl)methacrylate (TMSMA), Triphenylcarbenium pentachlorostannate (TPCPCS), 3-Vinylbenzaldehyde (3VBA), 4-Vinylpyridinium chloride (4VP), Vinyl acrylate (VA), Vinyl acetate (VAT), 4-Vinylbenzoic acid (VBA), (Vinylbenzyl)trimethylammonium chloride (VBTAC), 1-Vinylimidazole (VI), 1-Vinyl-2-pyrrolidone (VP), m-Xylylenediamine (XDA), Zinc stearate (ZS), and the like.

In some embodiments, the monomer units used to make Block 1 and/or Block 2 of RAFT copolymers as described herein are selected from the group consisting of 2-(dimethylamino) ethyl acrylate (DMAEEA), 2-(diethylamino) ethyl methacrylate (DEAEEA), 2-(diisopropylamino) ethyl methacrylate (DIEAMA), butyl methacrylate (BMA), ethyl acrylic acid (EAA), propyl acrylic acid (PAA), (hydroxyethyl)methacrylate, and methyl methacrylate (MMA).

In some embodiments, the RAFT copolymers provided herein can be described by the formula:

CTACap-[(B1M1)_{m-B1M2-B1M3}-(B1M2)-_{m-B1M1-B1M3}-(B1M3)-_{m-B1M1-B1M2}]ₘ-[(B2M1)_{n-B2M2-B2M3}-(B2M2)-_{n-B2M1-B2M3}-(B2M3)-_{n-B2M1-B2M2}]ₙ-CTACap

For example, a RAFT copolymer as described herein having a single monomer in Block 1 of 25 units and 3 different monomers in Block 2 having an average monomer unit ratio of 20:10:5 for a total n of 35, can be described by the general formula

CTACap-[B1M1]₂₅-[(B2M1)₂₀-(B2M2)₁₀-(B2M3)₅]-CTACap

It will be further appreciated that the polymers prepared using a RAFT polymerization are random polymers having a distribution of units and hence molecular weights. Therefore, the cartoon representation of Block 2 in the example above is a random copolymer comprising 35 monomer units of B2M1, B2M2, and B2M3 in the ratio described above.

In another illustrative embodiment, the polymer nanoparticle composition can be coated with one or more polymers to protect the compositions from immune responses or to enhance endosomal escape. In one embodiment, the one or more polymers comprise polyethylene glycol. In another embodiment, the one or more polymers comprise polyethylene glycol poly-L-lysine. In yet another embodiment, the one or more polymers comprise polyethylenimine In an additional embodiment, the one or more polymers comprise polyethylene glycol poly-L-lysine and polyethylenimine

It will be appreciated that tuning the parameters and properties of the RAFT copolymers described herein can be advantageous to their use in the compositions and methods as described herein. A ccordingly, the methods for preparing RAFT copolymers either in singleton or in library format as described herein are capable of providing particular parameters and properties of the RAFT copolymers.

In some embodiments, a RAFT block polymer as described herein has one or more of an overall molecular weight (Mₙ) (i.e. the total of all blocks) in the range of about 1 kDa to about 1000 kDa, or about 2 kDa to about 500 kDa, or about 2 kDa to about 160 kDa, and overall degree of polymerization in the range of about 10 to about 3500, or about 20 to about 2500, or about 30 to about 900, a size in the range of about of about 10 nm to about 10000 nm, and a maximum corona-to-core ratio (CCR) of about 1 to about 4. In some embodiments, the overall molecular weight (Mₙ) in the range of about 30 kDa to about 120 kDa, about 40 kDa to about 110 kDa about 50 kDa to about 100 kDa, about 60 kDa to about 90 kDa, about 40 kDa to about 80 kDa, and about 40 kDa to about 60 kDa. In some embodiments, the overall degree of polymerization in the range of about 40 to about 850, about 60 to about 800, about 100 to about 700, about 200 to about 600, or about 300 to about 500. In some embodiments, the size is in the range of about of about 10 nm to about 10000 nm, or about 20 nm to about 5000 nm, or about 50 nm to about 3000 nm, or about 20 nm to about 1000 nm, or about 50 nm to about 1000 nm, or about 30 nm to about 500 nm, or about 200 nm to about 2000 nm, or about 100 nm to about 5000 nm, or about 100 nm to about 500 nm, or about 10 nm to about 50 nm, about 15 nm to about 45 nm, about 20 nm to about 40 nm, or about 25 nm to about 35 nm. In some embodiments, the maximum corona-to-core ratio (CCR) is less than 4, or less than 3, about 1 to about 3.8, about 1.2 to about 3.5, about 1.5 to about 3, about 1.5 to about 2.5, or about 1 to about 2.

In some embodiments, a first block can be prepared from one or more monomer units and have a molecular weight (Mₙ) in the range of about 1 kDa to about 500 kDa, or about 10 kDa to about 200 kDa, or about 10 kDa to about 160 kDa, or about 1 kDa to about 80 kDa, and a degree of polymerization in the range of about 10 to about 3500, or about 10 to about 2500, or about 20 to about 2000, or about 50 to about 1200, or about 50 to about 1000. In some embodiments, a first block molecular weight (Mₙ) can be in the range of about 1 kDa to about 500 kDa, or about 2 kDa to about 400 kDa, or about 5 kDa to about 200 kDa, or about 10 kDa to about 160 kDa, or about 15 kDa to about 100 kDa, or about 25 kDa to about 60 kDa, or about 30 kDa to about 55 kDa, about 30 kDa to about 50 kDa, or about 30 kDa to about 40 kDa, and the like. In some embodiments, the first block degree of polymerization is in the range of about 30 to about 350, about 50 to about 300, about 70 to about 250, about 80 to about 240, about 100 to about 200, and the like.

In some embodiments, the second block can be prepared from one or more monomer units, and can have a molecular weight (Mₙ)in the range of about 1 kDa to about 500 kDa, or about 10 kDa to about 200 kDa, or about 10 kDa to about 160 kDa, or about 1 kDa to about 80 kDa, and a degree of polymerization in the range of about 10 to about 3500, or about 10 to about 2500, or about 20 to about 2000, or about 50 to about 1200, or about 50 to about 1000. In some embodiments, the second block molecular weight (Mₙ) is in the range of about 10 kDa to about 70 kDa, about 15 kDa to about 65 kDa, about 20 kDa to about 60 kDa, about 25 kDa to about 55 kDa, about 30 kDa to about 50 kDa, about 35 kDa to about 45 kDa, about 5 kDa to about 15 kDa, and the like. In some embodiments, the second block degree of polymerization is in the range of about 3 to about 2500; or about 20 to about 2000, or about 30 to about 1500, or about 40 to about 1200, or about 10 to about 500, or about 12 to about 450, or about 20 to about 400, or about 25 to about 350, or about 50 to about 300, or about 100 to about 250, or about 150 to about 200, or about 5 to about 50, or about 5 to about 20, and the like.

In some embodiments, a third, fourth, or subsequent block can be prepared from one or more monomer units, and each can have a molecular weight (Mₙ) in the range of about 1 kDa to about 500 kDa, or about 10 kDa to about 200 kDa, or about 10 kDa to about 160 kDa, or about 1 kDa to about 80 kDa, and a degree of polymerization in the range of about 10 to about 3500, or about 10 to about 2500, or about 20 to about 2000, or about 50 to about 1200, or about 50 to about 1000. In some embodiments, the third, fourth, or subsequent block molecular weight (Mₙ) is in the range of about 10 kDa to about 70 kDa, about 15 kDa to about 65 kDa, about 20 kDa to about 60 kDa, about 25 kDa to about 55 kDa, about 30 kDa to about 50 kDa, about 35 kDa to about 45 kDa, about 5 kDa to about 15 kDa, and the like. In some embodiments, the third, fourth, or subsequent block degree of polymerization is in the range of about 3 to about 2500; or about 20 to about 2000, or about 30 to about 1500, or about 40 to about 1200, or about 10 to about 500, or about 12 to about 450, or about 20 to about 400, or about 25 to about 350, or about 50 to about 300, or about 100 to about 250, or about 150 to about 200, or about 5 to about 50, or about 5 to about 20, and the like.

In some embodiments, a single chain transfer agent can be used in the RAFT polymerization process in connection with the present disclosure. In some embodiments, for a block polymer having more than one block, one or more single chain transfer agents can be used in the RAFT polymerization process in connection with the present disclosure. In some embodiments, for a block polymer having two blocks, a first chain transfer agent and a second chain transfer agent (which can be the same or different) can be used at each step of the RAFT polymerization process in connection with the present disclosure. In some embodiments, for a block polymer having three blocks, a first chain transfer agent, a second chain transfer agent, and a third chain transfer agent (which can be the same or different) can be used at each step of the RAFT polymerization process in connection with the present disclosure.

It will be appreciated that a variety of solvents can be used in the RAFT polymerization method steps and purification steps described herein. Suitable solvents include, but are not limited to, 2-Chloroethanol, Acetic Acid (Glacial), Acetone, Acetonitrile, Acetophenone, Aniline, Benzaldehyde, Benzyl Acetate, Carbon disulfide, Cyclohexane, Cyclohexanol, Di(ethylene glycol), Di(propylene glycol), Diacetone alcohol, Diethyl ether, Dimethylsulfoxide, Ethanol, Ethyl acetate, Ethylene glycol, Formaldehyde (37% solution), Formamide, Formic acid, Formic acid (96%), Hexanelsobutanol, Isopropanol, Isopropyl acetate, Isopropyl ether, m-Cresol, Methanol, Methyl acetate, Methyl ethyl ketone, Mineral Oil, N,N-Dimethylformamide, n-Butanol, n-Octane, n-Propanol, Propylene glycol, Pyridine, t-Butanol, Tetrahydrofuran, Trifluoroacetic acid, water, and the like, and combinations thereof.

In some embodiments, the one or more nanoparticle forming polymers are RAFT block copolymers comprising
a. a first terminus comprising a first capping unit derived from a first chain transfer agent in a RAFT copolymerization process;
b. a first block prepared from one or more monomer units covalently attached to the first reactive functional unit, and having a molecular weight (Mₙ) in the range of about 1 kDa to about 500 kDa and a degree of polymerization in the range of about 10 to about 2500;
c. optionally a second block prepared from one or more monomer units covalently attached to the first block, and having a molecular weight (Mₙ)in the range of about 1 kDa to about 500 kDa and a degree of polymerization in the range of about 10 to about 2500;
d. optionally a third block prepared from one or more monomer units covalently attached to the first and/or second blocks, and having a molecular weight (Mₙ)in the range of about 1 kDa to about 500 kDa and a degree of polymerization in the range of about 10 to about 2500;
e. optionally a fourth block prepared from one or more monomer units covalently attached to the first, second, and/or third blocks, and having a molecular weight (Mₙ)in the range of about 1 kDa to about 500 kDa and a degree of polymerization in the range of about 10 to about 2500;
f. optionally a fifth block prepared from one or more monomer units covalently attached to the first, second, third, and/or fourth blocks, and having a molecular weight (Mₙ) in the range of about 1 kDa to about 500 kDa and a degree of polymerization in the range of about 10 to about 2500; and
g. a second terminus comprising a second capping unit derived from a first, a second, a third, or a fourth chain transfer agent.

Illustrative payloads for the polymer nanoparticle described herein can include any one or a combination of compositions selected from the group comprising: nucleic acids (e.g., DNA or RNA), pDNA, oligodeoxyribonucleic acids (ODNs), dsDNA, ssDNA, antisense oligonucleotides, antisense RNA, siRNA, messenger RNA, guide RNA (e.g., small guide RNA), ribonucleoproteins, donor DNA strands used in the CRISPR/Cas9 system, and enzymes, such as CRISPR-associated enzymes, e.g., Cas9, enzymes used in other gene editing systems, such as ZFNs, custom designed homing endonucleases, TALENS systems, other gene editing endonucleases, and reverse transcriptase.

In another aspect, the present disclosure rapidly identifies top candidates using a machine learning model. In the illustrative embodiment, a graph neural network (GNN) is used for this process. Polymers can be characterized at three scales: monomer, block, and full polymer. Monomers combine to form blocks, and blocks combine to form full polymers. Polymer properties are dependent on characteristics of the polymer at all three scales. The relationships between monomers, blocks, and polymers can be captured with a directed graph. Information can then be shared between nodes in the graph to create a numerical representation of the full polymer at all three scales: monomer, block, and polymer. These numerical representations can then be used in a neural network to prediction properties of the polymer. The use of a GNN for polymer property prediction in the illustrative embodiment provides two primary benefits: first, the graph can model polymer characteristics at all three scales which is important for accurate prediction; second, the graph provides a flexible modeling structure that can accommodate several polymer structures.

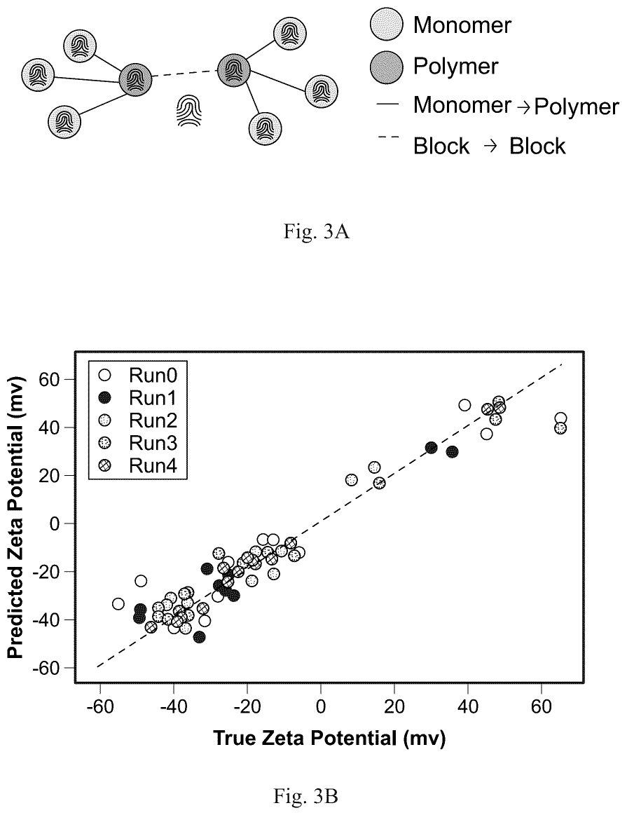

The machine learning model is first trained on a combination of public data and preliminary testing data, supplemented with the large data sets described above. The illustrative embodiment involves a three-loop deep learning cycle to accelerate high-throughput characterization and screening for PNPs. The three deep learning loops characterize the PNP physical properties, in vitro bioactivity, and in vivo bioactivity, respectively. Each loop utilizes a GNN deep learning model (see FIG. 3a) to characterize the candidate PNPs. The GNN takes the simplified molecular-input line-entry system (SMILES) strings defining the monomers as an input (the nodes of the graph), and the edges of the graph define the relationship between the monomers and how they combine to form the PNP. The edges of the graph also allow additional information about the polymer (e.g., ratios of monomers and degree of polymerization) to be incorporated into the GNN.

The presently disclosed architecture offers at least three distinct advantages. First, the deep learning model is not dependent on polymer fingerprinting. Rather, the deep learning model will learn an appropriate numerical embedding from the SMILES strings. Second, the graph allows the model the flexibility to represent various families of PNPs with ease. Third, using SMILES strings as inputs allows the limited training dataset to be augmented with enumerated SMILES strings, increasing the amount of training data available and improving the model performance. Testing of this GNN architecture has shown impressive ability to predict zeta potential (see FIG. 3b), a critical characteristic for non-viral gene delivery vehicles. Once trained, these deep learning models will be used to prioritize the synthesis and characterization of candidate PNPs in the high-throughput system to meet the requirements of a bioactivity. Iterative data can be used to fine-tune the models in an active learning cycle to improve future performance.

In some embodiments, data augmentation may be performed to artificially increase the size and variety of the data used to train the machine learning model (and, consequently, increase model performance). Deep learning models require relatively large datasets for training and can over-fit to small datasets. As discussed above, the GNN takes the SMILES strings defining the monomers as an input. A monomer (a building block of a polymer) has a single canonical SMILES string, but it also has multiple alternative SMILES string representations. SMILES enumeration can be performed to generate these alternative forms from the canonical SMILES string and, thus, increase the size of the training data set many times over. The neural network model is then able to leverage this increase in data size and variety of representations to improve performance.

In other embodiments, a modified Transformer model (rather than a GNN) may be used to predict polymer properties (and, thus, rapidly identify top candidates for non-viral carriers for delivering base editing proteins, among other applications). The modified Transformer model exploits relative positional information of inputs to create numerical embeddings for monomer string inputs. These numerical embeddings can then be used in deep learning and statistical models for polymer property prediction. Additionally, the Transformer model is more computationally efficient compared to many other deep learning architectures that can process sequential data. The original Transformer architecture consists of an encoding and decoding architecture. The encoder takes an input sequence of data and outputs a high dimensional embedding, while the decoder takes the high dimensional embedding as an input and tries to predict the original or similar sequence to the one input into the encoder. The present disclosure does not need to predict a sequential output, so it only uses the encoding portion of the Transformer to predict polymer properties, both physical and in-vitro/in-vivo.

In yet another aspect, an illustrative embodiment of the present disclosure allows for the selection the top candidates for PNP-mediated delivery of the SOD1-targeting CBE in a mouse model of ALS. Functional gene editing tests in a microglial cell line stably expressing EGFP or SOD1 can then be performed using these top candidates. Moreover, the efficacy and safety PNP-mediated CBE delivery can be assessed in the G93A-SOD1 mouse model of ALS. Prior success of CBE base editors for slowing ALS progression in mice shows a likelihood that they can also lead to clinical translation of a novel ALS gene editing therapy.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

### EXAMPLES

### Example 1

### Barcode Design

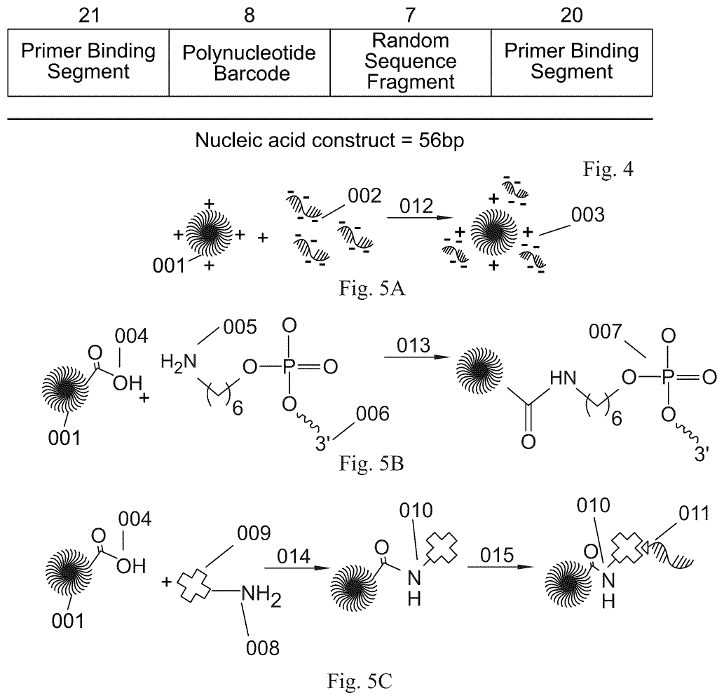

The nucleic acid constructs used in this example comprised a unique portion comprising 8-10 nucleotides in the center of the polynucleotide, the unique portion further constrained by the requirement of a hamming distance of at least 3 bases from any other barcodes to be pooled. Directly on the 3′ end of the barcode, 7-10 random bases are included for bioinformatic removal of PCR duplicates. This central sequence is flanked by universal primer annealing sites containing overhangs for the addition of index adapters during sequencing library preparation. FIG. 4 shows a representative illustration of these barcodes. These nucleic acid constructs were designed with either a biotin functional group or an amine functional group on the 5′ end.

### Example 2

### Polymer Nanoparticle Synthesis

A diblock copolymer was synthesized as described in PCTapp349529(21477779.1) using reversible addition-fragmentation chain transfer (RAFT) polymerization with reagents and amounts listed in Table 2. Block 1 reagents were combined in a round bottom flask, purged with argon, and heated to 60° C. for 6 hours using a heating mantle. The reaction product was purified using four 80:20 pentane:ether precipitation washes and centrifugation cycles and dried in vacuo. The Block 1 product was used as the macroRAFT agent for Block 2, and the calculated reagent volumes (as calculated based on theoretical molecular weight information for Block 1) were combined in a round bottom for the Block 2 reaction. The reaction mixture was argon purged before being heated at 60° C. for 24 hours. The reaction product was purified using the same purification process and dried in vacuo. The resulting polymer was dialyzed in deionized water for 4 days with multiple water changes each day. Finally, the dialyzed material was lyophilized for 4 days and stored at room temperature for experimental use.

TABLE 2
Reagents and amounts used to synthesize barcoded PNPs

| | | | |
|---|---|---|---|
| | | Lot 0001 | Lot 0002 |
| Reagent | Purpose | Amount | Amount |

Block 1

| | | | | | |
|---|---|---|---|---|---|
| 2-dimethylaminoethyl acrylate (DMAEMA) | Monomer | 15999.6 | mg | 32000.0 | mg |
| (4-cyano-4-(((ethylthio)carbonothioyl)thio)pentanoicacid) | Chain transfer | 76.9 | mg | 153.7 | mg |
| (ECT) | agent | | | | |
| Azobisisobutyronitrile (AIBN) | Initiator | 9.5 | mg | 19.05 | mg |
| Dimethylformamide (DMF) | Solvent | 24131.1 | mg | 48229.8 | mg |

| | | | |
|---|---|---|---|
| Block 1 Reaction Yield | % Yield | 31.75% | 39.55% |

Block 2

| | | | | | |
|---|---|---|---|---|---|
| 2-dimethylaminoethyl acrylate (DMAEMA) | Monomer | 713.0 | mg | 2110.6 | mg |
| butyl methacrylate (BMA) | Monomer | 1934.4 | mg | 5727.6 | mg |
| propyl acrylic acid (PAA) | Monomer | 518.2 | mg | 1540.1 | mg |
| Block 1 macroRAFT agent, meaning ECT + DMAEMA. | Macro Chain | 1528.4 | mg | 4526.2 | mg |
| The ECT end groups (R & Z) were still present to perform | transfer agent | | | | |
| their function, but they were on the end of the p(DMAEMA) | | | | | |
| polymer synthesized as block 1. For reference, here is ECT | | | | | |
| R & Z groups on either side of onyl group. | | | | | |
| 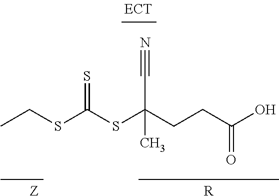 | | | | | |
| Azobisisobutyronitrile (AIBN) | Initiator | 1.7 | mg | 4.93 | mg |
| Dimethylformamide (DMF) | Solvent | 7045.0 | mg | 20828.8 | mg |

| | | | |
|---|---|---|---|
| Block 2 Reaction Yield | % Yield | 73.33% | 70.99% |

### Example 3

### Composition Example 1 (CE1): Electrostatic Attachment of Nucleic Acid Constructs (Containing DNA Barcodes) to Polymer Nanoparticles

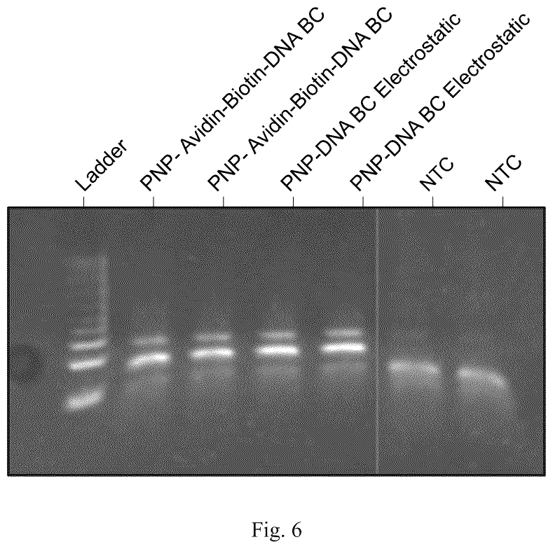

RAFT copolymers were synthesized according to the methods above and the reagents listed in Table 2. The polymer was dispersed in phosphate buffered saline at a concentration of ˜5 mg/ml. For electrostatic loading (FIG. 5a), nucleic acid constructs (according to the design shown in FIG. 4 including polynucleotide barcodes) were dissolved in tris EDTA buffer at a concentration of ˜100 μM (˜1.9 mg/mL). These stock solutions were mixed together with PBS to produce a solution with a final concentration of 0.05 mg/mL polymer and 0.00389 mg/mL nucleic acid construct. They were incubated at room temperature for at least 30 minutes to allow the positively charged polymer to associate with the negatively charged nucleic acid constructs. The sample was transferred to an amicon ultra-4 centrifuge tube (MWCO 30 kDa). (Max 3.5 mL/Tube) and centrifuged at 4,000×g for 15 minutes to remove any unbound nucleic acid constructs. The filtrate (containing unbound nucleic acid constructs) was discarded and sterile PBS was added to the retentate to final concentration of 0.05 mg/mL polymer and 0.00389 mg/mL nucleic acid constructs. The electrostatically bound nucleic acid constructs were amplified via PCR, using primers designed to bind to the universal primer binding segments on the nucleic acid constructs. The amplicons were detected via gel electrophoresis on agarose gel. The presence of a double band (FIG. 6) in a lane indicates the presence of amplicons, showing that nucleic acid constructs were bound to the PNPs.

### Example 4

Composition Example 2 (CE2): Nucleic Acid Construct Conjugation to PNPs Via Avidin-Biotin Linkage

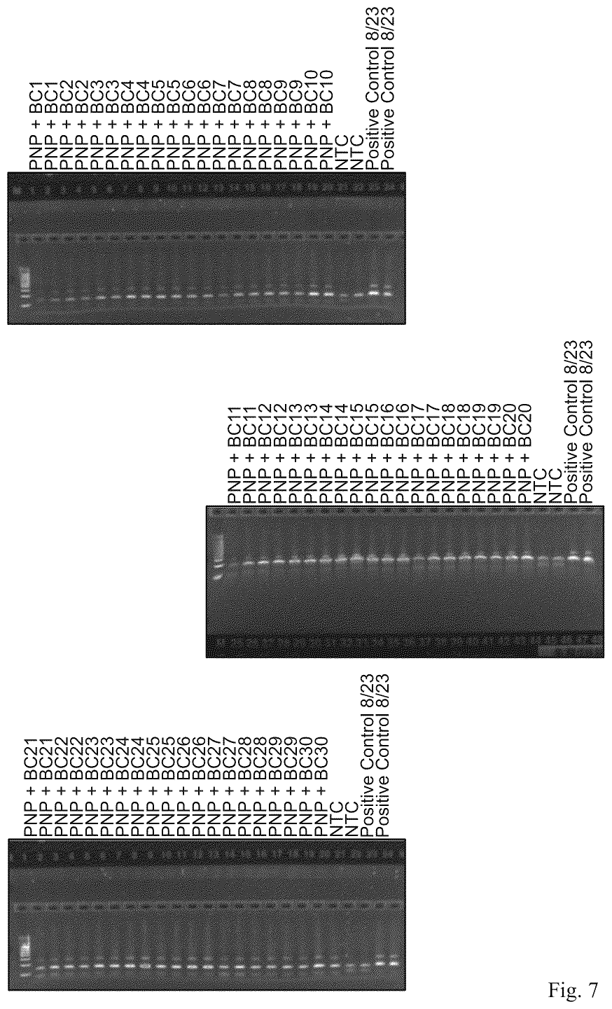

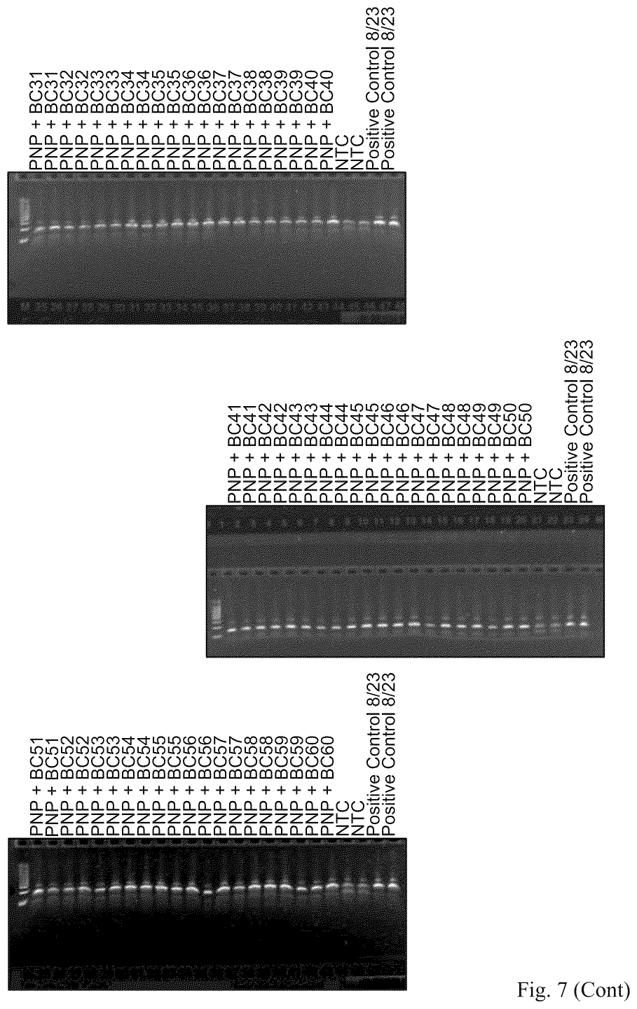

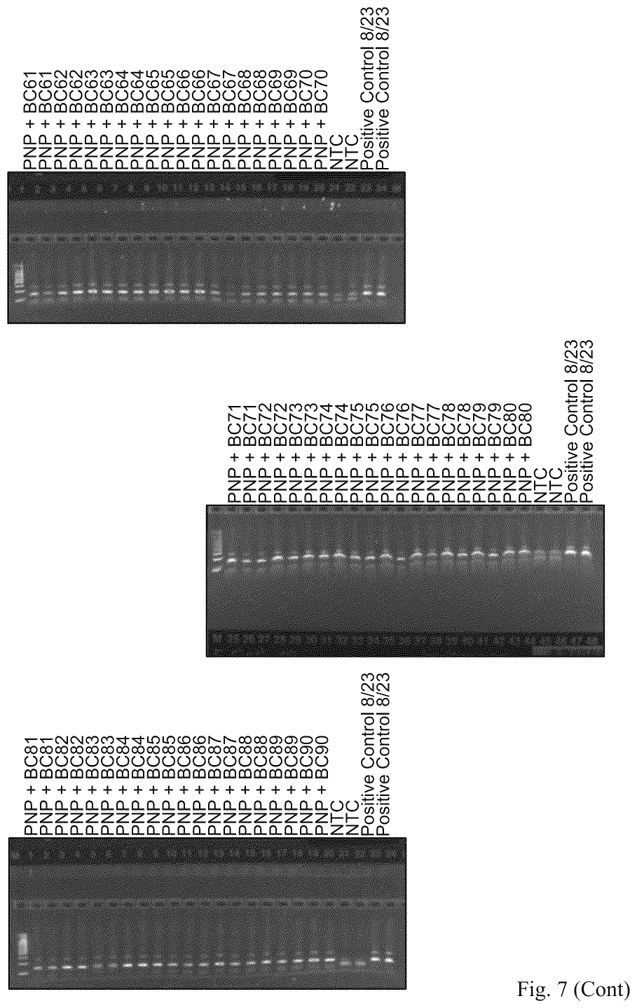

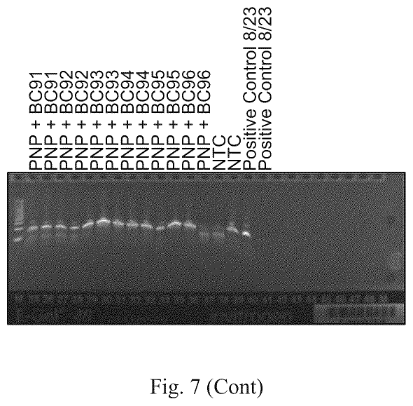

RAFT copolymers made using CTAs that contain at least one carboxyl terminal group were further functionalized with avidin (FIG. 5b). A RAFT copolymer was transferred into a MES buffer at ˜12 mg/mL. The sample was sonicated for 30 minutes. EDC reagent and Sulfo-NHS reagent was added to the polymer at a molar ratio of 10:1 and 25:1 respectively, reagent to PNP. The sample was incubated for at least 10 minutes at room temperature to allow the reaction to occur. The reaction volume was filtered through a membrane with a molecular weight cut off of 30 kDa via centrifuge at ˜3000×g for ˜15 minutes. The filtrate was discarded, and sterile PBS was added to the retentate to reconstitute to 10 mg/mL polymer. Avidin (36.9 mg) was added to the reaction and incubated for 15 minutes at room temperature. The sample was transferred to an amicon ultra-4 centrifuge tube (MWCO 30 kDa). (Max 3.5 mL/Tube) and centrifuged at 4,000×g for 15 minutes. The filtrate was discarded and sterile PBS was added to the retentate to final concentration of 8 mg/mL polymer. A nucleic acid constructs with biotin attached to the 5′ end was added to the avidin functionalized polymer at a molar ratio of 10 moles of polymer to 1 mole of nucleic acid constructs. The sample was incubated for at least 15 minutes. The sample was transferred to an amicon ultra-4 centrifuge tube (MWCO 30 kDa, Max 3.5 mL/Tube) and centrifuged at 4,000×g for 15 minutes to remove any unbound nucleic acid constructs. The filtrate (containing unbound nucleic acid constructs) was discarded and sterile PBS was added to the retentate to final concentration of 8 mg/mL polymer. The conjugated nucleic acid constructs were amplified via PCR, using primers designed to bind to the universal primer binding segments on the nucleic acid constructs. The amplicons were detected via gel electrophoresis on agarose gel. The presence of a double band (FIGS. 6 and 7) in a lane indicates the presence of amplicons, showing that nucleic acid constructs were bound to the PNPs.

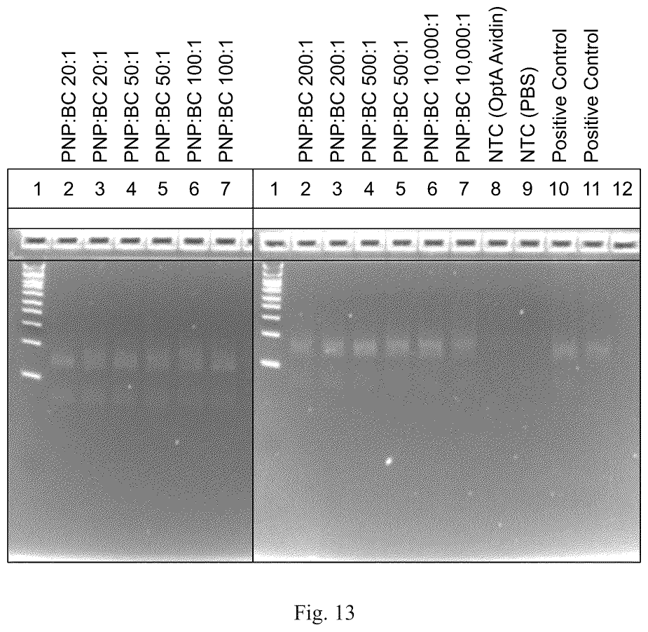

To test the range of PNP to barcode ratios that can be used in the reduction to practice of nucleic acid constructs labeled PNPs, the method above was used to attach the nucleic acid constructs to PNPs using the avidin-biotin linkage (FIG. 5a), varying the PNP to nucleic acid constructs ratio from as low as 20:1 to as high as 10,000 to 1 (moles polymer to moles nucleic acid constructs). FIG. 13 shows a gel electrophoresis graph with bands corresponding to the amplicons from nucleic acid constructs produced from a PCR reaction on the nucleic acid constructs with these various PNP to nucleic acid constructs ratios, indicating that these ratios are in the useable range for the reduction to practice of the nucleic acid constructs PNP composition.

The avidin-biotin conjugation method was used to attach 96 unique barcodes to 96 aliquots of the polymer described in Table 2, yielding 96 aliquots of the same polymer in which the population of nanoparticles in each aliquot has a unique barcode attached. These 96 aliquots were pooled by combining the aliquots in volumetrically equivalent amounts into a single vial, yielding a dispersion of 96 distinct populations of barcoded PNPs, in which all populations comprised a polymer micelle formed from the polymer described in Table 2 and a unique barcode from the population of 96 unique barcodes.

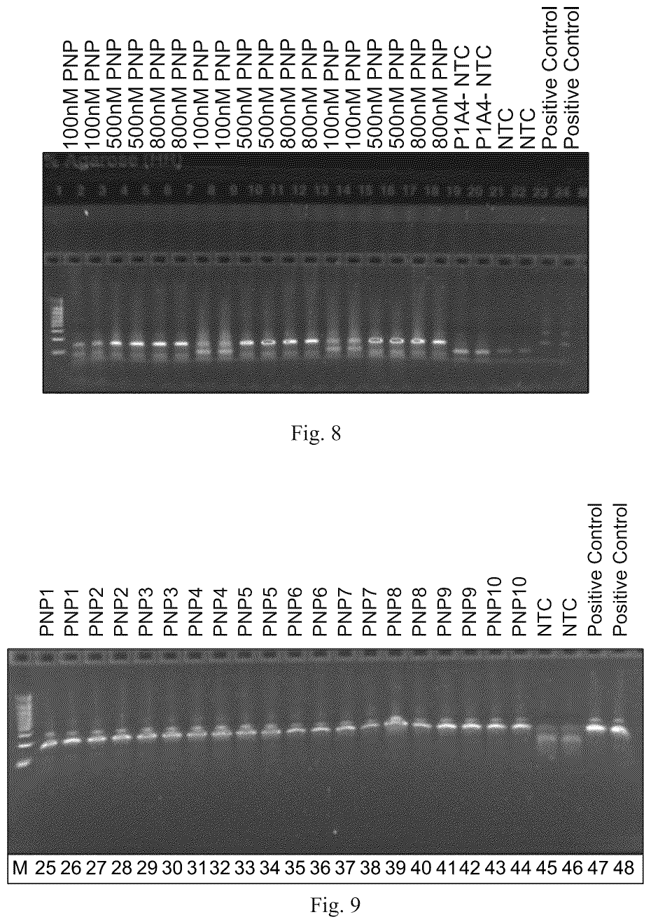

The pooled sample of avidin-biotin conjugated nucleic acid construct-PNPs were spiked into HEK-293T cells. The cells were seeded in 96 well plates at 20,000 cells per well, in 100 μl at of media and left to adhere overnight. Twenty-four hours after seeding, the pooled sample of PNPs with 96 unique barcodes were added at a dose of ˜0.024 mg/mL PNP in each well and placed in an incubator at 37° C. overnight. The next day, barcodes extracted from the samples, using the QlAamp 96 DNA extraction kit and a Qiacube HT instrument according to the manufacturer's protocol. The conjugated barcodes were amplified via PCR, using primers designed to bind to the universal primer binding sequences on the barcodes. The amplicons were detected via gel electrophoresis on agarose gel. The presence of a double band (FIG. 8) in a lane indicates the presence of amplicons, showing that nucleic acid constructs were bound to the PNPs.

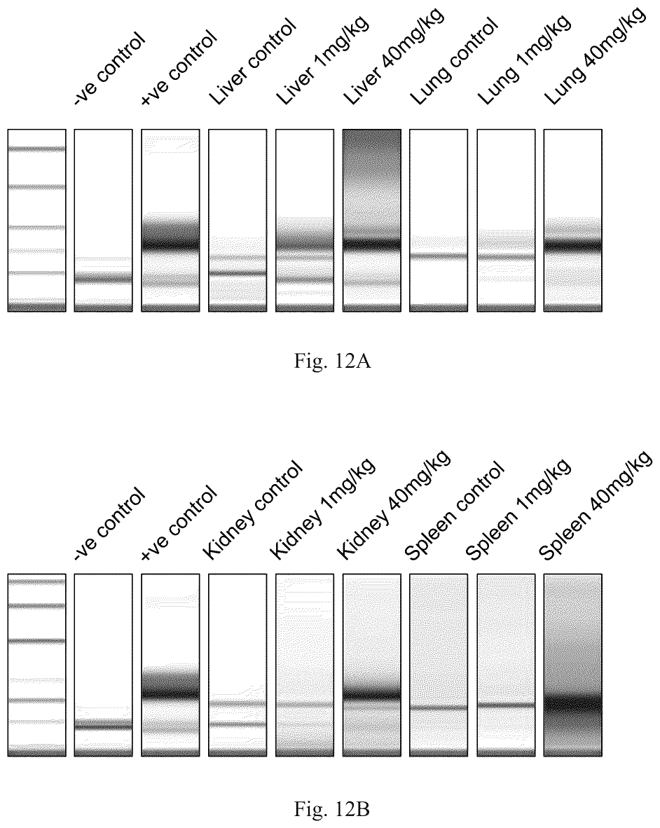

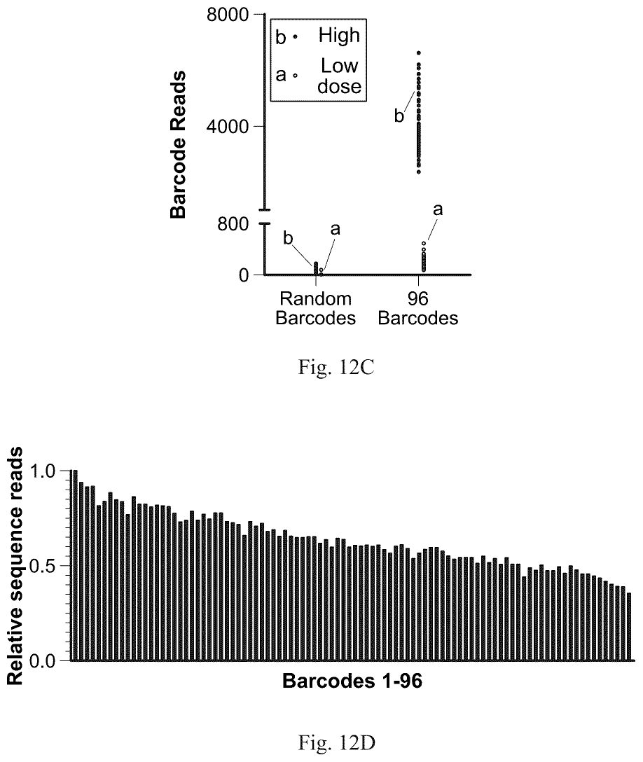

The pooled sample of PNPs with 96 unique barcodes conjugated via avidin-biotin linkage, was administered to mice (using the in vivo screening protocol described below). Twenty-four hours after dosing, the mice were sacrificed and the tissues were analyzed for the presence of the barcoded PNPs. PCR was used to amplify the barcodes from the tissue samples and agilent fragment analysis was used to detect the presence of nucleic acid constructs-PNPs, with a dark band matching the positive control as the indicator of the presence of nucleic acid constructs-PNPs (FIG. 12a). This experiment reduced to practice the ability to label PNPs with unique nucleic acid constructs, administer them to mice, and then detect their biodistribution via PCR. We further used deep sequencing techniques to distinguish all 96 unique barcodes in the liver tissue. Library preparations, sequencing and sequence analysis were performed as described below. All 96 unique barcodes were detected in a way that was countable and distinguishable from the others (FIG. 12c and FIG. 12d). This shows that our compositions allow not only the detection of uniquely barcoded PNPs in mouse tissue 24 hours after dosing, but it also allows us to quantitatively distinguish each uniquely barcoded PNP from all other uniquely barcoded PNPs in the animal tissue.

### Example 5

### Preparation of RAFT Copolymers for Polymer Nanoparticle Libraries

Briefly, Block 1 reagents (monomer(s), chain transfer agent, initiator, and solvent) were combined in wells of a polypropylene 96-well u-shaped bottom microplate (Greiner Bio-One), in polypropylene 96 well cluster tubes (Corning), in polypropylene Eppendorf microcentrifuge tubes (Sigma-Aldrich), or in polypropylene 50 mL or 15 mL conical tubes (VWR) and placed in a VWR 1400E Sheldon vacuum oven. A 20 mL glass vial was filled with approximately 10-15 mL of solvent (e.g., dimethylformamide), and the vial was placed in the oven to provide a source for atmosphere saturation. The oven was purged with argon at ˜3 L/min for approximately 45 minutes and heated to between 60° C. and 75° C. for 6-24 hours. Upon completion of the reaction, acetone was added to the wells or tubes to prevent polymer solidification and the wells or tubes were sealed and left at room temperature overnight. The next day, the reaction product solutions were transferred to 1.5 mL Eppendorf tubes (if necessary) and purified via at least three precipitation washes using an appropriate purification solvent solution (e.g., 80:20 pentane:ether, isopropyl alcohol, methanol, etc.) and centrifugation cycles and dried in vacuo. The Block 1 product was used as the macroRAFT agent for Block 2, and the calculated reagent volumes (as calculated based on theoretical or actual molecular weight information for Block 1) were combined in a polypropylene 96-well u-shaped bottom microplate (Greiner Bio-One), in polypropylene 96 well cluster tubes (Corning), in polypropylene Eppendorf microcentrifuge tubes (Sigma-Aldrich), or in polypropylene 50 mL or 15 mL conical tubes (VWR) for the Block 2 reaction. The reaction mixtures were placed in a VWR 1400E Sheldon vacuum oven, which was argon purged at ˜3 L/min for approximately 45 minutes before being heated to between 60° C. and 75° C. for 6-24 hours. The reaction products were purified using the same purification process as used for Block 1 library materials and dried in vacuo. The resulting polymers were resuspended in either acetone or chloroform and aliquoted as needed for experimental use (these transfer solvents evaporated prior to material use), stored in a dry state at room temperature, or dissolved in deionized water, frozen, and lyophilized prior to experimental use. Size was measured using a Wyatt Technology DynaPro Plate Reader III. Molecular weights for Block 1 materials were measured using a DynaPro Plate Reader III. Nanoparticle sizes above the DynaPro Plate Reader III molar mass capability threshold prevented measurement of Block 2 molecular weights for these polymer libraries. All molecular weights for high-throughput polymer libraries are reported as weight average molecular weight (M_{w}).

A summary of the reagents, amounts, and reaction conditions used to synthesize Block 1 and Block 2 of a pilot PNP library of 96 PNPs are shown in Tables 3 and 4, respectively, below. PNPs 22, 61, and 89-96 were used as 10 unique PNPs for HEK cell studies. PNPs 1-88 were used as unique PNPs for flow cytometry studies. Table 3 Abbreviations: ACVA, 4,4′-Azobis(4-cyanovaleric acid); AIBN, Azobisisobutyronitrile; BMA, butyl methacrylate; CTP, 4-Cyano-4-(thiobenzoylthio)pentanoic acid; DMAEMA, dimethylaminoethyl methacrylate; DMF, N,N-Dimethylformamide; ECT, 4-Cyano-4-Rethylsulfanylthiocarbonyl)sulfanyllpentanoic acid; MMA, methyl methacrylate. Table 4 Abbreviations: ACVA, 4,4′-Azobis(4-cyanovaleric acid); AIBN, Azobisisobutyronitrile; BMA, butyl methacrylate; CTP, 4-Cyano-4-(thiobenzoylthio)pentanoic acid; DMAEMA, dimethylaminoethyl methacrylate; DMF, N,N-Dimethylformamide; ECT, 4-Cyano-4-[(ethylsulfanylthiocarbonyl)sulfanyl]pentanoic acid; HEMA, 2-Hydroxyethyl methacrylate; MMA, methyl methacrylate. Table 5 Abbreviations: PDI, polydispersity index.

Static Light Scattering (SLS) and Dynamic Light Scattering (DLS) measurements to determine Block 1 molar mass and PNP size (e.g., diameter) were determined using a DynaPro Plate Reader III by Wyatt Technology. Data acquisition and handling were made with DYNAMICS software. SLS and DLS data were obtained under the following conditions:
SOLVENT: Water
TEMPERATURE: 25° C.
SAMPLE VOLUME: 200 μL
DATA ACQUISITION SETTINGS: 5 acquisitions of 5 seconds per acquisition

TABLE 3
Summary of Block 1 Reagents and Reaction Conditions Used in Pilot PNP Library

| | Block 1 Reagents, Purpose, Amounts, and Reaction Conditions | | | | | | | | | | |
|---|---|---|---|---|---|---|---|---|---|---|---|
| | | | Chain | | | | | | | | |
| | | | Transfer | | | | | | | | |
| | | | Agent (CTA) | | | | | | | | Block 1 |
| | Monomer and | | and Amount | | Initiator and | | Solvent | | Time | Temp | Rxn % |
| PNP | Amount (mol) | | (mol) | | Amount (mol) | | Amount (mol) | | (min) | (C.) | Yield |
| 1 | DMAEMA | 2.49E−04 | CTP | 2.49E−06 | ACVA | 2.49E−06 | DMF | 2.04E−03 | 363 | 75 | 56.4 |
| 2 | DMAEMA | 2.49E−04 | CTP | 1.09E−06 | ACVA | 1.09E−06 | DMF | 2.04E−03 | 363 | 75 | 62.1 |
| 3 | DMAEMA | 2.49E−04 | CTP | 8.54E−07 | ACVA | 8.55E−07 | DMF | 2.04E−03 | 363 | 75 | 59.2 |
| 4 | DMAEMA | 2.49E−04 | CTP | 7.00E−07 | ACVA | 7.01E−07 | DMF | 2.04E−03 | 363 | 75 | 57.9 |
| 5 | DMAEMA | 2.49E−04 | CTP | 5.18E−07 | ACVA | 5.16E−07 | DMF | 2.04E−03 | 363 | 75 | 66.7 |
| 6 | DMAEMA | 2.49E−04 | CTP | 4.61E−07 | ACVA | 4.57E−07 | DMF | 2.04E−03 | 363 | 75 | 66.6 |
| 7 | DMAEMA | 2.49E−04 | CTP | 4.13E−07 | ACVA | 4.08E−07 | DMF | 2.04E−03 | 363 | 75 | 61.5 |
| 8 | DMAEMA | 2.49E−04 | CTP | 3.65E−07 | ACVA | 3.68E−07 | DMF | 2.04E−03 | 363 | 75 | 50.6 |
| 9 | DMAEMA | 2.49E−04 | CTP | 3.36E−07 | ACVA | 3.37E−07 | DMF | 2.04E−03 | 363 | 75 | 57.2 |
| 10 | DMAEMA | 2.49E−04 | CTP | 3.07E−07 | ACVA | 3.12E−07 | DMF | 2.04E−03 | 363 | 75 | 56.7 |
| 11 | DMAEMA | 2.49E−04 | CTP | 1.52E−06 | ACVA | 1.52E−06 | DMF | 2.04E−03 | 363 | 75 | 74.3 |
| 12 | DMAEMA | 2.49E−04 | CTP | 1.09E−06 | ACVA | 1.09E−06 | DMF | 2.04E−03 | 363 | 75 | 73.0 |
| 13 | DMAEMA | 2.49E−04 | CTP | 8.54E−07 | ACVA | 8.55E−07 | DMF | 2.04E−03 | 363 | 75 | 68.1 |
| 14 | DMAEMA | 2.49E−04 | CTP | 7.00E−07 | ACVA | 7.01E−07 | DMF | 2.04E−03 | 363 | 75 | 64.6 |
| 15 | DMAEMA | 2.49E−04 | CTP | 5.18E−07 | ACVA | 5.16E−07 | DMF | 2.04E−03 | 363 | 75 | 69.5 |
| 16 | DMAEMA | 2.49E−04 | CTP | 4.61E−07 | ACVA | 4.57E−07 | DMF | 2.04E−03 | 363 | 75 | 57.0 |
| 17 | DMAEMA | 2.49E−04 | CTP | 4.13E−07 | ACVA | 4.08E−07 | DMF | 2.04E−03 | 363 | 75 | 53.4 |
| 18 | DMAEMA | 2.49E−04 | CTP | 3.65E−07 | ACVA | 3.68E−07 | DMF | 2.04E−03 | 363 | 75 | 66.4 |
| 19 | DMAEMA | 2.49E−04 | CTP | 3.36E−07 | ACVA | 3.37E−07 | DMF | 2.04E−03 | 363 | 75 | 55.0 |
| 20 | DMAEMA | 2.49E−04 | CTP | 2.49E−06 | ACVA | 2.49E−06 | DMF | 2.04E−03 | 363 | 75 | 66.6 |
| 21 | DMAEMA | 2.49E−04 | CTP | 1.52E−06 | ACVA | 1.52E−06 | DMF | 2.04E−03 | 363 | 75 | 75.4 |
| 22 | DMAEMA | 2.49E−04 | CTP | 1.09E−06 | ACVA | 1.09E−06 | DMF | 2.04E−03 | 363 | 75 | 73.6 |
| 23 | DMAEMA | 2.49E−04 | CTP | 8.54E−07 | ACVA | 8.55E−07 | DMF | 2.04E−03 | 363 | 75 | 65.1 |
| 24 | DMAEMA | 2.49E−04 | CTP | 7.00E−07 | ACVA | 7.01E−07 | DMF | 2.04E−03 | 363 | 75 | 71.4 |
| 25 | DMAEMA | 2.49E−04 | CTP | 5.95E−07 | ACVA | 5.94E−07 | DMF | 2.04E−03 | 363 | 75 | 60.7 |
| 26 | DMAEMA | 2.49E−04 | CTP | 5.18E−07 | ACVA | 5.16E−07 | DMF | 2.04E−03 | 363 | 75 | 66.6 |
| 27 | DMAEMA | 2.49E−04 | CTP | 4.61E−07 | ACVA | 4.57E−07 | DMF | 2.04E−03 | 363 | 75 | 55.2 |
| 28 | DMAEMA | 2.49E−04 | CTP | 4.13E−07 | ACVA | 4.08E−07 | DMF | 2.04E−03 | 363 | 75 | 55.7 |
| 29 | DMAEMA | 2.49E−04 | CTP | 3.65E−07 | ACVA | 3.68E−07 | DMF | 2.04E−03 | 363 | 75 | 51.4 |
| 30 | DMAEMA | 2.49E−04 | CTP | 3.36E−07 | ACVA | 3.37E−07 | DMF | 2.04E−03 | 363 | 75 | 53.3 |
| 31 | DMAEMA | 2.49E−04 | CTP | 3.07E−07 | ACVA | 3.12E−07 | DMF | 2.04E−03 | 363 | 75 | 47.2 |
| 32 | DMAEMA | 2.49E−04 | CTP | 1.52E−06 | ACVA | 1.52E−06 | DMF | 2.04E−03 | 363 | 75 | 88.7 |
| 33 | DMAEMA | 2.49E−04 | CTP | 1.09E−06 | ACVA | 1.09E−06 | DMF | 2.04E−03 | 363 | 75 | 72.0 |
| 34 | DMAEMA | 2.49E−04 | CTP | 8.54E−07 | ACVA | 8.55E−07 | DMF | 2.04E−03 | 363 | 75 | 65.0 |
| 35 | DMAEMA | 2.49E−04 | CTP | 5.95E−07 | ACVA | 5.94E−07 | DMF | 2.04E−03 | 363 | 75 | 69.4 |
| 36 | DMAEMA | 2.49E−04 | CTP | 4.61E−07 | ACVA | 4.57E−07 | DMF | 2.04E−03 | 363 | 75 | 46.1 |
| 37 | DMAEMA | 2.49E−04 | CTP | 4.13E−07 | ACVA | 4.08E−07 | DMF | 2.04E−03 | 363 | 75 | 47.5 |
| 38 | DMAEMA | 2.49E−04 | CTP | 3.36E−07 | ACVA | 3.37E−07 | DMF | 2.04E−03 | 363 | 75 | 52.3 |
| 39 | DMAEMA | 2.49E−04 | CTP | 3.07E−07 | ACVA | 3.12E−07 | DMF | 2.04E−03 | 363 | 75 | 55.9 |
| 40 | DMAEMA | 2.49E−04 | CTP | 2.49E−06 | ACVA | 2.49E−06 | DMF | 2.04E−03 | 363 | 75 | 82.3 |
| 41 | DMAEMA | 2.49E−04 | CTP | 8.54E−07 | ACVA | 8.55E−07 | DMF | 2.04E−03 | 363 | 75 | 76.5 |
| 42 | DMAEMA | 2.49E−04 | CTP | 7.00E−07 | ACVA | 7.01E−07 | DMF | 2.04E−03 | 363 | 75 | 71.7 |
| 43 | DMAEMA | 2.49E−04 | CTP | 4.13E−07 | ACVA | 4.08E−07 | DMF | 2.04E−03 | 363 | 75 | 54.9 |
| 44 | DMAEMA | 2.49E−04 | CTP | 3.65E−07 | ACVA | 3.68E−07 | DMF | 2.04E−03 | 363 | 75 | 54.3 |
| 45 | DMAEMA | 2.49E−04 | CTP | 3.36E−07 | ACVA | 3.37E−07 | DMF | 2.04E−03 | 363 | 75 | 54.6 |
| 46 | DMAEMA | 2.49E−04 | CTP | 3.07E−07 | ACVA | 3.12E−07 | DMF | 2.04E−03 | 363 | 75 | 55.0 |
| 47 | DMAEMA | 2.49E−04 | CTP | 1.09E−06 | ACVA | 1.09E−06 | DMF | 2.04E−03 | 363 | 75 | 93.5 |
| 48 | DMAEMA | 2.49E−04 | CTP | 8.54E−07 | ACVA | 8.55E−07 | DMF | 2.04E−03 | 363 | 75 | 79.7 |
| 49 | DMAEMA | 2.49E−04 | CTP | 5.18E−07 | ACVA | 5.16E−07 | DMF | 2.04E−03 | 363 | 75 | 56.9 |
| 50 | DMAEMA | 2.49E−04 | CTP | 4.61E−07 | ACVA | 4.57E−07 | DMF | 2.04E−03 | 363 | 75 | 52.0 |
| 51 | DMAEMA | 2.49E−04 | CTP | 4.13E−07 | ACVA | 4.08E−07 | DMF | 2.04E−03 | 363 | 75 | 55.8 |
| 52 | DMAEMA | 2.49E−04 | CTP | 3.36E−07 | ACVA | 3.37E−07 | DMF | 2.04E−03 | 363 | 75 | 51.8 |
| 53 | DMAEMA | 2.49E−04 | CTP | 3.07E−07 | ACVA | 3.12E−07 | DMF | 2.04E−03 | 363 | 75 | 53.1 |
| 54 | DMAEMA | 2.49E−04 | CTP | 1.52E−06 | ACVA | 1.52E−06 | DMF | 2.04E−03 | 363 | 75 | 86.5 |
| 55 | DMAEMA | 2.49E−04 | CTP | 8.54E−07 | ACVA | 8.55E−07 | DMF | 2.04E−03 | 363 | 75 | 72.9 |
| 56 | DMAEMA | 2.49E−04 | CTP | 7.00E−07 | ACVA | 7.01E−07 | DMF | 2.04E−03 | 363 | 75 | 71.5 |
| 57 | DMAEMA | 2.49E−04 | CTP | 5.95E−07 | ACVA | 5.94E−07 | DMF | 2.04E−03 | 363 | 75 | 59.9 |
| 58 | DMAEMA | 2.49E−04 | CTP | 5.18E−07 | ACVA | 5.16E−07 | DMF | 2.04E−03 | 363 | 75 | 63.7 |
| 59 | DMAEMA | 2.49E−04 | CTP | 4.13E−07 | ACVA | 4.08E−07 | DMF | 2.04E−03 | 363 | 75 | 52.0 |
| 60 | DMAEMA | 2.49E−04 | CTP | 3.65E−07 | ACVA | 3.68E−07 | DMF | 2.04E−03 | 363 | 75 | 54.9 |
| 61 | DMAEMA | 2.49E−04 | CTP | 3.36E−07 | ACVA | 3.37E−07 | DMF | 2.04E−03 | 363 | 75 | 55.3 |
| 62 | DMAEMA | 2.49E−04 | CTP | 3.07E−07 | ACVA | 3.12E−07 | DMF | 2.04E−03 | 363 | 75 | 42.2 |
| 63 | DMAEMA | 2.49E−04 | CTP | 1.52E−06 | ACVA | 1.52E−06 | DMF | 2.04E−03 | 363 | 75 | 82.9 |
| 64 | DMAEMA | 2.49E−04 | CTP | 1.09E−06 | ACVA | 1.09E−06 | DMF | 2.04E−03 | 363 | 75 | 71.2 |
| 65 | DMAEMA | 2.49E−04 | CTP | 5.95E−07 | ACVA | 5.94E−07 | DMF | 2.04E−03 | 363 | 75 | 60.4 |
| 66 | DMAEMA | 2.49E−04 | CTP | 5.18E−07 | ACVA | 5.16E−07 | DMF | 2.04E−03 | 363 | 75 | 64.9 |
| 67 | DMAEMA | 2.49E−04 | CTP | 3.65E−07 | ACVA | 3.68E−07 | DMF | 2.04E−03 | 363 | 75 | 54.0 |
| 68 | DMAEMA | 2.49E−04 | CTP | 3.36E−07 | ACVA | 3.37E−07 | DMF | 2.04E−03 | 363 | 75 | 44.2 |
| 69 | DMAEMA | 2.49E−04 | CTP | 3.07E−07 | ACVA | 3.12E−07 | DMF | 2.04E−03 | 363 | 75 | 52.4 |
| 70 | DMAEMA | 2.49E−04 | CTP | 2.49E−06 | ACVA | 4.97E−07 | DMF | 2.04E−03 | 363 | 75 | 52.5 |
| 71 | DMAEMA | 2.49E−04 | CTP | 1.52E−06 | ACVA | 3.03E−07 | DMF | 2.04E−03 | 363 | 75 | 46.6 |
| 72 | DMAEMA | 2.49E−04 | CTP | 1.09E−06 | ACVA | 2.19E−07 | DMF | 2.04E−03 | 363 | 75 | 40.2 |
| 73 | DMAEMA | 2.49E−04 | CTP | 8.54E−07 | ACVA | 1.71E−07 | DMF | 2.04E−03 | 363 | 75 | 37.7 |
| 74 | DMAEMA | 2.49E−04 | CTP | 5.18E−07 | ACVA | 1.03E−07 | DMF | 2.04E−03 | 363 | 75 | 27.3 |
| 75 | DMAEMA | 2.49E−04 | CTP | 4.61E−07 | ACVA | 9.05E−08 | DMF | 2.04E−03 | 363 | 75 | 26.7 |
| 76 | DMAEMA | 2.49E−04 | CTP | 4.13E−07 | ACVA | 8.21E−08 | DMF | 2.04E−03 | 363 | 75 | 25.1 |
| 77 | DMAEMA | 2.49E−04 | CTP | 3.65E−07 | ACVA | 7.37E−08 | DMF | 2.04E−03 | 363 | 75 | 23.0 |
| 78 | DMAEMA | 2.49E−04 | CTP | 3.36E−07 | ACVA | 6.74E−08 | DMF | 2.04E−03 | 363 | 75 | 22.8 |
| 79 | DMAEMA | 2.49E−04 | CTP | 2.49E−06 | ACVA | 4.97E−07 | DMF | 2.04E−03 | 363 | 75 | 52.7 |
| 80 | DMAEMA | 2.49E−04 | CTP | 1.52E−06 | ACVA | 3.03E−07 | DMF | 2.04E−03 | 363 | 75 | 47.6 |
| 81 | DMAEMA | 2.49E−04 | CTP | 1.09E−06 | ACVA | 2.19E−07 | DMF | 2.04E−03 | 363 | 75 | 39.3 |
| 82 | DMAEMA | 2.49E−04 | CTP | 8.54E−07 | ACVA | 1.71E−07 | DMF | 2.04E−03 | 363 | 75 | 37.1 |
| 83 | DMAEMA | 2.49E−04 | CTP | 7.00E−07 | ACVA | 1.41E−07 | DMF | 2.04E−03 | 363 | 75 | 34.0 |
| 84 | DMAEMA | 2.49E−04 | CTP | 5.95E−07 | ACVA | 1.18E−07 | DMF | 2.04E−03 | 363 | 75 | 31.8 |
| 85 | DMAEMA | 2.49E−04 | CTP | 5.18E−07 | ACVA | 1.03E−07 | DMF | 2.04E−03 | 363 | 75 | 25.1 |
| 86 | DMAEMA | 2.49E−04 | CTP | 4.61E−07 | ACVA | 9.05E−08 | DMF | 2.04E−03 | 363 | 75 | 25.7 |
| 87 | DMAEMA | 2.49E−04 | CTP | 4.13E−07 | ACVA | 8.21E−08 | DMF | 2.04E−03 | 363 | 75 | 23.9 |
| 88 | DMAEMA | 2.49E−04 | CTP | 7.00E−07 | ACVA | 1.41E−07 | DMF | 2.04E−03 | 363 | 75 | 27.7 |
| 89 | DMAEMA | 2.49E−04 | CTP | 8.54E−07 | ACVA | 1.71E−07 | DMF | 2.04E−03 | 363 | 75 | 32.8 |
| 90 | DMAEMA | 2.49E−04 | CTP | 3.65E−07 | ACVA | 7.37E−08 | DMF | 2.04E−03 | 363 | 75 | 21.0 |
| 91 | DMAEMA | 2.53E−04 | CTP | 8.42E−07 | ACVA | 8.44E−07 | DMF | 2.03E−03 | 360 | 70 | 103.4 |
| 92 | MMA | 3.95E−04 | CTP | 1.32E−06 | AIBN | 2.63E−07 | DMF | 2.04E−03 | 360 | 70 | 61.2 |
| 93 | BMA | 2.80E−04 | ECT | 9.32E−07 | AIBN | 1.86E−07 | DMF | 2.01E−03 | 360 | 70 | 32.4 |
| 94 | DMAEMA | 2.53E−04 | ECT | 8.43E−07 | AIBN | 8.44E−07 | DMF | 2.03E−03 | 360 | 65 | 101.0 |
| 95 | MMA | 3.96E−04 | ECT | 1.32E−06 | ACVA | 1.32E−07 | DMF | 2.04E−03 | 360 | 65 | 13.7 |
| 96 | BMA | 2.80E−04 | CTP | 9.31E−07 | AIBN | 9.32E−07 | DMF | 2.01E−03 | 360 | 65 | 32.1 |

TABLE 4
Summary of Block 2 Reagents and Reaction Conditions Used in Pilot PNP Library

| | Block 2 Reagents, Purpose, Amounts, and Reaction Conditions | | | | | | | | | | |
|---|---|---|---|---|---|---|---|---|---|---|---|
| | | | | | | | | | | | Diblock |
| | | | Macro Chain | | | | | | | | Post- |
| | | | Transfer Agent | | | | Solvent and | | | | Lyophilization |
| | Monomer and | | (mCTA) and | | Initiator and | | Amount | | Time | Temp | % |
| PNP | Amount (mol) | | Amount (mol) | | Amount (mol) | | (mol) | | (min) | (C.) | Yield |
| 1 | HEMA | 2.54E−05 | p(DMA | 1.27E−06 | ACVA | 1.27E−06 | DMF | 8.00E−04 | 1440 | 64 | 65.3 |
| | | | EMA), | | | | | | | | |
| | | | (006-A1 | | | | | | | | |
| | | | Block 1) | | | | | | | | |
| 2 | HEMA | 2.70E−05 | p(DMA | 1.33E−06 | ACVA | 1.33E−06 | DMF | 8.33E−04 | 1440 | 64 | 85.7 |
| | | | EMA), | | | | | | | | |
| | | | (006-A3 | | | | | | | | |
| | | | Block 1) | | | | | | | | |
| 3 | HEMA | 2.38E−05 | p(DMA | 1.19E−06 | ACVA | 1.19E−06 | DMF | 7.25E−04 | 1440 | 64 | 77.4 |
| | | | EMA), | | | | | | | | |
| | | | (006-A4 | | | | | | | | |
| | | | Block 1) | | | | | | | | |
| 4 | HEMA | 1.91E−05 | p(DMA | 9.60E−06 | ACVA | 9.60E−07 | DMF | 5.36E−04 | 1440 | 64 | 56.6 |
| | | | EMA), | | | | | | | | |
| | | | (006-A5 | | | | | | | | |
| | | | Block 1) | | | | | | | | |
| 5 | HEMA | 2.15E−05 | p(DMA | 1.08E−06 | ACVA | 1.08E−06 | DMF | 5.92E−04 | 1440 | 64 | 83.8 |
| | | | EMA), | | | | | | | | |
| | | | (006-A7 | | | | | | | | |
| | | | Block 1) | | | | | | | | |
| 6 | HEMA | 2.07E−05 | p(DMA | 1.03E−06 | ACVA | 1.03E−06 | DMF | 5.54E−04 | 1440 | 64 | 87.4 |
| | | | EMA), | | | | | | | | |
| | | | (006-A8 | | | | | | | | |
| | | | Block 1) | | | | | | | | |
| 7 | HEMA | 1.99E−05 | p(DMA | 9.78E−06 | ACVA | 9.78E−07 | DMF | 5.32E−04 | 1440 | 64 | 85.5 |
| | | | EMA), | | | | | | | | |
| | | | (006-A9 | | | | | | | | |
| | | | Block 1) | | | | | | | | |
| 8 | HEMA | 1.51E−05 | p(DMA | 7.57E−07 | ACVA | 7.56E−07 | DMF | 3.99E−04 | 1440 | 64 | 95.6 |
| | | | EMA), | | | | | | | | |
| | | | (006-A10 | | | | | | | | |
| | | | Block 1) | | | | | | | | |
| 9 | HEMA | 1.75E−05 | p(DMA | 8.71E−07 | ACVA | 8.71E−07 | DMF | 4.56E−04 | 1440 | 64 | 87.4 |
| | | | EMA), | | | | | | | | |
| | | | (006-A11 | | | | | | | | |
| | | | Block 1) | | | | | | | | |
| 10 | HEMA | 1.51E−05 | p(DMA | 7.48E−07 | ACVA | 7.49E−07 | DMF | 3.62E−04 | 1440 | 64 | 90.2 |
| | | | EMA), | | | | | | | | |
| | | | (006-A12 | | | | | | | | |
| | | | Block 1) | | | | | | | | |
| 11 | HEMA | 7.07E−05 | p(DMA | 1.55E−06 | ACVA | 1.56E−06 | DMF | 2.67E−03 | 1440 | 64 | 73.3 |
| | | | EMA), | | | | | | | | |
| | | | (006-B2 | | | | | | | | |
| | | | Block 1) | | | | | | | | |
| 12 | HEMA | 4.53E−05 | p(DMA | 9.94E−07 | ACVA | 9.95E−07 | DMF | 1.58E−03 | 1440 | 64 | 71.6 |
| | | | EMA), | | | | | | | | |
| | | | (006-B3 | | | | | | | | |
| | | | Block 1) | | | | | | | | |
| 13 | HEMA | 5.96E−05 | p(DMA | 1.31E−06 | ACVA | 1.31E−06 | DMF | 2.22E−03 | 1440 | 64 | 70.3 |
| | | | EMA), | | | | | | | | |
| | | | (006-B4 | | | | | | | | |
| | | | Block 1) | | | | | | | | |
| 14 | HEMA | 5.16E−05 | p(DMA | 1.12E−06 | ACVA | 1.12E−06 | DMF | 1.87E−03 | 1440 | 64 | 87.8 |
| | | | EMA), | | | | | | | | |
| | | | (006-B5 | | | | | | | | |
| | | | Block 1) | | | | | | | | |
| 15 | HEMA | 5.24E−05 | p(DMA | 1.15E−06 | ACVA | 1.15E−06 | DMF | 1.90E−03 | 1440 | 64 | 85.3 |
| | | | EMA), | | | | | | | | |
| | | | (006-B7 | | | | | | | | |
| | | | Block 1) | | | | | | | | |
| 16 | HEMA | 3.02E−05 | p(DMA | 6.62E−07 | ACVA | 6.60E−07 | DMF | 1.01E−03 | 1440 | 64 | 86.3 |
| | | | EMA), | | | | | | | | |
| | | | (006-B8 | | | | | | | | |
| | | | Block 1) | | | | | | | | |
| 17 | HEMA | 2.94E−05 | p(DMA | 6.35E−07 | ACVA | 6.35E−07 | DMF | 9.77E−04 | 1440 | 64 | 90.9 |
| | | | EMA), | | | | | | | | |
| | | | (006-B9 | | | | | | | | |
| | | | Block 1) | | | | | | | | |
| 18 | HEMA | 3.18E−05 | p(DMA | 6.93E−07 | ACVA | 6.92E−07 | DMF | 1.02E−03 | 1440 | 64 | 84.3 |
| | | | EMA), | | | | | | | | |
| | | | (006-B10 | | | | | | | | |
| | | | Block 1) | | | | | | | | |
| 19 | HEMA | 2.94E−05 | p(DMA | 6.50E−07 | ACVA | 6.49E−07 | DMF | 9.96E−04 | 1440 | 64 | 86.3 |
| | | | EMA), | | | | | | | | |
| | | | (006-B11 | | | | | | | | |
| | | | Block 1) | | | | | | | | |
| 20 | HEMA | 1.45E−04 | p(DMA | 2.03E−06 | ACVA | 2.03E−06 | DMF | 5.86E−03 | 1440 | 64 | 74.8 |
| | | | EMA), | | | | | | | | |
| | | | (006-C1 | | | | | | | | |
| | | | Block 1) | | | | | | | | |
| 21 | HEMA | 1.12E−04 | p(DMA | 1.57E−06 | ACVA | 1.57E−06 | DMF | 4.41E−03 | 1440 | 64 | 79.8 |
| | | | EMA), | | | | | | | | |
| | | | (006-C2 | | | | | | | | |
| | | | Block 1) | | | | | | | | |
| 22 | HEMA | 1.02E−04 | p(DMA | 1.44E−06 | ACVA | 1.44E−06 | DMF | 4.03E−03 | 1440 | 64 | 82.6 |
| | | | EMA), | | | | | | | | |
| | | | (006-C3 | | | | | | | | |
| | | | Block 1) | | | | | | | | |
| 23 | HEMA | 9.06E−05 | p(DMA | 1.27E−06 | ACVA | 1.27E−06 | DMF | 3.55E−03 | 1440 | 64 | 82.9 |
| | | | EMA), | | | | | | | | |
| | | | (006-C4 | | | | | | | | |
| | | | Block 1) | | | | | | | | |
| 24 | HEMA | 6.83E−05 | p(DMA | 9.54E−07 | ACVA | 9.53E−07 | DMF | 2.56E−03 | 1440 | 64 | 86.9 |
| | | | EMA), | | | | | | | | |
| | | | (006-C5 | | | | | | | | |
| | | | Block 1) | | | | | | | | |
| 25 | HEMA | 6.52E−05 | p(DMA | 9.16E−07 | ACVA | 9.17E−07 | DMF | 2.50E−03 | 1440 | 64 | 94.4 |
| | | | EMA), | | | | | | | | |
| | | | (006-C6 | | | | | | | | |
| | | | Block 1) | | | | | | | | |
| 26 | HEMA | 6.91E−05 | p(DMA | 9.62E−07 | ACVA | 9.63E−07 | DMF | 2.61E−03 | 1440 | 64 | 89.5 |
| | | | EMA), | | | | | | | | |
| | | | (006-C7 | | | | | | | | |
| | | | Block 1) | | | | | | | | |
| 27 | HEMA | 6.36E−05 | p(DMA | 8.91E−07 | ACVA | 8.92E−07 | DMF | 2.44E−03 | 1440 | 64 | 97.0 |
| | | | EMA), | | | | | | | | |
| | | | (006-C8 | | | | | | | | |
| | | | Block 1) | | | | | | | | |
| 28 | HEMA | 5.01E−05 | p(DMA | 6.99E−07 | ACVA | 6.99E−07 | DMF | 1.86E−03 | 1440 | 64 | 90.0 |
| | | | EMA), | | | | | | | | |
| | | | (006-C9 | | | | | | | | |
| | | | Block 1) | | | | | | | | |
| 29 | HEMA | 4.93E−05 | p(DMA | 6.95E−07 | ACVA | 6.96E−07 | DMF | 1.86E−03 | 1440 | 64 | 90.1 |
| | | | EMA), | | | | | | | | |
| | | | (006-C10 | | | | | | | | |
| | | | Block 1) | | | | | | | | |
| 30 | HEMA | 5.40E−05 | p(DMA | 7.51E−07 | ACVA | 7.53E−07 | DMF | 2.02E−03 | 1440 | 64 | 90.4 |
| | | | EMA), | | | | | | | | |
| | | | (006-C11 | | | | | | | | |
| | | | Block 1) | | | | | | | | |
| 31 | HEMA | 1.99E−05 | p(DMA | 2.83E−07 | ACVA | 2.82E−07 | DMF | 6.23E−04 | 1440 | 64 | 82.4 |
| | | | EMA), | | | | | | | | |
| | | | (006-C12 | | | | | | | | |
| | | | Block 1) | | | | | | | | |
| 32 | HEMA | 1.87E−04 | p(DMA | 1.92E−06 | ACVA | 1.92E−06 | DMF | 7.52E−03 | 1440 | 64 | 73.9 |
| | | | EMA), | | | | | | | | |
| | | | (006-D2 | | | | | | | | |
| | | | Block 1) | | | | | | | | |
| 33 | HEMA | 9.69E−05 | p(DMA | 9.94E−07 | ACVA | 9.95E−07 | DMF | 3.77E−03 | 1440 | 64 | 83.0 |
| | | | EMA), | | | | | | | | |
| | | | (006-D3 | | | | | | | | |
| | | | Block 1) | | | | | | | | |
| 34 | HEMA | 1.12E−04 | p(DMA | 1.15E−06 | ACVA | 1.15E−06 | DMF | 4.45E−03 | 1440 | 64 | 81.5 |
| | | | EMA), | | | | | | | | |
| | | | (006-D4 | | | | | | | | |
| | | | Block 1) | | | | | | | | |
| 35 | HEMA | 1.09E−04 | p(DMA | 1.12E−06 | ACVA | 1.12E−06 | DMF | 4.31E−03 | 1440 | 64 | 85.9 |
| | | | EMA), | | | | | | | | |
| | | | (006-D6 | | | | | | | | |
| | | | Block 1) | | | | | | | | |
| 36 | HEMA | 6.28E−05 | p(DMA | 6.48E−07 | ACVA | 6.46E−07 | DMF | 2.45E−03 | 1440 | 64 | 94.7 |
| | | | EMA), | | | | | | | | |
| | | | (006-D8 | | | | | | | | |
| | | | Block 1) | | | | | | | | |
| 37 | HEMA | 5.56E−05 | p(DMA | 5.74E−07 | ACVA | 5.74E−07 | DMF | 2.15E−03 | 1440 | 64 | 96.7 |
| | | | EMA), | | | | | | | | |
| | | | (006-D9 | | | | | | | | |
| | | | Block 1) | | | | | | | | |
| 38 | HEMA | 3.42E−05 | p(DMA | 3.54E−07 | ACVA | 3.53E−07 | DMF | 1.21E−03 | 1440 | 64 | 90.7 |
| | | | EMA), | | | | | | | | |
| | | | (006-D11 | | | | | | | | |
| | | | Block 1) | | | | | | | | |
| 39 | HEMA | 3.73E−05 | p(DMA | 3.88E−07 | ACVA | 3.89E−07 | DMF | 1.33E−03 | 1440 | 64 | 76.5 |
| | | | EMA), | | | | | | | | |
| | | | (006-D12 | | | | | | | | |
| | | | Block 1) | | | | | | | | |
| 40 | HEMA | 1.80E−04 | p(DMA | 1.46E−06 | ACVA | 1.47E−06 | DMF | 7.27E−03 | 1440 | 64 | 81.5 |
| | | | EMA), | | | | | | | | |
| | | | (006-E1 | | | | | | | | |
| | | | Block 1) | | | | | | | | |
| 41 | HEMA | 1.08E−04 | p(DMA | 8.80E−07 | ACVA | 8.81E−07 | DMF | 4.23E−03 | 1440 | 64 | 80.6 |
| | | | EMA), | | | | | | | | |
| | | | (006-E4 | | | | | | | | |
| | | | Block 1) | | | | | | | | |
| 42 | HEMA | 1.32E−04 | p(DMA | 1.07E−06 | ACVA | 1.07E−06 | DMF | 5.28E−03 | 1440 | 64 | 81.7 |
| | | | EMA), | | | | | | | | |
| | | | (006-E5 | | | | | | | | |
| | | | Block 1) | | | | | | | | |
| 43 | HEMA | 6.04E−05 | p(DMA | 4.94E−07 | ACVA | 4.92E−07 | DMF | 2.32E−03 | 1440 | 64 | 92.0 |
| | | | EMA), | | | | | | | | |
| | | | (006-E9 | | | | | | | | |
| | | | Block 1) | | | | | | | | |
| 44 | HEMA | 4.29E−05 | p(DMA | 3.52E−07 | ACVA | 3.53E−07 | DMF | 1.58E−03 | 1440 | 64 | 82.2 |
| | | | EMA), | | | | | | | | |
| | | | (006-E10 | | | | | | | | |
| | | | Block 1) | | | | | | | | |
| 45 | HEMA | 6.52E−05 | p(DMA | 5.32E−07 | ACVA | 5.32E−07 | DMF | 2.52E−03 | 1440 | 64 | 81.3 |
| | | | EMA), | | | | | | | | |
| | | | (006-E11 | | | | | | | | |
| | | | Block 1) | | | | | | | | |
| 46 | HEMA | 5.72E−05 | p(DMA | 4.66E−07 | ACVA | 4.67E−07 | DMF | 2.17E−03 | 1440 | 64 | 70.6 |
| | | | EMA), | | | | | | | | |
| | | | (006-E12 | | | | | | | | |
| | | | Block 1) | | | | | | | | |
| 47 | HEMA | 3.47E−05 | p(DMA | 2.34E−06 | ACVA | 2.34E−06 | DMF | 1.44E−02 | 1440 | 64 | 76.5 |
| | | | EMA), | | | | | | | | |
| | | | (006-F3 | | | | | | | | |
| | | | Block 1) | | | | | | | | |
| 48 | HEMA | 1.83E−04 | p(DMA | 1.23E−06 | ACVA | 1.23E−06 | DMF | 7.42E−03 | 1440 | 64 | 77.4 |
| | | | EMA), | | | | | | | | |
| | | | (006-F4 | | | | | | | | |
| | | | Block 1) | | | | | | | | |
| 49 | HEMA | 1.29E−04 | p(DMA | 8.68E−07 | ACVA | 8.67E−07 | DMF | 5.22E−03 | 1440 | 64 | 80.4 |
| | | | EMA), | | | | | | | | |
| | | | (006-F7 | | | | | | | | |
| | | | Block 1) | | | | | | | | |
| 50 | HEMA | 1.20E−04 | p(DMA | 8.09E−07 | ACVA | 8.10E−07 | DMF | 4.87E−03 | 1440 | 64 | 81.1 |
| | | | EMA), | | | | | | | | |
| | | | (006-F8 | | | | | | | | |
| | | | Block 1) | | | | | | | | |
| 51 | HEMA | 1.06E−04 | p(DMA | 7.13E−07 | ACVA | 7.14E−07 | DMF | 4.24E−03 | 1440 | 64 | 75.3 |
| | | | EMA), | | | | | | | | |
| | | | (006-F9 | | | | | | | | |
| | | | Block 1) | | | | | | | | |
| 52 | HEMA | 2.62E−05 | p(DMA | 1.79E−07 | ACVA | 1.78E−07 | DMF | 8.80E−04 | 1440 | 64 | 83.2 |
| | | | EMA), | | | | | | | | |
| | | | (006-F11 | | | | | | | | |
| | | | Block 1) | | | | | | | | |
| 53 | HEMA | 9.45E−05 | p(DMA | 6.36E−07 | ACVA | 6.35E−07 | DMF | 3.77E−03 | 1440 | 64 | 77.4 |
| | | | EMA), | | | | | | | | |
| | | | (006-F12 | | | | | | | | |
| | | | Block 1) | | | | | | | | |
| 54 | HEMA | 3.09E−04 | p(DMA | 1.77E−06 | ACVA | 1.77E−06 | DMF | 1.28E−02 | 1440 | 64 | 78.8 |
| | | | EMA), | | | | | | | | |
| | | | (006-G2 | | | | | | | | |
| | | | Block 1) | | | | | | | | |
| 55 | HEMA | 2.27E−04 | p(DMA | 1.30E−06 | ACVA | 1.30E−06 | DMF | 9.33E−03 | 1440 | 64 | 77.3 |
| | | | EMA), | | | | | | | | |
| | | | (006-G4 | | | | | | | | |
| | | | Block 1) | | | | | | | | |
| 56 | HEMA | 2.40E−04 | p(DMA | 1.38E−06 | ACVA | 1.38E−06 | DMF | 9.90E−03 | 1440 | 64 | 75.0 |
| | | | EMA), | | | | | | | | |
| | | | (006-G5 | | | | | | | | |
| | | | Block 1) | | | | | | | | |
| 57 | HEMA | 1.54E−04 | p(DMA | 8.86E−07 | ACVA | 8.85E−07 | DMF | 6.30E−03 | 1440 | 64 | 82.7 |
| | | | EMA), | | | | | | | | |
| | | | (006-G6 | | | | | | | | |
| | | | Block 1) | | | | | | | | |
| 58 | HEMA | 1.17E−04 | p(DMA | 6.72E−07 | ACVA | 6.71E−07 | DMF | 4.68E−03 | 1440 | 64 | 83.9 |
| | | | EMA), | | | | | | | | |
| | | | (006-G7 | | | | | | | | |
| | | | Block 1) | | | | | | | | |
| 59 | HEMA | 9.14E−05 | p(DMA | 5.26E−07 | ACVA | 5.24E−07 | DMF | 3.66E−03 | 1440 | 64 | 74.6 |
| | | | EMA), | | | | | | | | |
| | | | (006-G9 | | | | | | | | |
| | | | Block 1) | | | | | | | | |
| 60 | HEMA | 9.93E−05 | p(DMA | 5.70E−07 | ACVA | 5.71E−07 | DMF | 3.97E−03 | 1440 | 64 | N/A |
| | | | EMA), | | | | | | | | |
| | | | (006-G10 | | | | | | | | |
| | | | Block 1) | | | | | | | | |
| 61 | HEMA | 8.66E−05 | p(DMA | 4.97E−07 | ACVA | 4.96E−07 | DMF | 3.43E−03 | 1440 | 64 | 72.8 |
| | | | EMA), | | | | | | | | |
| | | | (006-G11 | | | | | | | | |
| | | | Block 1) | | | | | | | | |
| 62 | HEMA | 6.36E−05 | p(DMA | 3.65E−07 | ACVA | 3.64E−07 | DMF | 2.51E−03 | 1440 | 64 | 65.1 |
| | | | EMA), | | | | | | | | |
| | | | (006-G12 | | | | | | | | |
| | | | Block 1) | | | | | | | | |
| 63 | HEMA | 3.78E−04 | p(DMA | 1.89E−06 | ACVA | 1.89E−06 | DMF | 1.57E−02 | 1440 | 64 | 70.6 |
| | | | EMA), | | | | | | | | |
| | | | (006-H2 | | | | | | | | |
| | | | Block 1) | | | | | | | | |
| 64 | HEMA | 3.50E−04 | p(DMA | 1.75E−06 | ACVA | 1.75E−06 | DMF | 1.46E−02 | 1440 | 64 | 74.1 |
| | | | EMA), | | | | | | | | |
| | | | (006-H3 | | | | | | | | |
| | | | Block 1) | | | | | | | | |
| 65 | HEMA | 1.87E−04 | p(DMA | 9.35E−07 | ACVA | 9.35E−07 | DMF | 7.69E−03 | 1440 | 64 | 70.8 |
| | | | EMA), | | | | | | | | |
| | | | (006-H6 | | | | | | | | |
| | | | Block 1) | | | | | | | | |
| 66 | HEMA | 1.78E−04 | p(DMA | 8.90E−07 | ACVA | 8.92E−07 | DMF | 7.28E−03 | 1440 | 64 | 81.8 |
| | | | EMA), | | | | | | | | |
| | | | (006-H7 | | | | | | | | |
| | | | Block 1) | | | | | | | | |
| 67 | HEMA | 1.16E−04 | p(DMA | 5.78E−07 | ACVA | 5.78E−07 | DMF | 4.67E−03 | 1440 | 64 | 81.9 |
| | | | EMA), | | | | | | | | |
| | | | (006-H10 | | | | | | | | |
| | | | Block 1) | | | | | | | | |
| 68 | HEMA | 7.23E−05 | p(DMA | 3.60E−07 | ACVA | 3.60E−07 | DMF | 2.86E−03 | 1440 | 64 | 83.0 |
| | | | EMA), | | | | | | | | |
| | | | (006-H11 | | | | | | | | |
| | | | Block 1) | | | | | | | | |
| 69 | HEMA | 8.50E−05 | p(DMA | 4.23E−07 | ACVA | 4.25E−07 | DMF | 3.35E−03 | 1440 | 64 | 70.6 |
| | | | EMA), | | | | | | | | |
| | | | (006-H12 | | | | | | | | |
| | | | Block 1) | | | | | | | | |
| 70 | HEMA | 3.75E−05 | p(DMA | 1.88E−06 | ACVA | 3.75E−07 | DMF | 1.92E−03 | 1440 | 64 | 55.7 |
| | | | EMA), | | | | | | | | |
| | | | (007-A1 | | | | | | | | |
| | | | Block 1) | | | | | | | | |
| 71 | HEMA | 1.72E−05 | p(DMA | 8.63E−07 | ACVA | 1.72E−07 | DMF | 7.77E−04 | 1440 | 64 | 71.1 |
| | | | EMA), | | | | | | | | |
| | | | (007-A2 | | | | | | | | |
| | | | Block 1) | | | | | | | | |
| 72 | HEMA | 1.99E−05 | p(DMA | 9.99E−07 | ACVA | 2.00E−07 | DMF | 9.65E−04 | 1440 | 64 | 76.6 |
| | | | EMA), | | | | | | | | |
| | | | (007-A3 | | | | | | | | |
| | | | Block 1) | | | | | | | | |
| 73 | HEMA | 1.30E−05 | p(DMA | 6.51E−07 | ACVA | 1.30E−07 | DMF | 5.74E−04 | 1440 | 64 | 80.3 |
| | | | EMA), | | | | | | | | |
| | | | (007-A4 | | | | | | | | |
| | | | Block 1) | | | | | | | | |
| 74 | HEMA | 6.12E−06 | p(DMA | 3.07E−07 | ACVA | 6.14E−08 | DMF | 2.28E−04 | 1440 | 64 | 73.5 |
| | | | EMA), | | | | | | | | |
| | | | (007-A7 | | | | | | | | |
| | | | Block 1) | | | | | | | | |
| 75 | HEMA | 3.34E−06 | p(DMA | 1.68E−07 | ACVA | 3.35E−08 | DMF | 7.03E−05 | 1440 | 64 | 72.3 |
| | | | EMA), | | | | | | | | |
| | | | (007-A8 | | | | | | | | |
| | | | Block 1) | | | | | | | | |
| 76 | HEMA | 5.40E−06 | p(DMA | 2.71E−07 | ACVA | 5.42E−08 | DMF | 1.97E−04 | 1440 | 64 | 66.6 |
| | | | EMA), | | | | | | | | |
| | | | (007-A9 | | | | | | | | |
| | | | Block 1) | | | | | | | | |
| 77 | HEMA | 5.06E−06 | p(DMA | 2.52E−07 | ACVA | 5.03E−08 | DMF | 1.86E−04 | 1440 | 64 | 73.6 |
| | | | EMA), | | | | | | | | |
| | | | (007-A10 | | | | | | | | |
| | | | Block 1) | | | | | | | | |
| 78 | HEMA | 5.24E−06 | p(DMA | 2.61E−07 | ACVA | 5.21E−08 | DMF | 1.96E−04 | 1440 | 64 | 77.6 |
| | | | EMA), | | | | | | | | |
| | | | (007-A11 | | | | | | | | |
| | | | Block 1) | | | | | | | | |
| 79 | HEMA | 7.27E−05 | p(DMA | 1.59E−06 | ACVA | 3.18E−07 | DMF | 3.94E−03 | 1440 | 64 | 45.1 |
| | | | EMA), | | | | | | | | |
| | | | (006-B1 | | | | | | | | |
| | | | Block 1) | | | | | | | | |
| 80 | HEMA | 3.45E−05 | p(DMA | 7.54E−07 | ACVA | 1.51E−07 | DMF | 1.76E−03 | 1440 | 64 | 67.5 |
| | | | EMA), | | | | | | | | |
| | | | (007-B2 | | | | | | | | |
| | | | Block 1) | | | | | | | | |
| 81 | HEMA | 2.10E−05 | p(DMA | 4.58E−07 | ACVA | 9.17E−08 | DMF | 1.02E−03 | 1440 | 64 | 66.6 |
| | | | EMA), | | | | | | | | |
| | | | (007-B3 | | | | | | | | |
| | | | Block 1) | | | | | | | | |
| 82 | HEMA | 3.26E−05 | p(DMA | 7.13E−07 | ACVA | 1.43E−07 | DMF | 1.71E−03 | 1440 | 64 | 52.8 |
| | | | EMA), | | | | | | | | |
| | | | (007-B4 | | | | | | | | |
| | | | Block 1) | | | | | | | | |
| 83 | HEMA | 1.84E−05 | p(DMA | 4.02E−07 | ACVA | 8.06E−08 | DMF | 9.00E−04 | 1440 | 64 | 76.9 |
| | | | EMA), | | | | | | | | |
| | | | (007-B5 | | | | | | | | |
| | | | Block 1) | | | | | | | | |
| 84 | HEMA | 2.63E−05 | p(DMA | 5.76E−07 | ACVA | 1.15E−07 | DMF | 1.37E−03 | 1440 | 64 | 60.8 |
| | | | EMA), | | | | | | | | |
| | | | (007-B6 | | | | | | | | |
| | | | Block 1) | | | | | | | | |
| 85 | HEMA | 1.81E−05 | p(DMA | 3.96E−07 | ACVA | 7.92E−08 | DMF | 9.29E−04 | 1440 | 64 | 70.2 |
| | | | EMA), | | | | | | | | |
| | | | (007-B7 | | | | | | | | |
| | | | Block 1) | | | | | | | | |
| 86 | HEMA | 2.14E−05 | p(DMA | 4.67E−07 | ACVA | 9.35E−08 | DMF | 1.11E−03 | 1440 | 64 | 69.4 |
| | | | EMA), | | | | | | | | |
| | | | (007-B8 | | | | | | | | |
| | | | Block 1) | | | | | | | | |
| 87 | HEMA | 1.64E−05 | p(DMA | 3.58E−07 | ACVA | 7.17E−08 | DMF | 8.34E−04 | 1440 | 64 | 79.0 |
| | | | EMA), | | | | | | | | |
| | | | (007-B9 | | | | | | | | |
| | | | Block 1) | | | | | | | | |
| 88 | HEMA | 8.48E−05 | p(DMA | 6.90E−07 | ACVA | 1.38E−07 | DMF | 4.76E−03 | 1440 | 64 | 62.9 |
| | | | EMA), | | | | | | | | |
| | | | (007-E5 | | | | | | | | |
| | | | Block 1) | | | | | | | | |
| 89 | HEMA | 1.45E−04 | p(DMA | 9.74E−07 | ACVA | 1.95E−07 | DMF | 8.18E−03 | 1440 | 64 | 49.4 |
| | | | EMA), | | | | | | | | |
| | | | (007-F4 | | | | | | | | |
| | | | Block 1) | | | | | | | | |
| 90 | HEMA | 5.07E−05 | p(DMA | 4.12E−07 | ACVA | 8.24E−08 | DMF | 2.82E−03 | 1440 | 64 | 46.5 |
| | | | EMA), | | | | | | | | |
| | | | (007-E10 | | | | | | | | |
| | | | Block 1) | | | | | | | | |
| 91 | MMA | 3.17E−04 | p(DMA | 1.06E−06 | ACVA | 1.06E−06 | DMF | 9.27E−04 | 1440 | 60 | 30.5 |
| | | | EMA), | | | | | | | | |
| | | | (009-A10 | | | | | | | | |
| | | | Block 1) | | | | | | | | |
| 92 | DMAEMA | 8.08E−04 | p(MMA), | 2.69E−06 | AIBN | 5.39E−07 | DMF | 1.72E−03 | 1440 | 60 | 61.9 |
| | | | (009-E8 | | | | | | | | |
| | | | Block 1) | | | | | | | | |
| 93 | DMAEMA | 9.92E−04 | p(BMA), | 3.31E−06 | AIBN | 6.61E−07 | DMF | 2.11E−03 | 1440 | 60 | 74.4 |
| | | | (009-G2 | | | | | | | | |
| | | | Block 1) | | | | | | | | |
| 94 | BMA | 7.75E−04 | p(DMA | 2.58E−06 | AIBN | 2.58E−06 | DMF | 1.74E−03 | 1440 | 60 | 17.3 |
| | | | EMA), | | | | | | | | |
| | | | (011-B1 | | | | | | | | |
| | | | Block 1) | | | | | | | | |
| 95 | DMAEMA | 1.25E−04 | p(MMA), | 4.16E−07 | ACVA | 4.17E−08 | DMF | 2.66E−04 | 1440 | 60 | 21.2 |
| | | | (011-E6 | | | | | | | | |
| | | | Block 1) | | | | | | | | |
| 96 | DMAEMA | 2.71E−04 | p(BMA), | 9.04E−07 | AIBN | 9.04E−07 | DMF | 5.78E−04 | 1440 | 60 | 53.6 |
| | | | (011-G7 | | | | | | | | |
| | | | Block 1) | | | | | | | | |

TABLE 5
Summary of Pilot PNP Library Characterization Data

| | Block 1 | DynaPro DLS | |
|---|---|---|---|
| | DynaPro Result | Z-Avg Result | Size PDI |
| PNP | (molar mass, kDa) | (size, nm) | (goal < 0.3) |
| 1 | 16.3 | 386.9 | Multimodal |
| 2 | 16.8 | 574.3 | Multimodal |
| 3 | 17.9 | 331.8 | 0.121 |
| 4 | 21.6 | 345.1 | 0.152 |
| 5 | 22.3 | 355.8 | 0.483 |
| 6 | 23.3 | 379.7 | 0.268 |
| 7 | 22.7 | 365.7 | 0.219 |
| 8 | 23.9 | 317.8 | 0.040 |
| 9 | 24.1 | 296.2 | 0.137 |
| 10 | 27.2 | 323.9 | 0.254 |
| 11 | 17.4 | 1017.8 | 0.166 |
| 12 | 26.9 | 1368.9 | Multimodal |
| 13 | 18.7 | 450.2 | 0.087 |
| 14 | 20.9 | 389.2 | 0.152 |
| 15 | 21.9 | 291.5 | 0.111 |
| 16 | 31.2 | 361 | 0.243 |
| 17 | 30.3 | 317.7 | 0.201 |
| 18 | 35.0 | 345.3 | 0.114 |
| 19 | 30.7 | 267.1 | 0.137 |
| 20 | 12.1 | 1383 | 0.238 |
| 21 | 17.7 | 459 | 0.301 |
| 22 | 18.8 | 436.7 | 0.314 |
| 23 | 18.6 | 448 | 0.492 |
| 24 | 26.7 | 422.8 | 0.368 |
| 25 | 24.1 | 333 | 0.214 |
| 26 | 25.1 | 275.6 | Multimodal |
| 27 | 22.8 | 318.3 | 0.219 |
| 28 | 28.9 | 342 | 0.557 |
| 29 | 27.0 | 222.5 | Multimodal |
| 30 | 26.5 | 251.3 | 0.48 |
| 31 | 62.1 | 207.3 | 0.548 |
| 32 | 17.0 | 522.5 | 0.435 |
| 33 | 26.4 | 477.6 | Multimodal |
| 34 | 20.8 | 369.9 | 0.237 |
| 35 | 22.9 | 321.4 | 0.276 |
| 36 | 26.4 | 282.4 | 0.229 |
| 37 | 30.4 | 319.1 | 0.351 |
| 38 | 54.2 | 262.3 | 0.202 |
| 39 | 53.1 | 315.5 | 0.171 |
| 40 | 20.8 | 730.2 | Multimodal |
| 41 | 31.9 | 379.8 | 0.332 |
| 42 | 24.3 | 309.2 | 0.237 |
| 43 | 40.9 | 317.8 | 0.338 |
| 44 | 56.5 | 1186.1 | 0.271 |
| 45 | 37.7 | 386.4 | 0.153 |
| 46 | 43.2 | 279.6 | 0.138 |
| 47 | 14.7 | 413.3 | 0.29 |
| 48 | 23.6 | 351 | 0.218 |
| 49 | 24.2 | 266.7 | 0.524 |
| 50 | 24.0 | 249.3 | 0.401 |
| 51 | 29.0 | 269.9 | 0.371 |
| 52 | 105.1 | 271.2 | 0.074 |
| 53 | 30.4 | 302.5 | 0.145 |
| 54 | 18.0 | 375.2 | Multimodal |
| 55 | 20.5 | 440.4 | 0.496 |
| 56 | 19.0 | 296.3 | 0.109 |
| 57 | 24.8 | 277.1 | 0.475 |
| 58 | 34.7 | 299.6 | 0.393 |
| 59 | 36.0 | 269 | 0.351 |
| 60 | 34.9 | 264.8 | 0.181 |
| 61 | 40.5 | 353.1 | 0.138 |
| 62 | 41.3 | 230.5 | 0.151 |
| 63 | 16.2 | 354.5 | Multimodal |
| 64 | 15.0 | 355.5 | 0.334 |
| 65 | 23.7 | 332.6 | 0.231 |
| 66 | 26.6 | 253.4 | 0.217 |
| 67 | 34.6 | 294.3 | 0.151 |
| 68 | 44.3 | 275.2 | 0.207 |
| 69 | 45.6 | 404.8 | 0.287 |
| 70 | 9.4 | 2023.5 | 0.020 |
| 71 | 18.3 | 473.2 | 0.521 |
| 72 | 13.5 | 549.2 | 0.153 |
| 73 | 19.7 | 568 | Multimodal |
| 74 | 30.0 | 262 | 0.353 |
| 75 | 53.7 | 229.4 | Multimodal |
| 76 | 31.3 | 230.7 | 0.444 |
| 77 | 30.3 | 387.4 | Multimodal |
| 78 | 29.5 | 266.7 | Multimodal |
| 79 | 11.4 | 1155.7 | Multimodal |
| 80 | 21.3 | 619.2 | 0.198 |
| 81 | 28.9 | 337.7 | 0.053 |
| 82 | 17.5 | 336 | 0.13 |
| 83 | 28.8 | 90.5 | Multimodal |
| 84 | 19.0 | 251.3 | 0.164 |
| 85 | 21.1 | 360.6 | Multimodal |
| 86 | 18.8 | 225.3 | Multimodal |
| 87 | 22.4 | 168 | Multimodal |
| 88 | 13.1 | 298.6 | 0.217 |
| 89 | 11.0 | 281 | 0.314 |
| 90 | 16.3 | 300 | 0.503 |
| 91 | 29.0 | 214 | 0.293 |
| 92 | 9.1 | 14.2 | 0.412 |
| 93 | 3.9 | 21.2 | Multimodal |
| 94 | 15.6 | 432.8 | Multimodal |
| 95 | 13.2 | 268.9 | Multimodal |
| 96 | 14.2 | 657.2 | 0.356 |

### Example 6

### Composition Example 3 (CE3): Nucleic Acid Constructs Conjugation to PNPs Direct Amidification with an Amine Terminal Nucleic Acid Construct

RAFT copolymers made using CTAs that contain at least one carboxyl terminal group were further functionalized with amine terminal DNA barcodes. A RAFT copolymer was transferred into a MES buffer at ˜12 mg/mL. The sample was sonicated for 30 minutes. EDC reagent and Sulfo-NHS reagent was added to the polymer at a molar ratio of 10:1 and 25:1 respectively, reagent to PNP. The sample was incubated for at least 10 minutes at room temperature to allow the reaction to occur. The reaction volume was filtered through a membrane with a molecular weight cut off of 30 kDa via centrifuge at ˜3000×g for —15 minutes. The filtrate was discarded, and sterile PBS was added to the retentate to reconstitute to 10 mg/mL polymer. A nucleic acid constructs with a primary amine group attached to the 5′ end was added to polymer and the sample was incubated for at least 15 minutes. The sample was transferred to an amicon ultra-4 centrifuge tube (MWCO 30 kDa, Max 3.5 mL/Tube) and centrifuged at 4,000×g for 15 minutes to remove any unbound nucleic acid constructs. The filtrate (containing unbound nucleic acid constructs) was discarded and sterile PBS was added to the retentate to final concentration of 8 mg/mL polymer. The conjugated barcodes were amplified via PCR, using primers designed to bind to the primer binding segments on the nucleic acid constructs. The amplicons were detected via gel electrophoresis on agarose gel. The presence of a double band (FIG. 9) in a lane indicates the presence of amplicons, showing that nucleic acid constructs were bound to the PNPs.

The direct amidification method was used to attach 10 unique barcodes to 10 unique PNPs. The 10 unique PNPs were prepared according to the reagents shown in EXAMPLE 5.

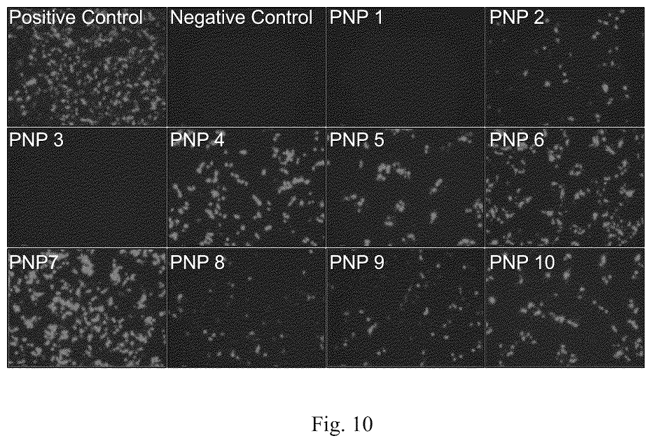

Subsequently, the direct amidification method was used to attach 10 unique DNA barcodes to each PNP, giving each a unique label. The nucleic acid construct-PNPs were loaded with a pDNA encoding for the expression of tdTomato red fluorescent protein, and then used to treat HEK-293T cells at ˜0.024 mg/mL PNP, 24 hours after seeding in a 96 well plate at 20,000 cells per well. They were left to incubate for another 48 hours, at which time, expression of the payload was observed via fluorescence microscopy using a Texas Red filter set (FIG. 10). This provides evidence that the nucleic acid constructs-PNPs are capable of being taken up by mammalian cells and delivering a payload, as indicated by the red fluorescent images.

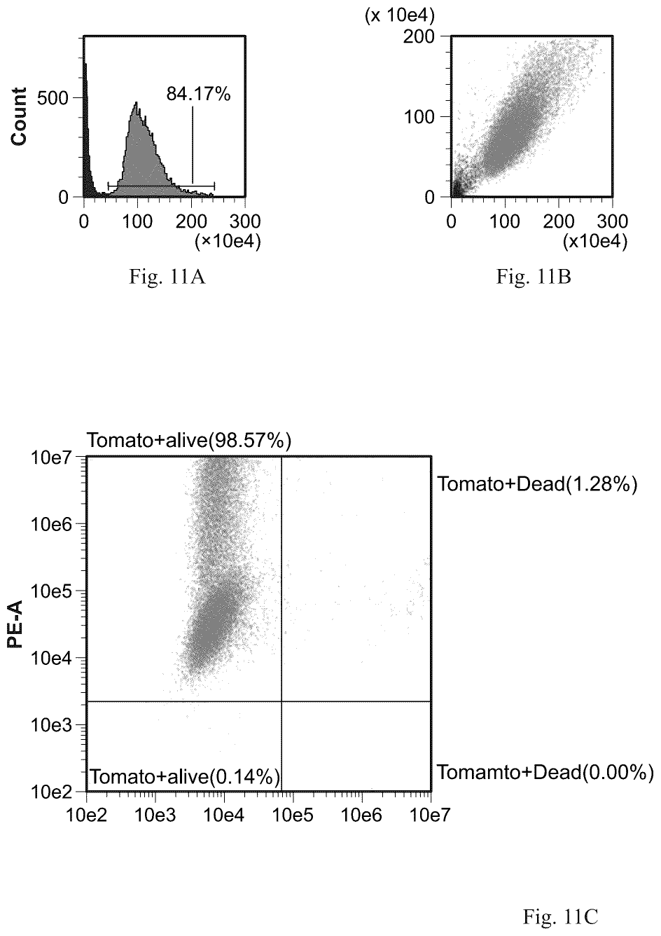

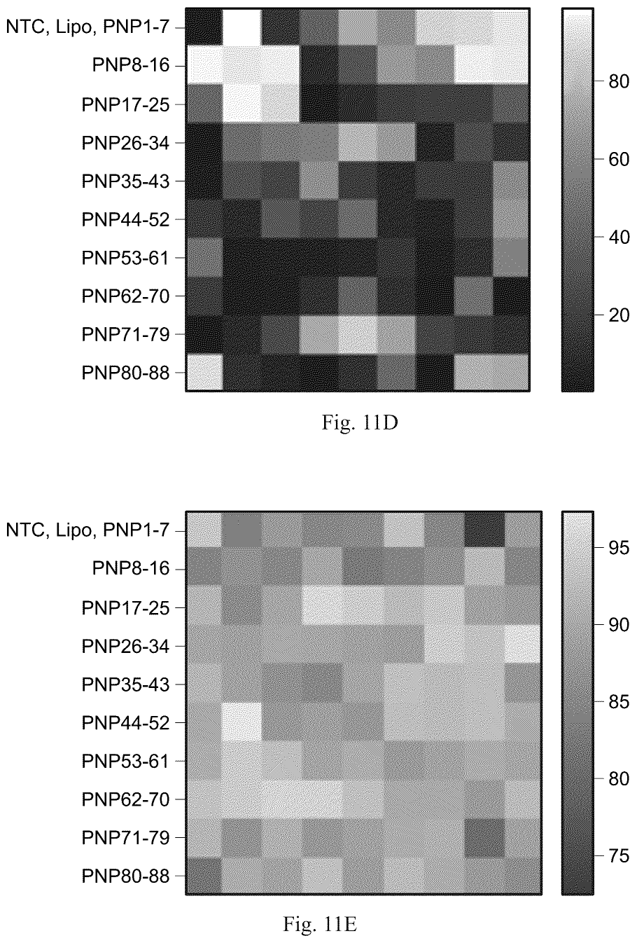

The direct amidification method was then used to attach 88 unique barcodes to 88 unique PNPs. The 88 unique PNPs were prepared according to the reagents shown in EXAMPLE 5. The amidification method was then used to attach 88 unique DAN barcodes to each PNP giving each a unique label. The nucleic acid construct-PNPs were loaded with a pDNA encoding for the expression of tdTomato red fluorescent protein, and then used to treat HEK-293T cells at ˜0.024 mg/mL PNP, 24 hours after seeding in a 96 well plate at 20,000 cells per well. They were left to incubate for another 48 hours, at which time, expression of the payload was measured via flow cytometry using a Cytoflex (FIGS. 11(a)-(c)). This provided evidence that some of the nucleic acid construct-PNPs were able to be taken up by mammalian cells. The cells were also given a live/dead stain using zombie dye, and cell viability was measured via flow cytometry (FIGS. 11(d)-(e)) showing that the PNPs were of relatively low cytotoxicity, with cell viability numbers of greater than 75% for the vast majority of PNPs.

### Example 7

### Methods for Screening Nucleic Acid Construct-PNPs in Vitro and in Vivo

The pooled sample of polymer nanoparticles produced from the recipe in Table 2, with 96 unique barcodes attached via avidin-biotin linkange, was used for measuring cell uptake and cytotoxicity in vitro in HEK293T cells, and for measuring biodistribution in vivo administration in mice. nucleic acid construct-PNPs were formulated in a sterile saline solution and stored at 4° C. for up to 1 month prior to in vivo dosing. Cell uptake efficiency and cytotoxicity are assessed in vitro using HEK293T cells with 0.024 mg/mL PNP at 250 ng or 150 ng/well pDNA treatment concentrations. Cell uptake was demonstrated by fluorescence microscopy. Animals are assigned to dose groups using a stratified randomization program designed to maintain similar group mean body weights by sex. Animals are administered either a control or test article via a single bolus intravenous tail-vein injections. Tested doses ranged from 0-150 mg/kg, with adverse clinical events being observed in 35% of animals at 150 mg/kg. Blood and tissue are collected from all animals and snap frozen in liquid nitrogen.

### Example 8

### Extraction of ucleic acAid Construct-PNPs From in Vitro Biological Samples

10-20 mg of tissues are placed in a Tris-EDTA lysis buffer and homogenized using the TissueLyser bead-beating system and a 5mm stainless steel bead. Homogenization is carried out at 25 Hz in 5-7 minute intervals until solution is homogenous in appearance. Proteinase K is then added to the lysate for protein digestion and incubated in a Thermomixer at 55° C. for 2-4 hours. DNA is extracted from the tissue lysate using the QlAamp 96 DNA extraction kit and a Qiacube HT instrument according to the manufacturer's protocol. Concentration and purity of isolated samples is determined using a NanoDrop.

### Example 9

### PCR Amplification of Polynucleotide Barcodes in Nucleic Acid Constructs

Polymerase chain reaction is used to produce amplicons from extracted nucleic acid constructs. PCR is performed using a single set of universal primers that anneal to the universal amplification sites on the barcode, thereby amplifying all unique barcodes within a sample in a single reaction. Positive amplification of barcode(s) within a sample is determined using electrophoresis (agarose gel or bioanalyzer) indicated by the presence of a band at ˜120 bp.

### Example 10

### Library Preparation and Sequencing of Nucleic Acid Constructs

Sequencing libraries are prepared from the amplicons generated during first stage PCR amplification. Our universal primers also contain overhang sequences that enable attachment of Index Adapters for sequencing. Illumina Unique Dual Indexes are annealed to the overhangs on the amplicon by PCR. Individual indexed libraries are then pooled in equal amounts and purified using a NucleoSpin Gel and PCR Clean-up kit according the manufacturer's protocols. The molar concentration of the final sequencing library is determined using a Qubit dsDNA High Sensitivity Assay kit and Qubit Fluorometer. The library is spiked with 2% PhiX, diluted to 1.8 μM and loaded onto a High Output 300 cycle NextSeq sequencing cardrige. Paired end sequencing is performed using a NextSeq550 instrument.

### Example 11

### Sequence Analysis and Bioinformatics

Merged reads from each Sample ID are demultiplexed into PE FASTQ files, and merged into a single file. The merged reads are processed to identify those containing both the 5′ and 3′ flanking adapters. Trimmed reads are then downselected for sequences containing the correct barcode length. Barcode counts are generated from these downselected sequences and tagged according to whether they are spiked or random. Barcode counts are then normalized to the number of FASTQ reads in the sample.

### Hypothetical Example

By way of example, in one illustrative embodiment, the presently disclosed rapid DBTL technologies may be used to develop a gene therapy for forms of amyotrophic lateral sclerosis (ALS) caused by toxic, gain-of-function mutations in superoxide dismutase 1 (SOD1). This gene therapy may involve delivering a CRISPR base-editing protein via a non-viral gene delivery vehicle to inactivate the production of mutant SOD1 protein in microglia, a cell type that modulates the progression of the disease but remains refractory to efficient viral transduction. This will enable safe and efficient therapeutic “hit-and-run” editing for ALS.

An exemplary disease that can be treated with the methods described herein is ALS. ALS is a rapidly progressive, paralytic, and invariably fatal disorder characterized by the selective loss of motor neurons in the spinal cord and brain. Though most cases of ALS are sporadic, dominantly inherited mutations in SOD1 (a ubiquitously expressed metalloenzyme that normally converts superoxide anions into oxygen and hydrogen peroxide) account for up to 20% of all inherited or familial forms of ALS. Base editors are a recently emerged gene-editing modality capable of introducing targeted single-base substitutions in DNA without the requirement for a double-strand break (DSB). Base editors consist of fusions of a catalytically impaired Cas9 nuclease variant, known as a Cas9 nickase, with a nucleobase deaminase enzyme. This example will rely on the ability of base editors, specifically cytidine base editors (CBEs), to catalyze C>T base transitions at CGA, CAG or CAA triplets in a target gene sequence, which creates an in-frame stop codon that triggers the degradation of a target mRNA by nonsense-mediated decay—a surveillance mechanism used by cells to prevent the formation of truncated proteins. Using this method, SOD1 will be inactivated in a manner that does not require a DSB and does not rely on the stochastic and mutagenic NHEJ repair pathway, thus overcoming two of the major limitations facing the clinical implementation of CRISPR-Cas9 for ALS. Thus, while first generation CRISPR is considered the “cut and paste” of gene editing, base editors are considered to be an “eraser and pencil” function, allowing for precise single base edits to a genome, opening new mechanisms for revolutionary ALS treatments. However, innovations in gene delivery have significantly lagged innovations in technologies for gene editing itself. Thus, efficient delivery of base editing systems to the specific cell types involved in driving the progression of ALS represents a key limitation impeding its safe and efficient implementation for treatment of the disorder. Non-viral delivery vehicles will be used to address many of these limitations.

In this illustrative example, the rapid DBTL technologies can iterate through hundreds of diverse polymer nanoparticle candidates, using automated high-throughput synthesis of diverse polymer nanoparticle libraries, parallel in vitro and in vivo screens of barcoded libraries, and a machine learning algorithm to analyze the large data sets and predict new libraries for rapid iteration. FIG. 1 presents a simplified flow diagram illustrating a DBTL cycle for non-viral gene delivery development based on automated synthesis, high throughput testing, and machine learning design.

### Hypothetical Example

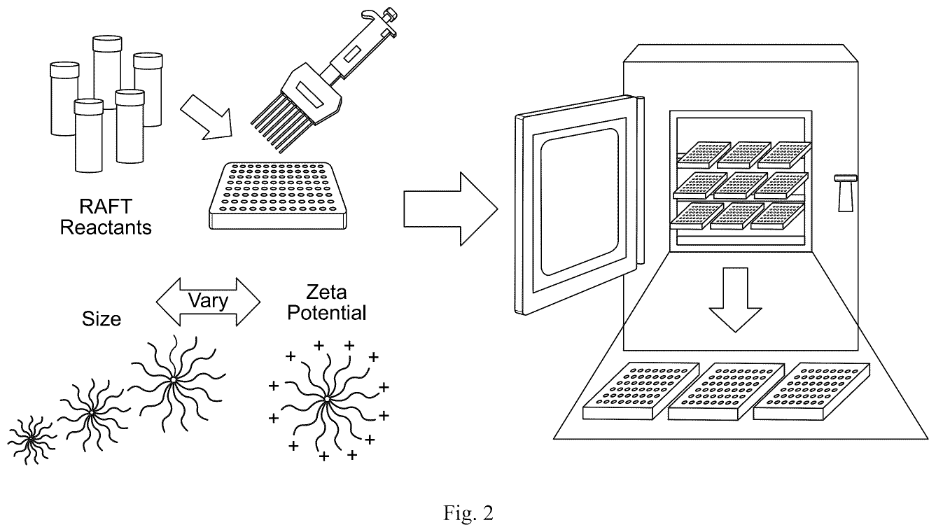

In one aspect, a library of 100s of polymer nanoparticles (PNPs) encapsulating CBE mRNA can be screened in a high throughput in vitro and in vivo platform. In the illustrative embodiment, over 500 PNPs are synthesized and uniquely labeled and tracked via DNA barcoding. A highly versatile PNP platform based on reversible addition-fragmentation chain transfer (RAFT) polymerization will be used due to its flexibility, reproducibility, and scalability. See K. Sims et al., “Rigor and reproducibility in polymer nanoparticle synthesis and characterization,” Rsc Advances 2020, 10 (5), 2513-2518 (incorporated herein by reference). As shown in FIG. 2, the RAFT polymerization platform can be used to generate highly monodisperse PNPs with a diverse variety of sizes, charges and chemical make-up. The PNPs can be functionalized to attach cell penetrating peptides to enable higher order functionality and protection to both the vehicle and the cargo. In some embodiments, PNPs may be labeled with quantum dots and other biomarkers via avidin-biotin conjugation. See A. Duong et al., “Scalable, Semicontinuous Production of Micelles Encapsulating Nanoparticles via Electrospray,” Langmuir 2014, 30 (14), 3939-3948. (incorporated herein by reference). This combination of microglia non-viral delivery vehicles with base editing payloads is highly innovative because it has the potential to lead to a new therapy for ALS. Moreover, the DBTL technologies of the present disclosure are generalizable to enable the creation of advanced non-viral delivery vehicles capable of accessing the other cell types involved in ALS.

After library synthesis, as described above, these PNPs can then be rapidly tested in vitro in a microglial cell line for toxicity, inflammation, and mRNA delivery efficiency via GFP expression. In parallel, the biodistribution and toxicity of the entire library can be assessed using loaded nanoparticles delivered via an intrathecal injection to the cerebrospinal fluid (CSF) of the G93A-SOD1 mouse model of ALS using an mRNA encoding a bioluminescent luciferase that can be tracked via in vitro imaging system (IVIS). This screen should result in three large data sets including particle physical characteristics, in vitro bioactivity, and in vivo biodistribution and toxicity, which, taken together, will provide the basis for an informed design of a novel non-viral delivery vehicle library which will be synthesized in a second iteration. This novel library can then be tested for functional gene editing tests in a microglial cell line modified to express a mutant SOD1 protein. The PNPs can be loaded with mRNA encoding CBE designed to inactivate GFP and SOD1, detected by fluorescence measurement and sequencing.

1. (canceled)

2. A composition comprising:
a non-viral delivery vehicle comprising one or more nanoparticle forming polymers and
a nucleic acid construct, comprising:
two primer binding segments; and
one or more unique polynucleotide barcodes between the two primer binding segments.

3. The composition of claim 2, wherein the one or more nanoparticle forming polymers are RAFT block copolymers comprising:
a. a first terminus comprising a first capping unit derived from a first chain transfer agent in a RAFT copolymerization process;
b. a first block prepared from one or more monomer units covalently attached to the first reactive functional unit, and having a molecular weight (Mₙ) in the range of about 1 kDa to about 200 kDa and a degree of polymerization in the range of about 10 to about 2500;
c. optionally a second block prepared from one or more monomer units covalently attached to the first block, and having a molecular weight (Mₙ)in the range of about 1 kDa to about 200 kDa and a degree of polymerization in the range of about 20 to about 2000; and
d. a second terminus comprising a second capping unit derived from a first or a second chain transfer agent.

4. The composition of claim 3, wherein the non-viral delivery vehicle has one or more of an overall molecular weight (Mₙ) in the range of about 25 kDa to about 60 kDa, an overall degree of polymerization in the range of about 700 to about 900, a target size in the range of about of about 10 to about 60 nm, and a maximum corona-to-core ratio (CCR) of about 1.5 to about 3.5.

5. The composition of claim 3, wherein the first block is prepared from one or more monomer units selected from the group consisting of 2-dimethylaminoethyl acrylate, 2-(diethylamino) ethyl methacrylate, 2-(diisopropylamino) ethyl methacrylate, butyl methacrylate, ethyl acrylic acid, propyl acrylic acid, (hydroxyethyl)methacrylate, and methyl methacrylate.

6. The composition of claim 3, wherein the first block is prepared from one of 2-dimethylaminoethyl acrylate, 2-(diethylamino) ethyl methacrylate, 2-(diisopropylamino) ethyl methacrylate, butyl methacrylate, ethyl acrylic acid, propyl acrylic acid, (hydroxyethyl)methacrylate, or methyl methacrylate.

7. The composition of claim 3, wherein the second block is prepared from one or more monomer units selected from the group consisting of 2-dimethylaminoethyl acrylate, 2-(diethylamino) ethyl methacrylate, 2-(diisopropylamino) ethyl methacrylate, butyl methacrylate, ethyl acrylic acid, propyl acrylic acid, (hydroxy ethyl methacrylate, and methyl methacrylate.

8. The composition of claim 3, wherein the second block is a random copolymer prepared from two different monomer units independently selected from the group consisting of 2-dimethylaminoethyl acrylate, 2-(diethylamino) ethyl methacrylate, 2-(diisopropylamino) ethyl methacrylate, butyl methacrylate, ethyl acrylic acid, propyl acrylic acid, hydroxy ethyl methacrylate, and methyl methacrylate.

9. The composition of claim 3, wherein the second block is a random copolymer prepared from three different monomer units independently selected from the group consisting of 2-dimethylaminoethyl acrylate, 2-(diethylamino) ethyl methacrylate, 2-(diisopropylamino) ethyl methacrylate, butyl methacrylate, ethyl acrylic acid, propyl acrylic acid, (hydroxy ethyl)methacrylate, and methyl methacrylate.

10. The composition of claim 3, wherein the second block is a random copolymer prepared from 2-dimethylaminoethyl acrylate, butyl methacrylate, and propyl acrylic acid; or 2-dimethylaminoethyl acrylate and butyl methacrylate; or 2-dimethylaminoethyl acrylate, butyl methacrylate, and ethyl acrylic acid.

11. The composition of claim 2, wherein the nucleic acid construct is wherein the nucleic acid construct is electrostatically associated with the nanoparticle forming polymers.

12. The composition of claim 2, wherein the nucleic acid construct is bonded to the nanoparticle forming polymers via complexation of biotin and a biotin binding molecule.

13. The composition of claim 2, wherein the nucleic acid construct is covalently bonded to the nanoparticle forming polymers.

14. The composition of claim 2, wherein the primer binding segments range in length from about 15 base pairs to about 30 base pairs.

15. The composition of claim 2, wherein the primer binding segments are a universal primer binding set.

16. The composition of claim 2, wherein the one or more polynucleotide barcodes comprise unique sequences of 6-20 nucleotides in length.

17. The composition of claim 16, wherein the polynucleotide barcodes further comprise a hamming distance of at least 2-6 bases between any two unique polynucleotide barcode sequences.

18. The composition of claim 2, wherein the nucleic acid construct further comprises from about 6 to about 12 random bases at the 3′ end of the polynucleotide barcode.

19. The composition of claim 18, wherein the about 6 to about 12 random bases at the 3′ end of the polynucleotide barcode are for bioinformatic removal of PCR duplicates.

20. The composition of claim 2, wherein the nucleic acid construct ranges in length from about 42 nucleotides to about 210 nucleotides.

21. A method of in vivo screening for a nanoparticle forming polymer for use as a delivery vehicle, the method comprising:
(a) preparing a library comprising two or more types of polymer nanoparticles, wherein each polymer nanoparticle is associated with a nucleic acid construct comprising a different polynucleotide barcode;
(b) administering the library to an animal;
(c) removing cells or tissues from the animal;
(d) isolating the nucleic acid constructs from the cells or the tissues of the animal;
(e) detecting the nucleic acid constructs in the cells or the tissues of the animal; and
(f) identifying the polymer nanoparticle for use as a delivery vehicle.
